(19) United States
(12) Patent Application Publication
(10) Pub. No.: US 2026/0232662 A1
(43) Pub. Date: Aug. 13, 2026

(54) MIR-124 INDUCER, AND PREPARATION METHOD THEREFOR AND USE THEREOF

(71) Applicant: NANJING SYNNOCARE PHARMACEUTICAL TECHNOLOGY CO., LTD., Nanjing, Jiangsu (CN)

(72) Inventors: Houxing FAN, Nanjing, Jiangsu (CN); Qiangqiang JIANG, Nanjing, Jiangsu (CN); Tiancai WANG, Nanjing, Jiangsu (CN)

(21) Appl. No.: 19/157,461

(22) PCT Filed: Feb. 7, 2024
(86) PCT No.: PCT/CN2024/076609

§ 371 Date: Aug. 18, 2025

(30) Foreign Application Priority Data
Feb. 17, 2023 (CN) 202310130954.3
Apr. 18, 2023 (CN) 202310415260.4

(51) Int. Cl. A61K 31/4985; A61K 31/498; A61P 1/04; A61P 29/00; C07D 241/44; C07D 405/12; C07D 487/04

(52) U.S. Cl. CPC A61K 31/4985; A61K 31/498; A61P 1/04; A61P 29/00; C07D 241/44; C07D 405/12; C07D 487/04

(57) ABSTRACT

A compound represented by formula (1) and a preparation method therefor are provided. A composition containing the compound represented by formula (1), and/or a pharmaceutically acceptable salt thereof, a preparation method therefor are also provided. The compound can be used as a miR-124 inducer in the preparation of a medication for treating miR-124 mediated diseases.

### FIELD OF THE INVENTION

The invention belongs to the field of medicinal chemistry, and more specifically, relates to a kind of miR-124 inducers as shown in Formula (1), and preparation methods therefor, as well as the use of these compounds for preparing pharmaceutical compositions for treating, regulating, and/or preventing diseases mediated by miR-124.

### BACKGROUND OF THE INVENTION

MicroRNAs (miRNAs) are a class of endogenous small RNAs, approximately 20-24 nucleotides in length, that play diverse and critical regulatory roles within cells. Each miRNA can target multiple genes, and several miRNAs may collectively regulate a single gene, forming a complex regulatory network. This complex regulatory network can either regulate the expression of multiple genes through a single miRNA, or finely regulate the expression of a certain gene through the combination of several miRNAs. It is estimated that miRNAs regulate approximately one-third of all human genes. Although miRNAs account for only about 2% of the human genome, they control the expression of over 30% of human genes, making them as central players in gene regulatory networks [Stem Cells, 2013, 31: 2205-2217]. miRNAs regulate cellular processes such as proliferation, differentiation, and apoptosis, playing crucial roles in individual growth and development, as well as in the initiation and progression of various diseases, including inflammation and cancer. A growing body of research indicates that miRNAs participate in nearly all biological life processes [Trends Mol Med, 2014, 331: 1-10].

MiR-124 is a highly conserved miRNA expressed across species, from lower organisms (e.g., Caenorhabditis elegans) to higher organisms (e.g., humans). Current research indicates that the three encoding genes of human miR-124 are located at chromosomal loci 8p23.1, 8912.3, and 20q13.33, respectively. The promoters of these genes contain CpG islands, whose methylation silences miR-124 transcriptionally [Oncogene, 2012, 31: 1609-1622]. MiR-124 is the most abundantly expressed miRNA in the central nervous system (CNS). Evidence suggests that miR-124 is closely associated with damage repair in the CNS. Niina Vuokila et al. found that after brain injury, the hippocampal gene expression network is subject to long-term regulation by miR-124, indicating that miR-124 acts as a chronic modulator of gene expression following brain injury [Cell Mol Life Sci, 2018, 75: 4557-4581]. MiR-124 demonstrates significant therapeutic potential in Alzheimer's disease (AD). It is highly and specifically expressed in the brain, yet its levels are markedly downregulated in the brains of AD patients. MiR-124 regulates the expression of multiple genes (e.g., BACE1, Caveolin-1, GSK-3β, PTPN1, DACT1), thereby influencing synaptic plasticity, neuroinflammation, Aβ production, and Tau protein phosphorylation. Moreover, miR-124 is found to be under-expressed in various cancers, including brain tumors, gastric cancer, breast cancer, cervical cancer, and prostate cancer, suggesting its potential as a therapeutic target for multiple malignancies.

In recent years, miR-124 has attracted significant attention due to its anti-inflammatory activity. ABX464, a small-molecule drug, exerts potent anti-inflammatory and antiviral effects by modulating RNA splicing, along with the ability to inhibit HIV virus replication. The anti-inflammatory mechanism of ABX464 is triggered by its binding to the cap-binding complex, which is located at the 5′ end of non-coding RNA molecules within cells. This binding induces splicing of a long non-coding RNA, leading to the overexpression of a specific microRNA product-miR-124. Subsequently, miR-124 initiates a cascade of events that ultimately generates a robust anti-inflammatory response [Retrovirology, 2015, 12: 1-15]. ABX464 is currently in development for treating inflammatory and viral diseases, such as Crohn's disease, ulcerative colitis, rheumatoid arthritis, HIV/AIDS, and COVID-19.

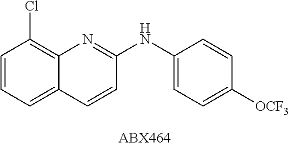 [SMILES: FC(F)(F)Oc1ccc(Nc2ccc3cccc(Cl)c3n2)cc1]

However, ABX464 still suffers from poor physicochemical properties and unsatisfactory oral absorption, leaving significant room for improvement. Therefore, there is an urgent need in the field to develop novel miR-124 inducers with higher potency, improved metabolic stability, and/or enhanced safety profiles.

### SUMMARY OF THE INVENTION

The objective of the present invention is to provide a novel miR-124 inducer with profiles of potent activity, good metabolic stability, and/or good safety, as well as preparation therefor and uses thereof.

In the first aspect of the present invention, it provides a kind of compounds as shown in Formula (1), or an isomer, crystal form, pharmaceutically acceptable salt, hydrate, or solvate thereof:

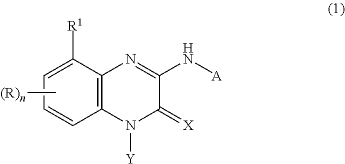

wherein:
n is 0, 1, 2 or 3;
each R is independently selected from the group consisting of: halogen, CN, nitro, C₁₋₃ alkyl, C₁₋₃ haloalkyl, C₁₋₃ alkoxy or C₁₋₃ haloalkoxy;
R¹ is selected from the group consisting of: H, CN, C₁₋₃ alkyl, C₁₋₃ alkoxy or halogen; A is selected from the group consisting of: 6-10 membered aryl, 5-7 membered heteroaryl, benzo-5-7-membered cycloalkyl or benzo 5-7-membered heterocyclyl, wherein the aryl, heteroaryl, benzo cycloalkyl and benzo heterocyclyl can be further substituted by 0-4 substituents selected from the group consisting of: halogen, CN, C₁₋₃ alkyl, C₃₋₆ cycloalkyl, C₁₋₃ alkoxy, C₁₋₃ alkylthio, C₁₋₃ haloalkyl, C₁₋₃ haloalkoxy, C₁₋₃ haloalkylthio or SF₅;
X is O, and Y is C₁₋₃ alkyl, C₁₋₃ haloalkyl or C₃₋₆ cycloalkyl; or
X and Y are taken together with the atoms to which they are bound to form a 5-membered heteroaryl or a 5-membered heterocyclyl, wherein the heteroaryl and heterocyclyl can be further substituted by 0-2 substituents of the following groups: C₁₋₃ alkyl or C₁₋₃ haloalkyl.

In another preferred embodiment, wherein at least one of R and R′ is halogen.

In another preferred embodiment, wherein each R is independently selected from the group consisting of: F, Cl, Br, Me, Et, OMe, OEt, CF₃, CN, OCF₃ or NO₂.

In another preferred embodiment, wherein R¹ is selected from the group consisting of: H, CN, Me, Et, OMe, OEt, F, Cl, or Br.

In another preferred embodiment, wherein A is selected from the group consisting of:

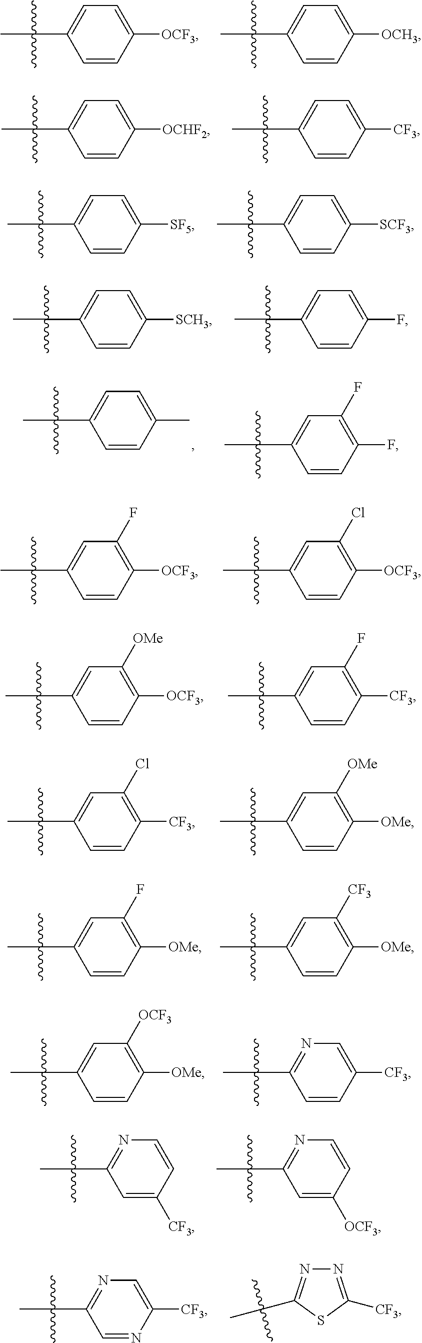

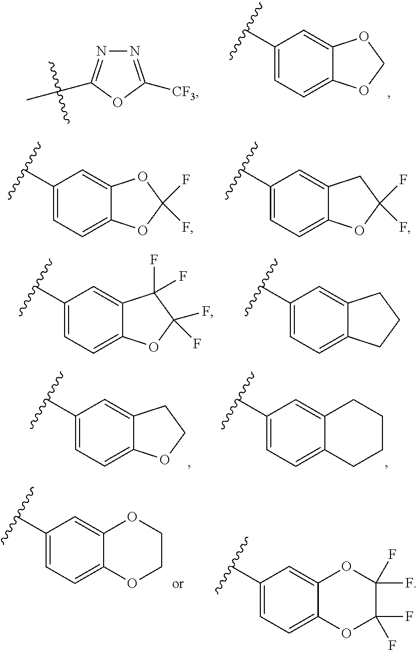

In another preferred embodiment, the compound has the structure as shown in Formula (1A):

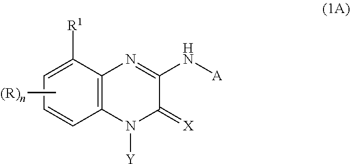

wherein:
n is 0, 1 or 2;
R is selected from the group consisting of: halogen, CN, nitro, C₁₋₃ alkyl, C₁₋₃ haloalkyl or C₁₋₃ alkoxy; R¹ is a halogen;
A is selected from the group consisting of: 6-10 membered aryl, 5-7 membered heteroaryl or benzo-5-7-membered heterocyclyl, wherein the aryl, heteroaryl and benzo heterocyclyl can be further substituted by 0-4 substituents selected from the group consisting of: halogen, CN, C₁₋₃ alkyl, C₃₋₆ cycloalkyl, C₁₋₃ alkoxy, C₁₋₃ alkylthio, C₁₋₃ haloalkyl, C₁₋₃ haloalkoxy, C₁₋₃ haloalkylthio or SF₅;
X is O, and Y is C₁₋₃ alkyl, C₁₋₃ haloalkyl or C₃₋₆ cycloalkyl; or
X and Y are taken together with the atoms to which they are bound to form a 5-membered heteroaryl or a 5-membered heterocyclyl, wherein the heteroaryl and heterocyclyl can be further substituted by 0-2 substituents of the following groups: C₁₋₃ alkyl or C₁₋₃ haloalkyl.

In another preferred embodiment, wherein

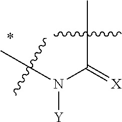

has a structure selected from the group consisting of

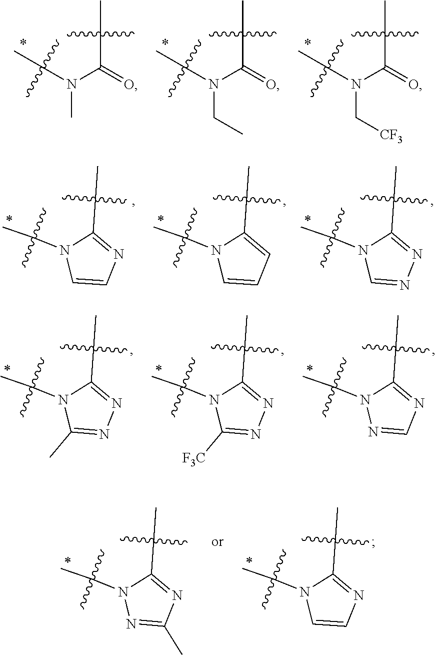

wherein “*” represents the position connected to the benzene ring.

In another preferred embodiment, the compound has a structure selected from the group consisting of:

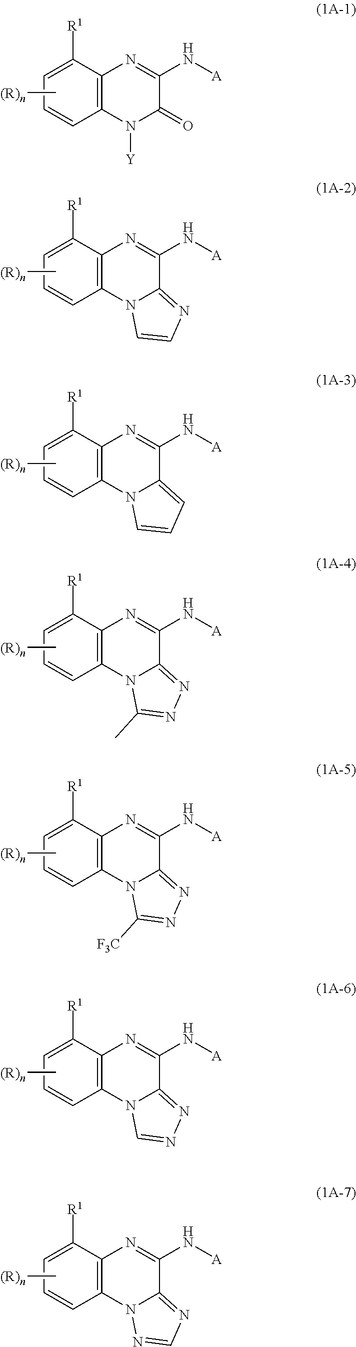

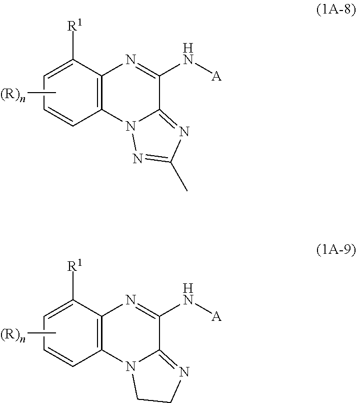

Wherein n, Y, R, R¹ and A are as described in the first aspect of the present invention.

In another preferred embodiment, the compound has the structure as shown in Formula (1B):

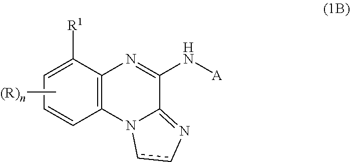

wherein:
“ [symbol] ” indicates a single bond or a double bond;
n is 1, 2 or 3;
each R is independently selected from the group consisting of: halogen, CN, C₁₋₃ alkyl, C₁₋₃ haloalkyl, C₁₋₃ alkoxy or C₁₋₃ haloalkoxy;
R¹ is selected from the group consisting of: H, CN, C₁₋₃ alkyl or C₁₋₃ alkoxy;
A is selected from the group consisting of: 6-10 membered aryl, 5-7 membered heteroaryl, benzo 5-7 membered cycloalkyl or benzo 5-7-membered heterocyclyl, wherein the aryl heteroaryl, benzo-cycloalkyl and benzo heterocyclyl can be further substituted by 0-4 substituents selected from the group consisting of: halogen, CN, C₁₋₃ alkyl, C₃₋₆ cycloalkyl, C₁₋₃ alkoxy, C₁₋₃ alkylthio, C₁₋₃ haloalkyl, C₁₋₃ haloalkoxy, C₁₋₃ haloalkylthio or SF₅.

In another preferred embodiment, the compound has the structure as shown in Formula (2):

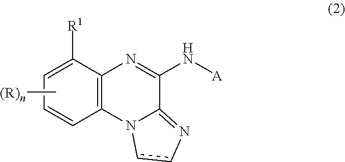

wherein:
“ [symbol] ” indicates a single bond or a double bond;
n is 0, 1 or 2;
R¹ is selected from the group consisting of: H, F, Cl or Br;
each R is independently selected from the group consisting of: F, Cl or Br.

In another preferred embodiment, wherein A is selected from the group consisting of:

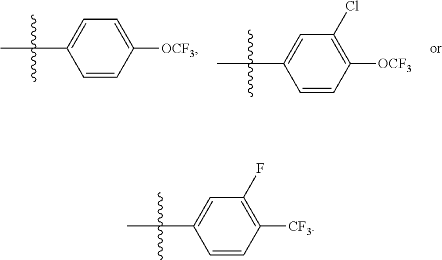 [SMILES: *c1ccc(OC(F)(F)F)cc1 ; *c1ccc(OC(F)(F)F)c(Cl)c1 ; *c1ccc(C(F)(F)F)c(F)c1]

In another preferred embodiment, wherein n is 1 or 2 when R¹ is H.

In another preferred embodiment, wherein the compound has a structure selected from the group consisting of:

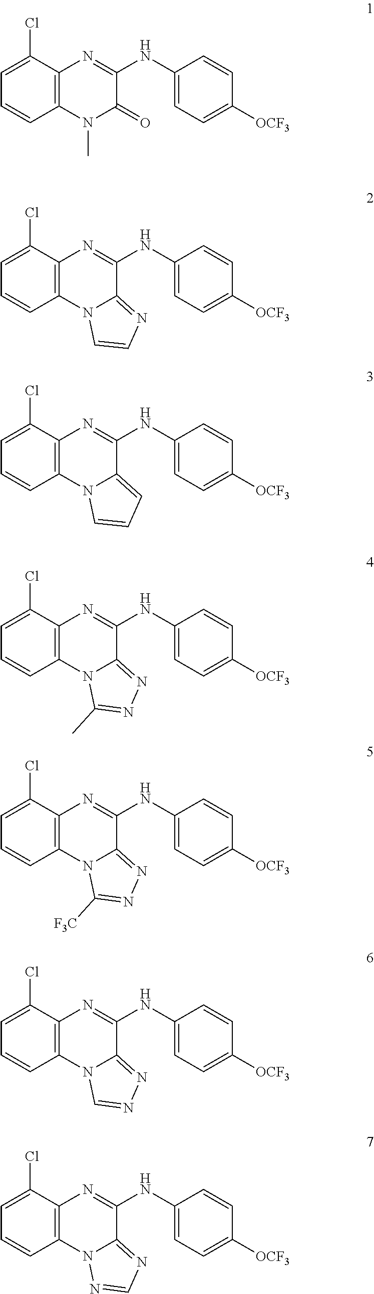

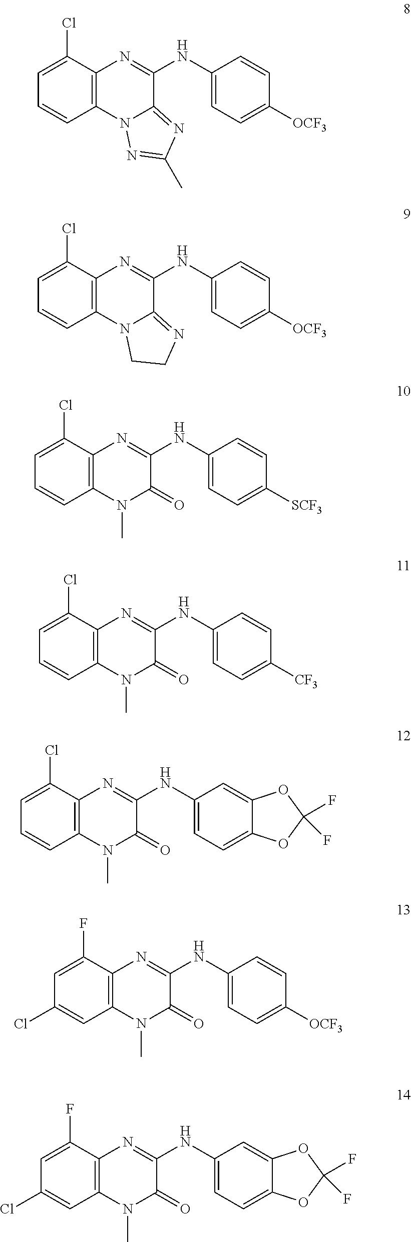

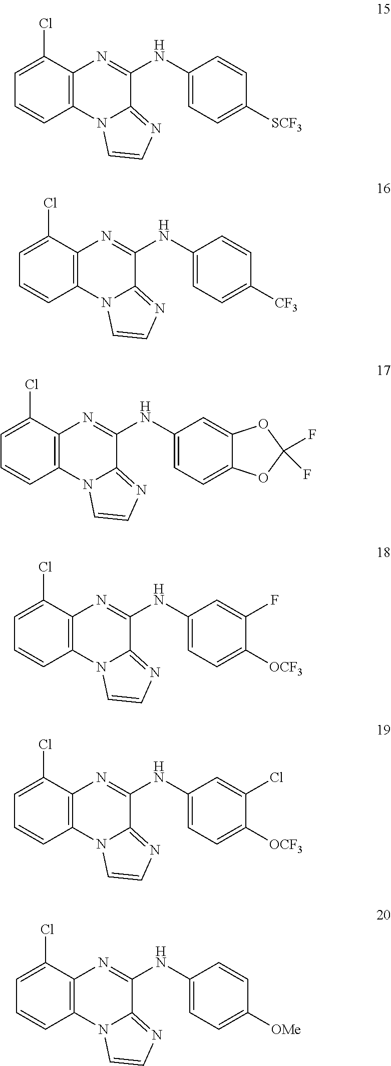

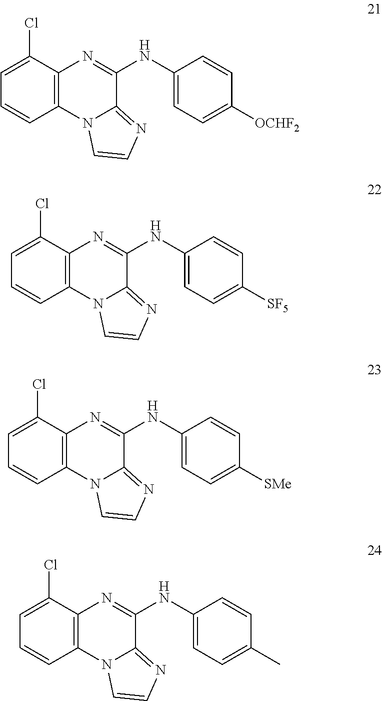

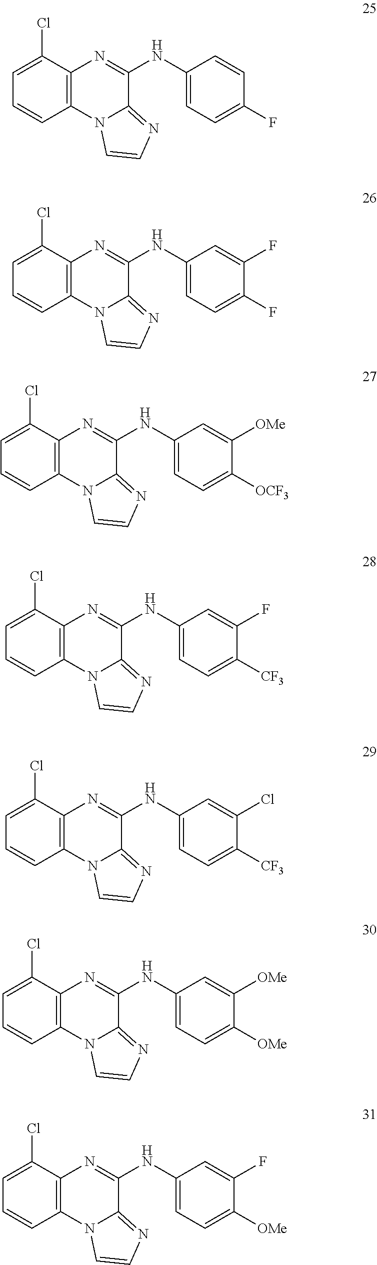

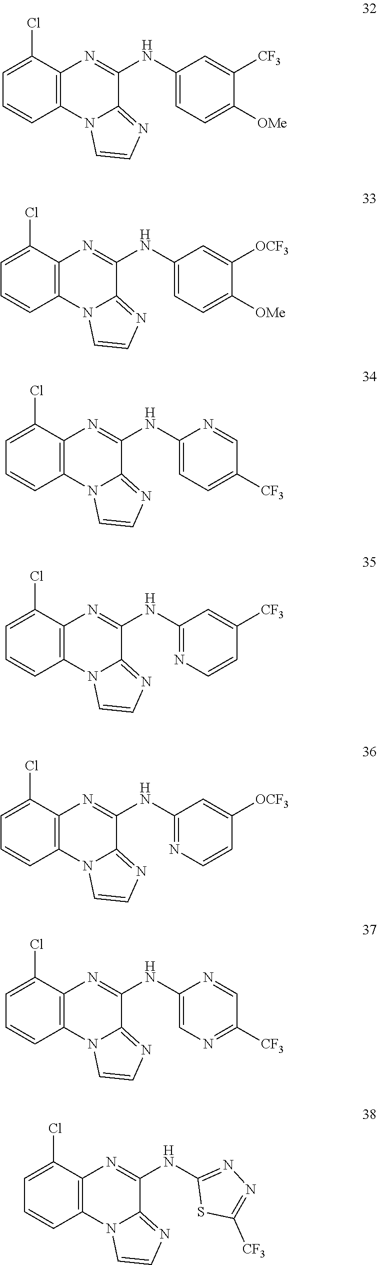

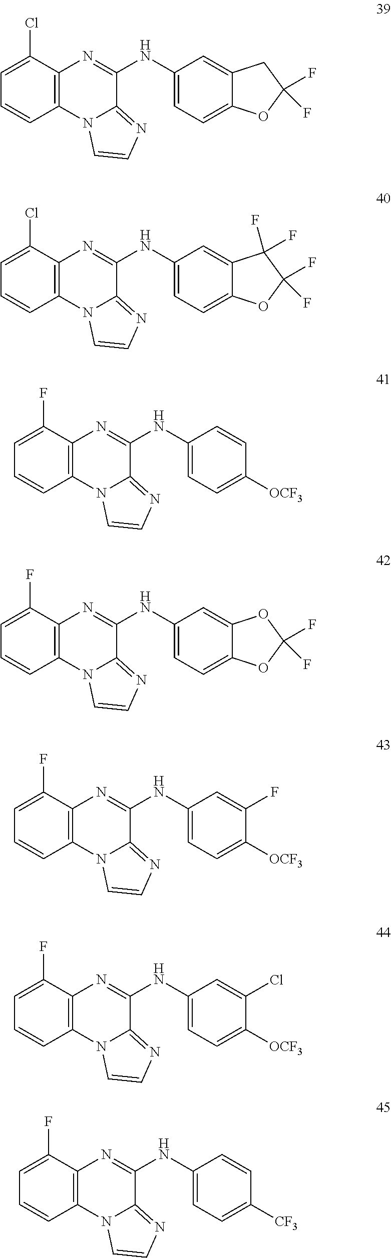

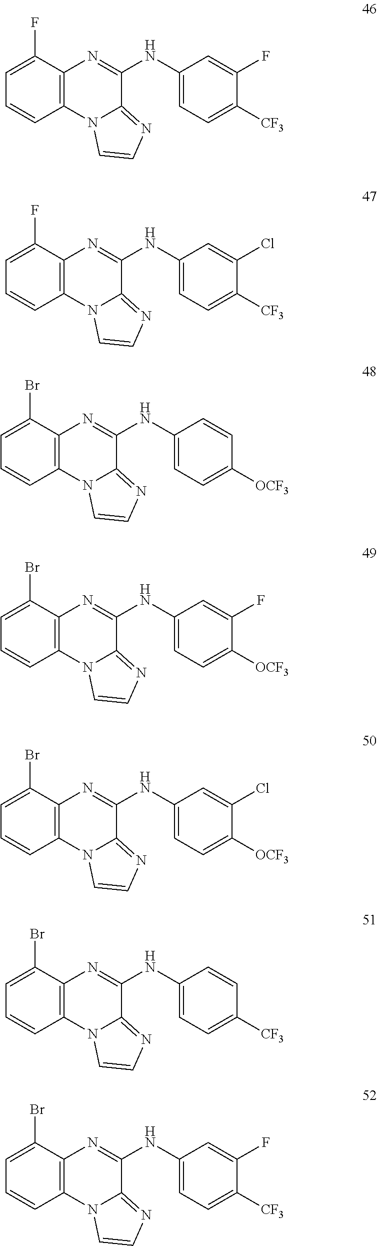

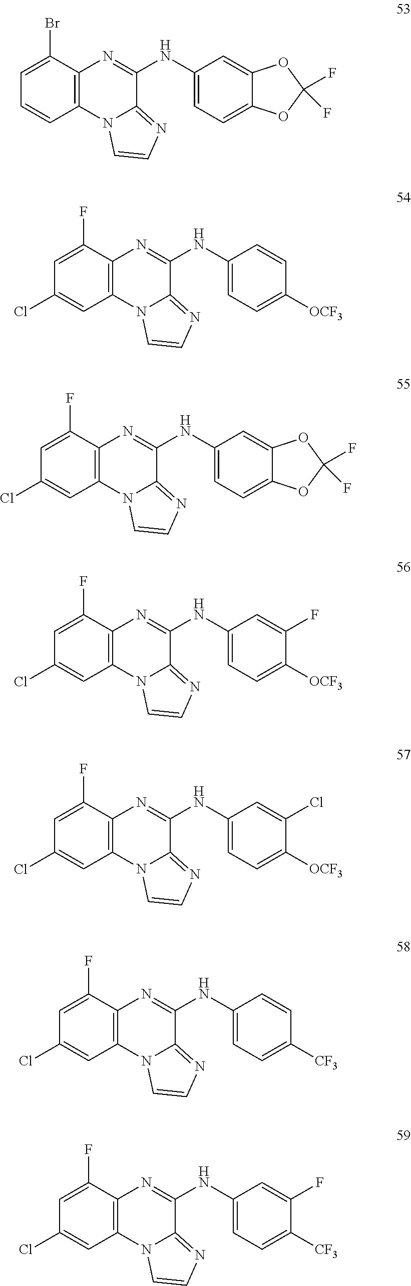

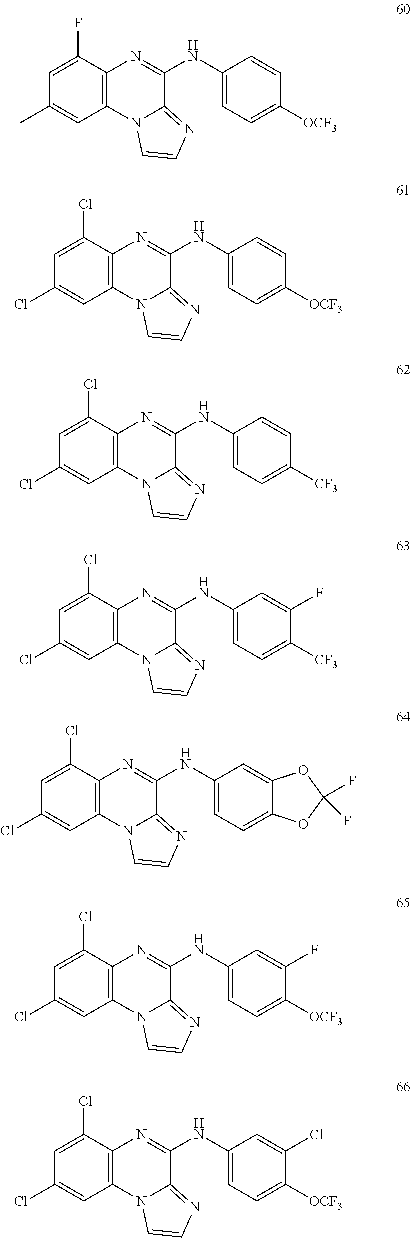

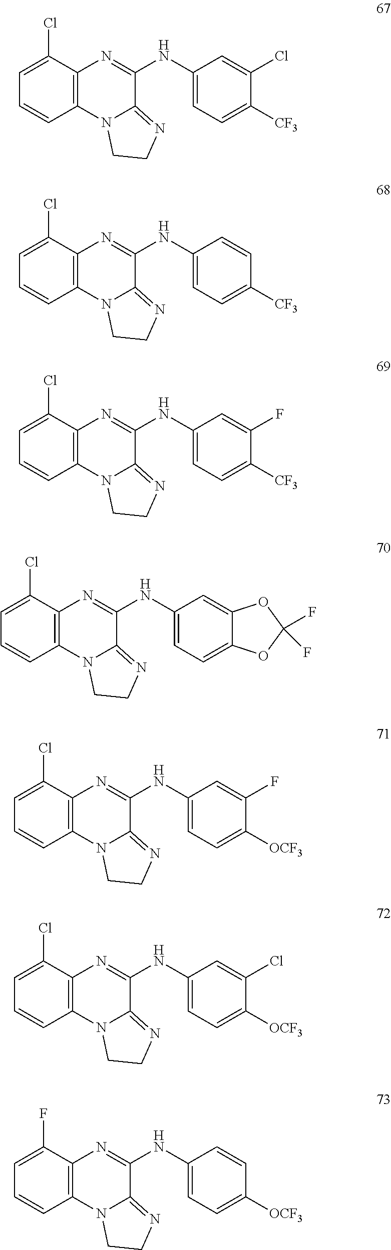

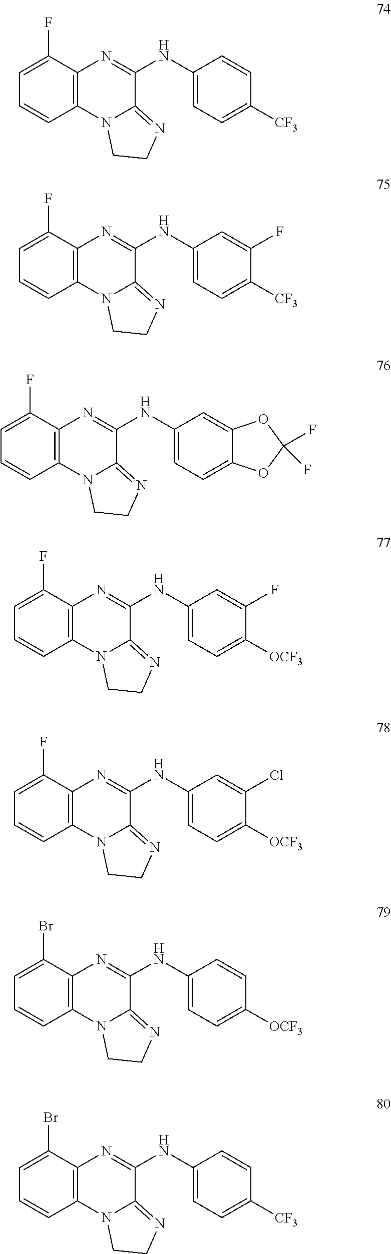

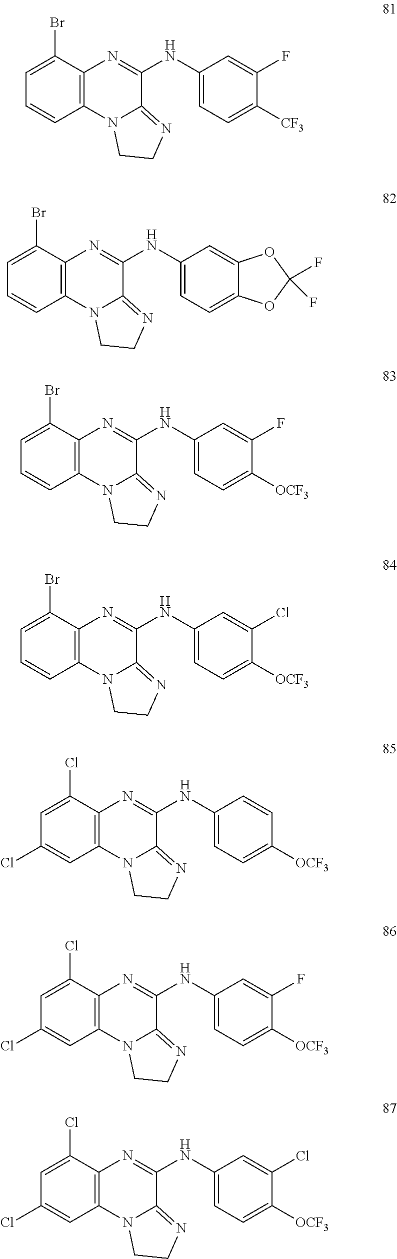

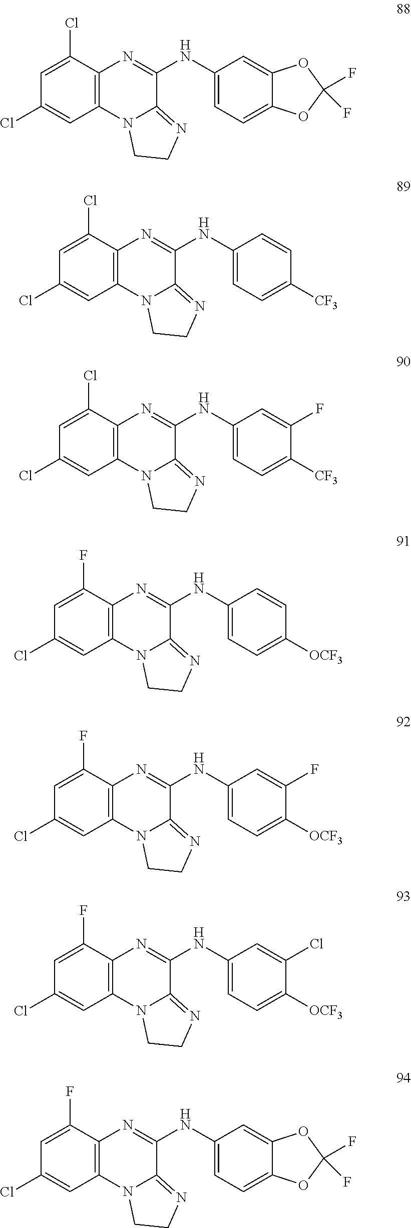

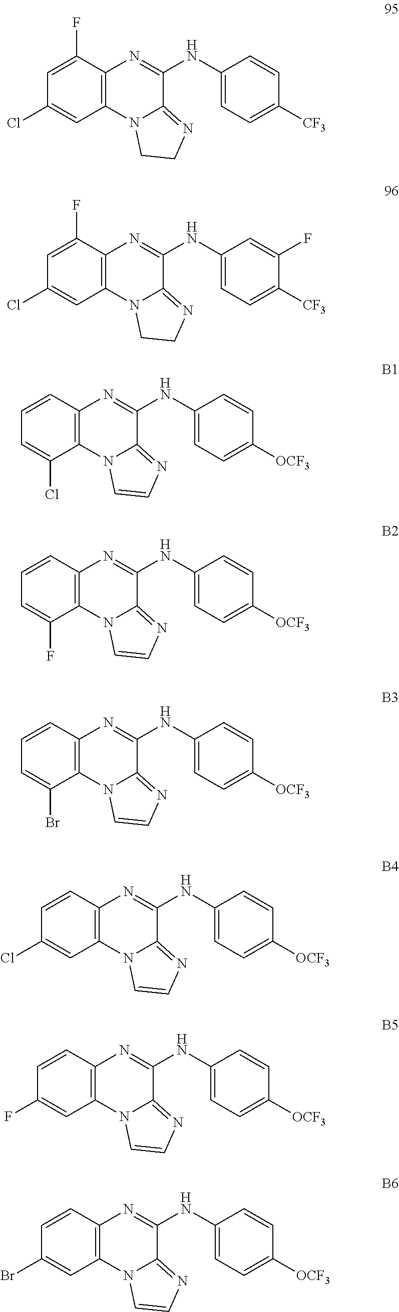

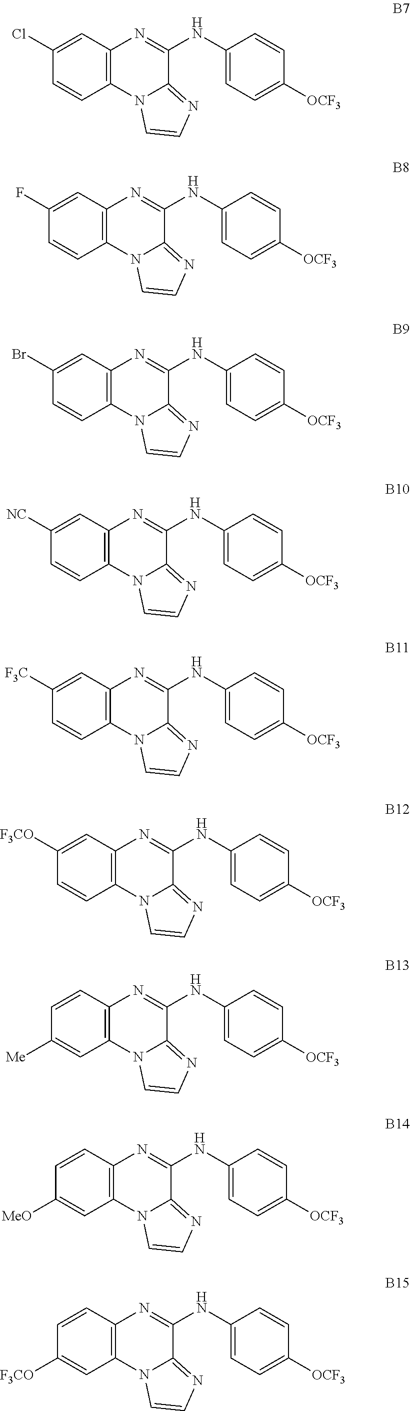

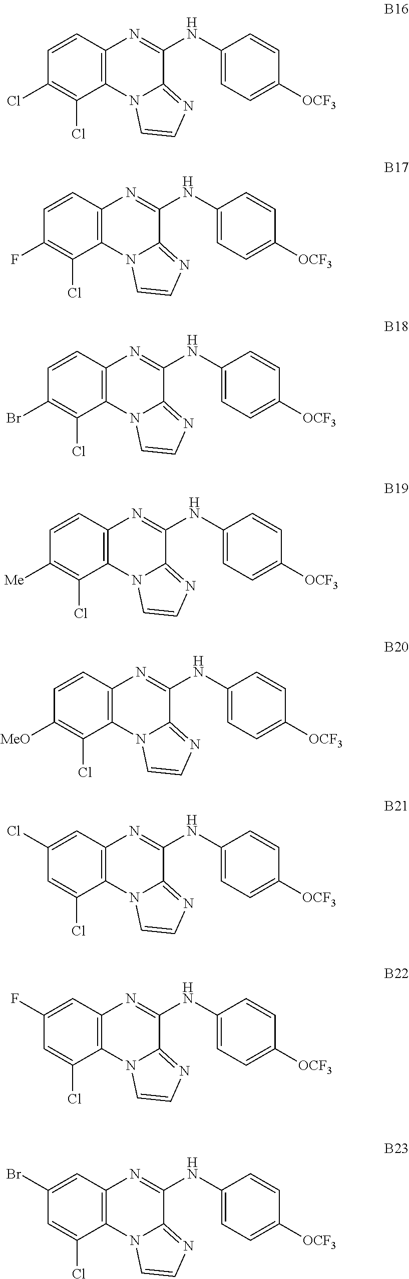

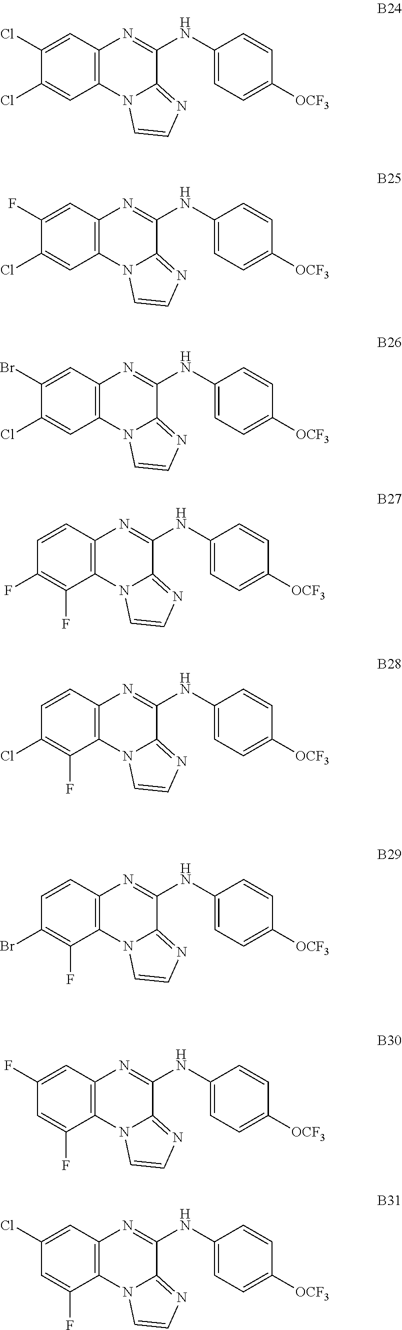

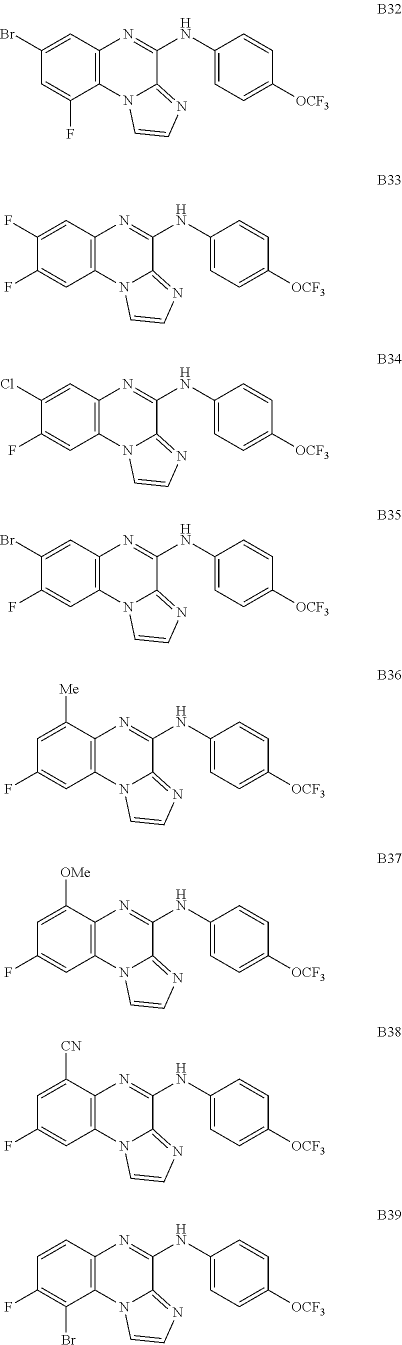

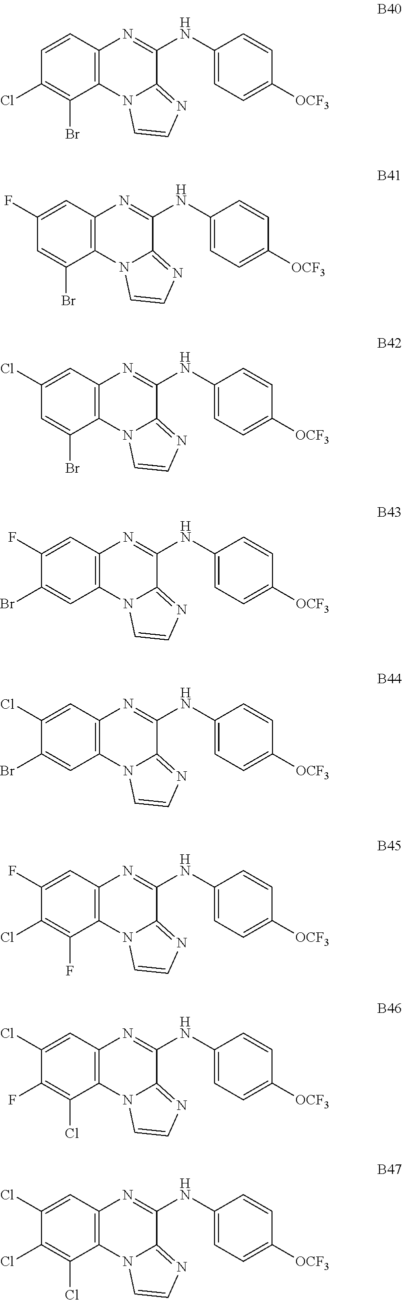

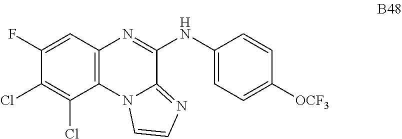 [SMILES: Fc1cc2nc(Nc3ccc(OC(F)(F)F)cc3)c3nccn3c2c(Cl)c1Cl]

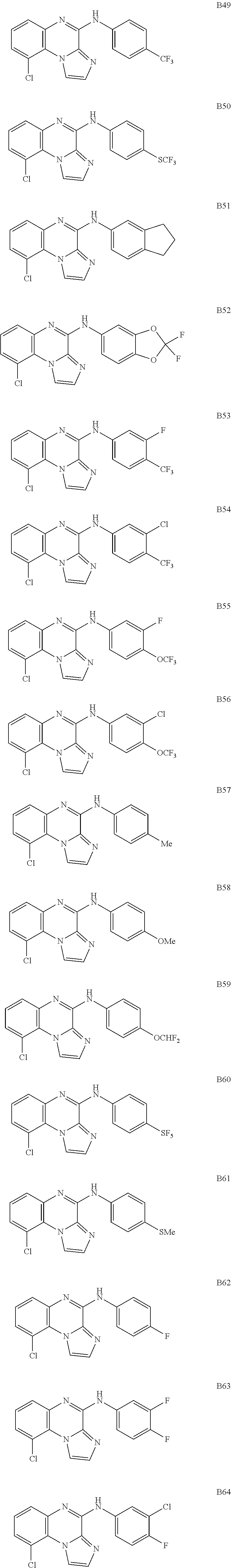

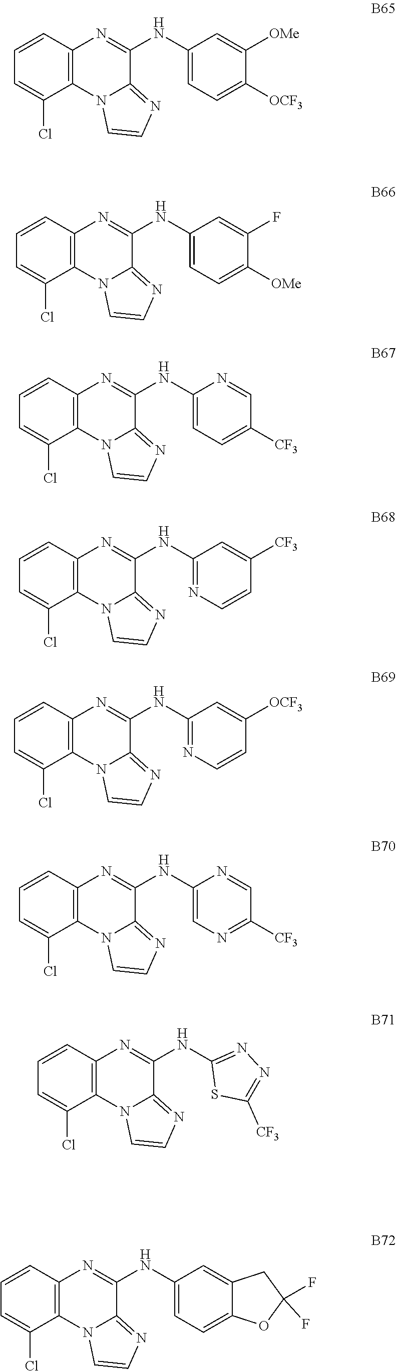

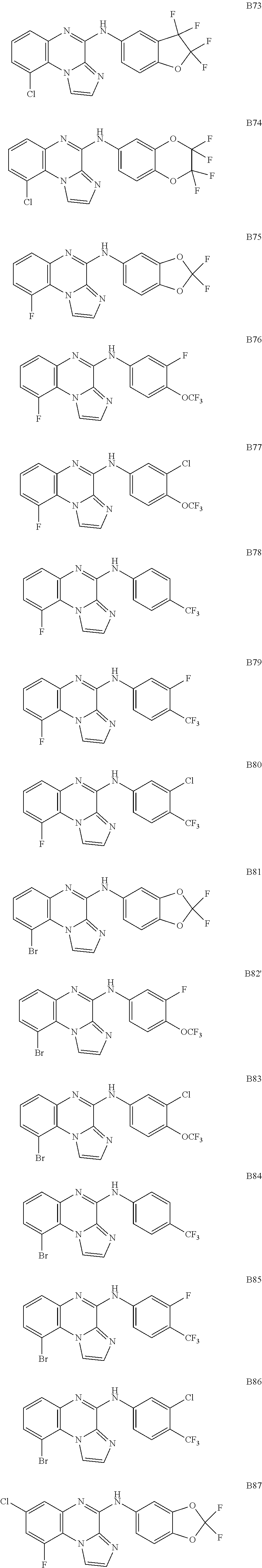

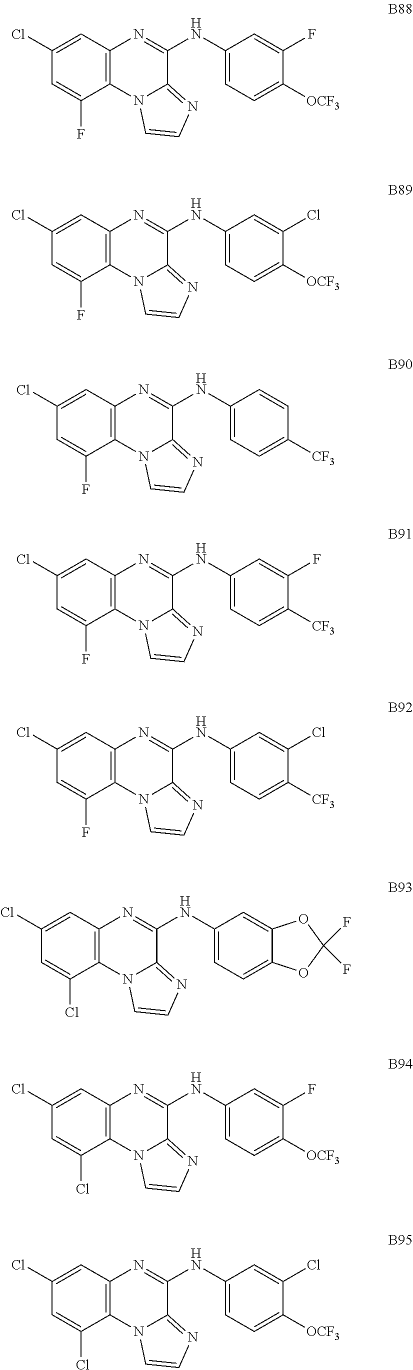

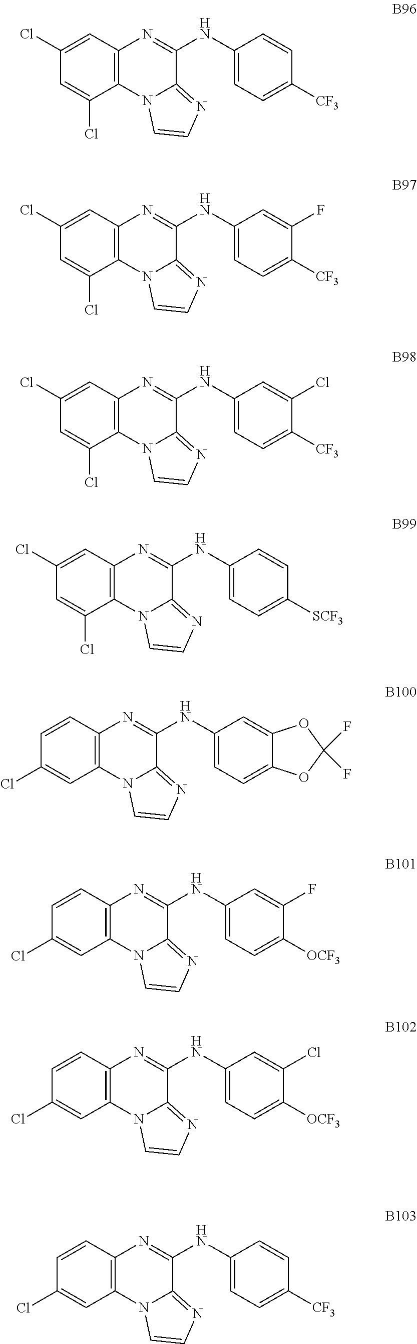

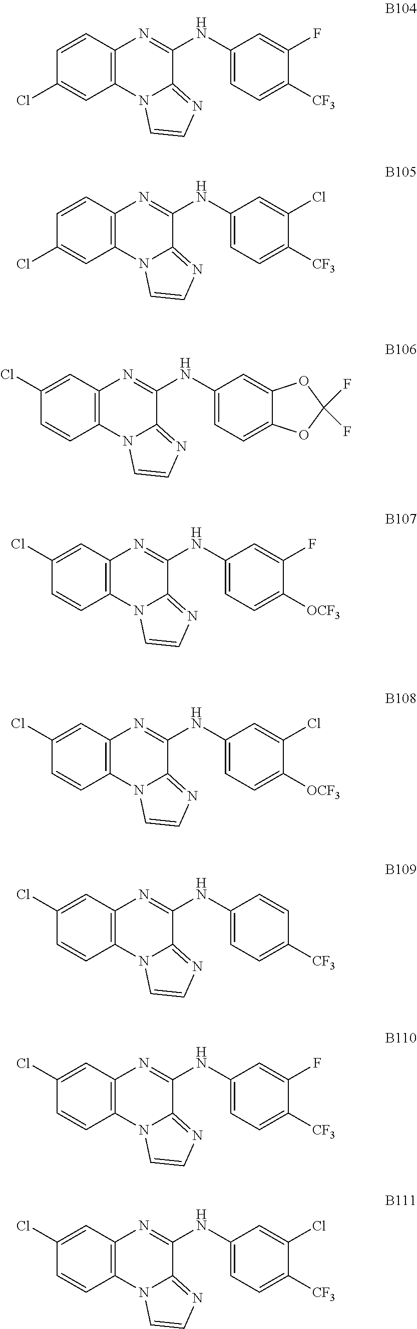

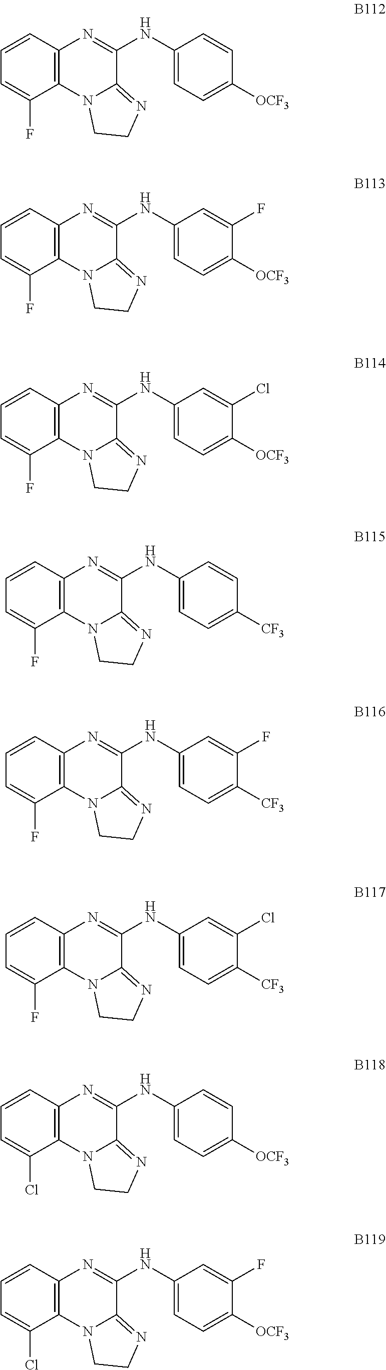

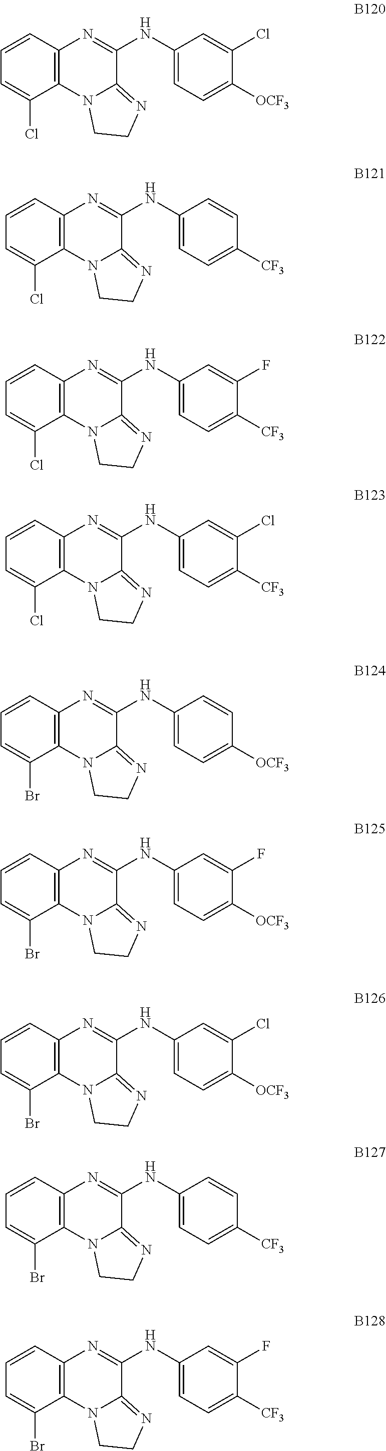

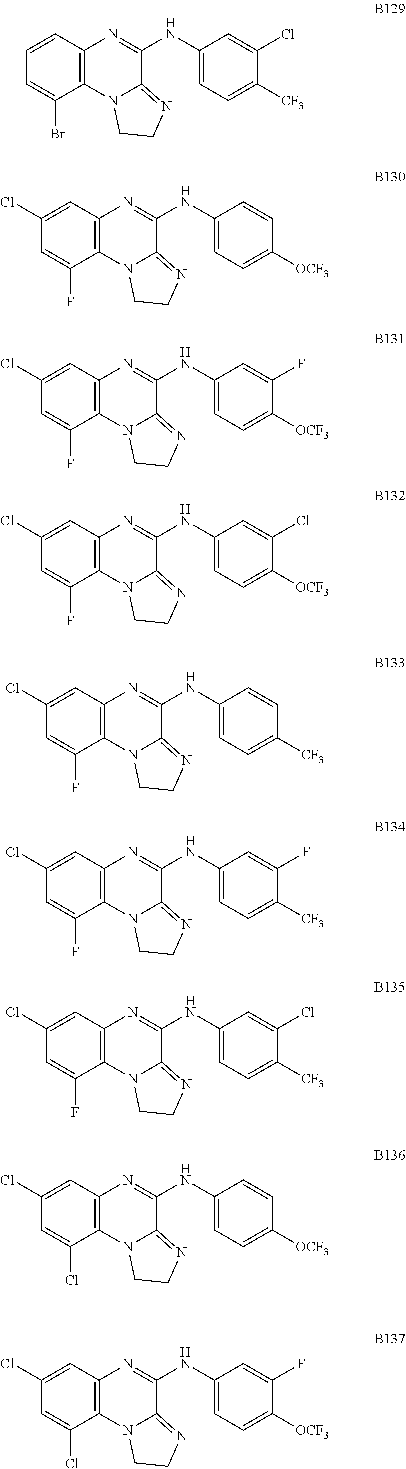

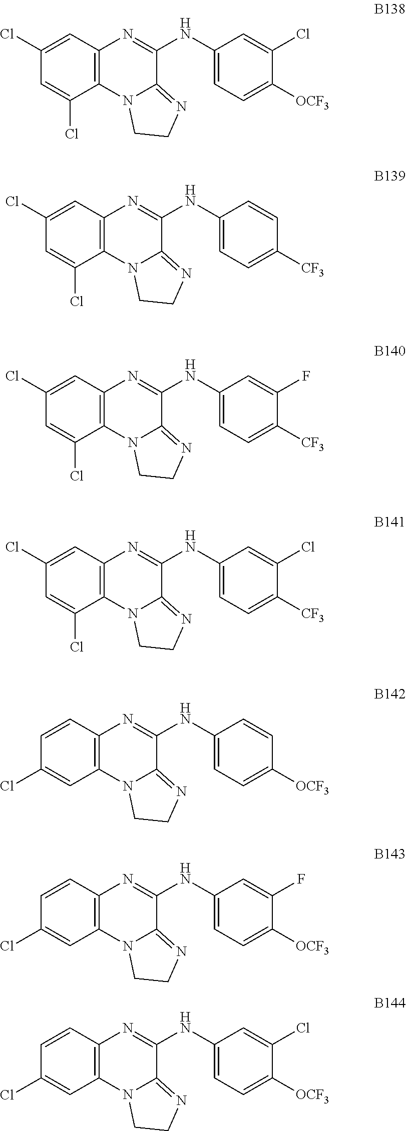

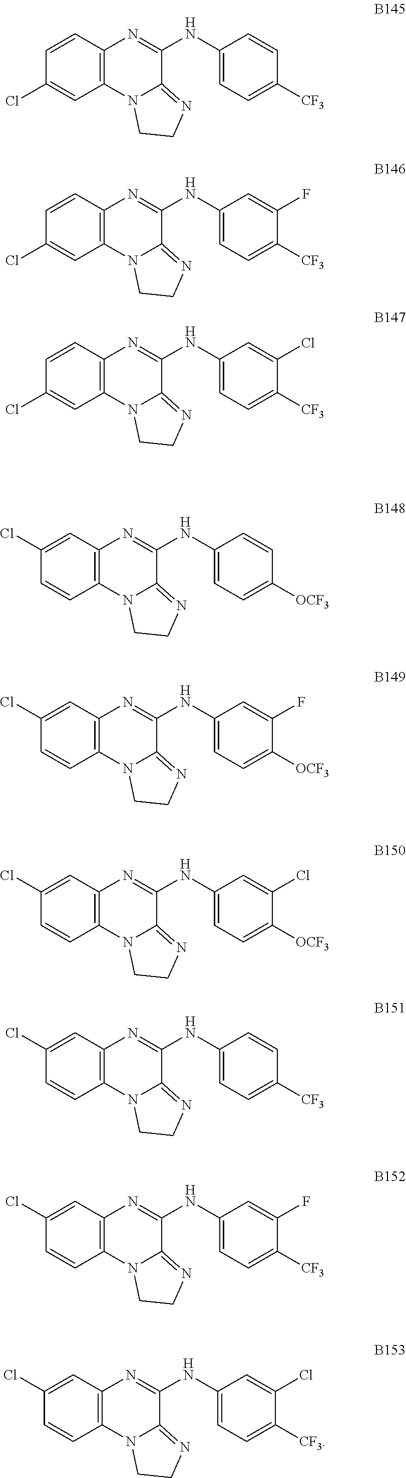

In the second aspect of the present invention, it provides a pharmaceutical composition for treating, regulating, and/or preventing a disease associated with miR-124, characterized in that the pharmaceutical composition comprises:
(1) the compound as an active ingredient according to any one of claims 1-13, or an isomer, crystal form, pharmaceutically acceptable salt, hydrate, or solvate thereof; and
optionally (2) a pharmaceutically acceptable excipient, or carrier.

In the third aspect of the present invention, it provides use of the compound as described in the first aspect of the present invention, or a isomer, crystal form, pharmaceutically acceptable salt, hydrate, or solvate thereof, or the pharmaceutical composition as described in the second aspect of the present invention for preparing a pharmaceutical composition for treating, regulating, and/or preventing a disease mediated by miR-124.

In another preferred embodiment, wherein the disease mediated by miR-124 is selected from the group consisting of: Alzheimer's disease, Parkinson's disease, brain tumor, gastric cancer, liver cancer, lung cancer, colorectal cancer, pancreatic cancer, breast cancer, cervical cancer, endometrial cancer, prostate cancer, Crohn's disease, ulcerative colitis, rheumatoid arthritis, allergic rhinitis, osteoarthritis, fibrosis disease, AIDS and/or COVID-19.

In the forth aspect of the present invention, it provides a method for preparing a compound of Formula (1), characterized in that the method includes the steps of:
(1) in an inert solvent, the compound of formula (A) and its tautomer react to obtain the compound of formula (B):

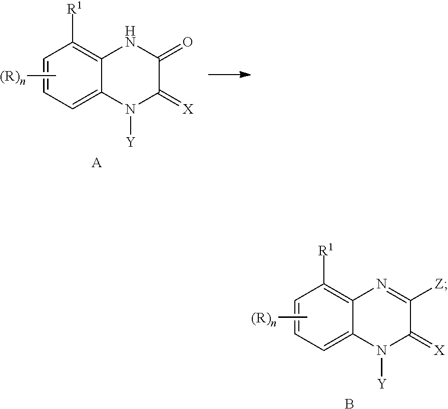

(2) in an inert solvent, the compound of formula (B) reacts with the compound of formula (d) to obtain the compound of formula (1):

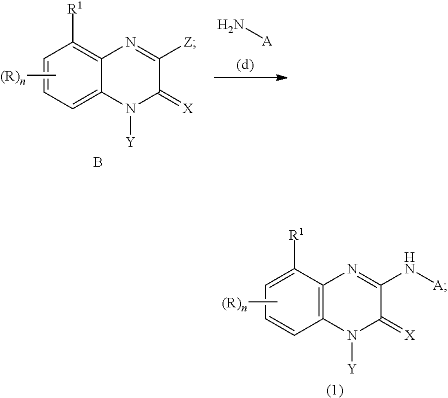

wherein Z is a halogen or OTf; X, Y, R, R¹, A and n are as defined in the first aspect of the present invention.

In another preferred embodiment, wherein the compound of formula (1) is a compound of formula (1a) or (1b),
and the preparation method includes the following steps:
(s1) in an inert solvent, the compound of formula (A1) and its tautomer react to obtain the compound of formula (B1):

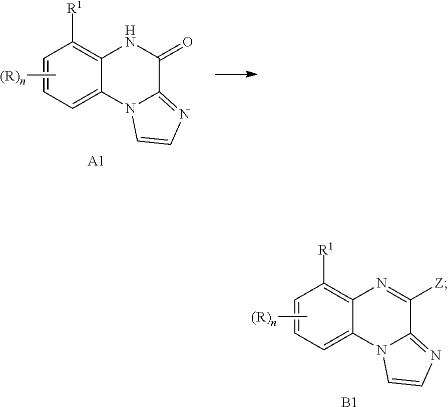

(s2) in an inert solvent, the compound of formula (B1) reacts with the compound of formula (d) to obtain the compound of formula (1a):
wherein Z is a halogen or OTf; R, R¹, A and n are as defined in the first aspect of the present invention.

In another preferred embodiment, the preparation method includes the following steps:
(t1) in an inert solvent, the compound of formula (C) undergoes a cyclization reaction to yield the compound of formula (D):

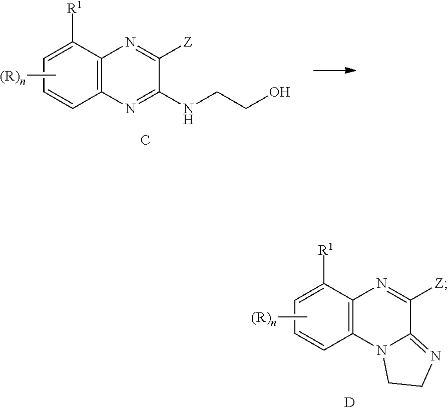

(t2) in an inert solvent, the compound of formula (D) reacts with the compound of formula (d) to obtain the compound of formula (1b):

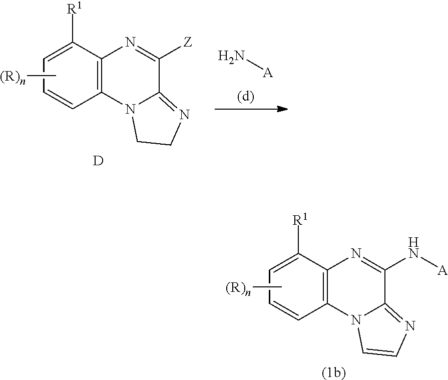

wherein Z is a halogen or OTf, R, R¹, A and n are as defined in the first aspect of the present invention.

In the fifth aspect of the present invention, it provides a method for treating, regulating, and/or preventing a disease mediated by miR-124, which includes the steps of: administering to an individual in need thereof an amount of the compound as described in the first aspect of the present invention, or an isomer, crystal form, pharmaceutically acceptable salt, hydrate, or solvate thereof, or the pharmaceutical composition described in the second aspect of the present invention.

In another preferred embodiment, the individual includes humans and non-human mammals.

It should be understood that within the scope of the present invention, all the technical features of the present invention and those specifically described in the following text (e.g., examples) can be combined with each other to form new or preferred technical solutions. Due to the limited space, we will not enumerate them one by one here.

### FIGURE DESCRIPTION

FIG. 1 shows the body weight changes of mice over time after the administration of the compound of each experimental group.

### DETAILED DESCRIPTION OF THE ILLUSTRATED EMBODIMENTS

The inventor, after extensive and intensive research, and through a large number of screenings and tests, first discovered a kind of compounds of Formula (1), which have a significant therapeutic effect on diseases mediated by miR-124. The compounds of the present invention have a potent ability to induce the expression of miR-124 in PBMC cells stimulated by PMA/IO, and the compounds have excellent pharmacokinetic properties. At the same time, the compounds have significant in vivo activity and good pharmaceutical properties. On this basis, the present invention was completed.

### The Compound of the Present Invention and its Synthesis

The present invention provides an inducer of miR-124, i.e., a compound of Formula (1), or an isomer, crystal form, pharmaceutically acceptable salt (inorganic salt or organic salt), hydrate, or solvate thereof. Preferably, the compound of the present invention is as described in the first aspect of the present invention.

The present invention also provides a method for preparing the compound of Formula (1) of the present invention. The following specifically describes the method for preparing the compound of Formula (1) of the present invention, but these specific methods do not constitute any limitation on the present invention.

In one aspect, the compounds described herein are prepared according to the well-known methods. However, the conditions of the process, such as reactants, solvents, bases, amounts of compounds used, reaction temperature, time required for the reactions, etc., are not limited to the following explanations. The compounds of the present invention may also be conveniently prepared, optionally in combination with various synthesis methods described in this specification or well-known in the art, and such combinations are readily carried out by those skilled technicians in the art. On the other aspect, the present invention also provides the preparation methods of the compounds represented by the general formula (1A) or the general formula (1B), which are prepared by the following Schemes 1-9:

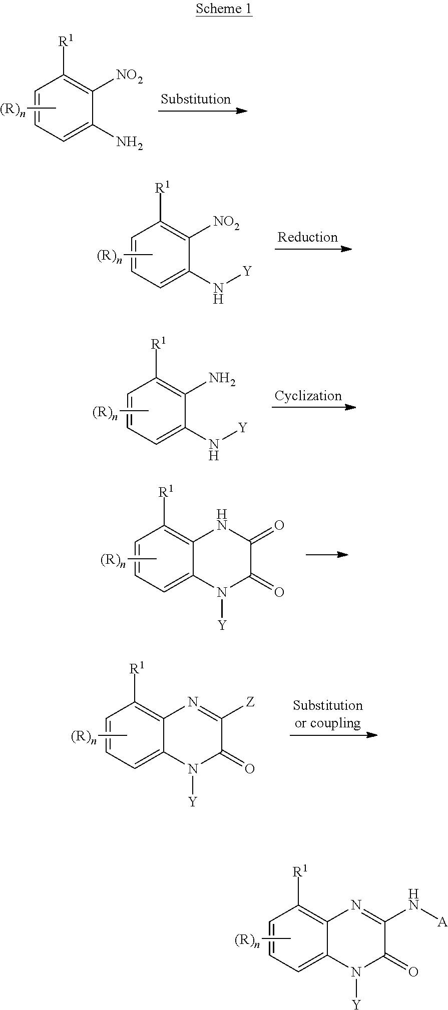

The embodiments of the compound of Formula (1) can be prepared according to the scheme 1, wherein Z is a halogen or OTf, and R, R¹, Y, A and n are as defined above.

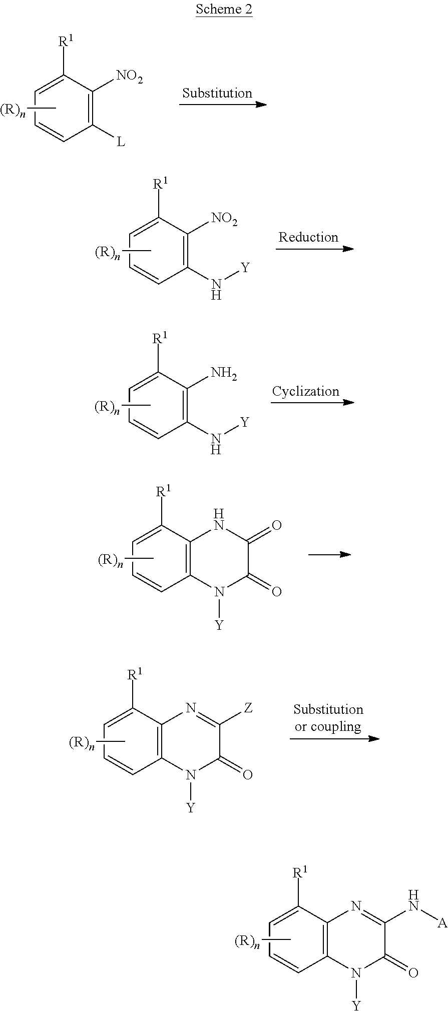

The embodiments of the compound of Formula (1) can be prepared according to the scheme 2, wherein L is a halogen, Z is a halogen or OTf, and R, R¹, Y, A and n are as defined above.

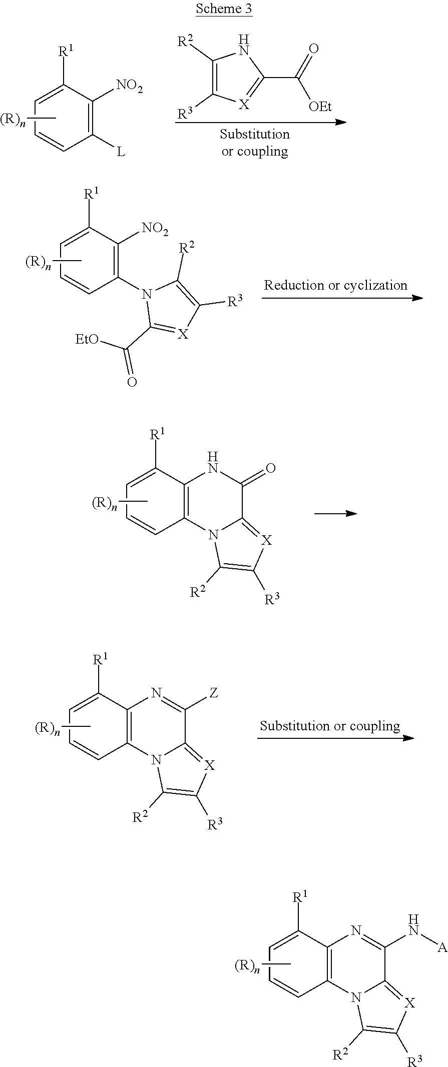

The embodiments of compounds of Formula (1A) or Formula (1B) can be prepared according to the scheme 3, wherein L is a halogen, X is N or CH, Z is a halogen or OTf, R² and R³ are independently selected from H, C₁₋₃ alkyl or C₁₋₃ haloalkyl, and R, R¹, A and n are as defined above.

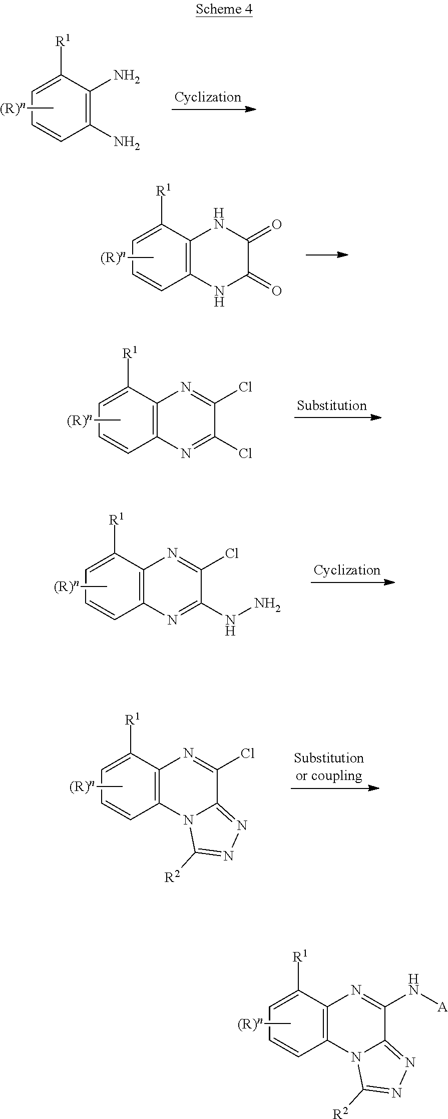

The embodiments of the compound of Formula (1) can be prepared according to the scheme 4, wherein R² is H, C₁₋₃ alkyl or C₁₋₃ haloalkyl, and R, R¹, Y, A and n are as defined above.

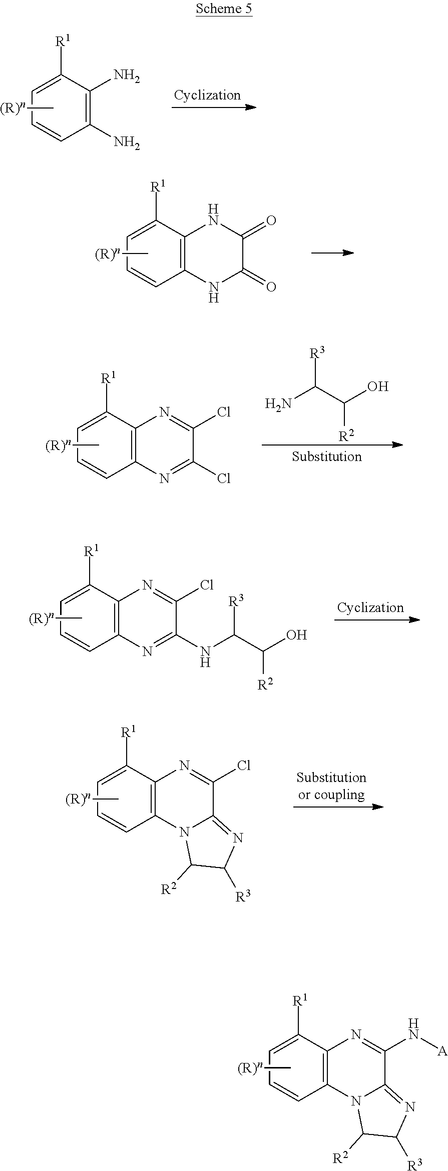

The embodiments of the compound of Formula (1) can be prepared according to the scheme 5, wherein R² and R³ are independently selected from H, C₁₋₃ alkyl or C₁₋₃ haloalkyl, and R, R¹, A and n are as defined above.

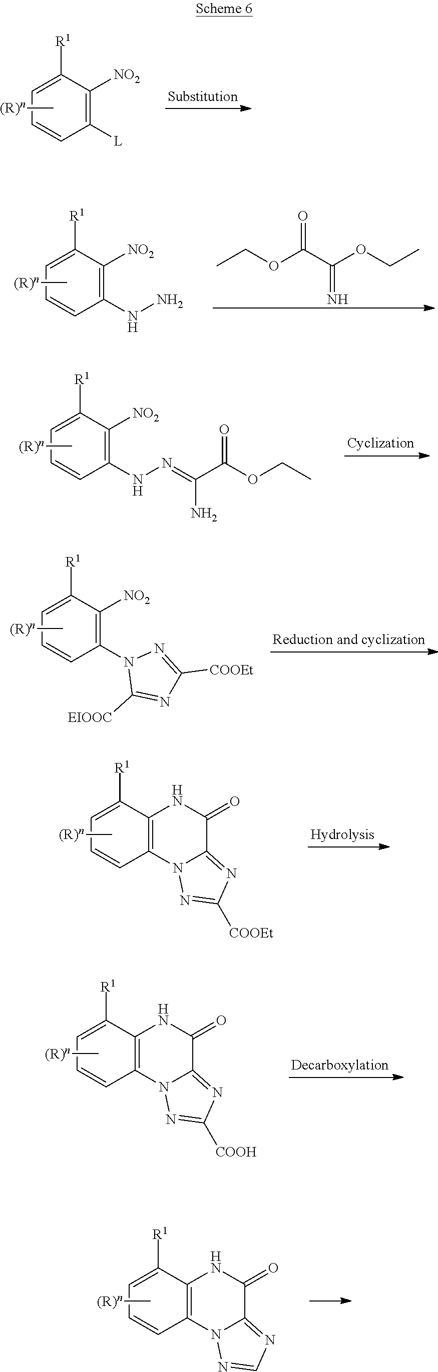

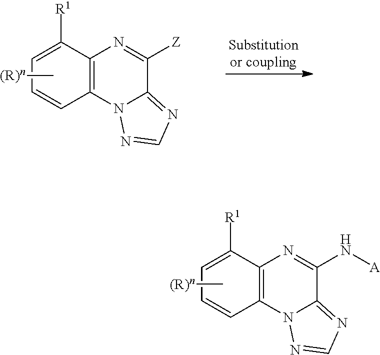

The embodiments of the compound of Formula (1) can be prepared according to the scheme 6, wherein L is a halogen, Z is a halogen or OTf, and R, R¹, Y, A and n are as defined above.

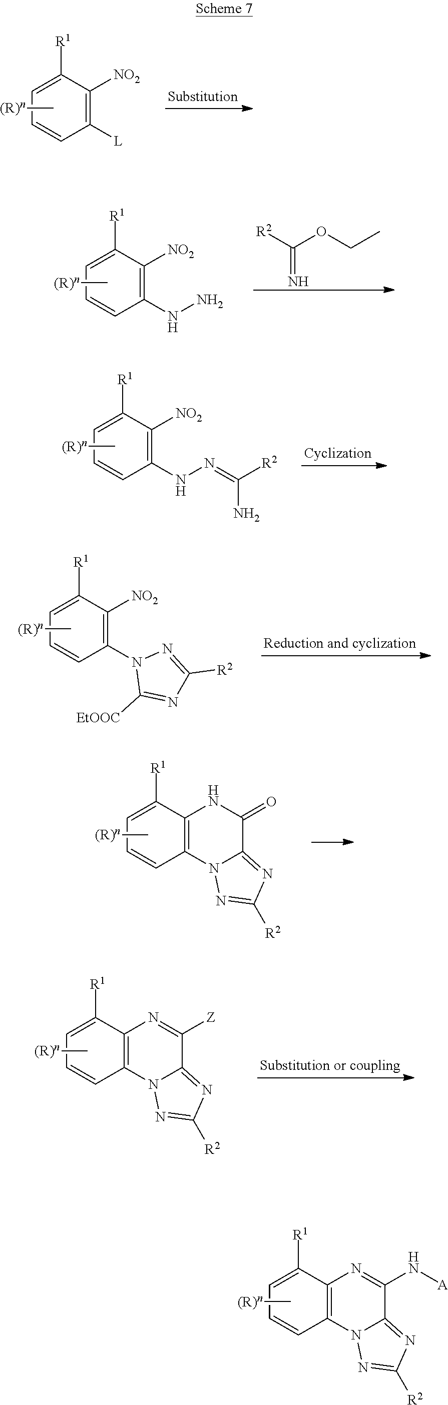

The embodiments of the compound of Formula (1) can be prepared according to the scheme 7, wherein L is a halogen, Z is a halogen or OTf, R² is C₁₋₃ alkyl or C₁₋₃ haloalkyl, and R, R¹, Y, A and n are as defined above.

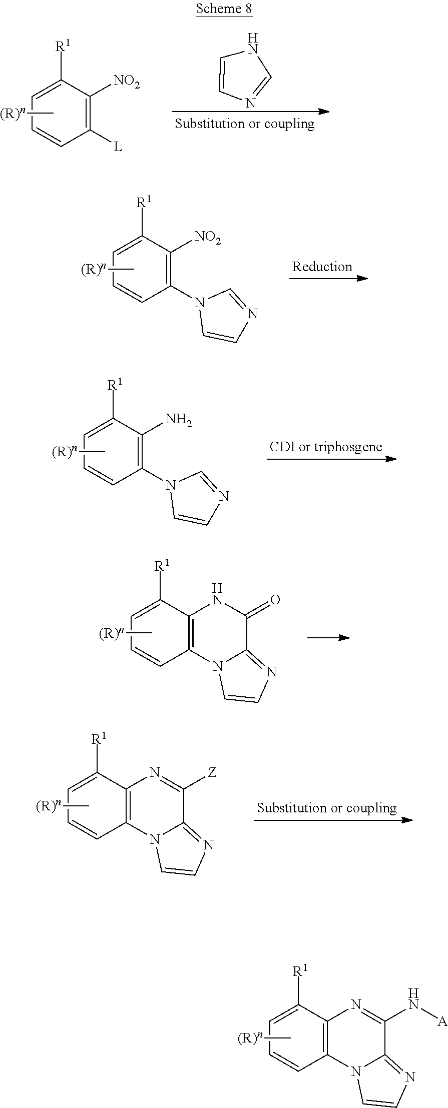

The embodiments of the compound of Formula (1B) can be prepared according to the scheme 8, wherein L is a halogen, Z is a halogen or OTf, and R, R¹, Y, A and n are as defined above.

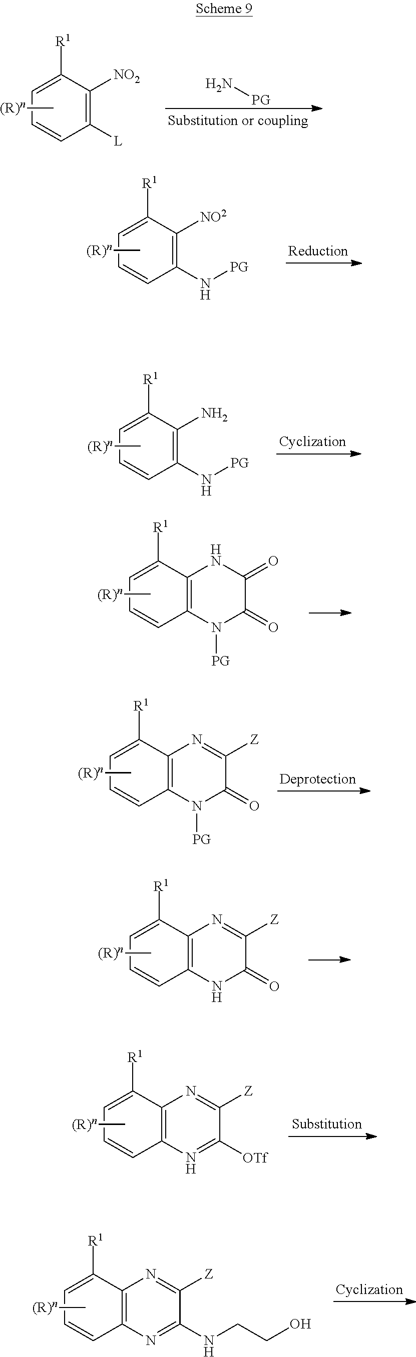

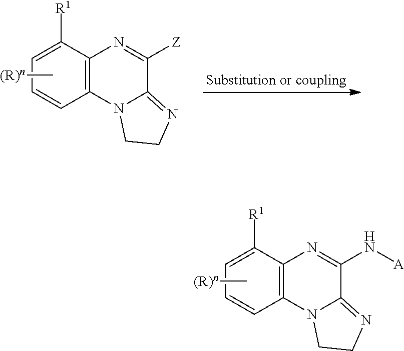

The embodiments of the compound of Formula (1B) can be prepared according to the scheme 9, wherein L is a halogen, Z is a Cl or Br, PG is an amino protecting group such as Boc, Cbz, Bn, PMB, etc., and R, R¹, Y, A and n are as defined above.

### Related Definitions

Unless otherwise specified, the following terms and phrases used herein are intended to have the following meanings. A specific term or phrase should not be considered indefinite in the absence of a particular definition, but should be understood according to its ordinary meaning. When a trade name appears herein, it is intended to refer to its corresponding commercial product or active ingredient thereof.

The term “pharmaceutically acceptable” used herein in terms of those compounds, materials, compositions, and/or dosage forms, which are suitable for use in contact with human and animal tissues within the scope of reliable medical judgment, without excessive toxicity, irritation, allergic reactions or other problems or complications, commensurate with a reasonable benefit/risk ratio.

The term “pharmaceutically acceptable salt” refers to a salt of the compound of the present disclosure that is prepared by reacting the compound having a specific substituent of the present invention with a relatively non-toxic acid or base. When the compound of the present disclosure contains a relatively acidic functional group, a base addition salt can be obtained by contacting the compound with a sufficient amount of a base in a pure solution or a suitable inert solvent. The pharmaceutically acceptable base addition salts includes a salt of sodium, potassium, calcium, ammonium, organic amine or magnesium, or similar salts. When the compound of the present disclosure contains a relatively basic functional group, an acid addition salt can be obtained by contacting the compound with a sufficient amount of acid in a pure solution or a suitable inert solvent. Examples of pharmaceutically acceptable acid addition salts include an inorganic acid salt, wherein the inorganic acid include, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphinic acid and the like; and an organic acid salt, wherein the organic acid includes, for example, acetic acid, propionic acid, isobutyric acid, maleic acid, malonic acid, benzoic acid, succinic acid, octanedioic acid, trans-butenedioic acid, lactic acid, mandelic acid, phthalic acid, benzenesulfonic acid, p-toluenesulfonic acid, citric acid, tartaric acid, and methanesulfonic acid, and the like; and salts of amino acids (such as arginine and the like), and a salt of organic acids such as glucuronic acid and the like. Certain specific compounds of the present invention contain both basic and acidic functional groups, thus can be converted to any base or acid addition salt.

The pharmaceutically acceptable salt of the present disclosure can be prepared from the parent compound that contains an acid or basic moiety by conventional chemical method. Generally, such salt can be prepared by reacting the free acid or base form of the compound with a stoichiometric amount of an appropriate base or acid in water or an organic solvent or a mixture thereof.

The compounds of the present disclosure may exist in specific geometric or stereoisomeric forms. The present disclosure contemplates all such compounds, including cis and trans isomers, (−)- and (+)-enantiomers, (R)- and (S)-enantiomers, diastereoisomers, (D)-isomers, (L)-isomers, and racemic and other mixtures thereof, such as enantiomers or diastereomer enriched mixtures, all of which are within the scope of the present disclosure. Additional asymmetric carbon atoms may be present in substituents such as alkyl. All these isomers and their mixtures are encompassed within the scope of the present disclosure.

Unless otherwise specified, the term “enantiomer” or “optical isomer” refers to stereoisomers that are mirror images of each other.

Unless otherwise specified, the term “cis-trans isomer” or “geometric isomer” is caused by the inability to rotate freely double bonds or single bonds of ring-forming carbon atoms.

Unless otherwise specified, the term “diastereomer” refers to a stereoisomer in which a molecule has two or more chiral centers and the relationship between the molecules is not mirror images.

Unless otherwise specified, “(D)” or “(+)” refers to dextrorotation, “(L)” or “(−)” refers to levorotation, and “(DL)” or “(±)” refers to racemic.

Unless otherwise specified, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ), a wave line ( [symbol] ) is used to represent a wedged solid bond ( [symbol] ) or a wedged dashed bond ( [symbol] ), or the wave line ( [symbol] ) is used to represent a straight solid bond ( [symbol] ) or a straight dashed bond ( [symbol] ).

Unless otherwise specified, the term “isomer excess” or “enantiomeric excess” refers to the difference between the relative percentages of two isomers or two enantiomers. For example, if the content of one isomer or enantiomer is 90%, and the content of the other isomer or enantiomer is 10%, then the isomer or enantiomer excess (ee value) is 80%.

Optically active (R)- and (S)-isomers, or D and L isomer can be prepared using chiral synthesis or chiral reagents or other conventional techniques. If one kind of enantiomer of certain compound of the present disclosure is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (such as an amino) or an acidic functional group (such as a carboxyl), the compound reacts with an appropriate optically active acid or base to form a salt of the diastereomeric isomer which is then subjected to diastereomeric resolution through the conventional method to diastereomeric resolution through the conventional method in the art to give the pure enantiomer. In additional, the enantiomers and diastereoisomer are generally isolated through chromatography which uses a chiral stationary phase and optionally combines with a chemical derivative method (such as carbamate generated from amine).

Compounds disclosed herein may contain unnatural proportions of atomic isotopes at one or more of the atoms that make up the compounds. For example, a compound may be labeled with a radioisotope such as tritium (³H), iodine-125 (¹²⁵I) or C-14 (¹⁴C). For another example, hydrogen can be replaced by heavy hydrogen to form a deuterated drug. The bond between deuterium and carbon is stronger than that between ordinary hydrogen and carbon. Compared with undeuterated drugs, deuterated drugs usually have the advantages of reduced toxic side effects, increased drug stability, enhanced efficacy, and prolonged biological half-life of the drug. All changes in the isotopic compositions of compounds disclosed herein, regardless of radioactivity are included within the scope of the present disclosure.

The term “optional” or “optionally” means that the subsequent event or condition may occur but not requisite, that the term includes the instance in which the event or condition occurs and the instance in which the event or condition does not occur.

The term “substituted” refers to that one or more than one hydrogen atoms on a specific atom are substituted by a substituent, including deuterium and hydrogen variants, as long as the valence of the specific atom is normal and the substituted compound is stable. When the substituent is oxo (i.e., ═O), it means that two hydrogen atoms are substituted. Positions on an aromatic ring cannot be substituted by oxo. The term “optionally substituted” means an atom can be substituted with a substitutent or not, unless otherwise specified, the type and the number of the substitutent may be arbitrary so long as being chemically achievable.

When any variable (e.g., R) occurs in the constitution or structure of the compound more than once, the definition of the variable at each occurrence is independent. Thus, for example, if a group is substituted by 0-2 R, the group can be optionally substituted by up to two R, wherein the option of R at each occurrence is independent. Moreover, the combination of the substituent and/or the variant thereof is allowed only when the combination results in a stable compound.

When the number of a linking group is 0, such as —(CH₂)₀—, it means that the linking group is a single bond.

When one of the variables is a single bond, it means that the two groups linked by the single bond are connected directly. For example, when L in X-L-Y represents a single bond, the structure of X-L-Y is actually X—Y.

Unless otherwise specified, Cₙ₋ₙ₊ₘ or Cₙ-Cₙ₊ₘ includes any specific case of n to n+m carbons, for example, C₁₋₁₂ includes C₁, C₂, C₃, C₄, C₅, C₆, C₇, C₈, C₉, C₁₀, C₁₁ and C₁₂, also includes any range from n to n+m, for example, C₁₋₁₂ includes C₁₋₃, C₁₋₆, C₁₋₉, C₃₋₆, C₃₋₉, C₃₋₁₂, C₆₋₉, C₆₋₁₂ and C₉₋₁₂, etc.; Similarly, n membered to n+m membered indicates the number of atoms on a ring is n to n+m, for example, 3-12 membered ring includes 3 membered ring, 4 membered ring, 5 membered ring, 6 membered ring, 7 membered ring, 8 membered ring, 9 membered ring, membered ring, 11 membered ring, and 12 membered ring, also includes any range from n to n+m, for example, 3-12 membered ring includes 3-6 membered ring, 3-9 membered ring, 5-6 membered ring, 5-7 membered ring, 6-7 membered ring, 6-8 membered ring, and 6-10 membered ring, etc.

Unless otherwise specified, “C₁₋₃ alkyl” refers to a linear or branched saturated hydrocarbon group composed of 1 to 3 carbon atoms. The C₁₋₃ alkyl includes C₁₋₂ and C₂₋₃ alkyl and the like, it can be monovalent (such as methyl), divalent (such as methylene), or multivalent (such as methine). Examples of C₁₋₃ alkyl include but not limited to methyl, ethyl, propyl, n-propyl, isopropyl and the like. In the present disclosure, “alkyl” also intends to include substituted alkyl, that is, one or more positions in alkyl are substituted, especially by 1-4 substituents, which can be substituted at any position. “Haloalkyl” refers to alkyl defined herein in which one or more hydrogens are replaced by the same or different halogens. Examples of haloalkyl include —CH₂Cl, —CH₂CF₃, —CH₂CCl₃, perfluoroalkyl (for example, —CF₃) and the like.

Unless otherwise specified, “C₃₋₆ cycloalkyl” refers to a saturated cyclic hydrocarbon group composed of 3 to 6 carbon atoms, which includes monocyclic and bicyclic systems, and the C₃₋₆ cycloalkyl includes C₃₋₅, C₄₋₅, and C₅₋₆ cycloalkyl and the like, it can be monovalent, divalent, or multivalent. Examples of C₃₋₆ cycloalkyl include but are not limited to cyclopropyl, cyclobutyl, cyclopentyl, cyclohexyl and the like. The cycloalkyl group can be optionally substituted by one or more substituents, wherein each substituent is independently selected from hydroxyl, alkyl, alkoxy, halogen, haloalkyl, amino, monoalkylamino, or dialkylamino.

Unless otherwise specified, “C₁₋₃ alkoxy” refers to an alkyl containing 1 to 3 carbon atoms that are connected to the rest of the molecule through an oxygen atom. The C₁₋₃ alkoxy includes C₁₋₂, C₂, and C₃ alkoxy and the like. Examples of C₁₋₃ alkoxy include but are not limited to methoxy, ethoxy, propoxy, n-propyloxy, isopropyl and the like.

Unless otherwise specified, “C₁₋₃ alkylthio” refers to an alkyl containing 1 to 3 carbon atoms connected to the rest of the molecule through a sulfur atom. The C₁₋₃ alkylthio includes C₁₋₂, C₂, and C₃ alkylthio and the like. Examples of C₁₋₃ alkylthio include but are not limited to methylthio, ethylthio, propylthio, n-propylthio, isopropylthio and the like.

Unless otherwise specified, “aryl” refers to a 6 to 14-membered, preferably 6- to 10-membered, all-carbon monocyclic or fused polycyclic (i.e., rings that shares a pair of adjacent carbon atoms) group having a conjugated n-electron system, for example, phenyl and naphthyl.

Unless otherwise specified, “benzene 5-7-membered heterocyclyl” refers to a benzene heterocyclyl, which has a benzene structure fused to a heterocyclyl ring. The “heterocyclyl” in “benzene heterocyclyl” represents a saturated cyclic group composed of 5-7 ring atoms, one or two of which are heteroatoms independently selected from O, S and N, and the rest are carbon atoms. Non-limiting Examples of benzene heterocyclyl include but are not limited to

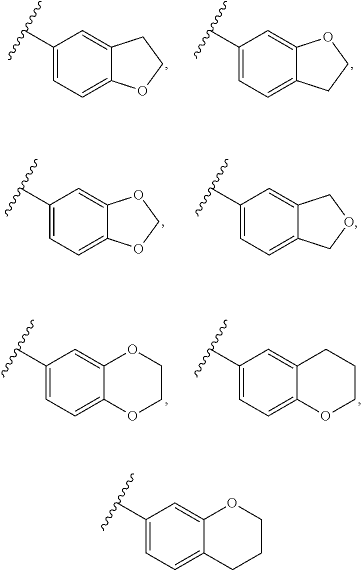

and the like.

Unless otherwise specified, “benzene 5-7-membered cycloalkyl” refers to benzene cycloalkyl, which has a benzene structure fused to a cycloalkyl ring. “cycloalkyl” in “benzene cycloalkyl” represents a saturated cyclic group composed of 5-7 carbon atoms. Examples of benzene-cycloalkyl include but are not limited to

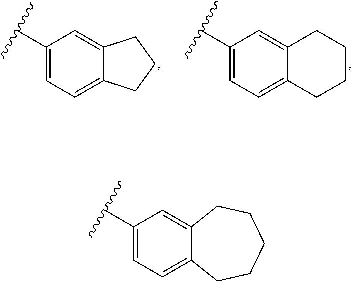 [SMILES: *c1ccc2c(c1)CCC2 ; *c1ccc2c(c1)CCCC2 ; *c1ccc2c(c1)CCCCC2]

and the like.

“Heteroaryl” refers to a heteroaromatic system containing 1 to 4 heteroatoms and 5 to 14 ring atoms, wherein the heteroatoms are selected from the group consisting of oxygen, sulfur and nitrogen. Heteroaryl is preferably 5 to 10-membered, and more preferably 5-membered or 6-membered, such as furanyl, thiophenyl, pyridinyl, pyrrolyl, N-alkylpyrrolyl, isoxazolyl, thiazolyl, oxazolyl, pyrimidinyl, pyrazinyl, dazinyl, imidazolyl, pyrazolyl, triazolyl, tetrazolyl,

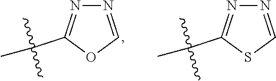 [SMILES: *c1nnco1 ; *c1nncs1]

and the like.

Unless otherwise specified, “heterocyclyl” itself or in combination with other terms separately represents a saturated or partially saturated cyclic group composed of 4-14 ring atoms, in which 1, 2, 3 or 4 ring atoms are heteroatoms independently selected from O, S and N, and the rest are carbon atoms, wherein the nitrogen atom is optionally quaternized, and the carbon, nitrogen and sulfur atoms can be optionally oxidized (such as C(═O), SO and SO₂). The ring comprises monocyclic, bicyclic and tricyclic systems, wherein the bicyclic and tricyclic systems include spirocyclic, fused cyclic and bridged cyclic rings. In addition, heteroatoms can occupy the connection position between the heterocyclyl and the rest of the molecule, and the heterocyclyl includes saturated as well as partially unsaturated heterocyclyl. Examples of heterocyclyl include but are not limited to azetidinyl, oxazetidinyl, thiazetidinyl, pyrrolidinyl, pyrazolyl, imidazolyl, tetrahydrothiophenyl (including tetrahydrothiophen-2-yl and tetrahydrothiophen-3-yl, etc.), tetrahydrofuranyl (including tetrahydrofuranyl-2-yl, etc.), tetrahydropyranyl, piperidinyl (including 1-piperidinyl, 2-piperidinyl, and 3-piperidinyl, etc.), piperazinyl (including 1-piperazinyl and 2-piperazinyl, etc.), morpholinyl (including 3-morpholinyl and 4-morpholinyl, etc.), dioxanyl, dithianyl, isoxazolidinyl, isothiazolidinyl, 1,2-oxazinyl, 1,2-thiazinyl, hexahydropyridazinyl, homopiperazinyl, homopiperidinyl,

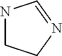

and the like.

The term “halo” or “halogen” refers to fluorine, chlorine, bromine and iodine atom.

The term “cyano” refers to —CN.

### Specific Pharmaceutical and Medical Terms

The term “acceptable,” as used herein, means that a prescription component or active ingredient does not have excessively harmful effects on the health of subject.

As used herein, the term “treatment,” “course of treatment” or “therapy” includes alleviating, inhibiting or improving symptoms or conditions of diseases; inhibiting the occurrence of complications; improving or preventing potential metabolic syndrome; suppressing the occurrence of diseases or symptoms, such as controlling the development of diseases or conditions; alleviating diseases or symptoms; reducing disease or symptoms; alleviating complications caused by diseases or symptoms, or preventing or treating symptoms caused by diseases or symptoms. As used herein, a certain compound or pharmaceutical composition, which can improve a certain disease, symptom or condition after administration, especially reduce its severity, delay the onset, slow down the progress of the disease, or shorten the duration of the disease. Whether fixed administration or temporary administration, continuous administration or intermittent administration, which can be attributed to related administration.

The term “active ingredient” refers to the compounds represented by the general formula (1) and the pharmaceutically acceptable inorganic or organic salts of the compounds of the general formula (1). Compounds of the present invention may contain one or more asymmetric centers, and thus appear in the form of racemates, racemic mixtures, single enantiomers, diastereomeric compounds or single diastereomers. The asymmetric centers that can exist depend on the properties of various substituents on the molecule. Each such asymmetric center will independently produce two optical isomers, and all possible optical isomers, diastereomer mixtures and pure or partially pure compounds are included in the scope of the present disclosure. The present disclosure is meant to include all such isomeric forms of these compounds.

The terms such as “compound,” “composition,” “agent” or “medicine or medicament” can be used interchangeably here, and all refer to a compound or composition which can induce the desired pharmaceutical and/or physiological response through local and/or systemic action when administered to a person (human or animal).

The term “administered, administering or administration” here refers to the direct administration of the compounds or compositions, or the administration of prodrugs, derivatives or analogs of the active compounds.

Although the numerical ranges and parameters used to define the wide range of the present invention are approximate values, the relevant values in the specific embodiments have been presented here as accurately as possible. However, any numerical value inevitably contains standard deviation caused by individual test methods. Here, “about” usually means that the actual value is within plus or minus 10%, 5%, 1% or 0.5% of a specific value or range. Alternatively, the word “about” means that the actual value falls within the acceptable standard error of the average value, depending on the consideration of those skilled in the art. Except for experimental examples, or unless otherwise specified, it is understood that all ranges, quantities, values and percentages used herein (for example, to describe the amount of materials, the length of time, the temperatures, the operating conditions, the proportions of quantities and other similar ones) are modified by “about”. Therefore, unless otherwise stated, the numerical parameters disclosed in this specification and the appended claims are approximate values, and can be changed as required. At least these numerical parameters should be understood as the indicated effective digits and the numerical values obtained by applying the general carry method.

Unless otherwise defined in this specification, the meanings of scientific and technical terms used herein are the same as those understood and used by those skilled in the art. In addition, the singular noun used in this specification covers the plural form of the noun without conflict with the context; the plural nouns used also cover the singular form of the noun.

### Route of Administration

The compound of the present invention and its pharmaceutically acceptable salts can be made into various preparations, which contain the compound of the present invention or its pharmaceutically acceptable salts and pharmaceutically acceptable excipients or carriers in a safe and effective amount range. Among them, “safe and effective amount” means that the amount of the compound is capable of obviously improving the condition without causing serious side effects. The safe and effective dose of the compound is determined according to the age, illness, course of treatment and other specific conditions of the subject.

“Pharmaceutically acceptable excipient or carrier” refers to one or more compatible solid or liquid fillers or gel substances, which are suitable for human use and must have sufficient purity and low toxicity. “Compatibility” here means that each component in the composition can be mixed with the compounds of the present invention and between them, without significantly reducing the efficacy of the compound. Examples of pharmaceutically acceptable excipients or carriers include cellulose and its derivatives (such as sodium carboxymethyl cellulose, sodium ethyl cellulose, cellulose acetate, etc.), gelatin, talc, solid lubricants (such as stearic acid and magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween®), wetting agent (such as sodium dodecyl sulfate), coloring agent, flavoring agent, stabilizer, antioxidant, preservative, pyrogen-free water, etc.

The compounds of the present invention can be administered orally, rectally, parenterally (intravenously, intramuscularly or subcutaneously) or topically.

Solid dosage forms for oral administration include capsules, tablets, pills, powders and granules. In these solid dosage forms, the active compound is mixed with at least one conventional inert excipient (or carrier), such as sodium citrate or dicalcium phosphate, or with: (a) a filler or compatibilizer, such as starch, lactose, sucrose, glucose, mannitol, and silicic acid; (b) binders such as hydroxymethyl cellulose, alginates, gelatin, polyvinyl pyrrolidone, sucrose and acacia; (c) humectant, such as glycerol; (d) disintegrant such as agar, calcium carbonate, potato or tapioca starch, alginic acid, certain complex silicates, and sodium carbonate: (e) slow solvents, such as paraffin; (f) an absorption accelerator, such as a quaternary amine compound; (g) wetting agents, such as cetyl alcohol and glyceryl monostearate; (h) adsorbent, such as kaolin; and (i) a lubricant such as talc, calcium stearate, magnesium stearate, solid polyethylene glycol, sodium lauryl sulfate, or mixtures thereof. In capsule, tablets and pill, the dosage form may also contain a buffer.

Solid dosage forms such as tablets, sugar pills, capsules, pills and granules may be prepared using coatings and shell materials such as casings and other materials well-known in the art. They may comprise an opacifying agent and the release of the active compound or compound in such a composition may be released in a delayed manner in a portion of the digestive tract. Examples of embedding components that may be used are polymeric substances and waxes. If desired, the active compound may also form microcapsules with one or more of the above excipients.

Liquid dosage forms for oral administration include pharmaceutically acceptable emulsions, solutions, suspensions, syrups or tinctures. In addition to the active compound, the liquid dosage form may comprise inert diluents conventionally used in the art, such as water or other solvents, solubilizers and emulsifiers, for example ethanol, isopropanol, ethyl carbonate, ethyl acetate, propylene glycol, 1,3-butanediol, dimethylformamide and oils, particularly cottonseed oil, peanut oil, corn germ oil, olive oil, castor oil, sesame oil or mixtures of these and the like.

In addition to these inert diluents, the composition may also contain adjuvants such as wetting agents, emulsifiers and suspending agents, sweeteners, flavoring agents and flavorants.

In addition to the active compounds, the suspension may comprise suspending agents such as ethoxylated isostearyl alcohol, polyoxyethylene sorbitol and sorbitan esters, microcrystalline cellulose, aluminum methoxide, agar or mixtures of these and the like.

The composition for parenteral injection may comprise a physiologically acceptable sterile aqueous or non-aqueous solution, dispersion, suspension or emulsion, and a sterile powder for reconstitution into a sterile injectable solution or dispersion. Suitable aqueous and non-aqueous carriers, diluents, solvents or excipients include water, ethanol, polyols and suitable mixtures thereof.

Dosage forms of the compounds of the present invention for topical administration include ointments, powders, patches, sprays and inhalants. The active ingredient is mixed under sterile conditions with a physiologically acceptable carrier and any preservatives, buffers, or propellants as may be required.

The compound of the present invention may be administered alone or in combination with other pharmaceutically acceptable compounds.

When a pharmaceutical composition is used, a safe and effective amount of a compound of the present invention is applied to a mammal (e.g., a human) in need of treatment, wherein the dose at the time of administration is a pharmaceutically acceptable effective dose, and for a human of 60 kg body weight, the daily dose is generally 1 to 2000 mg, preferably 50 to 1000 mg. Of course, specific dose should also take into account factors such as route of administration, patient health, etc., which are within the skill scope of a skilled physician.

The main advantages of the present disclosure include.
(a) Unexpectedly, compared to ABX464, the compound of the present disclosure showed excellent in vitro activity, and also demonstrated better in vivo pharmacokinetic properties, making it more suitable for development as a drug.
(b) The compound of the present disclosure has better pharmaceutical properties.
(c) Compared to ABX464, the compound of the present disclosure has significantly better efficacy in the treatment of inflammatory bowel disease in a mouse model induced by DSS.

The above features mentioned in the present disclosure, or the features mentioned in the examples, may be combined at random. All of the features disclosed in this specification may be used in any composition form and the various features disclosed in the specification may be replaced with any alternative feature that provides the same, equivalent, or similar purpose. Thus, unless otherwise specified, the disclosed features are merely generic examples of equivalent or similar features.

Various specific aspects, features and advantages of the above compounds, methods, and pharmaceutical compositions will be set forth in detail as following. It is to be understood that the following detailed description and examples describe specific embodiments for reference only. Various changes or modifications may occur to those skilled in the art after reading the description of the invention, and such equivalents fall within the scope of the application.

The structures of compounds disclosed herein can be confirmed by conventional methods well known to those skilled in the art. If the present disclosure relates to an absolute configuration of a compound, the absolute configuration can be confirmed by conventional techniques in the field. For example, single crystal X-ray diffraction (SXRD), the diffraction intensity data of the cultivated single crystal is collected using a Bruker D8 venture diffractometer with a light source of CuKα radiation in a scanning mode of φ/ω scan. After collecting the relevant data, the crystal structure is further analyzed by the direct method (Shelxs97) to conform the absolute configuration.

The solvent used in the present invention can be obtained commercially. The compounds of the present disclosure are named according to the conventional naming principles in the art or by ChemDraw® software, and the commercially available compounds use the supplier catalog names.

In all examples, ¹H-NMR was recorded with a Varian Mercury 400 NMR spectrometer and that chemical shift was expressed as δ (ppm); Silica gel for separation is 200-300 mesh without specific statement, and the ratio of eluents is volume ratio.

Abbreviations: CHCl₃ represents chloroform; Cu represents copper; Cs₂CO₃ represents cesium carbonate; EA (EtOAc) represents ethyl acetate; DCM represents dichloromethane; Dioxane represents 1,4-dioxane; DMF represents N,N-dimethylformamide; DMSO represents dimethyl sulfoxide; EtOH represents ethanol; Fe represents iron; h represents hour(s); K₂CO₃ represents potassium carbonate; LC-MS represents liquid chromatography-mass spectrometry; MeI (CH₃I) represents iodomethane; MeOH represents methanol; MS represents mass spectrometry; Na₂CO₃ represents sodium carbonate; NaH represents sodium hydride; NaOH represents sodium hydroxide: NMR represents nuclear magnetic resonance; Pd/C represents palladium on carbon; Pd(OAc)₂ represents palladium(II) acetate; PE represents petroleum ether; POCl₃ represents phosphorus oxychloride; PtO₂ represents platinum(IV) oxide; r. t. represents room temperature; SOCl₂ represents thionyl chloride; TEA (Et₃N) represents triethylamine; TLC represents thin-layer chromatography; THF represents tetrahydrofuran; Tf₂O represents trifluoromethanesulfonic anhydride; Tol represents toluene; TsCl represents p-toluenesulfonyl chloride; Xantphos represents 4,5-bis(diphenylphosphino)-9,9-dimethylxanthene; AcOH (HOAc) represents acetic acid; CDI represents carbonyldiimidazole; DIEA (DIPEA) represents diisopropylethylamine; NH₄Cl represents ammonium chloride.

The present invention is further described through the following examples. It should be understood that these examples are only used to illustrate the invention, but not indented to impose any limitations on the present invention. The experimental methods not specified in the following examples are usually carried out under conventional conditions, or according to the conditions recommended by the manufacturer. Unless otherwise stated, percentages and parts are weight percentages and weight parts.

### Preparation Example 1: Synthesis of 3,5-Dichloro-1-methylquinoxalin-2(1H)-one (Intermediate 1)

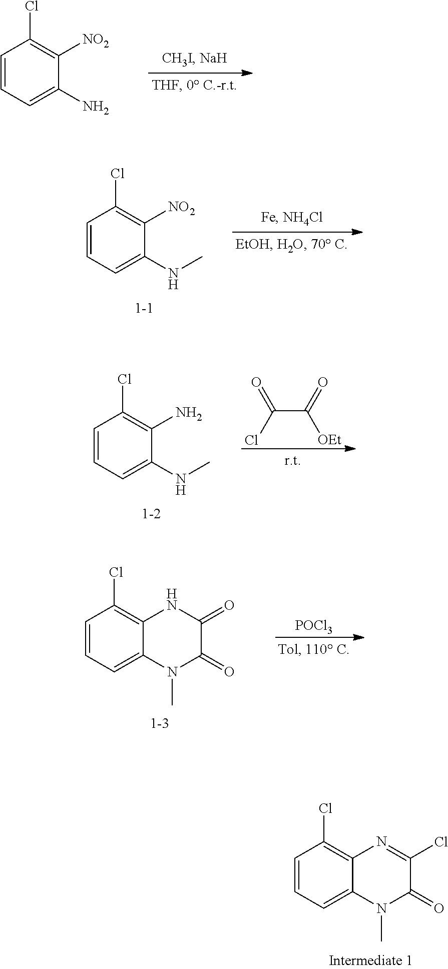

### Step 1: Synthesis of Compound 1-1

To a solution of 3-chloro-2-nitroaniline (3 g, 17.38 mmol) in THF (50 mL) was added NaH (765 mg, 19.12 mmol) slowly at 0° C. The resulting mixture was stirred for 30 min at 0° C. Then a solution of MeI (2.47 g, 17.38 mmol) in THF was added dropwise. The reaction was stirred at room temperature until the starting material was consumed. The reaction was then quenched with water and extracted with EA (50 mL*3) and dried over anhydrous Na₂SO₄. After filtration, the filtrate was concentrated under reduced pressure. The residue was purified by silica gel column chromatography (PE/EA=30/1 to 10/1) to afford a red solid 1-1 (1.2 g, yield: 37%), ESI-MS m/z: 186.9 [M+H]⁺.

### Step 2: Synthesis of Compound 1-2

To a solution of compound 1-1 (1.2 g, 6.45 mmol) in EtOH (30 mL) and H₂O (10 mL) was added Fe powder (1.81 g, 32.25 mmol) and NH₄Cl (3.45 g, 64.5 mmol). The resulting mixture was stirred at 70° C. until the starting material is consumed. The reaction was filtrated and then extracted with EA (30 mL*3) and dried over anhydrous Na₂SO₄. After filtration, the filtrate was concentrated under reduced pressure. The residue was purified by silica gel column chromatography (PE/EA=10/1 to 2/1) to afford yellow oil 1-2 (500 mg, yield: 50%), ESI-MS m/z: 156.9 [M+H]⁺.

### Step 3: Synthesis of Compound 1-3

Compound 1-2 (500 mg, 3.21 mmol) was dissolved in ethyl oxalyl monochloride (10 mL). The resulting mixture was stirred at room temperature until the starting material is consumed. The reaction was concentrated under reduced pressure. The residue was purified by silica gel column chromatography (PE/EA=30/1 to 10/1) to afford a yellow solid 1-3 (320 mg, yield: 47%), ESI-MS m/z: 210.8 [M+H]⁺.

### Step 4: Synthesis of Intermediate 1

Compound 1-3 (320 mg, 1.52 mmol) was dissolved in POCl₃ (10 mL). The resulting mixture was stirred at 110° C. until the starting material is consumed. The reaction was concentrated under reduced pressure and then quenched with water (50 mL). The resulting mixture was neutralized with saturated sodium bicarbonate and then extracted with EA (30 mL*3) and dried over anhydrous Na₂SO₄. After filtration, the filtrate was concentrated under reduced pressure. The residue was purified by silica gel column chromatography (PE/EA=10/1 to 1/1) to afford a yellow solid intermediate 1 (220 mg, yield: 63%), ESI-MS m/z: 228.8 [M+H]⁺.

### Preparation Example 2: Synthesis of 3,7-Dichloro-5-fluoro-1-methylquinolizine-2(11H)-one (Intermediate 2)

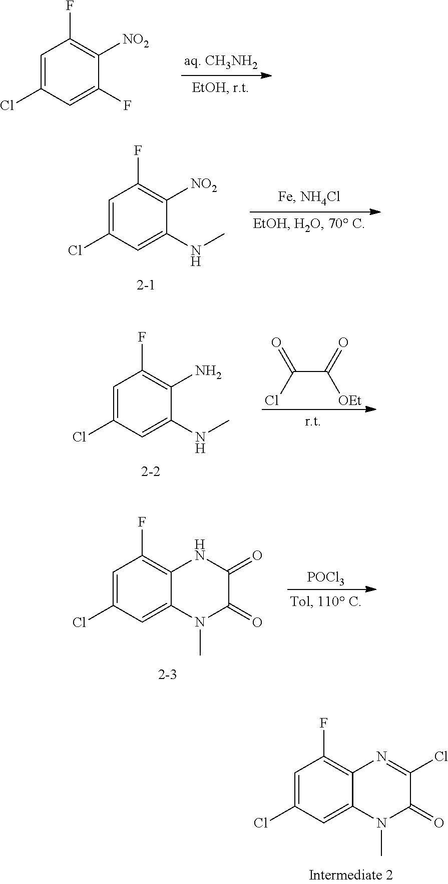

### Step 1: Synthesis of Compound 2-1

To a solution of 4-chloro-2,6-difluoronitrobenzene (2 g, 10.33 mmol) in EtOH (50 mL) was added methylamine water solution (1.07 g, 10.33 mmol, 30%). The resulting mixture was stirred at room temperature until the starting material is consumed. The reaction was concentrated under reduced pressure and then quenched with water (100 mL) and extracted with EA (50 mL*3) and dried over anhydrous Na₂SO₄. After filtration, the filtrate was concentrated under reduced pressure. The residue was purified by silica gel column chromatography (PE/EA=30/1 to 10/1) to afford a yellow solid 2-1 (1.5 g, yield: 71%), ESI-MS m/z: 204.8 [M+H]⁺.

Using compound 2-1 as the raw material, intermediate 2 can be synthesized by a method similar to the synthesis of intermediate 1, ESI-MS m/z: 247.0 [M+H]⁺.

### Preparation Example 3: Synthesis of 6-Chloroimidazo[1,2-a]quinoxalin-4-yl trifluoromethanesulfonate (Intermediate 3)

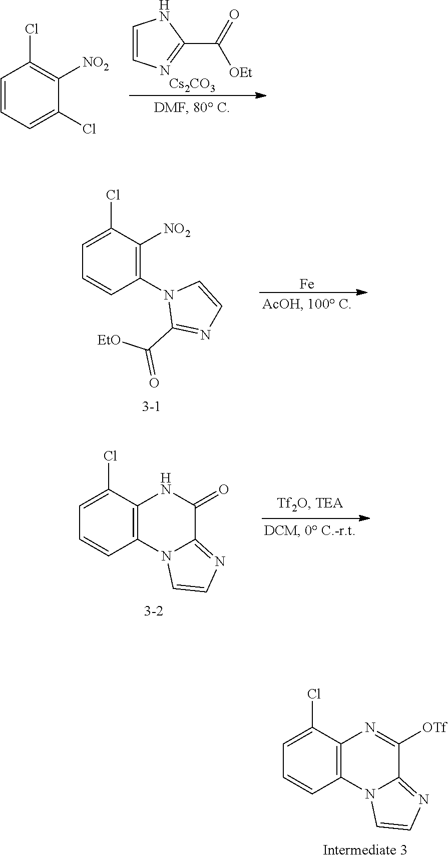

### Step 1: Synthesis of Compound 3-1

To a solution of 2,6-dichloronitrobenzene (2 g, 10.42 mmol) in DMF (50 mL) was added imidazole-2-carboxylic acid ethyl ester (1.46 g, 10.42 mmol) and Cs₂CO₃ (5.08 g, 15.63 mmol). The resulting mixture was stirred at 80° C. until the starting material is consumed. The reaction was then quenched with water (200 mL) and extracted with EA (50 mL*3) and dried over anhydrous Na₂SO₄. After filtration, the filtrate was concentrated under reduced pressure. The residue was purified by silica gel column chromatography (PE/EA=30/1 to 10/1) to afford a white solid 3-1 (2.7 g, yield: 88%), ESI-MS m/z: 295.9 [M+H]⁺.

### Step 2: Synthesis of Compound 3-2

To a solution of compound 3-1 (2.7 g, 9.15 mmol) in HOAc (30 mL) was added Fe powder (2.56 g, 45.76 mmol). The resulting mixture was stirred at 100° C. until the starting material is consumed. The reaction was concentrated under reduced pressure to remove HOAc and neutralized with saturated sodium bicarbonate. The resulting mixture was extracted with EA (50 mL*3) and dried over anhydrous Na₂SO₄. After filtration, the filtrate was concentrated under reduced pressure. The residue was purified by silica gel column chromatography (PE/EA=10/1 to 2/1) to afford a yellow solid 3-2 (1.1 g, yield: 55%), ESI-MS m/z: 219.8 [M+H]⁺.

### Step 3: Synthesis of Intermediate 3

To a solution of compound 3-2 (1.1 g, 5.02 mmol) in DCM (30 mL) was added TEA (1.01 g, 10.04 mmol) and Tf₂O (2.12 g, 7.53 mmol) slowly at 0° C. under an argon atmosphere. The resulting mixture was stirred at room temperature until the starting material is consumed. The reaction was then quenched with water (100 mL) and extracted with DCM (50 mL*3) and dried over anhydrous Na₂SO₄. After filtration, the filtrate was concentrated under reduced pressure. The residue was purified by silica gel column chromatography (PE/EA=50/1 to 20/1) to afford a white solid intermediate 3 (1.3 g, yield: 74%), ESI-MS m/z: 351.8 [M+H]⁺.

Using imidazole-2-carboxylic acid ethyl ester and corresponding substituted nitrobenzene as raw materials, intermediate 4-54 can be synthesized by a method similar to intermediate 3.

| | | | ESI-MS: |
|---|---|---|---|
| Compound | Structure | Name | [M + H]⁺ |
| Intermediate 4 | 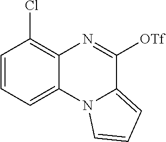 [SMILES: O=S(=O)(Oc1nc2c(Cl)cccc2n2cccc12)C(F)(F)F] | 6-chloropyrrolo[1,2-a] quinoxalin-4-yl trifluoromethanesulfonate | 350.8 |
| Intermediate 5 | 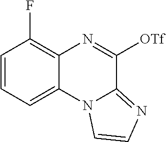 [SMILES: O=S(=O)(Oc1nc2c(F)cccc2n2ccnc12)C(F)(F)F] | 6-fluoroimidazo[1,2-a] quinoxalin-4-yl trifluoromethanesulfonate | 335.8 |
| Intermediate 6 | 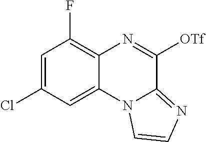 [SMILES: O=S(=O)(Oc1nc2c(F)cc(Cl)cc2n2ccnc12)C(F)(F)F] | 8-chloro-6-fluoroimidazo[1,2-a] quinoxalin-4-yl trifluoromethanesulfonate | 369.8 |
| Intermediate 7 | 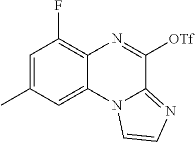 [SMILES: Cc1cc(F)c2nc(OS(=O)(=O)C(F)(F)F)c3nccn3c2c1] | 6-fluoro-8-methylimidazolo [1,2-a]quinoxalin-4-yl trifluoromethanesulfonate | 349.9 |
| Intermediate 8 | 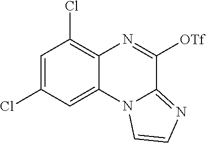 [SMILES: O=S(=O)(Oc1nc2c(Cl)cc(Cl)cc2n2ccnc12)C(F)(F)F] | 6,8-dichloroimidazolo [1,2-a]quinoxalin-4-yl trifluoromethanesulfonate | 385.9 |
| Intermediate 9 | 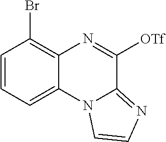 [SMILES: O=S(=O)(Oc1nc2c(Br)cccc2n2ccnc12)C(F)(F)F] | 6-bromoimidazo [1,2-a]quinoxalin-4-yl trifluoromethanesulfonate | 395.9 |
| Intermediate 10 | 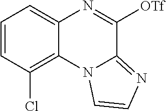 [SMILES: O=S(=O)(Oc1nc2cccc(Cl)c2n2ccnc12)C(F)(F)F] | 9-chloroimidazo [1,2-a]quinoxalin-4-yl trifluoromethanesulfonate | 351.8 |
| Intermediate 11 | 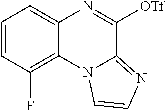 [SMILES: O=S(=O)(Oc1nc2cccc(F)c2n2ccnc12)C(F)(F)F] | 9-fluoroimidazo [1,2-a]quinoxalin-4-yl trifluoromethanesulfonate | 335.9 |
| Intermediate 12 | 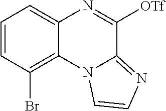 [SMILES: O=S(=O)(Oc1nc2cccc(Br)c2n2ccnc12)C(F)(F)F] | 9-bromoimidazolo [1,2-a]quinoxalin-4-yl trifluoromethanesulfonate | 395.8 |
| Intermediate 13 | 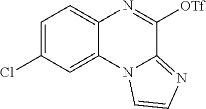 [SMILES: O=S(=O)(Oc1nc2ccc(Cl)cc2n2ccnc12)C(F)(F)F] | 8-chloroimidazo [1,2-a]quinoxalin-4-yl trifluoromethanesulfonate | 351.8 |
| Intermediate 14 | 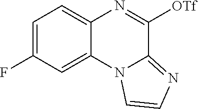 [SMILES: O=S(=O)(Oc1nc2ccc(F)cc2n2ccnc12)C(F)(F)F] | 8-fluoroimidazo [1,2-a]quinoxalin-4-yl trifluoromethanesulfonate | 335.9 |
| Intermediate 15 | 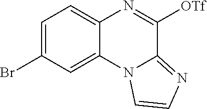 [SMILES: O=S(=O)(Oc1nc2ccc(Br)cc2n2ccnc12)C(F)(F)F] | 8-bromoimidazo [1,2-a]quinoxalin-4-yl trifluoromethanesulfonate | 395.8 |
| Intermediate 16 | 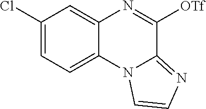 [SMILES: O=S(=O)(Oc1nc2cc(Cl)ccc2n2ccnc12)C(F)(F)F] | 7-chloroimidazo [1,2-a]quinoxalin-4-yl trifluoromethanesulfonate | 351.8 |
| Intermediate 17 | 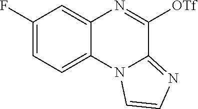 [SMILES: O=S(=O)(Oc1nc2cc(F)ccc2n2ccnc12)C(F)(F)F] | 7-fluoroimidazo [1,2-a]quinoxalin-4-yl trifluoromethanesulfonate | 335.9 |
| Intermediate 18 | 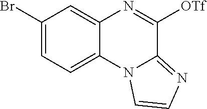 [SMILES: O=S(=O)(Oc1nc2cc(Br)ccc2n2ccnc12)C(F)(F)F] | 7-bromoimidazolo [1,2-a]quinoxalin-4-yl trifluoromethanesulfonate | 395.8 |
| Intermediate 19 | 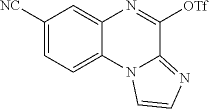 [SMILES: N#Cc1ccc2c(c1)nc(OS(=O)(=O)C(F)(F)F)c1nccn12] | 7-cyanoimidazolo [1,2-a]quinoxalin-4-yl trifluoromethanesulfonate | 342.8 |
| Intermediate 20 | 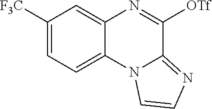 [SMILES: O=S(=O)(Oc1nc2cc(C(F)(F)F)ccc2n2ccnc12)C(F)(F)F] | 7-trifluoromethylimidazolo [1,2-a]quinoxalin-4-yl trifluoromethanesulfonate | 385.9 |
| Intermediate 21 | 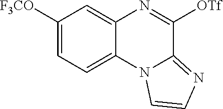 [SMILES: O=S(=O)(Oc1nc2cc(OC(F)(F)F)ccc2n2ccnc12)C(F)(F)F] | 7-trifluoromethoxyimidazo [1,2-a]quinoxalin-4-yl trifluoromethanesulfonate | 401.9 |
| Intermediate 22 | 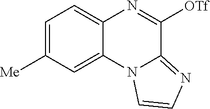 [SMILES: Cc1ccc2nc(OS(=O)(=O)C(F)(F)F)c3nccn3c2c1] | 8-methylimidazolo [1,2-a]quinoxalin-4-yl trifluoromethanesulfonate | 331.8 |
| Intermediate 23 | 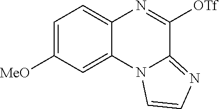 [SMILES: COc1ccc2nc(OS(=O)(=O)C(F)(F)F)c3nccn3c2c1] | 8-methoxyimidazo [1,2-a]quinoxalin-4-yl trifluoromethanesulfonate | 347.8 |
| Intermediate 24 | 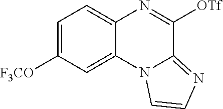 [SMILES: O=S(=O)(Oc1nc2ccc(OC(F)(F)F)cc2n2ccnc12)C(F)(F)F] | 8-trifluoromethoxyimidazo [1,2-a]quinoxalin-4-yl trifluoromethanesulfonate | 401.9 |
| Intermediate 25 | 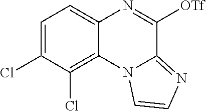 [SMILES: O=S(=O)(Oc1nc2ccc(Cl)c(Cl)c2n2ccnc12)C(F)(F)F] | 8,9-dichloroimidazo [1,2-a]quinoxalin-4-yl trifluoromethanesulfonate | 385.8 |
| Intermediate 26 | 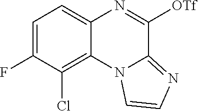 [SMILES: O=S(=O)(Oc1nc2ccc(F)c(Cl)c2n2ccnc12)C(F)(F)F] | 9-chloro-8-fluoroimidazo [1,2-a]quinoxalin-4-yl trifluoromethanesulfonate | 369.8 |
| Intermediate 27 | 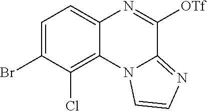 [SMILES: O=S(=O)(Oc1nc2ccc(Br)c(Cl)c2n2ccnc12)C(F)(F)F] | 8-bromo-7-chloroimidazolo [1,2-a]quinoxalin-4-yl trifluoromethanesulfonate | 429.7 |
| Intermediate 28 | 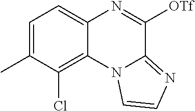 [SMILES: Cc1ccc2nc(OS(=O)(=O)C(F)(F)F)c3nccn3c2c1Cl] | 9-chloro-8-methylimidazolo [1,2-a]quinoxalin-4-yl trifluoromethanesulfonate | 365.9 |
| Intermediate 29 | 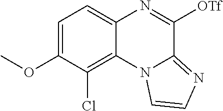 [SMILES: COc1ccc2nc(OS(=O)(=O)C(F)(F)F)c3nccn3c2c1Cl] | 9-chloro-8-methoxyimidazo [1,2-a]quinoxalin-4-yl trifluoromethanesulfonate | 381.9 |
| Intermediate 30 | 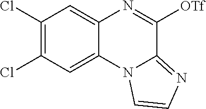 [SMILES: O=S(=O)(Oc1nc2cc(Cl)c(Cl)cc2n2ccnc12)C(F)(F)F] | 7,8-dichloroimidazolo [1,2-a]quinoxalin-4-yl trifluoromethanesulfonate | 385.8 |
| Intermediate 31 | 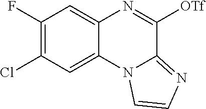 [SMILES: O=S(=O)(Oc1nc2cc(F)c(Cl)cc2n2ccnc12)C(F)(F)F] | 8-chloro-7-fluoroimidazo [1,2-a]quinoxalin-4-yl trifluoromethanesulfonate | 369.8 |
| Intermediate 32 | 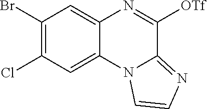 [SMILES: O=S(=O)(Oc1nc2cc(Br)c(Cl)cc2n2ccnc12)C(F)(F)F] | 7-bromo-8-chloroimidazolo [1,2-a]quinoxalin-4-yl trifluoromethanesulfonate | 429.7 |
| Intermediate 33 | 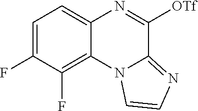 [SMILES: O=S(=O)(Oc1nc2ccc(F)c(F)c2n2ccnc12)C(F)(F)F] | 8,9-difluoroimidazo [1,2-a]quinoxalin-4-yl trifluoromethanesulfonate | 353.9 |
| Intermediate 34 | 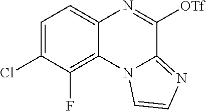 [SMILES: O=S(=O)(Oc1nc2ccc(Cl)c(F)c2n2ccnc12)C(F)(F)F] | 8-chloro-9-fluoroimidazo [1,2-a]quinoxalin-4-yl trifluoromethanesulfonate | 369.8 |
| Intermediate 35 | 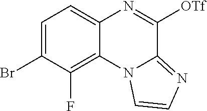 [SMILES: O=S(=O)(Oc1nc2ccc(Br)c(F)c2n2ccnc12)C(F)(F)F] | 8-bromo-9-fluoroimidazolo [1,2-a]quinoxalin-4-yl trifluoromethanesulfonate | 413.8 |
| Intermediate 36 | 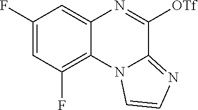 [SMILES: O=S(=O)(Oc1nc2cc(F)cc(F)c2n2ccnc12)C(F)(F)F] | 7,9-difluoroimidazo [1,2-a]quinoxalin-4-yl trifluoromethanesulfonate | 353.9 |
| Intermediate 37 | 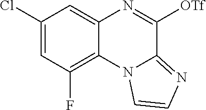 [SMILES: O=S(=O)(Oc1nc2cc(Cl)cc(F)c2n2ccnc12)C(F)(F)F] | 7-chloro-9-fluoroimidazo [1,2-a]quinoxalin-4-yl trifluoromethanesulfonate | 369.8 |
| Intermediate 38 | 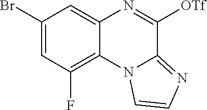 [SMILES: O=S(=O)(Oc1nc2cc(Br)cc(F)c2n2ccnc12)C(F)(F)F] | 7-bromo-9-fluoroimidazolo [1,2-a]quinoxalin-4-yl trifluoromethanesulfonate | 413.8 |
| Intermediate 39 | 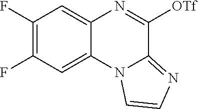 [SMILES: O=S(=O)(Oc1nc2cc(F)c(F)cc2n2ccnc12)C(F)(F)F] | 7,8-difluoroimidazolo [1,2-a]quinoxalin-4-yl trifluoromethanesulfonate | 353.9 |
| Intermediate 40 | 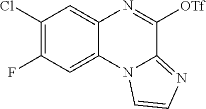 [SMILES: O=S(=O)(Oc1nc2cc(Cl)c(F)cc2n2ccnc12)C(F)(F)F] | 7-chloro-8-fluoroimidazo [1,2-a]quinoxalin-4-yl trifluoromethanesulfonate | 369.8 |
| Intermediate 41 | 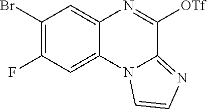 [SMILES: O=S(=O)(Oc1nc2cc(Br)c(F)cc2n2ccnc12)C(F)(F)F] | 7-bromo-8-fluoroimidazolo [1,2-a]quinoxalin-4-yl trifluoromethanesulfonate | 413.8 |
| Intermediate 42 | 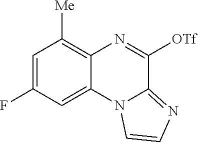 [SMILES: Cc1cc(F)cc2c1nc(OS(=O)(=O)C(F)(F)F)c1nccn12] | 8-fluoro-6-methylimidazolo [1,2-a]quinoxalin-4-yl trifluoromethanesulfonate | 349.8 |
| Intermediate 43 | 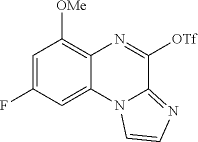 [SMILES: COc1cc(F)cc2c1nc(OS(=O)(=O)C(F)(F)F)c1nccn12] | 8-fluoro-6-methoxyimidazo [1,2-a]quinoxalin-4-yl trifluoromethanesulfonate | 365.8 |
| Intermediate 44 | 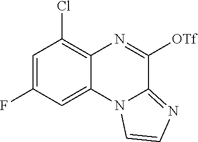 [SMILES: O=S(=O)(Oc1nc2c(Cl)cc(F)cc2n2ccnc12)C(F)(F)F] | 8-fluoro-6-cyanoimidazolo [1,2-a]quinoxalin-4-yl trifluoromethanesulfonate | 360.8 |
| Intermediate 45 | 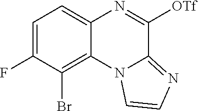 [SMILES: O=S(=O)(Oc1nc2ccc(F)c(Br)c2n2ccnc12)C(F)(F)F] | 9-bromo-8-fluoroimidazo [1,2-a]quinoxalin-4-yl trifluoromethanesulfonate | 413.8 |
| Intermediate 46 | 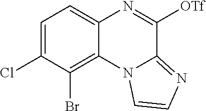 [SMILES: O=S(=O)(Oc1nc2ccc(Cl)c(Br)c2n2ccnc12)C(F)(F)F] | 9-bromo-8-chloroimidazolo [1,2-a]quinoxalin-4-yl trifluoromethanesulfonate | 429.7 |
| Intermediate 47 | 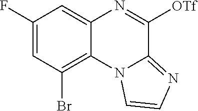 [SMILES: O=S(=O)(Oc1nc2cc(F)cc(Br)c2n2ccnc12)C(F)(F)F] | 9-bromo-7-fluoroimidazolo [1,2-a]quinoxalin-4-yl trifluoromethanesulfonate | 413.8 |
| Intermediate 48 | 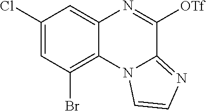 [SMILES: O=S(=O)(Oc1nc2cc(Cl)cc(Br)c2n2ccnc12)C(F)(F)F] | 9-bromo-7-chloroimidazolo [1,2-a]quinoxalin-4-yl trifluoromethanesulfonate | 429.7 |
| Intermediate 49 | 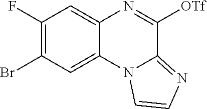 [SMILES: O=S(=O)(Oc1nc2cc(F)c(Br)cc2n2ccnc12)C(F)(F)F] | 8-bromo-7-fluoroimidazolo [1,2-a]quinoxalin-4-yl trifluoromethanesulfonate | 413.8 |
| Intermediate 50 | 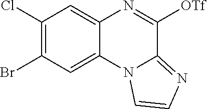 [SMILES: O=S(=O)(Oc1nc2cc(Cl)c(Br)cc2n2ccnc12)C(F)(F)F] | 8-bromo-7-chloroimidazo [1,2-a]quinoxalin-4-yl trifluoromethanesulfonate | 429.7 |
| Intermediate 51 | 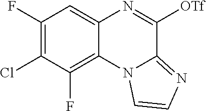 [SMILES: O=S(=O)(Oc1nc2cc(F)c(Cl)c(F)c2n2ccnc12)C(F)(F)F] | 8-chloro-7,9-difluoroimidazo [1,2-a]quinoxalin-4-yl trifluoromethanesulfonate | 387.8 |
| Intermediate 52 | 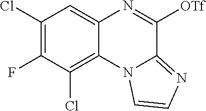 [SMILES: O=S(=O)(Oc1nc2cc(Cl)c(F)c(Cl)c2n2ccnc12)C(F)(F)F] | 7,9-dichloro-8-fluoroimidazo [1,2-a]quinoxalin-4-yl trifluoromethanesulfonate | 403.8 |
| Intermediate 53 | 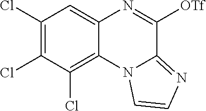 [SMILES: O=S(=O)(Oc1nc2cc(Cl)c(Cl)c(Cl)c2n2ccnc12)C(F)(F)F] | 7,8,9-trichloroimidazo [1,2-a]quinoxalin-4-yl trifluoromethanesulfonate | 419.7 |
| Intermediate 54 | 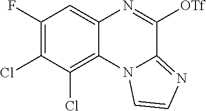 [SMILES: O=S(=O)(Oc1nc2cc(F)c(Cl)c(Cl)c2n2ccnc12)C(F)(F)F] | 8,9-dichloro-7-fluoroimidazo [1,2-a]quinoxalin-4-yl trifluoromethanesulfonate | 403.8 |

### Preparation Example 4: Synthesis of 4,6-dichloro-1,2,4-triazolo [4,3-a] quinoxaline (Intermediate 55)

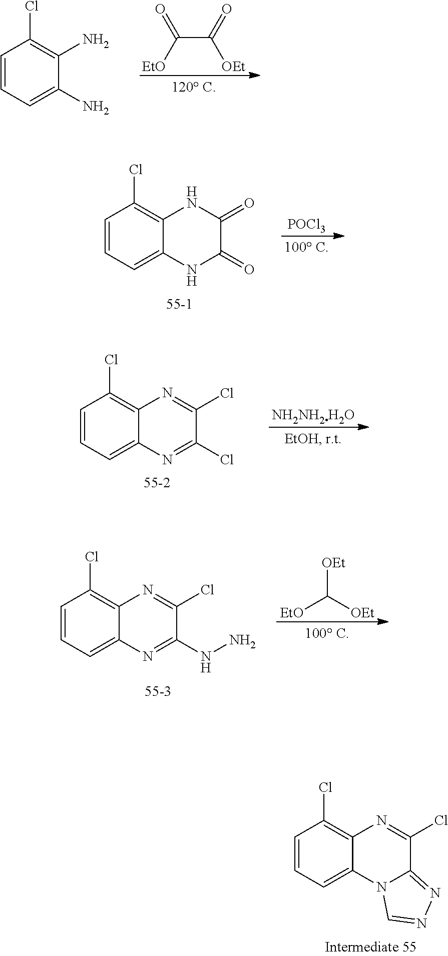

### Step 1: Synthesis of Compound 55-1

To a solution of 3-chloro-phenylenediamine (2 g, 14.03 mmol) in diethyl oxalate (20 mL) was stirred at 120° C. until the starting material was consumed. The reaction was concentrated under reduced pressure. The residue was purified by silica gel column chromatography (PE/EA=10/1 to 1/1) to afford a light yellow solid 55-1 (2.2 g, yield: 80%4), ESI-MS m/z: 196.9 [M+H]⁺.

### Step 2: Synthesis of Compound 55-2

Compound 55-1 (2.2 g, 11.22 mmol) was dissolved in POCl₃ (20 mL). The resulting mixture was stirred at 100° C. until the starting material is consumed. The reaction was concentrated under reduced pressure and then quenched with water (50 mL). The resulting mixture was neutralized with saturated sodium bicarbonate and then extracted with EA (30 mL*3) and dried over anhydrous Na₂SO₄. After filtration, the filtrate was concentrated under reduced pressure. The residue was purified by silica gel column chromatography (PE/EA=10/1 to 3/1) to afford a yellow solid 55-2 (1.6 g, yield: 62%), ESI-MS m/z: 232.8 [M+H]⁺.

### Step 3: Synthesis of Compound 55-3

To a solution of compound 55-2 (1.6 g, 6.87 mmol) in EtOH (20 mL) was added hydrazinium hydroxide solution (808 mg, 13.74 mmol, 85%). The resulting mixture was stirred at room temperature until the starting material is consumed. The reaction was concentrated under reduced pressure and then quenched with water (50 mL). The resulting mixture was extracted with EA (30 mL*3) and dried over anhydrous Na₂SO₄. After filtration, the filtrate was concentrated under reduced pressure to afford a light yellow solid 55-3 (1.8 g, crude), ESI-MS m/z: 228.9 [M+H]⁺.

### Step 4: Synthesis of Intermediate 55

Compound 55-3 (1.8 g, crude) was dissolved in triethyl orthoformate (20 mL). The resulting mixture was stirred at 100° C. until the starting material is consumed. The reaction was concentrated under reduced pressure and then quenched with water (50 mL). The resulting mixture was extracted with EA (30 mL*3) and dried over anhydrous Na₂SO₄. After filtration, the filtrate was concentrated under reduced pressure. The residue was purified by silica gel column chromatography (PE/EA=10/1 to 1/1) to afford a yellow solid 55 (1.1 g, yield: 67%), ESI-MS m/z: 238.8 [M+H]⁺.

Intermediate 55-3 was used as a common starting material and reacted with triethyl orthoacetate and trifluoroacetic acid, respectively, to afford Intermediate 56 and Intermediate 57.

| | | | ESI-MS: |
|---|---|---|---|
| Compound | Structure | Name | [M + H]⁺ |
| Intermediate 56 | 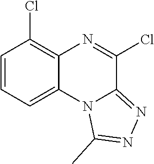 [SMILES: Cc1nnc2c(Cl)nc3c(Cl)cccc3n12] | 4,6-dichloro- 1-methyl-[1,2,4] triazolo[4,3-a] quinoxaline | 252.9 |
| Intermediate 57 | 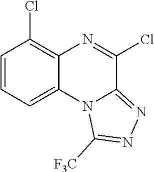 [SMILES: FC(F)(F)c1nnc2c(Cl)nc3c(Cl)cccc3n12] | 4,6-dichloro-1- (trifluoromethyl)- [1,2,4]triazolo[4,3-a] quinoxaline | 306.9 |

### Preparation Example 5: Synthesis of 4,6-dichloro-1,2-dihydroimidazo [1,2-a] quinoxaline (intermediate 58)

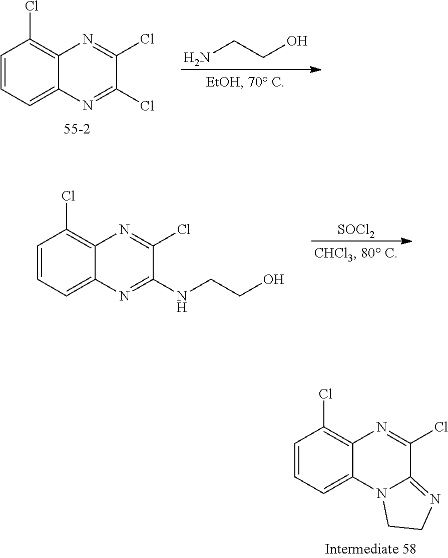

### Step 1: Synthesis of Compound 58-1

To a solution of 55-2 (1 g, 4.31 mmol) in EtOH (20 mL) was added ethanolamine (526 mg, 8.62 mmol). The resulting mixture was stirred at 70° C. until the starting material is consumed. The reaction was concentrated under reduced pressure and diluted with water (50 mL). The resulting mixture was extracted with EA (30 mL*3) and dried over anhydrous Na₂SO₄. After filtration, the filtrate was concentrated under reduced pressure. The residue was purified by silica gel column chromatography (PE/EA=10/1 to 1/1) to afford a red solid 58-1 (950 mg, yield: 86%), ESI-MS m/z: 257.9 [M+H]⁺.

### Step 2: Synthesis of Intermediate 58

Compound 58-1 (950 mg, 3.70 mmol) was dissolved in CHCl₃ (20 mL) and SOCl₂ (10 mL). The resulting mixture was stirred at 80° C. until the starting material is consumed. The reaction was poured to water (50 mL) and then neutralized with saturated sodium bicarbonate. The resulting mixture was extracted with CHCl₃ (30 mL*3) and dried over anhydrous Na₂SO₄. After filtration, the filtrate was concentrated under reduced pressure. The residue was purified by silica gel column chromatography (PE/EA=5/1 to 0/1) to afford a yellow solid intermediate 58 (500 mg, yield: 57%), ESI-MS m/z: 239.9 [M+H]⁺.

Intermediates 59-61 can be synthesized by a method similar to Intermediate 58, using variously substituted 1,2-diaminobenzenes as starting materials.

| | | | ESI-MS: |
|---|---|---|---|
| Compound | Structure | Name | [M + H]⁺ |
| Intermediate 59 | 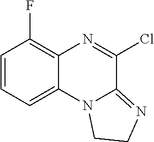 [SMILES: Fc1cccc2c1N=C(Cl)C1=NCCN12] | 4-chloro-6-fluoro-1,2- dihydroimidazo[1,2-a] quinoxaline | 223.9 |
| Intermediate 60 | 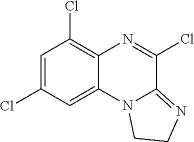 [SMILES: ClC1=Nc2c(Cl)cc(Cl)cc2N2CCN=C12] | 4,6,8-trichloro-1,2- dihydroimidazo[1,2-a] quinoxaline | 273.9 |
| Intermediate 61 | 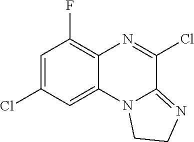 [SMILES: Fc1cc(Cl)cc2c1N=C(Cl)C1=NCCN12] | 4,8-dichloro-6-fluoro-1,2- dihydroimidazo[1,2-a] quinoxaline | 257.9 |

### Preparation Example 6: Synthesis of 6-Bromo-1,2-Dihydroimidazolo [1,2-a] quinoxalin-4-yl trifluoromethanesulfonate (Intermediate 62)

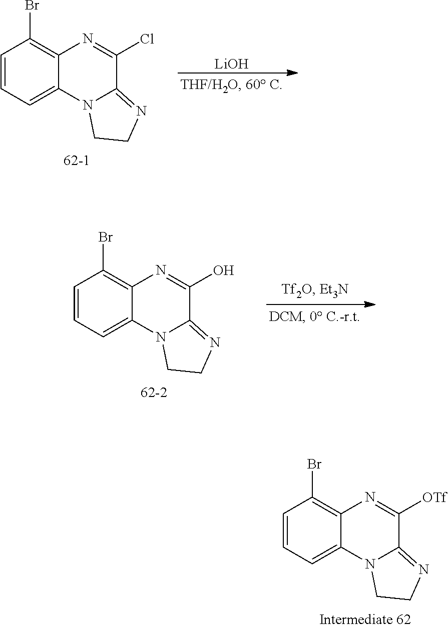 [SMILES: ClC1=Nc2c(Br)cccc2N2CCN=C12 ; "LiOH" ; OC1=Nc2c(Br)cccc2N2CCN=C12 ; O=S(=O)(OC1=Nc2c(Br)cccc2N2CCN=C12)C(F)(F)F]

Compound 62-1 can be synthesized by a method similar to Intermediate 58, using 3-bromo-1,2-diaminobenzene as a starting material.

### Step 1: Synthesis of Compound 62-2

To a solution of 62-1 (1 g, 3.51 mmol) in THF/H₂O (20 mL/4 mL) was added LiOH (281 mg, 7.02 mmol). The resulting mixture was stirred at 60° C. until the starting material is consumed. The reaction was then quenched with water (50 mL) and neutralized with 1 M HCl, and then extracted with EA (30 mL*3) and dried over anhydrous Na₂SO₄. After filtration, the filtrate was concentrated under reduced pressure to afford a yellow solid 62-2 (910 mg, yield: 97%), ESI-MS m/z: 266.0 [M+H]⁺.

### Step 2: Synthesis of Intermediate 62

To a solution of 62-2 (910 mg, 3.42 mmol) in DCM (20 mL) was added TEA (691 mg, 6.84 mmol) and Tf₂O (1.45 g, 5.13 mmol) slowly at 0° C. under an argon atmosphere. The resulting mixture was stirred at room temperature until the starting material is consumed. The reaction was then quenched with water (100 mL) and extracted with DCM (50 mL*3) and dried over anhydrous Na₂SO₄. After filtration, the filtrate was concentrated under reduced pressure. The residue was purified by silica gel column chromatography (PE/EA=10/1 to 3/1) to afford a white solid intermediate 62 (830 mg, yield: 61%), ESI-MS m/z: 397.9 [M+H]⁺.

### Preparation Example 7: Synthesis of 6-Chloro-1,2,4-triazolo [1,5-a] quinoxalin-4-yl trifluoromethanesulfonate (Intermediate 63)

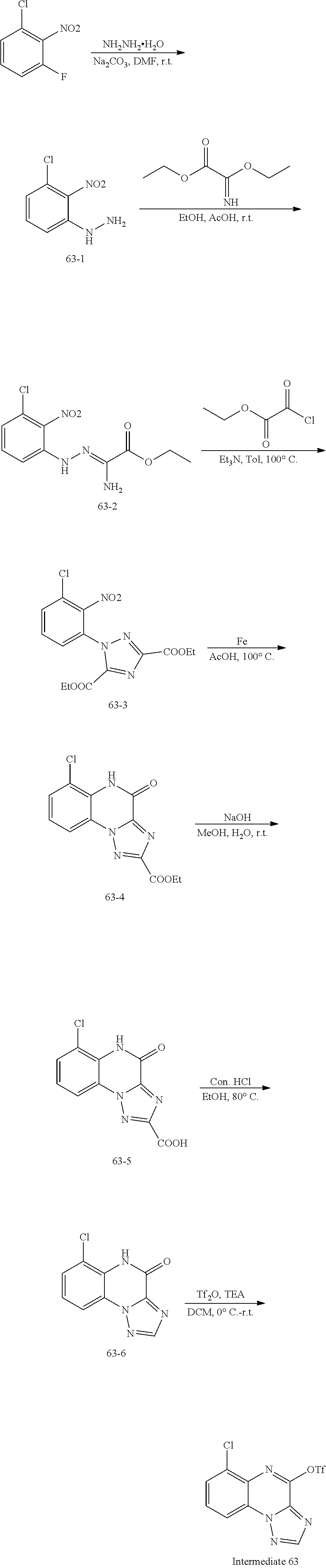

### Step 1: Synthesis of Compound 63-1

To a solution of 2-chloro-6-fluoronitrobenzene (2 g, 11.40 mmol) in DMF (50 mL) was added hydrazinium hydroxide solution (1.34 g, 22.80 mmol) and Na₂CO₃ (2.42 g, 22.80 mmol). The resulting mixture was stirred at room temperature until the starting material is consumed. The reaction was quenched with water (50 mL) and extracted with EA (30 mL*3) and dried over anhydrous Na₂SO₄. After filtration, the filtrate was concentrated to afford a light red solid 63-1 (1.7 g, yield: 80%), ESI-MS m/z: 187.9 [M+H]⁺.

### Step 2: Synthesis of Compound 63-2

To a solution of 63-1 (900 mg, 4.80 mmol) and ethyl 2-ethoxy-2-iminoacetate (835 mg, 5.76 mmol) in EtOH (20 mL) was added HOAc (1 mL). The resulting mixture was stirred at room temperature until the starting material is consumed. The reaction was concentrated under reduced pressure. The reaction was then diluted with water (50 mL) and extracted with EA (30 mL*3) and dried over anhydrous Na₂SO₄. After filtration, the filtrate was concentrated under reduced pressure. The residue was purified by silica gel column chromatography (PE/EA=20/1 to 5/1) to afford a light red solid 63-2 (1.2 g, yield: 88%), ESI-MS m/z: 286.9 [M+H]⁺.

### Step 3: Synthesis of Compound 63-3

To a solution of 63-2 (1.2 g, 4.2 mmol) in toluene (30 mL) was added TEA (1.27 g, 12.6 mmol) and ethyl oxalyl monochloride (1.15 g, 8.4 mmol). The resulting mixture was stirred at 110° C. until the starting material is consumed. The reaction was concentrated under reduced pressure. The reaction was then diluted with water (50 mL) and extracted with EA (30 mL*3) and dried over anhydrous Na₂SO₄. After filtration, the filtrate was concentrated under reduced pressure. The residue was purified by silica gel column chromatography (PE/EA=20/1 to 3/1) to afford a yellow solid 63-3 (1.2 g, yield: 77%), ESI-MS m/z: 368.9 [M+H]⁺.

### Step 4: Synthesis of Compound 63-4

To a solution of 63-3 (1.2 g, 3.26 mmol) in HOAc (30 mL) was added Fe powder (913 mg, 16.30 mmol). The resulting mixture was stirred at 100° C. until the starting material is consumed. The reaction was filtrated and concentrated under reduced pressure to remove HOAc, then extracted with EA (50 mL*3) and dried over anhydrous Na₂SO₄. After filtration, the filtrate was concentrated under reduced pressure. The residue was purified by silica gel column chromatography (PE/EA=10/1 to 2/1) to afford a yellow solid 63-4 (510 mg, yield: 54%), ESI-MS m/z: 292.9 [M+H]⁺.

### Step 5: Synthesis of Compound 63-5

To a solution of 63-4 (510 mg, 1.75 mmol) in MeOH (10 mL) and H₂O (2 mL) was added NaOH (140 mg, 3.50 mmol). The resulting mixture was stirred at room temperature until the starting material is consumed. The reaction was concentrated under reduced pressure and neutralized with 1 M HCl, then extracted with EA (50 mL*3) and dried over anhydrous Na₂SO₄. After filtration, the filtrate was concentrated under reduced pressure to afford a yellow solid 63-5 (500 mg, crude), ESI-MS m/z: 264.8 [M+H]⁺.

### Step 6: Synthesis of Compound 63-6

To a solution of 63-5 (500 mg, crude) in EtOH (10 mL) was added con·HCl (2 mL). The resulting mixture was stirred at 80° C. until the starting material is consumed. The reaction was concentrated under reduced pressure to remove EtOH and neutralized with saturated sodium bicarbonate, then extracted with EA (50 mL*3) and dried over anhydrous Na₂SO₄. After filtration, the filtrate was concentrated under reduced pressure. The residue was purified by silica gel column chromatography (PE/EA=10/1 to 1/1) to afford a yellow solid 63-6 (300 mg, yield: 78%), ESI-MS m/z: 220.8 [M+H]⁺.

### Step 7: Synthesis of Intermediate 63

To a solution of 63-6 (300 mg, 1.36 mmol) in DCM (10 mL) was added TEA (275 mg, 2.72 mmol) and Tf₂O (575 mg, 2.04 mmol) slowly at 0° C. under an argon atmosphere. The resulting mixture was stirred at room temperature until the starting material is consumed. The reaction was then quenched with water (100 mL) and extracted with DCM (50 mL*3) and dried over anhydrous Na₂SO₄. After filtration, the filtrate was concentrated under reduced pressure. The residue was purified by silica gel column chromatography (PE/EA=10/1 to 3/1) to afford a white solid intermediate 63 (330 mg, yield: 69%), ESI-MS m/z: 352.8 [M+H]⁺.

### Preparation Example 8: Synthesis of 6-Chloro-1,2,4-triazolo [1,5-a] quinoxalin-4-yl trifluoromethanesulfonate (Intermediate 64)

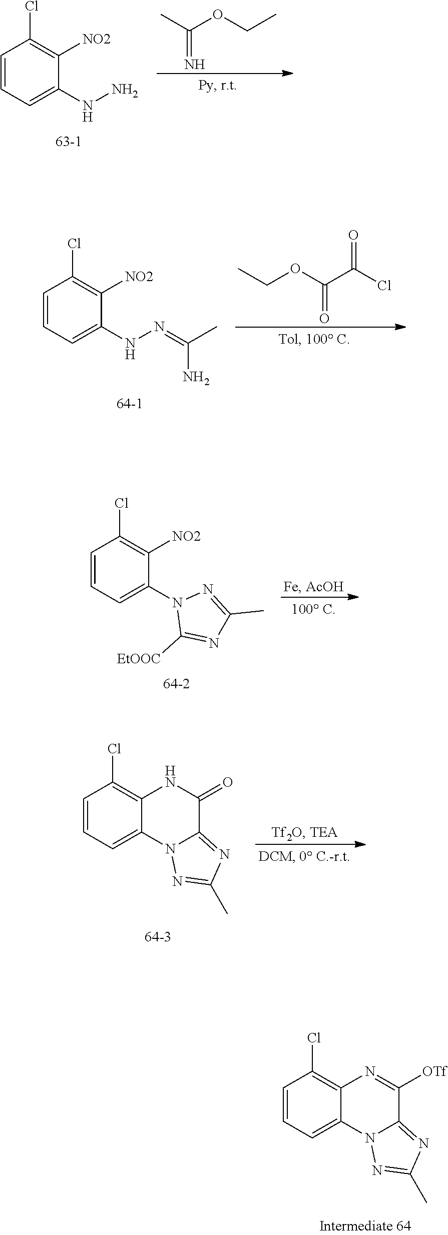

### Step 1: Synthesis of Compound 64-1

To a solution of 63-1 (1.5 g, 8.02 mmol) in Py (20 mL) was added ethyl acetamidate hydrochloride (990 mg, 8.02 mmol). The resulting mixture was stirred at room temperature until the starting material is consumed. The reaction was concentrated under reduced pressure and diluted with water (50 mL), then extracted with EA (30 mL*3) and dried over anhydrous Na₂SO₄. After filtration, the filtrate was concentrated under reduced pressure. The residue was purified by silica gel column chromatography (PE/EA=20/1 to 5/1) to afford a light red solid 64-1 (800 mg, yield: 44%), ESI-MS m/z: 228.9 [M+H]⁺.

### Step 2: Synthesis of Compound 64-2

To a solution of 64-1 (800 mg, 3.51 mmol) in toluene (20 mL) was added ethyl acetamidate hydrochloride (958 mg, 7.02 mmol). The resulting mixture was stirred at 110° C. until the starting material is consumed. The reaction was concentrated under reduced pressure and diluted with water (50 mL), then extracted with EA (30 mL*3) and dried over anhydrous Na₂SO₄. After filtration, the filtrate was concentrated under reduced pressure. The residue was purified by silica gel column chromatography (PE/EA=10/1 to 2/1) to afford a yellow solid 64-2 (680 mg, yield: 63%), ESI-MS m/z: 310.8 [M+H]⁺.

### Step 3: Synthesis of Compound 64-3

To a solution of 64-2 (680 mg, 2.19 mmol) in HOAc (10 mL) was added Fe powder (614 mg, 10.97 mmol). The resulting mixture was stirred at 100° C. until the starting material is consumed. The reaction was filtrated and concentrated under reduced pressure, then extracted with EA (50 mL*3) and dried over anhydrous Na₂SO₄. After filtration, the filtrate was concentrated under reduced pressure. The residue was purified by silica gel column chromatography (PE/EA=10/1 to 2/1) to afford a yellow solid 64-3 (210 mg, yield: 41%), ESI-MS m/z: 234.9 [M+H]⁺.

### Step 4: Synthesis of Intermediate 64

To a solution of 64-3 (210 mg, 0.90 mmol) in DCM (10 mL) was added TEA (182 mg, 1.80 mmol) and Tf₂O (381 mg, 1.35 mmol) slowly at 0° C., the resulting mixture was stirred at room temperature until the starting material is consumed. The reaction was then quenched with water (50 mL) and extracted with DCM (50 mL*3) and dried over anhydrous Na₂SO₄. After filtration, the filtrate was concentrated under reduced pressure. The residue was purified by silica gel column chromatography (PE/EA=10/1 to 3/1) to afford a white solid intermediate 64 (170 mg, yield: 52%), ESI-MS m/z: 366.8 [M+H]⁺.

### Preparation Example 9: Synthesis of 7,9-Dichloroimidazolo [1,2-a] quinoxalin-4-yl trifluoromethanesulfonate (Intermediate 65)

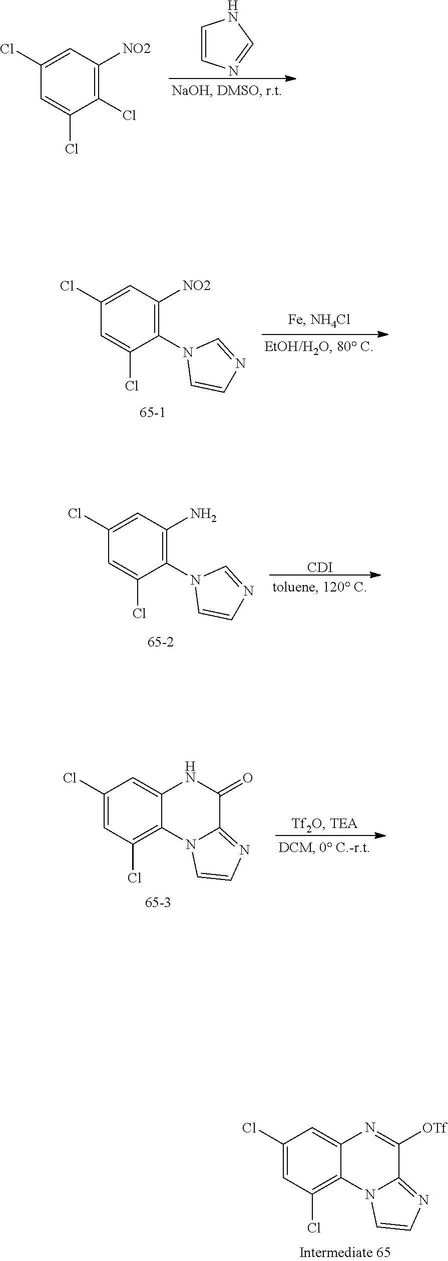

### Step 1: Synthesis of Compound 65-1

To a solution of 2,3,5-trichloronitrobenzene (2.00 g, 8.83 mmol) in DMSO (50 mL) was added imidazole (0.6 g, 8.83 mmol) and NaOH (706 mg, 17.66 mmol). The resulting mixture was stirred at room temperature until the starting material is consumed. The reaction was then quenched with water (100 mL) and extracted with EA (50 mL*3) and dried over anhydrous Na₂SO₄. After filtration, the filtrate was concentrated under reduced pressure. The residue was purified by silica gel column chromatography (PE/EA=30/1 to 10/1) to afford a white solid 65-1 (1.86 g, yield: 82%), ESI-MS m/z: 257.8 [M+H]⁺.

### Step 2: Synthesis of Compound 65-2

To a solution of 65-1 (1.86 g, 7.24 mmol) in EtOH/H₂O (10/1, 33 mL) was added Fe powder (2.03 g, 36.2 mmol) and NH₄Cl (1.94 g, 36.2 mmol). The resulting mixture was stirred at 80° C. until the starting material is consumed. The reaction was filtrated and then extracted with EA (50 mL*3) and dried over anhydrous Na₂SO₄. After filtration, the filtrate was concentrated under reduced pressure. The residue was purified by silica gel column chromatography (PE/EA=10/1 to 1/1) to afford a yellow solid 65-2 (1.4 g, yield: 85%), ESI-MS m/z: 227.8 [M+H]⁺.

### Step 3: Synthesis of Compound 65-3

To a solution of 65-2 (1.4 g, 6.17 mmol) in toluene (30 mL) was added CDI (1.5 g, 9.26 mmol). The resulting mixture was stirred at 120° C. until the starting material is consumed. The reaction was concentrated under reduced pressure and diluted with water (100 mL), then extracted with EA (50 mL*3) and dried over anhydrous Na₂SO₄. After filtration, the filtrate was concentrated under reduced pressure. The residue was purified by silica gel column chromatography (PE/EA=10/1 to 1/1) to afford a yellow solid 65-3 (1.17 g, yield: 75%), ESI-MS m/z: 253.8 [M+H]_{.}

### Step 4: Synthesis of Intermediate 65

To a solution of 65-3 (1.17 g, 4.62 mmol) in DCM (30 mL) was added TEA (934 mg, 9.25 mmol) and Tf₂O (1.56 g, 5.54 mmol) slowly at 0° C. under an argon atmosphere. The resulting mixture was stirred at room temperature until the starting material is consumed. The reaction was then quenched with water (100 mL) and extracted with DCM (50 mL*3) and dried over anhydrous Na₂SO₄. After filtration, the filtrate was concentrated under reduced pressure. The residue was purified by silica gel column chromatography (PE/EA=20/1 to 10/1) to afford a white solid intermediate 65 (1.46 g, yield: 82%), ESI-MS m/z: 385.7 [M+H]⁺.

Intermediates 66-67 can be synthesized by a method similar to Intermediate 65, using imidazole and variously substituted nitrobenzene as starting materials.

| | | | ESI-MS: |
|---|---|---|---|
| Compound | Structure | Name | [M + H]⁺ |
| Intermediate 66 | 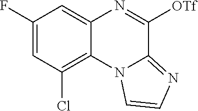 [SMILES: O=S(=O)(Oc1nc2cc(F)cc(Cl)c2n2ccnc12)C(F)(F)F] | 9-chloro-7-fluoroimidazo[1,2- a]quinoxalin-4-yl trifluoromethanesulfonate | 369.8 |
| Intermediate 67 | 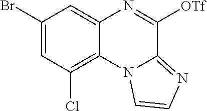 [SMILES: O=S(=O)(Oc1nc2cc(Br)cc(Cl)c2n2ccnc12)C(F)(F)F] | 7-bromo-9-chloroimidazolo [1,2-a]quinoxalin-4-yl trifluoromethanesulfonate | 429.7 |

### Preparation Example 10: Synthesis of 4-chloro-9-fluoro-1,2-dihydroimidazo [1,2-a] quinoxaline (Intermediate 68)

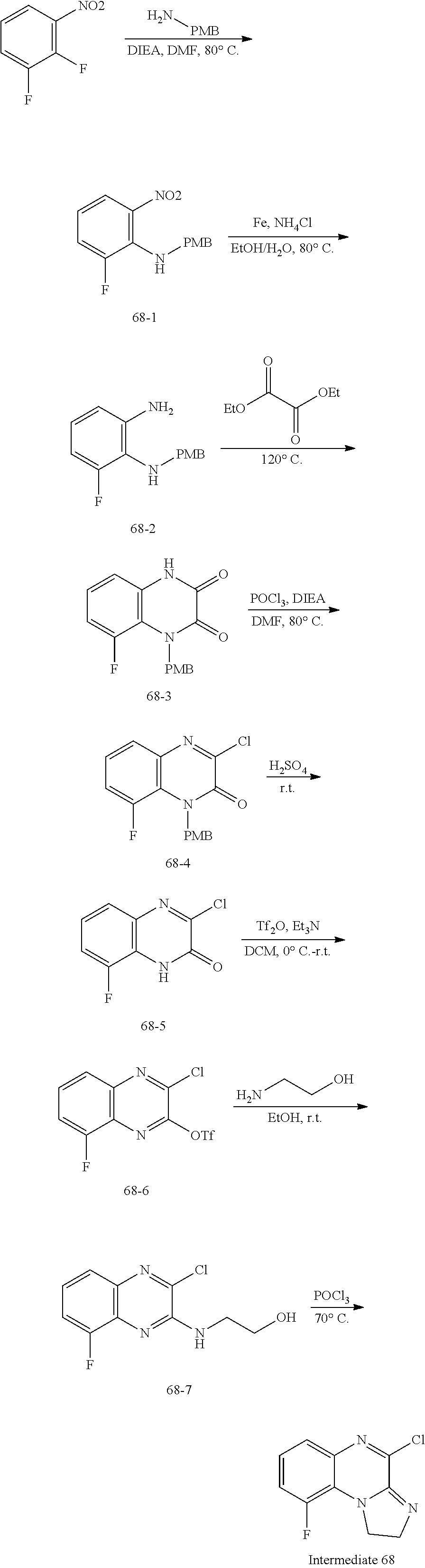

### Step 1: Synthesis of Compound 68-1

To a solution of 2,3-difluoronitrobenzene (5.00 g, 31.45 mmol) in DMF (100 mL) was added 4-methoxybenzylamine (4.31 g, 31.45 mmol) and DIEA (8.11 g, 62.9 mmol). The resulting mixture was stirred at 80° C. until the starting material is consumed. The reaction was then diluted with water (200 mL) and extracted with EA (50 mL*3) and dried over anhydrous Na₂SO₄. After filtration, the filtrate was concentrated under reduced pressure. The residue was purified by silica gel column chromatography (PE/EA=30/1 to 10/1) to afford a yellow solid 68-1 (5.9 g, yield: 68%), ESI-MS m/z: 276.9 [M+H]⁺.

### Step 2: Synthesis of Compound 68-2

To a solution of 68-1 (5.9 g, 21.38 mmol) in EtOH/H₂O (10/1, 110 mL) was added Fe powder (5.99 g, 106.9 mmol) and NH₄Cl (5.72 g, 106.9 mmol). The resulting mixture was stirred at 80° C. until the starting material is consumed. The reaction was filtrated and then extracted with EA (30 mL*3) and dried over anhydrous Na₂SO₄. After filtration, the filtrate was concentrated under reduced pressure. The residue was purified by silica gel column chromatography (PE/EA=30/1 to 10/1) to afford a yellow solid 68-2 (7.62 g, yield: 88%), ESI-MS m/z: 276.9 [M+H]⁺.

### Step 3: Synthesis of Compound 68-3

68-2 (7.62 g, 27.61 mmol) was dissolved in diethyl oxalate (50 mL). The resulting mixture was stirred at 120° C. until the starting material is consumed. The reaction was cooled to room temperature and filtrated to afford a white solid 68-3 (5.72 g, yield: 69%), ESI-MS m/z: 300.9 [M+H]⁺.

### Step 4: Synthesis of Compound 68-4

To a solution of 68-3 (5.72 g, 19.07 mmol) in DMF (50 mL) was added DIEA (4.92 g, 38.14 mmol) and POCl₃ (8.77 g, 57.21 mmol). The resulting mixture was stirred at 80° C. until the starting material is consumed. The reaction was quenched with water (200 mL) and extracted with EA (50 mL*3) and dried over anhydrous Na₂SO₄. After filtration, the filtrate was concentrated under reduced pressure. The residue was purified by silica gel column chromatography (PE/EA=10/1 to 5/1) to afford a yellow solid 68-4 (3.27 g, yield: 54%), ESI-MS m/z: 318.9 [M+H]⁺.

### Step 5: Synthesis of Compound 68-5

68-4 (3.27 g, 10.28 mmol) was dissolved in con·H₂SO₄ (10 mL). The resulting mixture was stirred at room temperature until the starting material is consumed. The reaction was quenched with water and then extracted with EA (50 mL*3) and dried over anhydrous Na₂SO₄. After filtration, the filtrate was concentrated under reduced pressure. The residue was purified by silica gel column chromatography (PE/EA=5/1 to 1/1) to afford a yellow solid 68-5 (1.32 g, yield: 65%), ESI-MS m/z: 198.8 [M+H]⁺.

### Step 6: Synthesis of Compound 68-6

To a solution of 68-5 (1.32 g, 6.67 mmol) in DCM (30 mL) was added TEA (1.35 g, 13.33 mmol) and Tf₂O (2.26 g, 8.00 mmol) slowly at 0° C. under an argon atmosphere. The resulting mixture was stirred at room temperature until the starting material is consumed. The reaction was then quenched with water (50 mL) and extracted with DCM (30 mL*3) and dried over anhydrous Na₂SO₄. After filtration, the filtrate was concentrated under reduced pressure. The residue was purified by silica gel column chromatography (PE/EA=10/1 to 3/1) to afford a yellow solid 68-6 (1.94 g, yield: 88%), ESI-MS m/z: 330.8 [M+H]⁺.

### Step 7: Synthesis of Compound 68-7

To a solution of 68-6 (1.94 g, 5.88 mmol) in EtOH (30 mL) was added ethanolamine (717 mg, 11.76 mmol). The resulting mixture was stirred at room temperature until the starting material is consumed. The reaction was concentrated under reduced pressure and diluted with water (200 mL). The resulting mixture was extracted with EA (50 mL*3) and dried over anhydrous Na₂SO₄. After filtration, the filtrate was concentrated under reduced pressure. The residue was purified by silica gel column chromatography (PE/EA=3/1 to 1/1) to afford a yellow solid 68-7 (1.08 g, yield: 76%), ESI-MS m/z: 241.8 [M+H]⁺.

### Step 8: Synthesis of Intermediate 68

68-7 (1.08 g, 4.48 mmol) was dissolved in POCl₃ (20 mL). The resulting mixture was stirred at 70° C. until the starting material is consumed. The reaction was concentrated under reduced pressure and quenched with water (100 mL), then extracted with EA (30 mL*3) and dried over anhydrous Na₂SO₄. After filtration, the filtrate was concentrated under reduced pressure. The residue was purified by silica gel column chromatography (PE/EA=5/1 to 0/1) to afford a yellow solid intermediate 68 (759 mg, yield: 76%), ESI-MS m/z: 223.8 [M+H]⁺.

Intermediates 69-74 can be synthesized by a method similar to Intermediate 68, using variously substituted nitrobenzene as starting materials.

| Compound | Structure | Name | ESI-MS: [M + H]⁺ |
|---|---|---|---|
| Intermediate 69 | 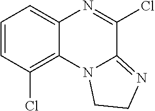 [SMILES: ClC1=Nc2cccc(Cl)c2N2CCN=C12] | 4,9-dichloro-1,2- dihydroimidazo[1,2-a] quinoxaline | 239.8 |
| Intermediate 70 | 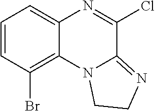 [SMILES: ClC1=Nc2cccc(Br)c2N2CCN=C12] | 4-chloro-9-bromo-1,2- dihydroimidazo[1,2-a] quinoxaline | 283.8 |
| Intermediate 71 | 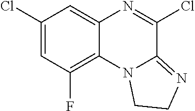 [SMILES: Fc1cc(Cl)cc2c1N1CCN=C1C(Cl)=N2] | 4,7-dichloro-9-fluoro-1,2- dihydroimidazo[1,2-a] quinoxaline | 257.8 |
| Intermediate 72 | 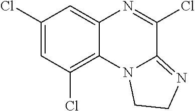 [SMILES: ClC1=Nc2cc(Cl)cc(Cl)c2N2CCN=C12] | 4,7,9-trichloro-1,2- dihydroimidazo[1,2-a] quinoxalin-4-yl trifluoromethanesulfonate | 273.8 |
| Intermediate 73 | 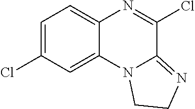 [SMILES: ClC1=Nc2ccc(Cl)cc2N2CCN=C12] | 4,8-dichloro-1,2- dihydroimidazo[1,2-a] quinoxaline | 239.8 |
| Intermediate 74 | 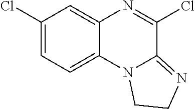 [SMILES: ClC1=Nc2cc(Cl)ccc2N2CCN=C12] | 4,7-dichloro-1,2- dihydroimidazo[1,2-a] quinoxaline | 239.8 |

### Example 1: Synthesis of 5-Chloro-1-methyl-3-((4-(trifluoromethoxy)phenyl)amino) quinoxalin-2(1H)-one (Compound 1)

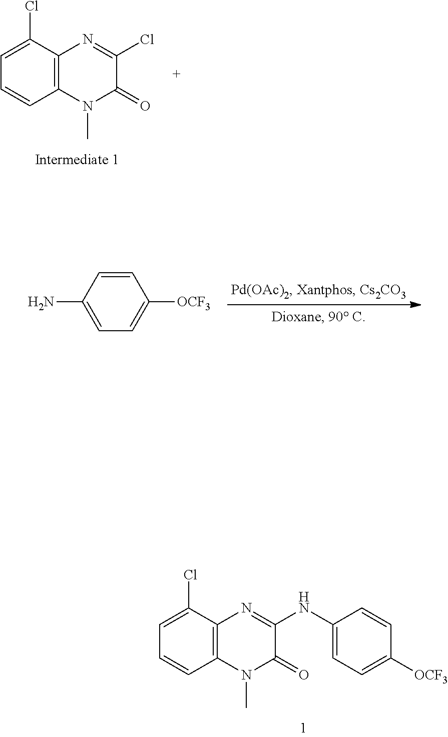 [SMILES: Cn1c(=O)c(Cl)nc2c(Cl)cccc21 ; Nc1ccc(OC(F)(F)F)cc1 ; Cn1c(=O)c(Nc2ccc(OC(F)(F)F)cc2)nc2c(Cl)cccc21]

To a solution of intermediate 1 (50 mg, 0.22 mmol) and 4-(trifluoromethoxy) aniline (39 mg, 0.22 mmol) in Dioxane (3 mL) was added Pd(OAc)₂ (5 mg, 0.022 mmol), Xantphos (26 mg, 0.044 mmol) and Cs₂CO₃ (143 mg, 0.44 mmol) under an argon atmosphere. The resulting mixture was stirred at 90° C. until the starting material is consumed. The reaction was diluted with water (20 mL) and extracted with EA (20 mL*3) and dried over anhydrous Na₂SO₄. After filtration, the filtrate was concentrated under reduced pressure. The residue was purified by silica gel column chromatography (PE/EA=10/1 to 1/1) to afford a yellow solid 1 (51 mg, yield: 64%).

¹H NMR (400 MHz, DMSO-d₆): δ 9.99 (s, 1H), 8.48 (d, J=9.2 Hz, 2H), 7.50-7.45 (m, 2H), 7.39 (d, J=8.7 Hz, 2H), 7.37-7.32 (m, 1H), 3.71 (s, 3H); ESI-MS m/z: 369.9 [M+H]⁺.

### Examples 2-94: Synthesis of Compounds 2-38 and 41-96

Compounds 2-38 and 41-96 can be synthesized by a method similar to Example 1, using Intermediates 1-9, 55-64 and corresponding amines as starting materials.

| | | ¹H NMR | ESI-MS: |
|---|---|---|---|
| Compound | Structure | (400 MHz, DMSO-d₆) δ | [M + H]⁺ |
| 2 | 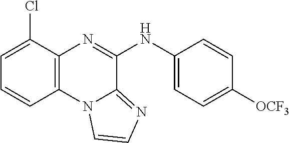 [SMILES: FC(F)(F)Oc1ccc(Nc2nc3c(Cl)cccc3n3ccnc23)cc1] | 10.33 (s, 1H), 8.79 (s, 1H), 8.57 (d, J = 9.2 Hz, 2H), 8.23 (d, J = 8.2 Hz, 1H), 7.81 (s, 1H), 7.70 (d, J = 6.9 Hz, 1H), 7.43 (dd, J = 17.2, 8.9 Hz, 3H) | 379.0 |
| 3 | 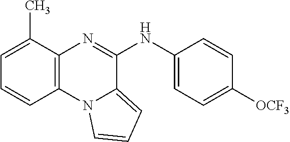 [SMILES: Cc1cccc2c1nc(Nc1ccc(OC(F)(F)F)cc1)c1cccn12] | 10.13 (s, 1H), 8.59 (s, 1H), 8.27 (d, J = 9.2 Hz, 2H), 8.03 (d, J = 8.2 Hz, 1H), 7.71 (s, 1H), 7.50 (d, J = 6.9 Hz, 1H), 7.03 (m, 4H) | 377.9 |
| 4 | 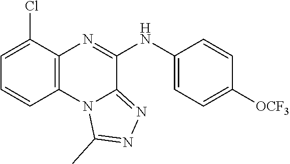 [SMILES: Cc1nnc2c(Nc3ccc(OC(F)(F)F)cc3)nc3c(Cl)cccc3n12] | 10.58 (s, 1H), 10.11 (s, 1H), 8.78 (d, J = 2.2 Hz, 1H), 8.26 (dd, J = 8.2, 1.1 Hz, 1H), 8.03 (dd, J = 8.9, 2.2 Hz, 1H), 7.74 (m, 2H), 7.42 (dd, J = 8.4, 7.6 Hz, 1H), 2.68 (s, 3H) | 394.0 |
| 5 | 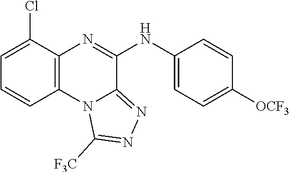 [SMILES: FC(F)(F)Oc1ccc(Nc2nc3c(Cl)cccc3n3c(C(F)(F)F)nnc23)cc1] | 10.97 (s, 1H), 8.65 (d, J = 2.1 Hz, 1H), 8.04 (dd, J = 8.9, 2.1 Hz, 1H), 7.82 (dd, J = 15.2, 8.1 Hz, 2H), 7.49 (t, J = 8.3 Hz, 1H), 7.41 (m, 2H) | 447.9 |
| 6 | 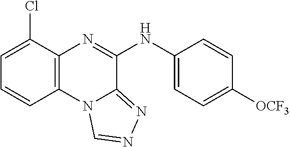 [SMILES: FC(F)(F)Oc1ccc(Nc2nc3c(Cl)cccc3n3cnnc23)cc1] | 10.88 (s, 1H), 10.10 (s, 1H), 8.78 (d, J = 2.1 Hz, 1H), 8.25 (dd, J = 8.2, 1.1 Hz, 1H), 8.09 (dd, J = 8.9, 2.2 Hz, 1H), 7.74 (m, 2H), 7.48 (dd, J = 8.4, 7.6 Hz, 2H) | 379.9 |
| 7 | 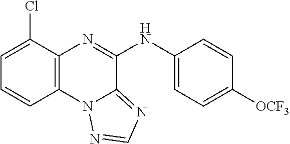 [SMILES: FC(F)(F)Oc1ccc(Nc2nc3c(Cl)cccc3n3ncnc23)cc1] | 10.58 (s, 1H), 10.15 (s, 1H), 8.77 (d, J = 2.2 Hz, 1H), 8.20 (dd, J = 8.2, 1.3 Hz, 1H), 8.01 (dd, J = 8.9, 2.3 Hz, 1H), 7.84 (m, 2H), 7.45 (dd, J = 8.5, 7.6 Hz, 2H) | 379.9 |
| 8 | 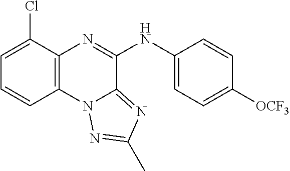 [SMILES: Cc1nc2c(Nc3ccc(OC(F)(F)F)cc3)nc3c(Cl)cccc3n2n1] | 10.64 (s, 1H), 8.82 (d, J = 2.2 Hz, 1H), 8.17 (dd, J = 8.2, 1.3 Hz, 1H), 8.13 (dd, J = 8.9, 2.1 Hz, 1H), 7.82 (dd, J = 7.9, 1.2 Hz, 1H), 7.55 (m, 3H), 2.60 (s, 3H) | 394.0 |
| 9 | 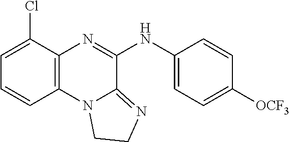 [SMILES: FC(F)(F)Oc1ccc(NC2=Nc3c(Cl)cccc3N3CCN=C23)cc1] | 9.76 (s, 1H), 8.42 (d, J = 9.1 Hz, 2H), 7.37 (d, J = 8.6 Hz, 2H), 7.20 (d, J = 4.6 Hz, 2H), 6.96-6.84 (m, 1H), 4.27- 4.11 (m, 4H) | 381.1 |
| 10 | 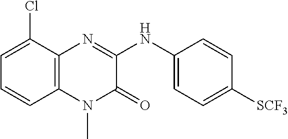 | 10.11 (s, 1H), 8.53 (d, J = 8.8 Hz, 2H), 7.72 (d, J = 8.7 Hz, 2H), 7.52-7.46 (m, 2H), 7.38 (d, J = 7.9 Hz, 1H), 3.71 (s, 3H) | 385.9 |
| 11 | 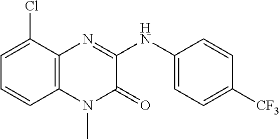 [SMILES: Cn1c(=O)c(Nc2ccc(C(F)(F)F)cc2)nc2c(Cl)cccc21] | 10.51 (s, 1H), 9.13 (d, J = 8.8 Hz, 2H), 8.52 (d, J = 8.7 Hz, 2H), 7.96 (m, 2H), 7.58 (d, J = 7.9 Hz, 1H), 3.91 (s, 3H) | 353.9 |
| 12 | 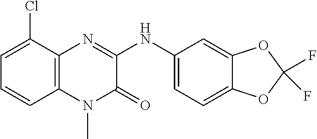 [SMILES: Cn1c(=O)c(Nc2ccc3c(c2)OC(F)(F)O3)nc2c(Cl)cccc21] | 10.06 (s, 1H), 8.71 (d, J = 2.1 Hz, 1H), 8.06 (dd, J = 8.9, 2.2 Hz, 1H), 7.47 (td, J = 7.9, 1.1 Hz, 2H), 7.42 (d, J = 8.9 Hz, 1H), 7.35 (d, J = 8.0 Hz, 1H), 3.70 (s, 3H) | 365.9 |
| 13 | 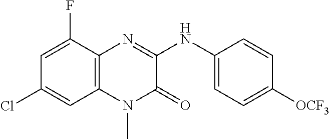 [SMILES: Cn1c(=O)c(Nc2ccc(OC(F)(F)F)cc2)nc2c(F)cc(Cl)cc21] | 10.11 (s, 1H), 8.58 (d, J = 9.2 Hz, 2H), 7.55 (m, 1H), 7.39 (m, 3H), 3.81 (s, 3H) | 387.9 |
| 14 | 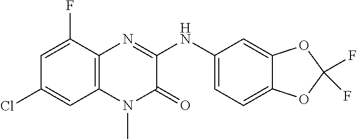 [SMILES: Cn1c(=O)c(Nc2ccc3c(c2)OC(F)(F)O3)nc2c(F)cc(Cl)cc21] | 10.03 (s, 1H), 8.40 (d, J = 2.1 Hz, 1H), 7.97 (dd, J = 8.9, 2.1 Hz, 1H), 7.44- 7.36 (m, 3H), 3.66 (s, 3H) | 383.9 |
| 15 | 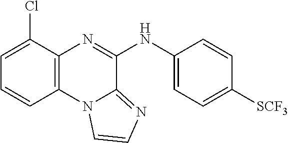 | 10.43 (s, 1H), 8.78 (d, J = 1.0 Hz, 1H), 8.60 (d, J = 8.8 Hz, 2H), 8.22 (dd, J = 8.2, 0.9 Hz, 1H), 7.81 (d, J = 1.0 Hz, 1H), 7.74-7.66 (m, 3H), 7.45 (t, J = 8.1 Hz, 1H) | 394.9 |
| 16 | 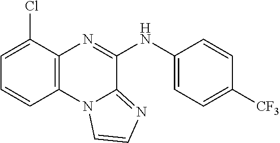 [SMILES: FC(F)(F)c1ccc(Nc2nc3c(Cl)cccc3n3ccnc23)cc1] | 10.50 (s, 1H), 8.81 (d, J = 1.0 Hz, 1H), 8.65 (d, J = 8.7 Hz, 2H), 8.24 (d, J = 7.4 Hz, 1H), 7.82 (d, J = 0.9 Hz, 1H), 7.72 (dd, J = 16.5, 7.9 Hz, 3H), 7.46 (t, J = 8.1 Hz, 1H) | 363.0 |
| 17 | 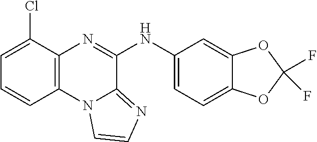 [SMILES: FC1(F)Oc2ccc(Nc3nc4c(Cl)cccc4n4ccnc34)cc2O1] | 10.33 (s, 1H), 8.80 (d, J = 2.1 Hz, 1H), 8.74 (d, J = 1.0 Hz, 1H), 8.20-8.16 (m, 1H), 8.05 (dd, J = 8.9, 2.1 Hz, 1H), 7.77 (d, J = 1.0 Hz, 1H), 7.65 (dd, J = 7.9, 0.9 Hz, 1H), 7.43-7.38 (m, 2H) | 374.9 |
| 18 | 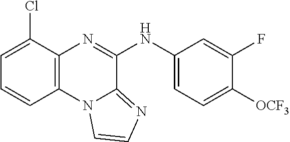 [SMILES: Fc1cc(Nc2nc3c(Cl)cccc3n3ccnc23)ccc1OC(F)(F)F] | 10.53 (s, 1H), 8.89 (dd, J = 14.2, 2.4 Hz, 1H), 8.78 (d, J = 0.9 Hz, 1H), 8.21 (d, J = 8.3 Hz, 1H), 8.16 (d, J = 8.4 Hz, 1H), 7.80 (d, J = 0.9 Hz, 1H), 7.68 (d, J = 7.9 Hz, 1H), 7.56 (t, J = 9.0 Hz, 1H), 7.45 (t, J = 8.1 Hz, 1H) | 396.9 |
| 19 | 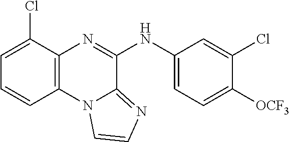 [SMILES: FC(F)(F)Oc1ccc(Nc2nc3c(Cl)cccc3n3ccnc23)cc1Cl] | 10.54 (s, 1H), 9.16 (d, J = 2.6 Hz, 1H), 8.79 (d, J = 1.1 Hz, 1H), 8.29 (dd, J = 9.1, 2.6 Hz, 1H), 8.23 (dd, J = 8.3, 1.0 Hz, 1H), 7.81 (d, J = 1.1 Hz, 1H), 7.70 (dd, J = 7.9, 1.0 Hz, 1H), 7.60-7.56 (m, 1H), 7.46 (t, J = 8.1 Hz, 1H) | 412.8 |
| 20 | 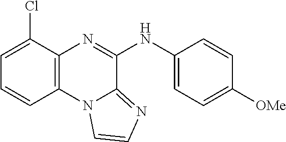 [SMILES: COc1ccc(Nc2nc3c(Cl)cccc3n3ccnc23)cc1] | 10.13 (s, 1H), 8.70 (s, 1H), 8.27 (d, J = 9.1 Hz, 2H), 8.03 (d, J = 8.1 Hz, 1H), 7.71 (s, 1H), 7.60 (d, J = 6.7 Hz, 1H), 7.43 (m, 3H), 3.93 (s, 3H) | 325.1 |
| 21 | 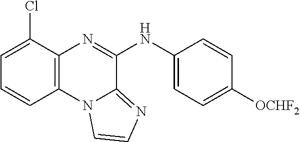 [SMILES: FC(F)Oc1ccc(Nc2nc3c(Cl)cccc3n3ccnc23)cc1] | 10.23 (s, 1H), 8.75 (s, 1H), 8.47 (d, J = 9.2 Hz, 2H), 8.13 (d, J = 8.1 Hz, 1H), 7.81 (s, 1H), 7.60 (d, J = 6.9 Hz, 1H), 7.43-7.01 (m, 4H) | 361.1 |
| 22 | 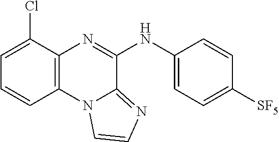 | 10.43 (s, 1H), 8.89 (s, 1H), 8.67 (d, J = 9.2 Hz, 2H), 8.33 (d, J = 8.4 Hz, 1H), 7.91 (s, 1H), 7.60 (m, 1H), 7.43 (m, 3H) | 420.8 |
| 23 | 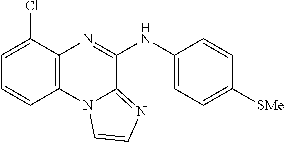 [SMILES: CSc1ccc(Nc2nc3c(Cl)cccc3n3ccnc23)cc1] | 10.03 (s, 1H), 8.50 (s, 1H), 8.17 (d, J = 9.1 Hz, 2H), 7.83 (d, J = 8.1 Hz, 1H), 7.51 (s, 1H), 7.20 (d, J = 6.7 Hz, 1H), 7.03 (m, 3H), 2.45 (s, 3H) | 340.9 |
| 24 | 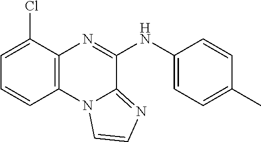 [SMILES: Cc1ccc(Nc2nc3c(Cl)cccc3n3ccnc23)cc1] | 10.23 (s, 1H), 8.70 (s, 1H), 8.27 (m, 2H), 7.99 (d, J = 8.1 Hz, 1H), 7.71 (s, 1H), 7.50 (d, J = 6.9 Hz, 1H), 7.33 (m, 3H), 2.15 (s, 3H) | 309.1 |
| 25 | 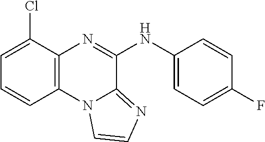 [SMILES: Fc1ccc(Nc2nc3c(Cl)cccc3n3ccnc23)cc1] | 10.43 (s, 1H), 8.90 (s, 1H), 8.47 (m, 2H), 8.29 (d, J = 8.2 Hz, 1H), 7.91 (s, 1H), 7.70 (d, J = 6.6 Hz, 1H), 7.43 (m, 3H) | 313.1 |
| 26 | 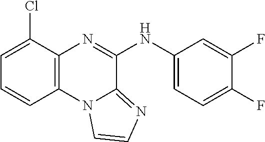 [SMILES: Fc1ccc(Nc2nc3c(Cl)cccc3n3ccnc23)cc1F] | 10.63 (s, 1H), 9.10 (s, 1H), 8.67 (m, 2H), 8.39 (d, J = 8.6 Hz, 1H), 7.99 (s, 1H), 7.75 (d, J = 6.7 Hz, 1H), 7.63 (m, 2H) | 331.1 |
| 27 | 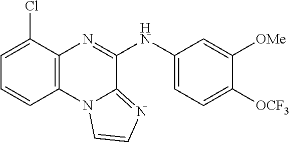 [SMILES: COc1cc(Nc2nc3c(Cl)cccc3n3ccnc23)ccc1OC(F)(F)F] | 10.33 (s, 1H), 8.70 (d, J = 2.1 Hz, 1H), 8.54 (d, J = 1.0 Hz, 1H), 8.26 (m, 1H), 8.01 (dd, J = 8.9, 2.1 Hz, 1H), 7.72 (d, J = 1.0 Hz, 1H), 7.60 (dd, J = 7.9, 0.9 Hz, 1H), 7.38 (m, 2H), 3.68 (s, 3H) | 409.1 |
| 28 | 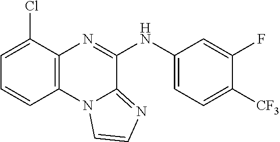 [SMILES: Fc1cc(Nc2nc3c(Cl)cccc3n3ccnc23)ccc1C(F)(F)F] | 10.75 (s, 1H), 8.91-8.79 (m, 2H), 8.25 (d, J = 8.2 Hz, 2H), 7.83 (s, 1H), 7.75 (dd, J = 19.5, 8.3 Hz, 2H), 7.49 (t, J = 8.1 Hz, 1H) | 381.1 |
| 29 | 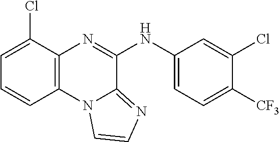 [SMILES: FC(F)(F)c1ccc(Nc2nc3c(Cl)cccc3n3ccnc23)cc1Cl] | 10.72 (s, 1H), 9.18 (s, 1H), 8.82 (s, 1H), 8.37 (d, J = 9.8 Hz, 1H), 8.25 (d, J = 8.2 Hz, 1H), 7.84 (d, J = 7.5 Hz, 2H), 7.72 (d, J = 7.8 Hz, 1H), 7.49 (t, J = 8.1 Hz, 1H) | 396.8 |
| 30 | 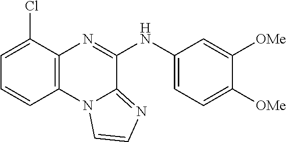 [SMILES: COc1ccc(Nc2nc3c(Cl)cccc3n3ccnc23)cc1OC] | 10.03 (s, 1H), 8.50 (d, J = 2.1 Hz, 1H), 8.24 (d, J = 1.2 Hz, 1H), 8.06 (m, 1H), 7.81 (dd, J = 8.9, 2.1 Hz, 1H), 7.42 (d, J = 1.0 Hz, 1H), 7.20 (dd, J = 7.9, 0.9 Hz, 1H), 7.08 (m, 2H), 3.78 (s, 3H), 3.68 (s, 3H) | 355.1 |
| 31 | 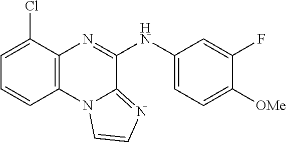 [SMILES: COc1ccc(Nc2nc3c(Cl)cccc3n3ccnc23)cc1F] | 10.13 (s, 1H), 9.01 (s, 1H), 8.57 (m, 2H), 8.19 (d, J = 8.6 Hz, 1H), 7.79 (s, 1H), 7.55 (d, J = 6.7 Hz, 1H), 7.33 (m, 2H), 3.88 (s, 3H) | 343.1 |
| 32 | 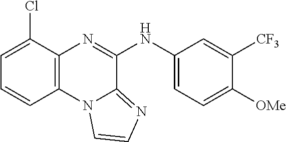 [SMILES: COc1ccc(Nc2nc3c(Cl)cccc3n3ccnc23)cc1C(F)(F)F] | 10.33 (s, 1H), 9.31 (s, 1H), 8.87 (m, 2H), 8.49 (d, J = 8.6 Hz, 1H), 7.89 (s, 1H), 7.65 (d, J = 6.7 Hz, 1H), 7.43 (m, 2H), 4.08 (s, 3H) | 393.1 |
| 33 | 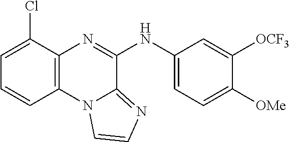 [SMILES: COc1ccc(Nc2nc3c(Cl)cccc3n3ccnc23)cc1OC(F)(F)F] | 10.03 (s, 1H), 9.11 (s, 1H), 8.77 (m, 2H), 8.29 (d, J = 8.6 Hz, 1H), 7.81 (s, 1H), 7.62 (d, J = 6.7 Hz, 1H), 7.33 (m, 2H), 3.68 (s, 3H) | 409.1 |
| 34 | 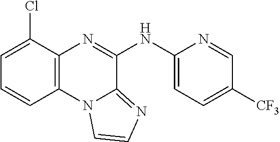 [SMILES: FC(F)(F)c1ccc(Nc2nc3c(Cl)cccc3n3ccnc23)nc1] | 11.03 (s, 1H), 10.29 (s, 1H), 9.27 (m, 2H), 8.93 (d, J = 8.4 Hz, 1H), 8.31 (s, 1H), 8.05 (d, J = 6.9 Hz, 1H), 7.83 (m, 2H) | 364.1 |
| 35 | 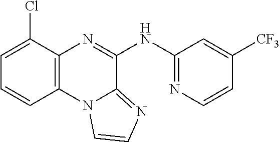 [SMILES: FC(F)(F)c1ccnc(Nc2nc3c(Cl)cccc3n3ccnc23)c1] | 11.13 (s, 1H), 10.19 (s, 1H), 9.37 (m, 2H), 8.83 (d, J = 8.4 Hz, 1H), 8.37 (s, 1H), 8.25 (d, J = 6.9 Hz, 2H), 7.93 (s, 1H) | 364.1 |
| 36 | 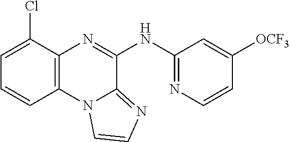 [SMILES: FC(F)(F)Oc1ccnc(Nc2nc3c(Cl)cccc3n3ccnc23)c1] | 10.53 (s, 1H), 9.69 (s, 1H), 9.07 (m, 2H), 8.53 (d, J = 8.4 Hz, 1H), 8.17 (s, 1H), 7.89 (d, J = 6.9 Hz, 2H), 7.63 (s, 1H) | 380.1 |
| 37 | 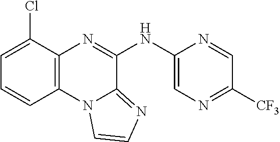 [SMILES: FC(F)(F)c1cnc(Nc2nc3c(Cl)cccc3n3ccnc23)cn1] | 11.13 (s, 1H), 10.39 (s, 1H), 9.37 (m, 2H), 8.95 (d, J = 8.4 Hz, 1H), 8.39 (s, 1H), 8.25 (d, J = 6.9 Hz, 1H), 7.93 (s, 1H) | 365.1 |
| 38 | 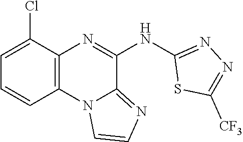 [SMILES: FC(F)(F)c1nnc(Nc2nc3c(Cl)cccc3n3ccnc23)s1] | 12.13 (s, 1H), 9.79 (s, 1H), 8.78 (d, J = 8.4 Hz, 1H), 8.31 (s, 1H), 8.10 (d, J = 6.5 Hz, 1H), 7.73 (m, 1H) | 370.8 |
| 41 | 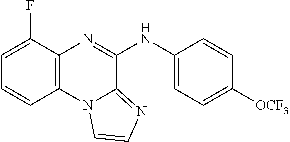 [SMILES: Fc1cccc2c1nc(Nc1ccc(OC(F)(F)F)cc1)c1nccn12] | 10.21 (s, 1H), 8.75 (s, 1H), 8.39 (d, J = 9.1 Hz, 2H), 8.05 (d, J = 7.9 Hz, 1H), 7.79 (s, 1H), 7.47-7.31 (m, 4H) | 363.1 |
| 42 | 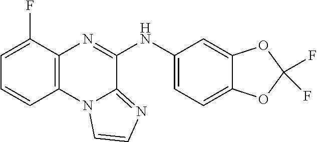 [SMILES: Fc1cccc2c1nc(Nc1ccc3c(c1)OC(F)(F)O3)c1nccn12] | 10.29 (s, 1H), 8.76 (s, 1H), 8.55 (d, J = 2.1 Hz, 1H), 8.09-7.97 (m, 2H), 7.79 (s, 1H), 7.51-7.30 (m, 3H) | 359.1 |
| 43 | 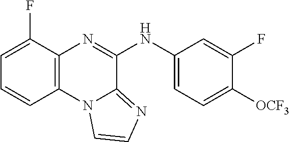 [SMILES: Fc1cc(Nc2nc3c(F)cccc3n3ccnc23)ccc1OC(F)(F)F] | 8.79 (d, J = 1.1 Hz, 1H), 8.69 (d, J = 2.5 Hz, 1H), 8.66 (d, J = 2.5 Hz, 1H), 8.15-8.06 (m, 2H), 7.82 (d, J = 1.0 Hz, 1H), 7.59 (t, J = 8.8 Hz, 1H), 7.51-7.38 (m, 2H) | 380.9 |
| 44 | 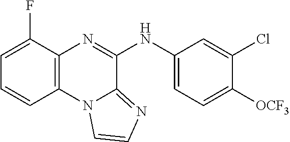 [SMILES: Fc1cccc2c1nc(Nc1ccc(OC(F)(F)F)c(Cl)c1)c1nccn12] | 10.46 (s, 1H), 8.86 (d, J = 2.5 Hz, 1H), 8.78 (s, 1H), 8.28 (dd, J = 9.1, 2.6 Hz, 1H), 8.07 (d, J = 8.0 Hz, 1H), 7.81 (s, 1H), 7.59 (d, J = 9.0 Hz, 1H), 7.50- 7.35 (m, 2H) | 396.9 |
| 45 | 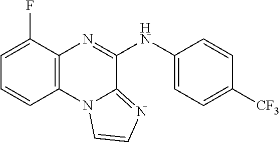 [SMILES: Fc1cccc2c1nc(Nc1ccc(C(F)(F)F)cc1)c1nccn12] | 10.41 (s, 1H), 8.79 (s, 1H), 8.51 (d, J = 8.6 Hz, 2H), 8.08 (d, J = 8.1 Hz, 1H), 7.82 (s, 1H), 7.74 (d, J = 8.7 Hz, 2H), 7.45 (dt, J = 18.7, 8.2 Hz, 2H) | 347.1 |
| 46 | 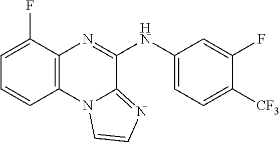 [SMILES: Fc1cc(Nc2nc3c(F)cccc3n3ccnc23)ccc1C(F)(F)F] | 10.66 (s, 1H), 8.79 (s, 1H), 8.65 (d, J = 14.5 Hz, 1H), 8.20 (d, J = 8.6 Hz, 1H), 8.08 (d, J = 8.1 Hz, 1H), 7.77 (dd, J = 21.8, 13.1 Hz, 2H), 7.52-7.35 (m, 2H) | 365.1 |
| 47 | 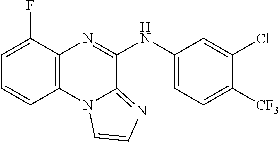 [SMILES: Fc1cccc2c1nc(Nc1ccc(C(F)(F)F)c(Cl)c1)c1nccn12] | 10.64 (d, J = 5.1 Hz, 1H), 8.89 (s, 1H), 8.80 (d, J = 2.7 Hz, 1H), 8.36 (d, J = 8.8 Hz, 1H), 8.12-8.05 (m, 1H), 7.84 (dd, J = 11.1, 2.2 Hz, 2H), 7.53-7.37 (m, 2H) | 381.1 |
| 48 | 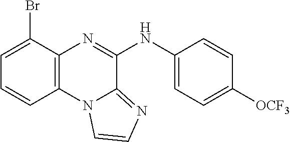 [SMILES: FC(F)(F)Oc1ccc(Nc2nc3c(Br)cccc3n3ccnc23)cc1] | 10.35 (s, 1H), 8.79 (s, 1H), 8.60 (d, J = 9.2 Hz, 2H), 8.26 (d, J = 8.2 Hz, 1H), 7.85 (d, J = 7.8 Hz, 1H), 7.81 (s, 1H), 7.42-7.34 (m, 3H) | 422.9 |
| 49 | 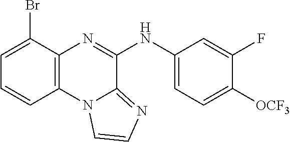 [SMILES: Fc1cc(Nc2nc3c(Br)cccc3n3ccnc23)ccc1OC(F)(F)F] | 10.56 (s, 1H), 8.95 (dd, J = 14.2, 2.5 Hz, 1H), 8.80 (s, 1H), 8.28 (d, J = 8.2 Hz, 1H), 8.21 (d, J = 9.8 Hz, 1H), 7.88 (d, J = 7.9 Hz, 1H), 7.82 (s, 1H), 7.57 (t, J = 8.9 Hz, 1H), 7.41 (t, J = 8.0 Hz, 1H) | 440.9 |
| 50 | 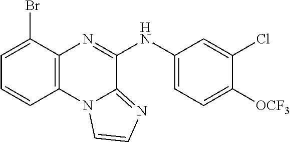 [SMILES: FC(F)(F)Oc1ccc(Nc2nc3c(Br)cccc3n3ccnc23)cc1Cl] | 10.55 (s, 1H), 9.23 (d, J = 2.6 Hz, 1H), 8.80 (s, 1H), 8.35-8.23 (m, 2H), 7.88 (d, J = 7.8 Hz, 1H), 7.82 (d, J = 1.0 Hz, 1H), 7.58 (d, J = 10.0 Hz, 1H), 7.40 (t, J = 8.1 Hz, 1H) | 456.9 |
| 51 | 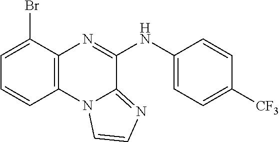 [SMILES: FC(F)(F)c1ccc(Nc2nc3c(Br)cccc3n3ccnc23)cc1] | 10.47 (s, 1H), 8.80 (s, 1H), 8.69 (d, J = 8.6 Hz, 2H), 8.27 (d, J = 8.2 Hz, 1H), 7.87 (d, J = 7.8 Hz, 1H), 7.82 (s, 1H), 7.74 (d, J = 8.7 Hz, 2H), 7.40 (t, J = 8.0 Hz, 1H) | 407.0 |
| 52 | 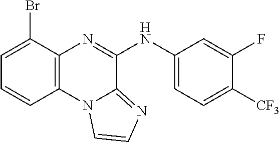 [SMILES: Fc1cc(Nc2nc3c(Br)cccc3n3ccnc23)ccc1C(F)(F)F] | 10.74 (s, 1H), 8.91 (d, J = 14.9 Hz, 1H), 8.81 (s, 1H), 8.29 (d, J = 8.3 Hz, 2H), 7.89 (d, J = 7.2 Hz, 1H), 7.84 (s, 1H), 7.76 (t, J = 8.7 Hz, 1H), 7.43 (t, J = 8.1 Hz, 1H) | 424.9 |
| 53 | 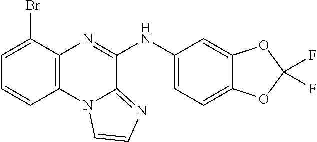 [SMILES: FC1(F)Oc2ccc(Nc3nc4c(Br)cccc4n4ccnc34)cc2O1] | 10.23 (s, 1H), 8.70 (d, J = 2.2 Hz, 1H), 8.64 (d, J = 1.1 Hz, 1H), 8.16 (m, 1H), 7.85 (dd, J = 8.8, 2.1 Hz, 1H), 7.67 (d, J = 1.0 Hz, 1H), 7.55 (m, 1H), 7.38 (m, 2H) | 418.9 |
| 54 | 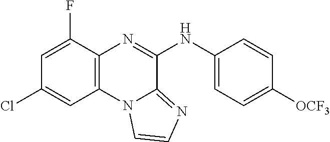 [SMILES: Fc1cc(Cl)cc2c1nc(Nc1ccc(OC(F)(F)F)cc1)c1nccn12] | 10.32 (s, 1H), 8.79 (d, J = 1.2 Hz, 1H), 8.39-8.33 (m, 2H), 8.31 (d, J = 1.8 Hz, 1H), 7.80 (d, J = 1.1 Hz, 1H), 7.59 (dd, J = 10.3, 2.1 Hz, 1H), 7.40 (d, J = 8.6 Hz, 2H) | 396.9 |
| 55 | 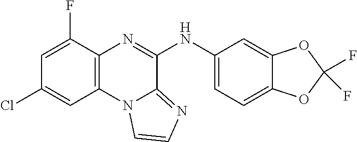 [SMILES: Fc1cc(Cl)cc2c1nc(Nc1ccc3c(c1)OC(F)(F)O3)c1nccn12] | 10.34 (s, 1H), 8.79 (d, J = 1.3 Hz, 1H), 8.49 (d, J = 2.1 Hz, 1H), 8.32-8.28 (m, 1H), 7.99 (dd, J = 8.9, 2.1 Hz, 1H), 7.79 (d, J = 1.2 Hz, 1H), 7.59 (dd, J = 10.3, 2.2 Hz, 1H), 7.43 (d, J = 8.9 Hz, 1H) | 392.9 |
| 56 | 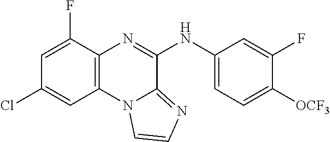 [SMILES: Fc1cc(Nc2nc3c(F)cc(Cl)cc3n3ccnc23)ccc1OC(F)(F)F] | 10.28 (s, 1H), 8.57 (s, 1H), 8.38 (d, J = 13.8 Hz, 1H), 8.08 (s, 1H), 7.87 (d, J = 8.9 Hz, 1H), 7.58 (s, 1H), 7.35 (t, J = 10.5 Hz, 2H) | 414.9 |
| 57 | 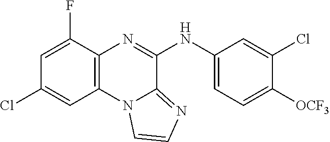 [SMILES: Fc1cc(Cl)cc2c1nc(Nc1ccc(OC(F)(F)F)c(Cl)c1)c1nccn12] | 10.52 (s, 1H), 8.81 (d, J = 2.4 Hz, 2H), 8.32 (d, J = 1.7 Hz, 1H), 8.25 (dd, J = 9.1, 2.6 Hz, 1H), 7.81 (d, J = 1.1 Hz, 1H), 7.60 (ddd, J = 7.2, 6.0, 1.6 Hz, 2H) | 430.9 |
| 58 | 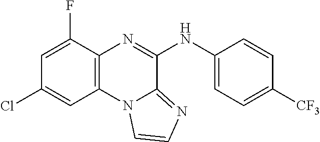 [SMILES: Fc1cc(Cl)cc2c1nc(Nc1ccc(C(F)(F)F)cc1)c1nccn12] | 10.42 (s, 1H), 8.80 (d, J = 0.9 Hz, 1H), 8.47 (d, J = 8.6 Hz, 2H), 8.30 (s, 1H), 7.81 (d, J = 1.0 Hz, 1H), 7.73 (d, J = 8.7 Hz, 2H), 7.58 (dd, J = 10.3, 1.9 Hz, 1H) | 380.9 |
| 59 | 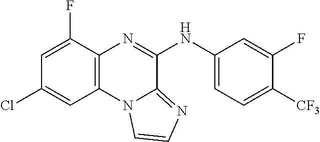 [SMILES: Fc1cc(Nc2nc3c(F)cc(Cl)cc3n3ccnc23)ccc1C(F)(F)F] | 10.71 (s, 1H), 8.81 (s, 1H), 8.59 (d, J = 14.5 Hz, 1H), 8.32 (s, 1H), 8.17 (d, J = 8.5 Hz, 1H), 7.82 (s, 1H), 7.75 (t, J = 8.7 Hz, 1H), 7.61 (d, J = 10.2 Hz, 1H) | 398.9 |
| 60 | 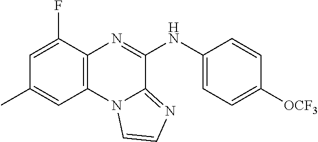 [SMILES: Cc1cc(F)c2nc(Nc3ccc(OC(F)(F)F)cc3)c3nccn3c2c1] | 9.42 (s, 1H), 7.80 (d, J = 0.9 Hz, 1H), 7.67 (m, 2H), 7.30 (s, 1H), 7.11 (d, J = 1.0 Hz, 1H), 7.03 (d, J = 8.7 Hz, 2H), 6.88 (m, 1H), 2.22 (s, 3H) | 377.1 |
| 61 | 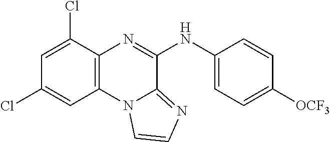 [SMILES: FC(F)(F)Oc1ccc(Nc2nc3c(Cl)cc(Cl)cc3n3ccnc23)cc1] | 10.37 (s, 1H), 8.80 (d, J = 1.0 Hz, 1H), 8.51 (d, J = 9.2 Hz, 2H), 8.44 (d, J = 2.2 Hz, 1H), 7.79 (d, J = 2.7 Hz, 2H), 7.39 (d, J = 8.7 Hz, 2H) | 412.9 |
| 62 | 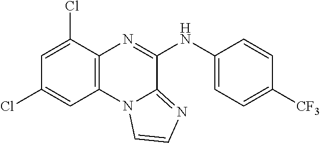 [SMILES: FC(F)(F)c1ccc(Nc2nc3c(Cl)cc(Cl)cc3n3ccnc23)cc1] | 10.54 (s, 1H), 8.84 (s, 1H), 8.62 (d, J = 8.6 Hz, 2H), 8.47 (d, J = 2.1 Hz, 1H), 7.83 (s, 2H), 7.75 (d, J = 8.7 Hz, 2H) | 396.9 |
| 63 | 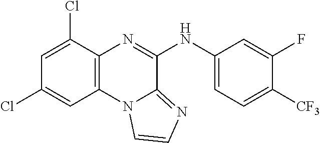 [SMILES: Fc1cc(Nc2nc3c(Cl)cc(Cl)cc3n3ccnc23)ccc1C(F)(F)F] | 10.71 (s, 1H), 8.79 (d, J = 1.0 Hz, 1H), 8.75 (d, J = 14.9 Hz, 1H), 8.41 (d, J = 2.2 Hz, 1H), 8.20 (d, J = 8.7 Hz, 1H), 7.79 (d, J = 1.0 Hz, 1H), 7.76 (d, J = 2.2 Hz, 1H), 7.72 (t, J = 8.7 Hz, 1H) | 414.9 |
| 64 | 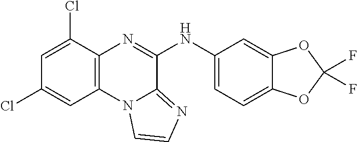 [SMILES: FC1(F)Oc2ccc(Nc3nc4c(Cl)cc(Cl)cc4n4ccnc34)cc2O1] | 10.22 (s, 1H), 8.69 (m, 1H), 8.39 (d, J = 2.0 Hz, 1H), 8.28 (m, 1H), 7.89 (m, 1H), 7.65 (d, J = 1.1 Hz, 1H), 7.47 (dd, J = 10.3, 2.1 Hz, 1H), 7.33 (d, J = 8.8 Hz, 1H) | 408.9 |
| 65 | 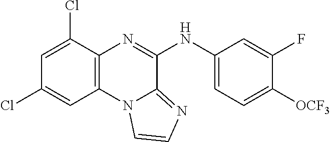 [SMILES: Fc1cc(Nc2nc3c(Cl)cc(Cl)cc3n3ccnc23)ccc1OC(F)(F)F] | 10.23 (s, 1H), 8.52 (s, 1H), 8.36 (d, J = 13.1 Hz, 1H), 8.05 (s, 1H), 7.86 (d, J = 8.8 Hz, 1H), 7.52 (s, 1H), 7.31 (m, 2H) | 430.9 |
| 66 | 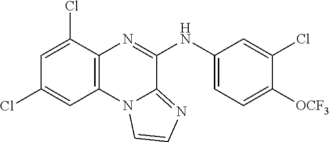 [SMILES: FC(F)(F)Oc1ccc(Nc2nc3c(Cl)cc(Cl)cc3n3ccnc23)cc1Cl] | 10.61 (s, 1H), 9.10 (d, J = 2.5 Hz, 1H), 8.83 (s, 1H), 8.47 (d, J = 1.9 Hz, 1H), 8.29 (dd, J = 9.1, 2.5 Hz, 1H), 7.82 (s, 2H), 7.59 (d, J = 9.1 Hz, 1H) | 446.9 |
| 67 | 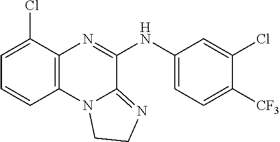 [SMILES: FC(F)(F)c1ccc(NC2=Nc3c(Cl)cccc3N3CCN=C23)cc1Cl] | 10.07 (s, 1H), 9.02 (s, 1H), 8.26 (d, J = 8.6 Hz, 1H), 7.80 (d, J = 8.9 Hz, 1H), 7.26-7.13 (m, 2H), 6.86 (dd, J = 7.6, 1.1 Hz, 1H), 4.15 (s, 4H) | 399.0 |
| 68 | 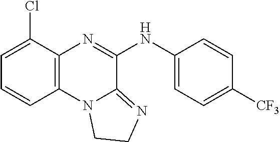 [SMILES: FC(F)(F)c1ccc(NC2=Nc3c(Cl)cccc3N3CCN=C23)cc1] | 9.82 (s, 1H), 8.52 (d, J = 8.6 Hz, 2H), 7.71 (d, J = 8.6 Hz, 2H), 7.22-7.13 (m, 2H), 6.85 (dd, J = 7.3, 1.8 Hz, 1H), 4.16 (s, 4H) | 365.1 |
| 69 | 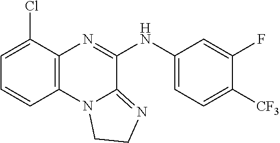 [SMILES: Fc1cc(NC2=Nc3c(Cl)cccc3N3CCN=C23)ccc1C(F)(F)F] | 10.10 (s, 1H), 8.72 (d, J = 14.4 Hz, 1H), 8.15 (d, J = 8.3 Hz, 1H), 7.72 (t, J = 8.7 Hz, 1H), 7.24-7.15 (m, 2H), 6.86 (dd, J = 7.6, 1.3 Hz, 1H), 4.15 (s, 4H) | 382.9 |
| 70 | 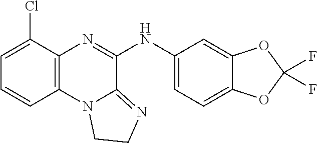 [SMILES: FC1(F)Oc2ccc(NC3=Nc4c(Cl)cccc4N4CCN=C34)cc2O1] | 9.51 (s, 1H), 8.29 (d, J = 2.1 Hz, 1H), 7.86 (dd, J = 8.9, 2.2 Hz, 1H), 7.39- 7.31 (m, 2H), 7.19 (dd, J = 8.0, 1.5 Hz, 1H), 6.96 (t, J = 7.9 Hz, 1H), 4.22 (s, 4H) | 377.1 |
| 71 | 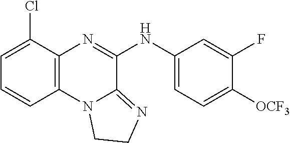 [SMILES: Fc1cc(NC2=Nc3c(Cl)cccc3N3CCN=C23)ccc1OC(F)(F)F] | 9.89 (s, 1H), 8.73 (dd, J = 14.1, 2.3 Hz, 1H), 8.05 (d, J = 9.1 Hz, 1H), 7.52 (t, J = 9.0 Hz, 1H), 7.19-7.11 (m, 2H), 6.82 (dd, J = 7.3, 1.7 Hz, 1H), 4.12 (s, 4H) | 399.1 |
| 72 | 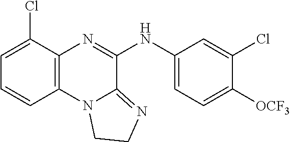 [SMILES: FC(F)(F)Oc1ccc(NC2=Nc3c(Cl)cccc3N3CCN=C23)cc1Cl] | 9.90 (s, 1H), 9.00 (d, J = 2.5 Hz, 1H), 8.17 (dd, J = 9.1, 2.5 Hz, 1H), 7.53 (d, J = 8.4 Hz, 1H), 7.20-7.12 (m, 2H), 6.83 (dd, J = 7.2, 1.9 Hz, 1H), 4.13 (s, 4H) | 414.9 |
| 73 | 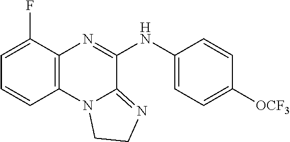 [SMILES: Fc1cccc2c1N=C(Nc1ccc(OC(F)(F)F)cc1)C1=NCCN12] | 9.57 (s, 1H), 8.27 (d, J = 9.1 Hz, 2H), 7.36 (d, J = 8.7 Hz, 2H), 7.16 (dt, J = 13.7, 6.9 Hz, 1H), 6.97-6.84 (m, 1H), 6.69 (d, J = 7.7 Hz, 1H), 4.13 (s, 4H) | 365.1 |
| 74 | 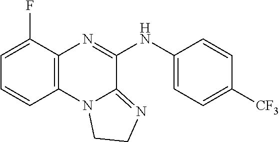 [SMILES: Fc1cccc2c1N=C(Nc1ccc(C(F)(F)F)cc1)C1=NCCN12] | 9.71 (s, 1H), 8.38 (d, J = 8.6 Hz, 2H), 7.70 (d, J = 8.7 Hz, 2H), 7.19 (td, J = 8.2, 5.6 Hz, 1H), 6.94-6.84 (m, 1H), 6.71 (d, J = 8.1 Hz, 1H), 4.23-4.08 (m, 4H) | 349.1 |
| 75 | 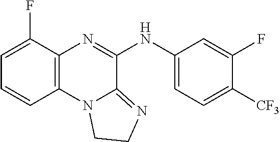 [SMILES: Fc1cc(NC2=Nc3c(F)cccc3N3CCN=C23)ccc1C(F)(F)F] | 10.18 (s, 1H), 8.62 (d, J = 14.1 Hz, 1H), 8.29 (d, J = 8.8 Hz, 1H), 7.89 (m, 1H), 7.46 (dd, J = 7.8, 1.3 Hz, 1H), 7.29 (dd, J = 8.0, 1.3 Hz, 1H), 7.18 (m, 1H), 4.46 (s, 4H) | 367.0 |
| 76 | 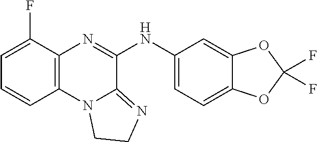 [SMILES: Fc1cccc2c1N=C(Nc1ccc3c(c1)OC(F)(F)O3)C1=NCCN12] | 10.11 (s, 1H), 8.59 (d, J = 2.1 Hz, 1H), 8.06 (m, 1H), 7.69 (m, 2H), 7.31 (m, 1H), 7.16 (t, J = 7.9 Hz, 1H), 4.27 (s, 4H) | 360.9 |
| 77 | 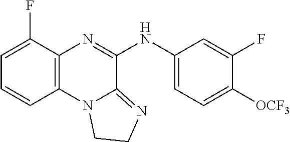 [SMILES: Fc1cc(NC2=Nc3c(F)cccc3N3CCN=C23)ccc1OC(F)(F)F] | 10.12 (s, 1H), 8.58 (m, 1H), 8.12 (d, J = 9.0 Hz, 1H), 7.63 (t, J = 8.7 Hz, 1H), 7.47 (dd, J = 7.8, 1.3 Hz, 1H), 7.24 (dd, J = 8.0, 1.3 Hz, 1H), 7.05 (t, J = 7.9 Hz, 1H), 4.19 (s, 4H) | 383.0 |
| 78 | 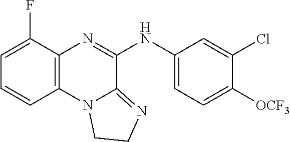 [SMILES: Fc1cccc2c1N=C(Nc1ccc(OC(F)(F)F)c(Cl)c1)C1=NCCN12] | 10.12 (s, 1H), 8.72 (d, J = 2.6 Hz, 1H), 8.50 (dd, J = 9.0, 2.6 Hz, 1H), 7.94 (dd, J = 9.1, 1.2 Hz, 1H), 7.51 (dd, J = 7.9, 1.4 Hz, 1H), 7.32 (dd, J = 8.0, 1.4 Hz, 1H), 7.12 (m, 1H), 4.37 (s, 4H) | 399.0 |
| 79 | 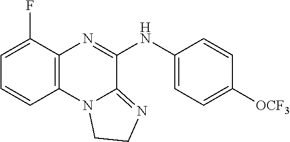 [SMILES: Fc1cccc2c1N=C(Nc1ccc(OC(F)(F)F)cc1)C1=NCCN12] | 9.67 (s, 1H), 8.46 (d, J = 9.2 Hz, 2H), 7.35 (d, J = 8.7 Hz, 2H), 7.30 (d, J = 7.9 Hz, 1H), 7.09 (t, J = 8.0 Hz, 1H), 6.87 (d, J = 8.0 Hz, 1H), 4.14 (s, 4H) | 424.9 |
| 80 | 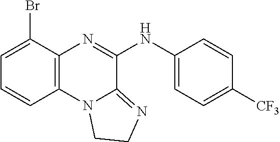 [SMILES: FC(F)(F)c1ccc(NC2=Nc3c(Br)cccc3N3CCN=C23)cc1] | 9.82 (s, 1H), 8.55 (d, J = 8.6 Hz, 2H), 7.70 (d, J = 8.7 Hz, 2H), 7.31 (d, J = 7.9 Hz, 1H), 7.11 (t, J = 8.0 Hz, 1H), 6.88 (d, J = 8.0 Hz, 1H), 4.15 (s, 4H) | 409.0 |
| 81 | 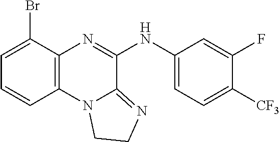 [SMILES: Fc1cc(NC2=Nc3c(Br)cccc3N3CCN=C23)ccc1C(F)(F)F] | 10.09 (s, 1H), 8.75 (d, J = 14.5 Hz, 1H), 8.17 (d, J = 8.5 Hz, 1H), 7.71 (t, J = 8.6 Hz, 1H), 7.32 (d, J = 7.9 Hz, 1H), 7.14 (t, J = 8.0 Hz, 1H), 6.88 (d, J = 7.9 Hz, 1H), 4.14 (s, 4H) | 427.0 |
| 82 | 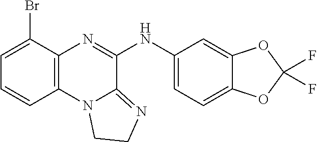 [SMILES: FC1(F)Oc2ccc(NC3=Nc4c(Br)cccc4N4CCN=C34)cc2O1] | 9.61 (s, 1H), 8.22 (d, J = 2.0 Hz, 1H), 7.81 (dd, J = 8.9, 2.1 Hz, 1H), 7.31 (m, 2H), 7.14 (dd, J = 8.0, 1.3 Hz, 1H), 6.91 (t, J = 7.8 Hz, 1H), 4.17 (s, 4H) | 421.0 |
| 83 | 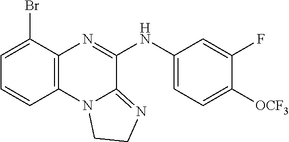 [SMILES: Fc1cc(NC2=Nc3c(Br)cccc3N3CCN=C23)ccc1OC(F)(F)F] | 9.92 (s, 1H), 8.79 (d, J = 12.5 Hz, 1H), 8.09 (d, J = 9.1 Hz, 1H), 7.53 (t, J = 9.0 Hz, 1H), 7.32 (d, J = 8.0 Hz, 1H), 7.12 (t, J = 8.0 Hz, 1H), 6.88 (d, J = 8.0 Hz, 1H), 4.15 (s, 4H) | 443.0 |
| 84 | 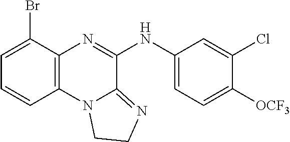 [SMILES: FC(F)(F)Oc1ccc(NC2=Nc3c(Br)cccc3N3CCN=C23)cc1Cl] | 9.92 (s, 1H), 9.08 (s, 1H), 8.19 (dd, J = 9.1, 2.6 Hz, 1H), 7.54 (d, J = 9.0 Hz, 1H), 7.32 (d, J = 8.0 Hz, 1H), 7.12 (t, J = 8.0 Hz, 1H), 6.88 (d, J = 8.0 Hz, 1H), 4.15 (s, 4H) | 458.9 |
| 85 | 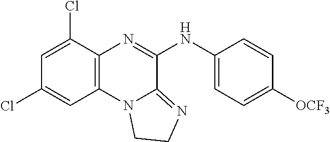 [SMILES: FC(F)(F)Oc1ccc(NC2=Nc3c(Cl)cc(Cl)cc3N3CCN=C23)cc1] | 9.75 (s, 1H), 8.38 (d, J = 9.1 Hz, 2H), 7.35 (d, J = 8.9 Hz, 2H), 7.20 (d, J = 2.1 Hz, 1H), 6.92 (d, J = 2.1 Hz, 1H), 4.12 (s, 4H) | 414.9 |
| 86 | 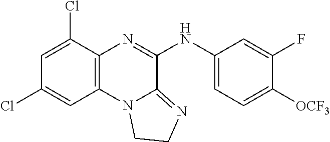 [SMILES: Fc1cc(NC2=Nc3c(Cl)cc(Cl)cc3N3CCN=C23)ccc1OC(F)(F)F] | 10.25 (s, 1H), 8.73 (d, J = 13.1 Hz, 1H), 8.44 (d, J = 8.8 Hz, 1H), 7.82 (d, J = 8.6 Hz, 1H), 7.30 (d, J = 2.0 Hz, 1H), 7.12 (d, J = 2.1 Hz, 1H), 4.27 (s, 4H) | 432.9 |
| 87 | 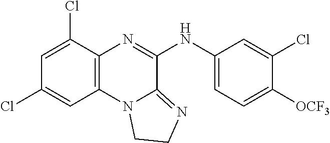 [SMILES: FC(F)(F)Oc1ccc(NC2=Nc3c(Cl)cc(Cl)cc3N3CCN=C23)cc1Cl] | 9.99 (s, 1H), 8.94 (d, J = 2.5 Hz, 1H), 8.17 (dd, J = 9.1, 2.6 Hz, 1H), 7.54 (d, J = 8.7 Hz, 1H), 7.22 (d, J = 2.1 Hz, 1H), 6.94 (d, J = 2.1 Hz, 1H), 4.13 (s, 4H) | 448.8 |
| 88 | 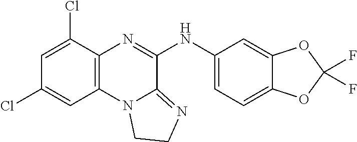 [SMILES: FC1(F)Oc2ccc(NC3=Nc4c(Cl)cc(Cl)cc4N4CCN=C34)cc2O1] | 10.12 (s, 1H), 8.59 (m, 1H), 8.19 (m, 1H), 8.02 (m, 1H), 7.47 (d, J = 2.1 Hz, 1H), 7.33 (d, J = 2.2 Hz, 1H), 4.15 (s, 4H) | 410.8 |
| 89 | 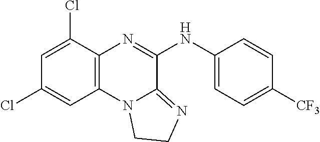 [SMILES: FC(F)(F)c1ccc(NC2=Nc3c(Cl)cc(Cl)cc3N3CCN=C23)cc1] | 9.89 (s, 1H), 8.48 (d, J = 8.6 Hz, 2H), 7.70 (d, J = 8.6 Hz, 2H), 7.22 (d, J = 2.1 Hz, 1H), 6.94 (d, J = 2.1 Hz, 1H), 4.13 (s, 4H) | 398.9 |
| 90 | 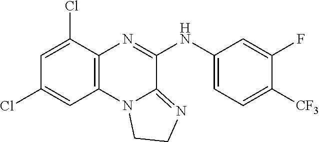 [SMILES: Fc1cc(NC2=Nc3c(Cl)cc(Cl)cc3N3CCN=C23)ccc1C(F)(F)F] | 10.16 (s, 1H), 8.64 (d, J = 14.4 Hz, 1H), 8.13 (d, J = 8.8 Hz, 1H), 7.71 (t, J = 8.6 Hz, 1H), 7.23 (d, J = 2.0 Hz, 1H), 6.95 (d, J = 2.0 Hz, 1H), 4.13 (s, 4H) | 416.9 |
| 91 | 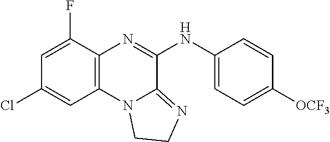 [SMILES: Fc1cc(Cl)cc2c1N=C(Nc1ccc(OC(F)(F)F)cc1)C1=NCCN12] | 9.68 (s, 1H), 8.25 (d, J = 9.1 Hz, 2H), 7.37 (d, J = 8.5 Hz, 2H), 7.05 (d, J = 10.2 Hz, 1H), 6.84 (s, 1H), 4.14 (s, 4H) | 399.0 |
| 92 | 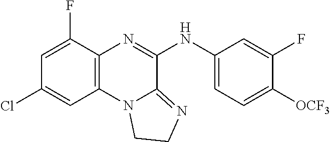 [SMILES: Fc1cc(NC2=Nc3c(F)cc(Cl)cc3N3CCN=C23)ccc1OC(F)(F)F] | 9.89 (s, 1H), 8.47 (dd, J = 13.7, 2.5 Hz, 1H), 8.00 (d, J = 9.1 Hz, 1H), 7.53 (t, J = 9.0 Hz, 1H), 7.04 (dd, J = 10.2, 2.1 Hz, 1H), 6.82 (s, 1H), 4.12 (s, 4H) | 416.9 |
| 93 | 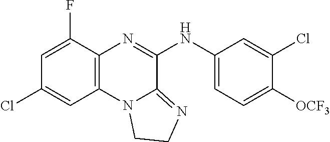 [SMILES: Fc1cc(Cl)cc2c1N=C(Nc1ccc(OC(F)(F)F)c(Cl)c1)C1=NCCN12] | 9.89 (s, 1H), 8.68 (d, J = 2.6 Hz, 1H), 8.15 (dd, J = 9.1, 2.6 Hz, 1H), 7.55 (d, J = 8.1 Hz, 1H), 7.05 (dd, J = 10.2, 2.1 Hz, 1H), 6.84 (s, 1H), 4.13 (s, 4H) | 432.9 |
| 94 | 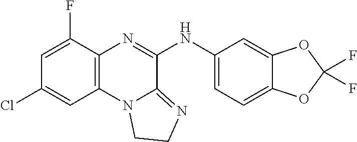 [SMILES: Fc1cc(Cl)cc2c1N=C(Nc1ccc3c(c1)OC(F)(F)O3)C1=NCCN12] | 10.22 (s, 1H), 8.67 (m, 1H), 8.29 (m, 1H), 8.11 (m, 1H), 7.55 (m, 1H), 7.43 (d, J = 2.2 Hz, 1H), 4.31 (s, 4H) | 394.9 |
| 95 | 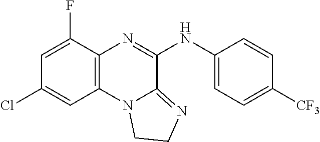 [SMILES: Fc1cc(Cl)cc2c1N=C(Nc1ccc(C(F)(F)F)cc1)C1=NCCN12] | 9.80 (s, 1H), 8.34 (d, J = 8.6 Hz, 2H), 7.70 (d, J = 8.7 Hz, 2H), 7.05 (dd, J = 10.2, 2.1 Hz, 1H), 6.83 (s, 1H), 4.13 (s, 4H) | 382.9 |
| 96 | 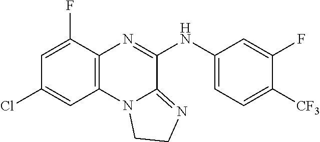 [SMILES: Fc1cc(NC2=Nc3c(F)cc(Cl)cc3N3CCN=C23)ccc1C(F)(F)F] | 10.13 (s, 1H), 8.51 (d, J = 14.5 Hz, 1H), 8.14 (d, J = 8.9 Hz, 1H), 7.78 (t, J = 8.7 Hz, 1H), 7.12 (dd, J = 10.2, 2.1 Hz, 1H), 6.90 (s, 1H), 4.19 (s, 4H) | 400.9 |

### Example 95: Synthesis of 6-Chloro-N-(2,2-difluoro-2,3-dihydrobenzofuran-5-yl) imidazolo [1,2-a] quinoxalin-4-amine (Compound 39)

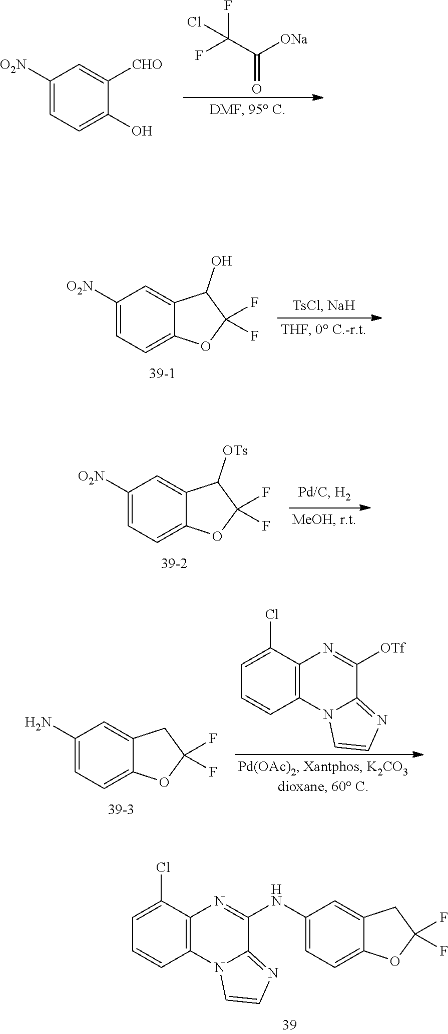

### Step 1: Synthesis of Intermediate 39-1

To a reaction flask was added DMF (30 mL). Under argon atmosphere, a solution of 2-hydroxy-5-nitrobenzaldehyde (500 mg, 2.99 mmol) and sodium difluoro(chloro) acetate (2.28 g, 14.96 mmol) in DMF (10 mL) was added dropwise at 95° C. with stirring. The mixture was stirred at 95° C. for 30 minutes, and the reaction was monitored by LC-MS until complete consumption of the starting material. The mixture was poured into water (50 mL) and with EA (30 mL*3) and dried over anhydrous Na₂SO₄. After filtration, the filtrate was concentrated under reduced pressure. The residue was purified by silica gel column chromatography (PE/EA=30/1 to 10/1) to afford a yellow solid intermediate 39-1 (409 mg, yield: 63%), ESI-MS m/z: 217.9 [M+H]⁺.

### Step 2: Synthesis of Intermediate 39-2

To a solution of 39-1 (409 mg, 1.88 mmol) in THF (30 mL) was added NaH (765 mg, 19.12 mmol) slowly at 0° C. under argon atmosphere The resulting mixture was stirred for 30 min at 0° C. Then TsCl (430 mg, 2.25 mmol) was added dropwise. The reaction was stirred at room temperature until the starting material is consumed. The reaction was then quenched with water (50 mL) and extracted with EA (30 mL*3) and dried over anhydrous Na₂SO₄. After filtration, the filtrate was concentrated under reduced pressure. The residue was purified by silica gel column chromatography (PE/EA=30/1 to 10/1) to afford a yellow solid intermediate 39-2 (520 mg, yield: 74%), ESI-MS m/z: 372.1 [M+H]⁺.

### Step 3: Synthesis of Intermediate 39-3

To a solution of 39-2 (520 mg, 1.4 mmol) in THF (30 mL) was added Pd/C (50 mg, 10%). The reaction was stirred at room temperature under a hydrogen atmosphere until the starting material is consumed. The reaction was filtrated and then the filtrate was concentrated under reduced pressure. The residue was purified by silica gel column chromatography (PE/EA=10/1 to 1/1) to afford a white solid intermediate 39-3 (195 mg, yield: 60%), ESI-MS m/z: 172.2 [M+H]⁺.

### Step 4: Synthesis of Compound 39

Compound 39 can be synthesized by a method similar to Example 1, using intermediate 3 and intermediate 39-3 as materials.

¹H NMR (400 MHz, DMSO-d₆) δ: 10.13 (s, 1H), 8.71 (d, J=2.2 Hz, 1H), 8.64 (d, J=1.1 Hz, 1H), 8.15 (m, 1H), 8.01 (m, 1H), 7.57 (d, J=1.1 Hz, 1H), 7.55 (m, 1H), 7.31 (m, 2H), 3.63 (t, J=13.2 Hz, 2H); ESI-MS m/z: 373.1 [M+H]⁺.

### Example 96: Synthesis of 6-Chloro-N-(2,2,3,3-tetrafluoro-2,3-dihydrobenzofuran-5-yl) imidazolo [1,2-a] quinoxalin-4-amine (Compound 40)

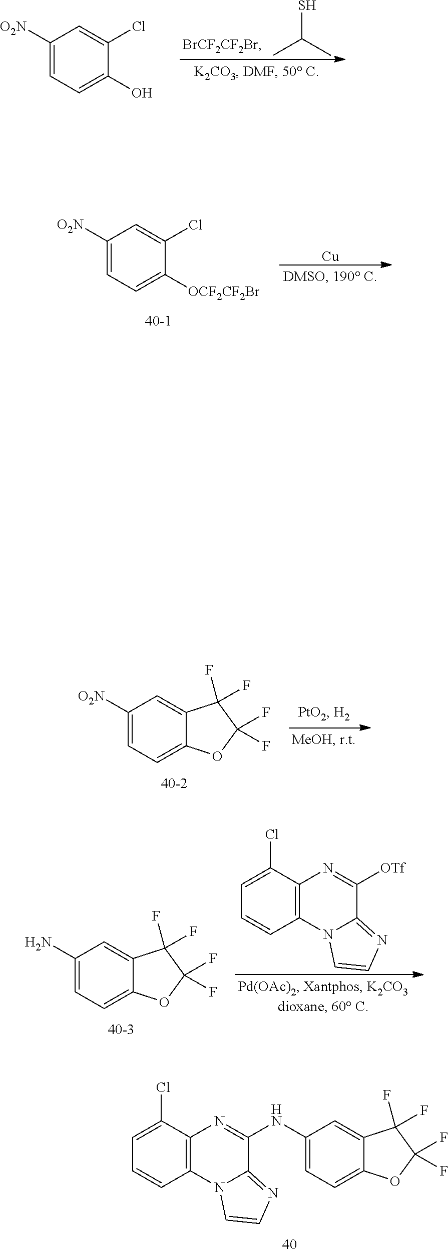

### Step 1: Synthesis of Intermediate 40-1

To a solution of 2-chloro-4-nitrophenol (500 mg, 2.88 mmol) in DMF (30 mL) was added 2-propanethiol (110 mg, 1.44 mmol), 1,2-dibromotetrafluoroethane (1.12 g, 4.32 mmol) and K₂CO₃ (476 mg, 3.45 mmol), and the mixture was heated at 50° C. for 48 hours. The reaction was monitored by LC-MS until complete consumption of the starting material. The mixture was poured into 2 N NaOH aqueous solution (50 mL) and extracted with EA (30 mL*3) and dried over anhydrous Na₂SO₄. After filtration, the filtrate was concentrated under reduced pressure. The residue was purified by silica gel column chromatography (PE/EA=100/1 to 20/1) to afford yellow oil intermediate 40-1 (426 mg, yield: 42%), ESI-MS m/z: 351.8 [M+H]⁺.

### Step 2: Synthesis of Intermediate 40-2

To a solution of 40-1 (426 mg, 1.21 mmol) in DMSO (30 mL) was added Copper powder (387 mg, 6.05 mmol), and the mixture was heated at 190° C. in a sealed tube for 6 hours. The reaction was monitored by LC-MS until complete consumption of the starting material. The mixture was poured into water and extracted with EA (30 mL*3) and dried over anhydrous Na₂SO₄. After filtration, the filtrate was concentrated under reduced pressure. The residue was purified by silica gel column chromatography (PE/EA=50/1 to 10/1) to afford a yellow solid intermediate 40-2 (236 mg, yield: 82%), ESI-MS m/z: 237.9 [M+H]⁺.

### Step 3: Synthesis of Intermediate 40-3

To a solution of 40-2 (236 mg, 1 mmol) in MeOH (30 mL) was added PtO₂ (20 mg). The reaction was stirred at room temperature under a hydrogen atmosphere until the starting material is consumed. The reaction was filtrated and then the filtrate was concentrated under reduced pressure to afford colorless oil intermediate 40-3 (220 mg, crude), ESI-MS m/z: 207.9 [M+H]⁺.

Step 4: Synthesis of Compound 40 Compound 40 can be synthesized by a method similar to Example 1, using intermediate 3 and intermediate 40-3 as materials.

¹H NMR (400 MHz, DMSO-d₆) δ: 10.23 (s, J H), 8.75 (m, 2H), 8.35 (m, 1H), 8.21 (m, 1H), 7.77 (d, J=1.2 Hz, 11H), 7.56 (m, 11H), 7.35 (m, 2H); ESI-MS m/z: 408.9 [M+H]⁺.

### Examples 97-249: Synthesis of Compounds B1-B153

Compounds B1-B153 can be synthesized by a method similar to Example 1, using Intermediates 10-54, 65-74 and corresponding amines as starting materials.

| | | | ESI-MS: |
|---|---|---|---|
| Compound | Structure | Name | [M + H]⁺ |
| B1 | 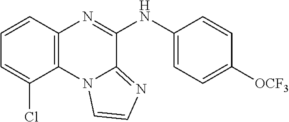 [SMILES: FC(F)(F)Oc1ccc(Nc2nc3cccc(Cl)c3n3ccnc23)cc1] | 9-chloro-N-(4- (trifluoromethoxy)- phenyl)imidazolo [1,2-a]quinoxalin- 4-amine | 378.9 |
| B2 | 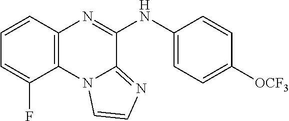 [SMILES: Fc1cccc2nc(Nc3ccc(OC(F)(F)F)cc3)c3nccn3c12] | 9-fluoro-N-(4- (trifluoromethoxy) phenyl)imidazolo [1,2-a]quinoxalin- 4-amine | 362.9 |
| B3 | 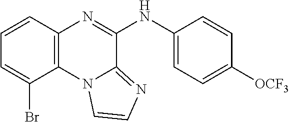 [SMILES: FC(F)(F)Oc1ccc(Nc2nc3cccc(Br)c3n3ccnc23)cc1] | 9-bromo-N-(4- (trifluoromethoxy) phenyl)imidazolo [1,2-a]quinoxalin- 4-amine | 422.8 |
| B4 | 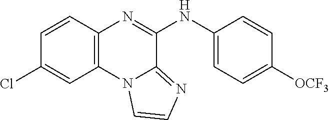 [SMILES: FC(F)(F)Oc1ccc(Nc2nc3ccc(Cl)cc3n3ccnc23)cc1] | 8-chloro-N-(4- (trifluoromethoxy) phenyl)imidazolo [1,2-a]quinoxalin- 4-amine | 378.9 |
| B5 | 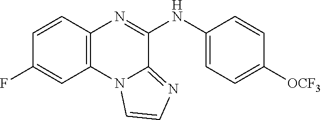 [SMILES: Fc1ccc2nc(Nc3ccc(OC(F)(F)F)cc3)c3nccn3c2c1] | 8-fluoro-N-(4- (trifluoromethoxy) phenyl)imidazolo [1,2-a]quinoxalin- 4-amine | 362.9 |
| B6 | 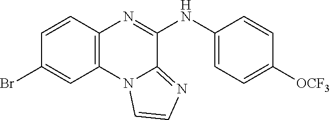 [SMILES: FC(F)(F)Oc1ccc(Nc2nc3ccc(Br)cc3n3ccnc23)cc1] | 8-bromo-N-(4- (trifluoromethoxy) phenyl)imidazolo [1,2-a]quinoxalin- 4-amine | 422.8 |
| B7 | 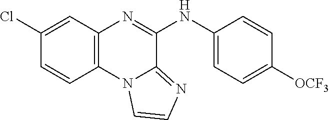 [SMILES: FC(F)(F)Oc1ccc(Nc2nc3cc(Cl)ccc3n3ccnc23)cc1] | 7-chloro-N-(4- (trifluoromethoxy) phenyl)imidazolo [1,2-a]quinoxalin- 4-amine | 378.9 |
| B8 | 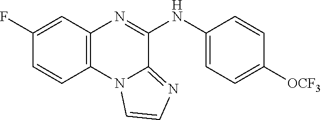 [SMILES: Fc1ccc2c(c1)nc(Nc1ccc(OC(F)(F)F)cc1)c1nccn12] | 7-fluoro-N-(4- (trifluoromethoxy) phenyl)imidazolo [1,2-a]quinoxalin- 4-amine | 362.9 |
| B9 | 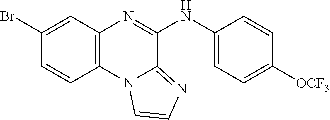 [SMILES: FC(F)(F)Oc1ccc(Nc2nc3cc(Br)ccc3n3ccnc23)cc1] | 7-bromo-N-(2- (trifluoromethoxy) phenyl)imidazolo [1,2-a]quinoxalin- 4-amine | 422.8 |
| B10 | 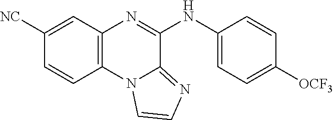 [SMILES: N#Cc1ccc2c(c1)nc(Nc1ccc(OC(F)(F)F)cc1)c1nccn12] | 7-cyano-N-(4- (trifluoromethoxy) phenyl)imidazolo [1,2-a]quinoxalin- 4-amine | 369.9 |
| B11 | 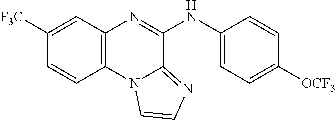 [SMILES: FC(F)(F)Oc1ccc(Nc2nc3cc(C(F)(F)F)ccc3n3ccnc23)cc1] | 7-trifluoromethyl- N-(4- (trifluoromethoxy) phenyl)imidazolo [1,2-a]quinoxalin- 4-amine | 412.9 |
| B12 | 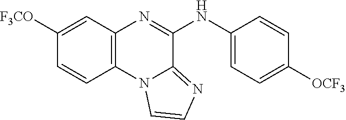 [SMILES: FC(F)(F)Oc1ccc(Nc2nc3cc(OC(F)(F)F)ccc3n3ccnc23)cc1] | 7-trifluoromethoxy- N-(4- (trifluoromethoxy) phenyl)imidazolo [1,2-a]quinoxalin- 4-amine | 428.9 |
| B13 | 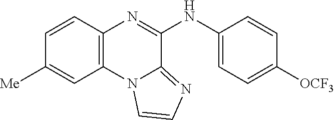 [SMILES: Cc1ccc2nc(Nc3ccc(OC(F)(F)F)cc3)c3nccn3c2c1] | 8-methyl-N-(4- (trifluoromethoxy) phenyl)imidazolo [1,2-a]quinoxalin- 4-amine | 358.9 |
| B14 | 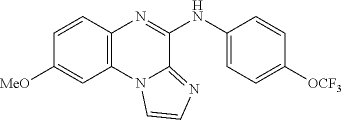 [SMILES: COc1ccc2nc(Nc3ccc(OC(F)(F)F)cc3)c3nccn3c2c1] | 8-methoxy-N-(4- (trifluoromethoxy) phenyl)imidazolo [1,2-a]quinoxalin- 4-amine | 374.9 |
| B15 | 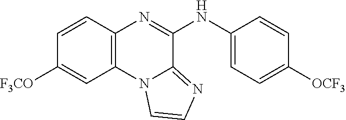 [SMILES: FC(F)(F)Oc1ccc(Nc2nc3ccc(OC(F)(F)F)cc3n3ccnc23)cc1] | 8-trifluoromethoxy- N-(4- (trifluoromethoxy) phenyl)imidazolo [1,2-a]quinoxalin- 4-amine | 428.9 |
| B16 | 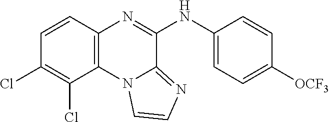 [SMILES: FC(F)(F)Oc1ccc(Nc2nc3ccc(Cl)c(Cl)c3n3ccnc23)cc1] | 8,9-dichloro-N-(4- (trifluoromethoxy) phenyl)imidazolo [1,2-a]quinoxalin- 4-amine | 412.8 |
| B17 | 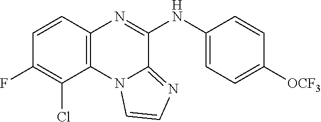 [SMILES: Fc1ccc2nc(Nc3ccc(OC(F)(F)F)cc3)c3nccn3c2c1Cl] | 9-chloro-8- fluoro-N-(4- (trifluoromethoxy) phenyl)imidazolo [1,2-a]quinoxalin- 4-amine | 396.8 |
| B18 | 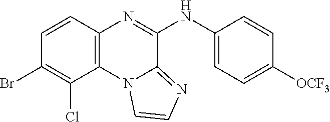 [SMILES: FC(F)(F)Oc1ccc(Nc2nc3ccc(Br)c(Cl)c3n3ccnc23)cc1] | 8-bromo-9- chloro-N-(4- (trifluoromethoxy) phenyl)imidazolo [1,2-a]quinoxalin- 4-amine | 456.8 |
| B19 | 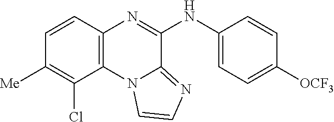 [SMILES: Cc1ccc2nc(Nc3ccc(OC(F)(F)F)cc3)c3nccn3c2c1Cl] | 9-chloro-8- methyl-N-(4- (trifluoromethoxy) phenyl)imidazolo [1,2-a]quinoxalin- 4-amine | 392.9 |
| B20 | 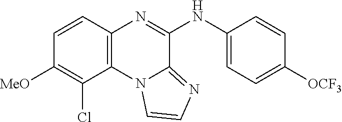 [SMILES: COc1ccc2nc(Nc3ccc(OC(F)(F)F)cc3)c3nccn3c2c1Cl] | 9-chloro-8- methoxy-N-(4- (trifluoromethoxy) phenyl)imidazolo [1,2-a]quinoxalin- 4-amine | 408.9 |
| B21 | 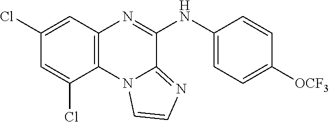 [SMILES: FC(F)(F)Oc1ccc(Nc2nc3cc(Cl)cc(Cl)c3n3ccnc23)cc1] | 7,9-dichloro- N-(4- (trifluoromethoxy) phenyl)imidazolo [1,2-a]quinoxalin- 4-amine | 412.8 |
| B22 | 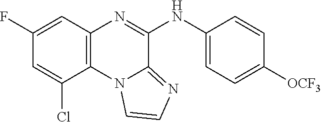 [SMILES: Fc1cc(Cl)c2c(c1)nc(Nc1ccc(OC(F)(F)F)cc1)c1nccn12] | 9-chloro- 7-fluoro-N-(4- (trifluoromethoxy) phenyl)imidazolo [1,2-a]quinoxalin- 4-amine | 396.8 |
| B23 | 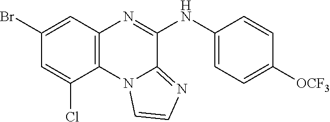 [SMILES: FC(F)(F)Oc1ccc(Nc2nc3cc(Br)cc(Cl)c3n3ccnc23)cc1] | 7-bromo-9- Chloro-N-(4- (trifluoromethoxy) phenyl)imidazolo [1,2-a]quinoxalin- 4-amine | 456.8 |
| B24 | 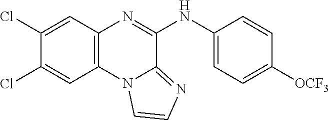 [SMILES: FC(F)(F)Oc1ccc(Nc2nc3cc(Cl)c(Cl)cc3n3ccnc23)cc1] | 7,8-dichloro- N-(4- (trifluoromethoxy) phenyl)imidazolo [1,2-a]quinoxalin- 4-amine | 412.8 |
| B25 | 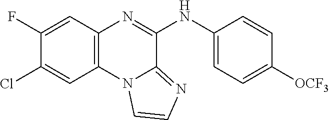 [SMILES: Fc1cc2nc(Nc3ccc(OC(F)(F)F)cc3)c3nccn3c2cc1Cl] | 8-chloro-7- fluoro-N-(4- (trifluoromethoxy) phenyl)imidazolo [1,2-a]quinoxalin- 4-amine | 396.8 |
| B26 | 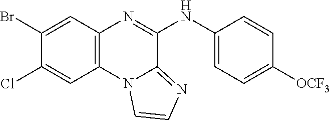 [SMILES: FC(F)(F)Oc1ccc(Nc2nc3cc(Br)c(Cl)cc3n3ccnc23)cc1] | 7-bromo-8- chloro-N-(4- (trifluoromethoxy) phenyl)imidazolo [1,2-a]quinoxalin- 4-amine | 456.8 |
| B27 | 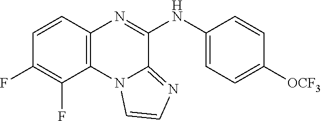 [SMILES: Fc1ccc2nc(Nc3ccc(OC(F)(F)F)cc3)c3nccn3c2c1F] | 8,9-difluoro- N-(4- (trifluoromethoxy) phenyl)imidazolo [1,2-a]quinoxalin- 4-amine | 380.9 |
| B28 | 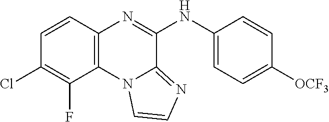 [SMILES: Fc1c(Cl)ccc2nc(Nc3ccc(OC(F)(F)F)cc3)c3nccn3c12] | 8-chloro-9- fluoro-N-(4- (trifluoromethoxy) phenyl)imidazolo [1,2-a]quinoxalin- 4-amine | 396.8 |
| B29 | 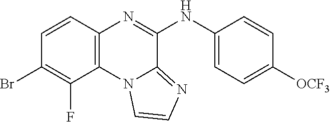 [SMILES: Fc1c(Br)ccc2nc(Nc3ccc(OC(F)(F)F)cc3)c3nccn3c12] | 8-bromo-9- fluoro-N-(4- (trifluoromethoxy) phenyl)imidazolo [1,2-a]quinoxalin- 4-amine | 440.8 |
| B30 | 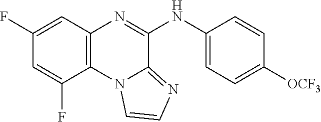 [SMILES: Fc1cc(F)c2c(c1)nc(Nc1ccc(OC(F)(F)F)cc1)c1nccn12] | 7,9-difluoro- N-(4- (trifluoromethoxy) phenyl)imidazolo [1,2-a]quinoxalin- 4-amine | 380.9 |
| B31 | 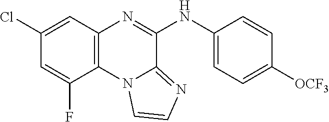 [SMILES: Fc1cc(Cl)cc2nc(Nc3ccc(OC(F)(F)F)cc3)c3nccn3c12] | 7-chloro-9- fluoro-N-(4- (trifluoromethoxy) phenyl)imidazolo [1,2-a]quinoxalin- 4-amine | 396.8 |
| B32 | 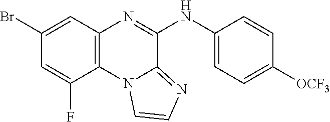 [SMILES: Fc1cc(Br)cc2nc(Nc3ccc(OC(F)(F)F)cc3)c3nccn3c12] | 7-bromo-9- fluoro-N-(4- (trifluoromethoxy) phenyl)imidazolo [1,2-a]quinoxalin- 4-amine | 440.8 |
| B33 | 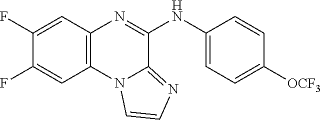 [SMILES: Fc1cc2nc(Nc3ccc(OC(F)(F)F)cc3)c3nccn3c2cc1F] | 7,8-difluoro- N-(4- (trifluoromethoxy) phenyl)imidazolo [1,2-a]quinoxalin- 4-amine | 380.9 |
| B34 | 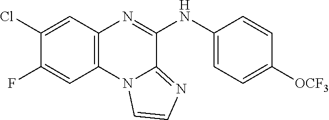 [SMILES: Fc1cc2c(cc1Cl)nc(Nc1ccc(OC(F)(F)F)cc1)c1nccn12] | 7-chloro-8- fluoro-N-(4- (trifluoromethoxy) phenyl)imidazolo [1,2-a]quinoxalin- 4-amine | 396.8 |
| B35 | 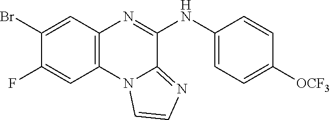 [SMILES: Fc1cc2c(cc1Br)nc(Nc1ccc(OC(F)(F)F)cc1)c1nccn12] | 7-bromo- 8-fluoro-N-(4- (trifluoromethoxy) phenyl)imidazolo [1,2-a]quinoxalin- 4-amine | 440.8 |
| B36 | 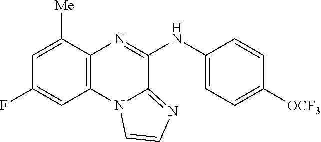 [SMILES: Cc1cc(F)cc2c1nc(Nc1ccc(OC(F)(F)F)cc1)c1nccn12] | 8-fluoro- 6-methyl-N-(4- (trifluoromethoxy) phenyl)imidazolo [1,2-a]quinoxalin- 4-amine | 376.9 |
| B37 | 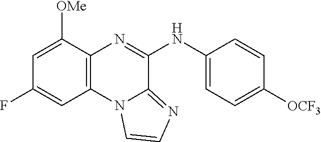 [SMILES: COc1cc(F)cc2c1nc(Nc1ccc(OC(F)(F)F)cc1)c1nccn12] | 8-fluoro-6- methoxy-N-(4- (trifluoromethoxy) phenyl)imidazolo [1,2-a]quinoxalin- 4-amine | 392.9 |
| B38 | 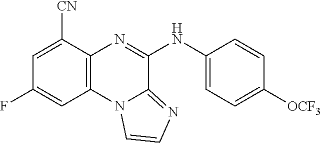 [SMILES: N#Cc1cc(F)cc2c1nc(Nc1ccc(OC(F)(F)F)cc1)c1nccn12] | 8-fluoro-6- Cyano-N-(4- (trifluoromethoxy) phenyl)imidazolo [1,2-a]quinoxalin- 4-amine | 387.9 |
| B39 | 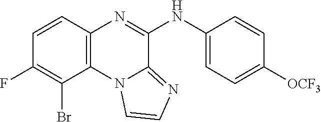 [SMILES: Fc1ccc2nc(Nc3ccc(OC(F)(F)F)cc3)c3nccn3c2c1Br] | 9-bromo-8- fluoro-N-(4- (trifluoromethoxy) phenyl)imidazolo [1,2-a]quinoxalin- 4-amine | 440.8 |
| B40 | 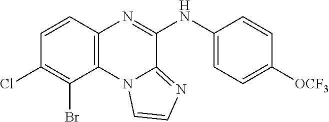 [SMILES: FC(F)(F)Oc1ccc(Nc2nc3ccc(Cl)c(Br)c3n3ccnc23)cc1] | 9-bromo-8- chloro-N-(4- (trifluoromethoxy) phenyl)imidazolo [1,2-a]quinoxalin- 4-amine | 456.8 |
| B41 | 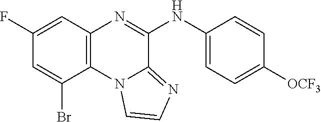 [SMILES: Fc1cc(Br)c2c(c1)nc(Nc1ccc(OC(F)(F)F)cc1)c1nccn12] | 9-bromo-7- fluoro-N-(4- (trifluoromethoxy) phenyl)imidazolo [1,2-a]quinoxalin- 4-amine | 440.8 |
| B42 | 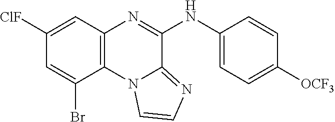 | 9-bromo-7- chloro-N-(4- (trifluoromethoxy) phenyl)imidazolo [1,2-a]quinoxalin- 4-amine | 456.8 |
| B43 | 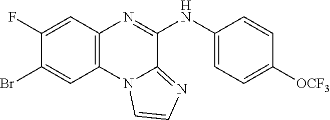 [SMILES: Fc1cc2nc(Nc3ccc(OC(F)(F)F)cc3)c3nccn3c2cc1Br] | 8-bromo-7- fluoro-N-(4- (trifluoromethoxy) phenyl)imidazolo [1,2-a]quinoxalin- 4-amine | 440.8 |
| B44 | 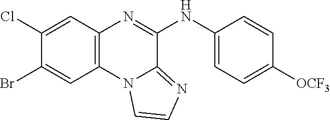 [SMILES: FC(F)(F)Oc1ccc(Nc2nc3cc(Cl)c(Br)cc3n3ccnc23)cc1] | 8-bromo-7- chloro-N-(4- (trifluoromethoxy) phenyl)imidazolo [1,2-a]quinoxalin- 4-amine | 456.8 |
| B45 | 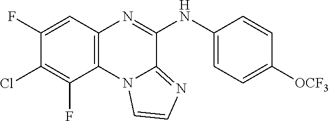 [SMILES: Fc1cc2nc(Nc3ccc(OC(F)(F)F)cc3)c3nccn3c2c(F)c1Cl] | 8-chloro-7,9- difluoro-N-(4- (trifluoromethoxy) phenyl)imidazolo [1,2-a]quinoxalin- 4-amine | 414.8 |
| B46 | 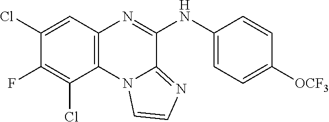 [SMILES: Fc1c(Cl)cc2nc(Nc3ccc(OC(F)(F)F)cc3)c3nccn3c2c1Cl] | 7,9-dichloro- 8-fluoro-N-(4- (trifluoromethoxy) phenyl)imidazolo [1,2-a]quinoxalin- 4-amine | 430.8 |
| B47 | 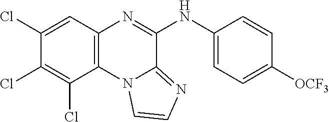 [SMILES: FC(F)(F)Oc1ccc(Nc2nc3cc(Cl)c(Cl)c(Cl)c3n3ccnc23)cc1] | 7,8,9-trichloro- N-(4- (trifluoromethoxy) phenyl)imidazolo [1,2-a]quinoxalin- 4-amine | 446.8 |
| B48 | 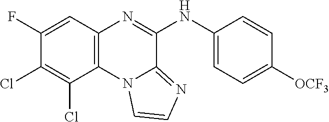 [SMILES: Fc1cc2nc(Nc3ccc(OC(F)(F)F)cc3)c3nccn3c2c(Cl)c1Cl] | 8,9-dichloro-7- fluoro-N-(4- (trifluoromethoxy) phenyl)imidazolo [1,2-a]quinoxalin- 4-amine | 430.8 |
| B49 | 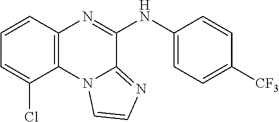 [SMILES: FC(F)(F)c1ccc(Nc2nc3cccc(Cl)c3n3ccnc23)cc1] | 9-chloro-N-(4- (trifluoromethyl) phenyl)imidazolo [1,2-a]quinoxalin- 4-amine | 362.9 |
| B50 | 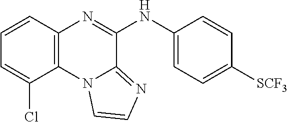 | 9-chloro-N-(4- (trifluoromethylthio) phenyl)imidazolo [1,2-a]quinoxalin- 4-amine | 394.8 |
| B51 | 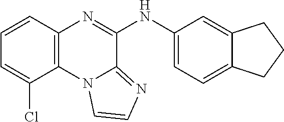 [SMILES: Clc1cccc2nc(Nc3ccc4c(c3)CCC4)c3nccn3c12] | 9-chloro-N-(2,3- dihydro-1H-inden-5- yl)imidazo[1,2-a] quinoxalin-4- amine | 334.9 |
| B52 | 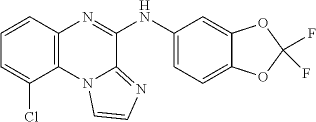 [SMILES: FC1(F)Oc2ccc(Nc3nc4cccc(Cl)c4n4ccnc34)cc2O1] | 9-chloro-N-(2,2- difluorobenzo[d] [1,3]dihydroxy-5-yl) imidazolo[1,2- a]quinoxalin-4-amine | 374.8 |
| B53 | 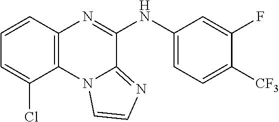 [SMILES: Fc1cc(Nc2nc3cccc(Cl)c3n3ccnc23)ccc1C(F)(F)F] | 9-chloro-N- (3-Fluoro-4- (trifluoromethyl) phenyl)imidazolo [1,2-a]quinoxalin- 4-amine | 380.9 |
| B54 | 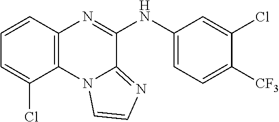 [SMILES: FC(F)(F)c1ccc(Nc2nc3cccc(Cl)c3n3ccnc23)cc1Cl] | 9-chloro-N- (3-chloro-4- (trifluoromethyl) phenyl)imidazolo [1,2-a]quinoxalin- 4-amine | 396.8 |
| B55 | 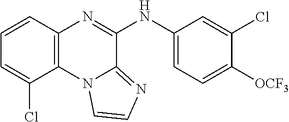 [SMILES: FC(F)(F)Oc1ccc(Nc2nc3cccc(Cl)c3n3ccnc23)cc1Cl] | 9-chloro-N- (3-Fluoro-4- (trifluoromethoxy) phenyl)imidazolo [1,2-a]quinoxalin- 4-amine | 396.8 |
| B56 | 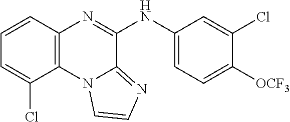 [SMILES: FC(F)(F)Oc1ccc(Nc2nc3cccc(Cl)c3n3ccnc23)cc1Cl] | 9-chloro-N- (3-chloro-4- (trifluoromethoxy) phenyl)imidazolo [1,2-a]quinoxalin- 4-amine | 412.8 |
| B57 | 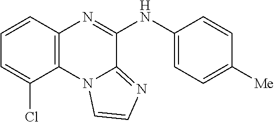 [SMILES: Cc1ccc(Nc2nc3cccc(Cl)c3n3ccnc23)cc1] | 9-chloro-N-(p- toluene)imidazolo [1,2-a]quinoxaline- 4-amine | 308.9 |
| B58 | 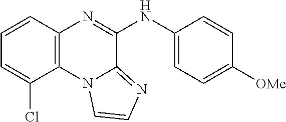 [SMILES: COc1ccc(Nc2nc3cccc(Cl)c3n3ccnc23)cc1] | 9-chloro-N-(4- (methoxy)phenyl) imidazolo [1,2-a]quinoxalin- 4-amine | 324.9 |
| B59 | 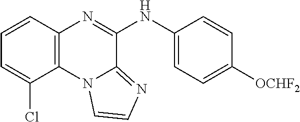 [SMILES: FC(F)Oc1ccc(Nc2nc3cccc(Cl)c3n3ccnc23)cc1] | 9-chloro-N-(4- (difluoromethoxy) phenyl)imidazolo [1,2-a]quinoxalin- 4-amine | 360.9 |
| B60 | 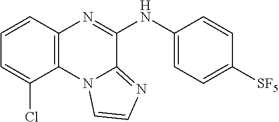 | 9-chloro-N-(4- (thiopentafluoride) phenyl)imidazolo[1,2- a]quinoxalin- 4-amine | 420.8 |
| B61 | 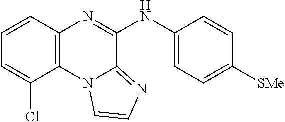 [SMILES: CSc1ccc(Nc2nc3cccc(Cl)c3n3ccnc23)cc1] | 9-chloro-N-(4- (methylthio)phenyl) imidazolo [1,2-a]quinoxalin- 4-amine | 340.9 |
| B62 | 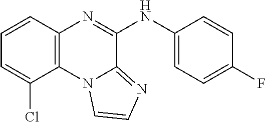 [SMILES: Fc1ccc(Nc2nc3cccc(Cl)c3n3ccnc23)cc1] | 9-chloro-N-(p- fluorophenyl) imidazolo [1,2-a]quinoxalin- 4-amine | 312.9 |
| B63 | 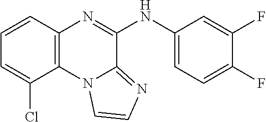 [SMILES: Fc1ccc(Nc2nc3cccc(Cl)c3n3ccnc23)cc1F] | 9-chloro-N-(3,4- difluorophenyl) imidazolo [1,2-a]quinoxalin- 4-amine | 330.9 |
| B64 | 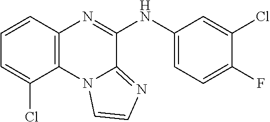 [SMILES: Fc1ccc(Nc2nc3cccc(Cl)c3n3ccnc23)cc1Cl] | 9-chloro-N-(3-chloro- 4-fluorophenyl) imidazolo [1,2-a]quinoxalin- 4-amine | 346.8 |
| B65 | 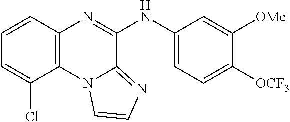 [SMILES: COc1cc(Nc2nc3cccc(Cl)c3n3ccnc23)ccc1OC(F)(F)F] | 9-chloro-N- (3-methoxy-4- (trifluoromethoxy) phenyl)imidazolo [1,2-a]quinoxalin- 4-amine | 408.9 |
| B66 | 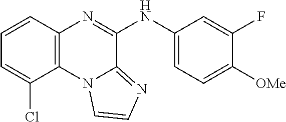 [SMILES: COc1ccc(Nc2nc3cccc(Cl)c3n3ccnc23)cc1F] | 9-chloro-N- (3-fluoro-4- methoxyphenyl) Imidazolo [1,2-a]quinoline- 4-amine | 342.9 |
| B67 | 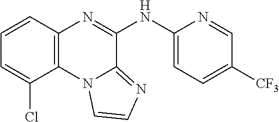 [SMILES: FC(F)(F)c1ccc(Nc2nc3cccc(Cl)c3n3ccnc23)nc1] | 9-chloro-N-(5- (trifluoromethyl) pyridin-2-yl) imidazolo [1,2-a]quinoxalin- 4-amine | 363.9 |
| B68 | 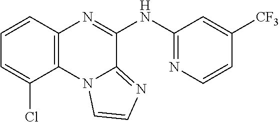 [SMILES: FC(F)(F)c1ccnc(Nc2nc3cccc(Cl)c3n3ccnc23)c1] | 9-chloro-N-(4- (trifluoromethyl) pyridin-2-yl) imidazolo [1,2-a]quinoxalin- 4-amine | 363.9 |
| B69 | 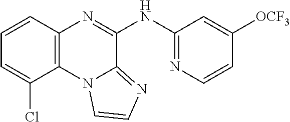 [SMILES: FC(F)(F)Oc1ccnc(Nc2nc3cccc(Cl)c3n3ccnc23)c1] | 9-chloro-N-(4- (trifluoromethoxy) pyridin-2-yl) imidazolo [1,2-a]quinoxalin- 4-amine | 379.8 |
| B70 | 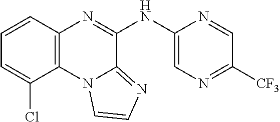 [SMILES: FC(F)(F)c1cnc(Nc2nc3cccc(Cl)c3n3ccnc23)cn1] | 9-chloro-N-(5- (trifluoromethyl) pyrazin-2-yl) imidazolo [1,2-a]quinoxalin- 4-amine | 364.9 |
| B71 | 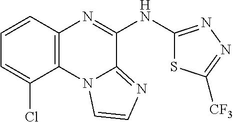 [SMILES: FC(F)(F)c1nnc(Nc2nc3cccc(Cl)c3n3ccnc23)s1] | N-(9- chloroimidazo[1,2-a] quinoxalin-4-yl)-5- (trifluoromethyl)- 1,3,4-thiadiazole- 2-amine | 370.8 |
| B72 | 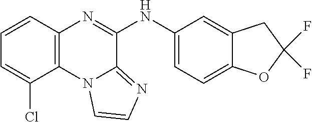 [SMILES: FC1(F)Cc2cc(Nc3nc4cccc(Cl)c4n4ccnc34)ccc2O1] | 9-chloro-N-(2,2- difluoro-2,3- dihydrobenzofuran- 5-yl) imidazolo[1,2-a] quinoxalin-4-amine | 373.1 |
| B73 | 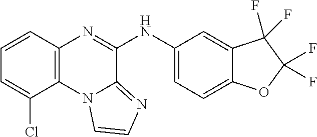 [SMILES: FC1(F)Oc2ccc(Nc3nc4cccc(Cl)c4n4ccnc34)cc2C1(F)F] | 9-chloro-N- (2,2,3,3-tetrafluoro- 2,3- dihydrobenzofuran- 5-yl) imidazolo[1,2-a] quinoxalin-4-amine | 409.0 |
| B74 | 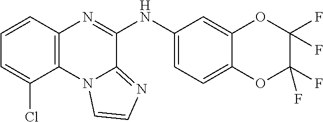 [SMILES: FC1(F)Oc2ccc(Nc3nc4cccc(Cl)c4n4ccnc34)cc2OC1(F)F] | 9-chloro-N- (2,2,3,3-tetrafluoro-2,3- dihydrobenzofuran [b][1,4]dioxolan-6-yl) imidazolo[1,2-a] quinoxalin-4-amine | 424.8 |
| B75 | 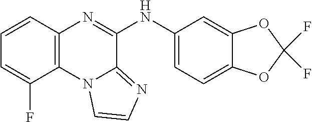 [SMILES: Fc1cccc2nc(Nc3ccc4c(c3)OC(F)(F)O4)c3nccn3c12] | 9-fluoro-N-(2,2- difluorobenzo[d] [1,3]dihydroxy-5-yl) imidazolo[1,2-a] quinoxalin-4-amine | 358.9 |
| B76 | 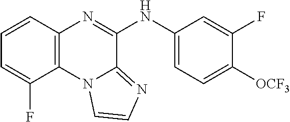 [SMILES: Fc1cc(Nc2nc3cccc(F)c3n3ccnc23)ccc1OC(F)(F)F] | 9-fluoro-N- (3-fluoro-4- (trifluoromethoxy) phenyl) imidazolo[1,2-a] quinoxalin-4-amine | 380.9 |
| B77 | 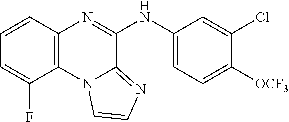 [SMILES: Fc1cccc2nc(Nc3ccc(OC(F)(F)F)c(Cl)c3)c3nccn3c12] | 9-fluoro-N-(3- chloro-4- (trifluoromethoxy) phenyl) imidazolo[1,2-a ]quinoxalin-4-amine | 396.8 |
| B78 | 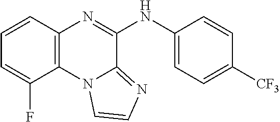 [SMILES: Fc1cccc2nc(Nc3ccc(C(F)(F)F)cc3)c3nccn3c12] | 9-fluoro-N-(4- (trifluoromethyl) phenyl) imidazolo[1,2-a] quinoxalin-4-amine | 346.9 |
| B79 | 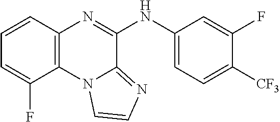 [SMILES: Fc1cc(Nc2nc3cccc(F)c3n3ccnc23)ccc1C(F)(F)F] | 9-fluoro-N- (3-fluoro-4- (trifluoromethyl)phenyl) imidazolo[1,2-a] quinoxalin-4-amine | 364.9 |
| B80 | 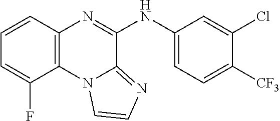 [SMILES: Fc1cccc2nc(Nc3ccc(C(F)(F)F)c(Cl)c3)c3nccn3c12] | 9-fluoro-N- (3-chloro-4- (trifluoromethyl)phenyl) imidazolo[1,2-a] quinoxalin-4-amine | 380.9 |
| B81 | 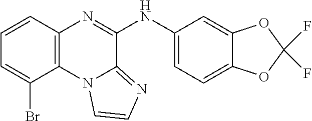 [SMILES: FC1(F)Oc2ccc(Nc3nc4cccc(Br)c4n4ccnc34)cc2O1] | 9-bromo-N- (2,2-difluorobenzo[d] [1,3]dihydroxy-5- yl)imidazolo[1,2- a]quinoxalin-4-amine | 418.8 |
| B82 | 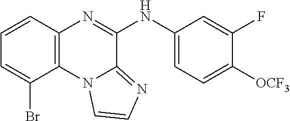 [SMILES: Fc1cc(Nc2nc3cccc(Br)c3n3ccnc23)ccc1OC(F)(F)F] | 9-bromo-N- (3-fluoro-4- (trifluoromethoxy) phenyl) imidazolo[1,2-a] quinoxalin-4-amine | 440.8 |
| B83 | 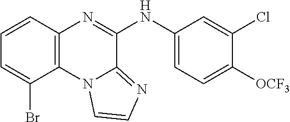 [SMILES: FC(F)(F)Oc1ccc(Nc2nc3cccc(Br)c3n3ccnc23)cc1Cl] | 9-bromo-N- (3-chloro-4- (trifluoromethoxy) phenyl) imidazolo[1,2-a] quinoxalin-4-amine | 456.8 |
| B84 | 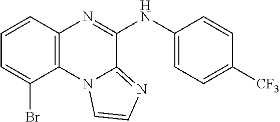 [SMILES: FC(F)(F)c1ccc(Nc2nc3cccc(Br)c3n3ccnc23)cc1] | 9-bromo-N-(4- (trifluoromethyl) phenyl) imidazolo[1,2-a] quinoxalin-4-amine | 406.8 |
| B85 | 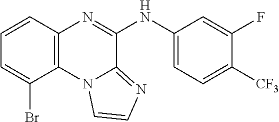 [SMILES: Fc1cc(Nc2nc3cccc(Br)c3n3ccnc23)ccc1C(F)(F)F] | 9-bromo-N- (3-fluoro-4- (trifluoromethyl)phenyl) imidazolo[1,2-a] quinoxalin-4-amine | 424.8 |
| B86 | 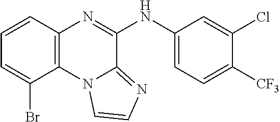 [SMILES: FC(F)(F)c1ccc(Nc2nc3cccc(Br)c3n3ccnc23)cc1Cl] | 9-bromo-N- (3-chloro-4- (trifluoromethyl)phenyl) imidazolo[1,2-a] quinoxalin-4-amine | 440.8 |
| B87 | 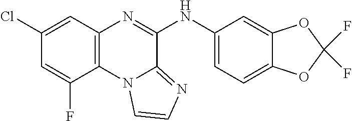 [SMILES: Fc1cc(Cl)cc2nc(Nc3ccc4c(c3)OC(F)(F)O4)c3nccn3c12] | 7-chloro-9- fluoro-N-(2,2- difluorobenzo[d] [1,3]dihydroxy-5- yl)imidazo[1,2-a] quinoxalin-4- amine | 392.8 |
| B88 | 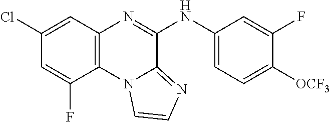 [SMILES: Fc1cc(Nc2nc3cc(Cl)cc(F)c3n3ccnc23)ccc1OC(F)(F)F] | 7-chloro-9-fluoro- N-(3-fluoro-4- (trifluoromethoxy)phenyl) imidazolo[1,2-a] quinoxalin-4-amine | 414.8 |
| B89 | 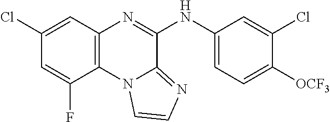 [SMILES: Fc1cc(Cl)cc2nc(Nc3ccc(OC(F)(F)F)c(Cl)c3)c3nccn3c12] | 7-chloro-9-fluoro- N-(3-chloro-4- (trifluoromethoxy)phenyl) imidazolo[1,2-a] quinoxalin-4-amine | 430.8 |
| B90 | 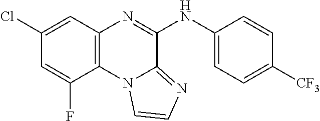 [SMILES: Fc1cc(Cl)cc2nc(Nc3ccc(C(F)(F)F)cc3)c3nccn3c12] | 7-chloro-9-fluoro-N-(4- (trifluoromethyl)phenyl) imidazolo[1,2-a] quinoxalin-4-amine | 380.9 |
| B91 | 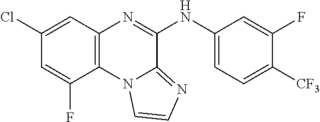 [SMILES: Fc1cc(Nc2nc3cc(Cl)cc(F)c3n3ccnc23)ccc1C(F)(F)F] | 7-chloro-9-fluoro- N-(3-fluoro-4- (trifluoromethyl)phenyl) imidazolo[1,2-a] quinoxalin-4-amine | 398.8 |
| B92 | 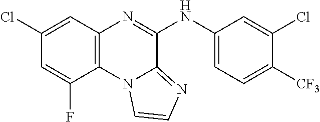 [SMILES: Fc1cc(Cl)cc2nc(Nc3ccc(C(F)(F)F)c(Cl)c3)c3nccn3c12] | 7-chloro-9-fluoro- N-(3-chloro-4- (trifluoromethyl)phenyl) imidazolo[1,2-a] quinoxalin-4-amine | 414.8 |
| B93 | 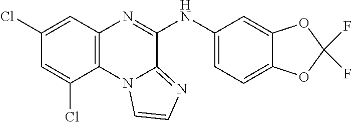 [SMILES: FC1(F)Oc2ccc(Nc3nc4cc(Cl)cc(Cl)c4n4ccnc34)cc2O1] | 7,9-dichloro-N- (2,2-difluorobenzo [d][1,3]dihydroxy-5-yl) imidazolo[1,2-a] quinoxalin-4-amine | 408.8 |
| B94 | 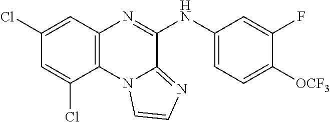 [SMILES: Fc1cc(Nc2nc3cc(Cl)cc(Cl)c3n3ccnc23)ccc1OC(F)(F)F] | 7,9-dichloro- N-(3-fluoro-4- (trifluoromethoxy)phenyl) imidazolo[1,2-a] quinoxalin-4-amine | 430.8 |
| B95 | 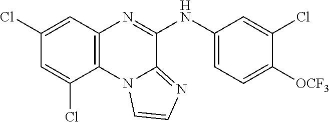 [SMILES: FC(F)(F)Oc1ccc(Nc2nc3cc(Cl)cc(Cl)c3n3ccnc23)cc1Cl] | 7,9-dichloro-N- (3-chloro-4- (trifluoromethoxy)phenyl) imidazolo[1,2-a] quinoxalin-4-amine | 446.8 |
| B96 | 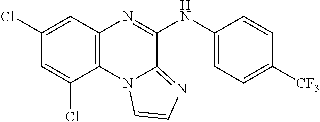 [SMILES: FC(F)(F)c1ccc(Nc2nc3cc(Cl)cc(Cl)c3n3ccnc23)cc1] | 7,9-dichloro-N- (4-(trifluoromethyl) phenyl) imidazolo[1,2-a] quinoxalin-4-amine | 396.8 |
| B97 | 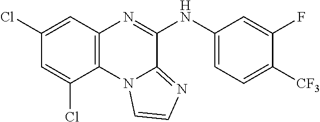 [SMILES: Fc1cc(Nc2nc3cc(Cl)cc(Cl)c3n3ccnc23)ccc1C(F)(F)F] | 7,9-dichloro- N-(3-fluoro-4- (trifluoromethyl)phenyl) imidazolo[1,2-a] quinoxalin-4-amine | 414.8 |
| B98 | 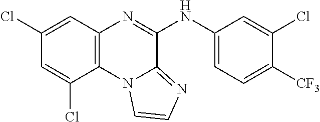 [SMILES: FC(F)(F)c1ccc(Nc2nc3cc(Cl)cc(Cl)c3n3ccnc23)cc1Cl] | 7,9-dichloro-N- (3-chloro-4- (trifluoromethyl)phenyl) imidazolo[1,2-a] quinoxalin-4-amine | 430.8 |
| B99 | 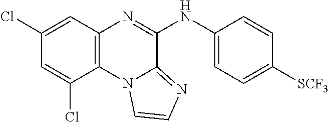 | 7,9-dichloro-N-(4- (trifluoromethylthio) phenyl) imidazolo[1,2-a] quinoxalin-4-amine | 428.8 |
| B100 | 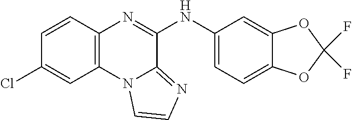 [SMILES: FC1(F)Oc2ccc(Nc3nc4ccc(Cl)cc4n4ccnc34)cc2O1] | 8-chloro-N-(2,2- difluorobenzo [d][1,3]dihydroxy-5-yl) imidazolo[1,2-a] quinoxalin-4-amine | 374.8 |
| B101 | 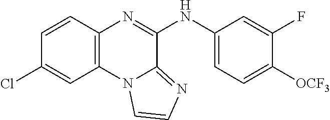 [SMILES: Fc1cc(Nc2nc3ccc(Cl)cc3n3ccnc23)ccc1OC(F)(F)F] | 8-chloro-N- (3-fluoro-4- (trifluoromethoxy)phenyl) imidazolo[1,2-a] quinoxalin-4-amine | 396.8 |
| B102 | 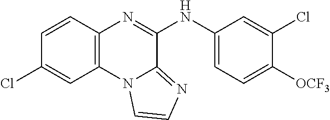 [SMILES: FC(F)(F)Oc1ccc(Nc2nc3ccc(Cl)cc3n3ccnc23)cc1Cl] | 8-chloro-N- (3-chloro-4- (trifluoromethoxy)phenyl) imidazolo[1,2-a] quinoxalin-4-amine | 412.8 |
| B103 | 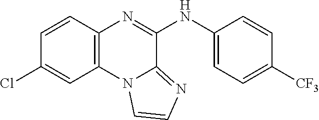 [SMILES: FC(F)(F)c1ccc(Nc2nc3ccc(Cl)cc3n3ccnc23)cc1] | 8-chloro-N-(4- (trifluoromethyl) phenyl) imidazolo[1,2-a] quinoxalin-4-amine | 362.9 |
| B104 | 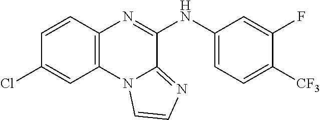 [SMILES: Fc1cc(Nc2nc3ccc(Cl)cc3n3ccnc23)ccc1C(F)(F)F] | 8-chloro-N- (3-fluoro-4- (trifluoromethyl)phenyl) imidazolo[1,2-a] quinoxalin-4-amine | 380.9 |
| B105 | 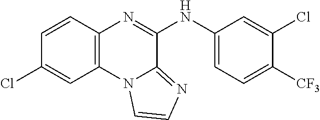 [SMILES: FC(F)(F)c1ccc(Nc2nc3ccc(Cl)cc3n3ccnc23)cc1Cl] | 8-chloro-N- (3-chloro-4- (trifluoromethyl)phenyl) imidazolo[1,2-a] quinoxalin-4-amine | 396.8 |
| B106 | 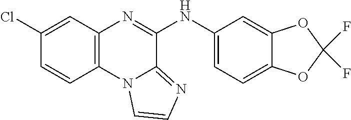 [SMILES: FC1(F)Oc2ccc(Nc3nc4cc(Cl)ccc4n4ccnc34)cc2O1] | 7-chloro-N-(2,2- difluorobenzo[d] [1,3]dihydroxy-5-yl) imidazolo[1,2-a] quinoxalin-4-amine | 374.8 |
| B107 | 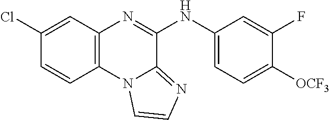 [SMILES: Fc1cc(Nc2nc3cc(Cl)ccc3n3ccnc23)ccc1OC(F)(F)F] | 7-chloro-N- (3-fluoro-4- (trifluoromethoxy)phenyl) imidazolo[1,2-a] quinoxalin-4-amine | 396.8 |
| B108 | 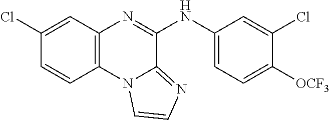 [SMILES: FC(F)(F)Oc1ccc(Nc2nc3cc(Cl)ccc3n3ccnc23)cc1Cl] | 7-chloro-N- (3-chloro-4- (trifluoromethoxy)phenyl) imidazolo[1,2-a] quinoxalin-4-amine | 412.8 |
| B109 | 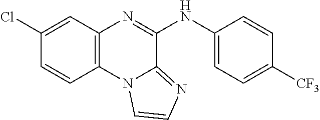 [SMILES: FC(F)(F)c1ccc(Nc2nc3cc(Cl)ccc3n3ccnc23)cc1] | 7-chloro-N-(4- (trifluoromethyl) phenyl) imidazolo[1,2-a] quinoxalin-4-amine | 362.9 |
| B110 | 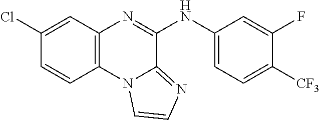 [SMILES: Fc1cc(Nc2nc3cc(Cl)ccc3n3ccnc23)ccc1C(F)(F)F] | 7-chloro-N- (3-fluoro-4- (trifluoromethyl)phenyl) imidazolo[1,2-a] quinoxalin-4-amine | 380.9 |
| B111 | 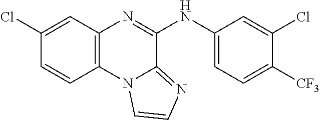 [SMILES: FC(F)(F)c1ccc(Nc2nc3cc(Cl)ccc3n3ccnc23)cc1Cl] | 7-chloro-N- (3-chloro-4- (trifluoromethyl)phenyl) imidazolo[1,2-a] quinoxalin-4-amine | 396.8 |
| B112 | 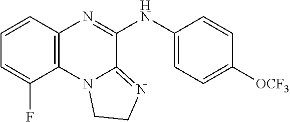 [SMILES: Fc1cccc2c1N1CCN=C1C(Nc1ccc(OC(F)(F)F)cc1)=N2] | 9-fluoro-N-(4- (trifluoromethoxy) phenyl)-1,2- dihydroimidazo[1,2-a] quinoxalin-4-amine | 364.9 |
| B113 | 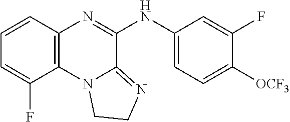 [SMILES: Fc1cc(NC2=Nc3cccc(F)c3N3CCN=C23)ccc1OC(F)(F)F] | 9-fluoro-N-(3-fluoro-4- (trifluoromethoxy)phenyl)- 1,2-dihydroimidazo[1,2-a] quinoxalin-4-amine | 382.9 |
| B114 | 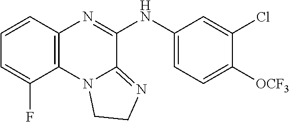 [SMILES: Fc1cccc2c1N1CCN=C1C(Nc1ccc(OC(F)(F)F)c(Cl)c1)=N2] | N-(3-chloro-4- (trifluoromethoxy) phenyl)-9-fluoro- 1,2-dihydroimidazo[1,2-a] quinoxalin-4-amine | 398.9 |
| B115 | 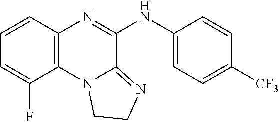 [SMILES: Fc1cccc2c1N1CCN=C1C(Nc1ccc(C(F)(F)F)cc1)=N2] | 9-fluoro-N-(4- (trifluoromethyl)phenyl)- 1,2-dihydroimidazo[1,2-a] quinoxalin-4-amine | 348.9 |
| B116 | 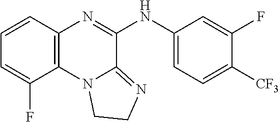 [SMILES: Fc1cc(NC2=Nc3cccc(F)c3N3CCN=C23)ccc1C(F)(F)F] | 9-fluoro-N-(3-fluoro-4- (trifluoromethyl)phenyl)- 1,2-dihydroimidazo[1,2-a] quinoxalin-4-amine | 366.9 |
| B117 | 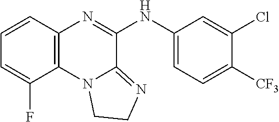 [SMILES: Fc1cccc2c1N1CCN=C1C(Nc1ccc(C(F)(F)F)c(Cl)c1)=N2] | N-(3-chloro-4- (trifluoromethyl) phenyl)-9-fluoro- 1,2-dihydroimidazo[1,2-a] quinoxalin-4-amine | 382.9 |
| B118 | 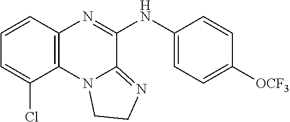 [SMILES: FC(F)(F)Oc1ccc(NC2=Nc3cccc(Cl)c3N3CCN=C23)cc1] | 9-chloro-N-(4- (trifluoromethoxy) phenyl)-1,2- dihydroimidazo[1,2-a] quinoxalin-4-amine | 380.9 |
| B119 | 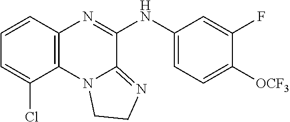 [SMILES: Fc1cc(NC2=Nc3cccc(Cl)c3N3CCN=C23)ccc1OC(F)(F)F] | 9-chloro-N-(3-fluoro-4- (trifluoromethoxy)phenyl)- 1,2-dihydroimidazo[1,2-a] quinoxalin-4-amine | 398.9 |
| B120 | 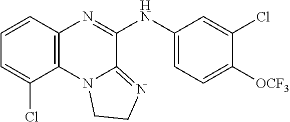 [SMILES: FC(F)(F)Oc1ccc(NC2=Nc3cccc(Cl)c3N3CCN=C23)cc1Cl] | 9-chloro-N-(3-chloro-4- (trifluoromethoxy)phenyl)- 1,2-dihydroimidazo[1,2-a] quinoxalin-4-amine | 414.8 |
| B121 | 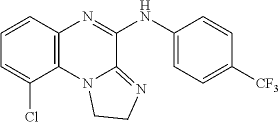 [SMILES: FC(F)(F)c1ccc(NC2=Nc3cccc(Cl)c3N3CCN=C23)cc1] | 9-chloro-N-(4- (trifluoromethyl) phenyl)-1,2- dihydroimidazo[1,2-a] quinoxalin-4-amine | 364.9 |
| B122 | 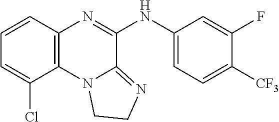 [SMILES: Fc1cc(NC2=Nc3cccc(Cl)c3N3CCN=C23)ccc1C(F)(F)F] | 9-chloro-N-(3-fluoro-4- (trifluoromethyl)phenyl)- 1,2-dihydroimidazo[1,2-a] quinoxalin-4-amine | 382.9 |
| B123 | 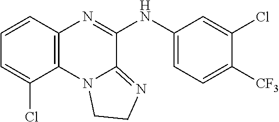 [SMILES: FC(F)(F)c1ccc(NC2=Nc3cccc(Cl)c3N3CCN=C23)cc1Cl] | 9-chloro-N-(3-chloro-4- (trifluoromethyl)phenyl)- 1,2-dihydroimidazo[1,2-a] quinoxalin-4-amine | 398.8 |
| B124 | 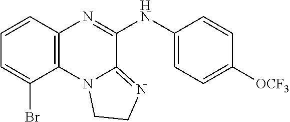 [SMILES: FC(F)(F)Oc1ccc(NC2=Nc3cccc(Br)c3N3CCN=C23)cc1] | 9-bromo-N-(4- (trifluoromethoxy) phenyl)-1,2- dihydroimidazo[1,2-a] quinoxalin-4-amine | 424.8 |
| B125 | 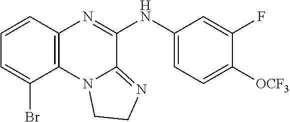 [SMILES: Fc1cc(NC2=Nc3cccc(Br)c3N3CCN=C23)ccc1OC(F)(F)F] | 9-bromo-N-(3-fluoro-4- (trifluoromethoxy)phenyl)- 1,2-dihydroimidazo[1,2-a] quinoxalin-4-amine | 442.8 |
| B126 | 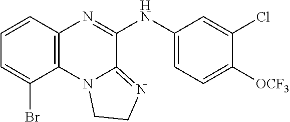 [SMILES: FC(F)(F)Oc1ccc(NC2=Nc3cccc(Br)c3N3CCN=C23)cc1Cl] | 9-bromo-N-(3-chloro-4- (trifluoromethoxy)phenyl)- 1,2-dihydroimidazo[1,2-a] quinoxalin-4-amine | 458.8 |
| B127 | 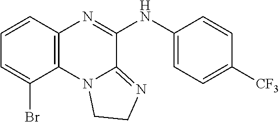 [SMILES: FC(F)(F)c1ccc(NC2=Nc3cccc(Br)c3N3CCN=C23)cc1] | 9-bromo-N-(4- (trifluoromethyl) phenyl)-1,2- dihydroimidazo[1,2-a] quinoxalin-4-amine | 408.8 |
| B128 | 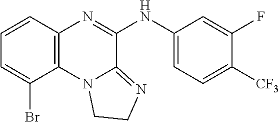 [SMILES: Fc1cc(NC2=Nc3cccc(Br)c3N3CCN=C23)ccc1C(F)(F)F] | 9-bromo-N-(3-fluoro-4- (trifluoromethyl)phenyl)- 1,2-dihydroimidazo[1,2-a] quinoxalin-4-amine | 426.8 |
| B129 | 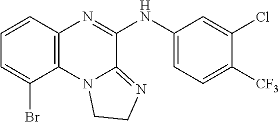 [SMILES: FC(F)(F)c1ccc(NC2=Nc3cccc(Br)c3N3CCN=C23)cc1Cl] | 9-bromo-N-(3-chloro-4- (trifluoromethyl)phenyl)- 1,2-dihydroimidazo[1,2-a] quinoxalin-4-amine | 442.8 |
| B130 | 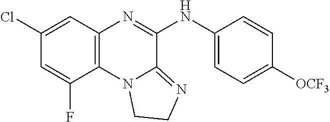 [SMILES: Fc1cc(Cl)cc2c1N1CCN=C1C(Nc1ccc(OC(F)(F)F)cc1)=N2] | 7-chloro-9-fluoro-N-(4- (trifluoromethoxy)phenyl)- 1,2-dihydroimidazo[1,2-a] quinoxalin-4-amine | 398.9 |
| B131 | 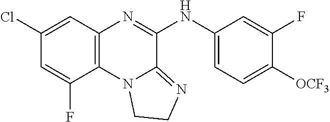 [SMILES: Fc1cc(NC2=Nc3cc(Cl)cc(F)c3N3CCN=C23)ccc1OC(F)(F)F] | 7-chloro-9-fluoro- N-(3-fluoro-4- (trifluoromethoxy)phenyl)- 1,2-dihydroimidazo[1,2-a] quinoxalin-4-amine | 416.9 |
| B132 | 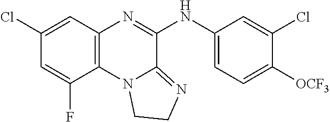 [SMILES: Fc1cc(Cl)cc2c1N1CCN=C1C(Nc1ccc(OC(F)(F)F)c(Cl)c1)=N2] | 7-chloro-N-(3-chloro-4- (trifluoromethoxy) phenyl)-9-fluoro- 1,2-dihydroimidazo[1,2-a] quinoxalin-4-amine | 432.8 |
| B133 | 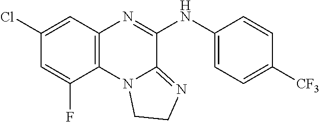 [SMILES: Fc1cc(Cl)cc2c1N1CCN=C1C(Nc1ccc(C(F)(F)F)cc1)=N2] | 7-chloro-9-fluoro-N-(4- (trifluoromethyl)phenyl)- 1,2-dihydroimidazo[1,2-a] quinoxalin-4-amine | 382.9 |
| B134 | 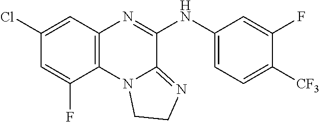 [SMILES: Fc1cc(NC2=Nc3cc(Cl)cc(F)c3N3CCN=C23)ccc1C(F)(F)F] | 7-chloro-9-fluoro-N- (3-fluoro-4- (trifluoromethyl)phenyl)- 1,2-dihydroimidazo[1,2-a] quinoxalin-4-amine | 400.9 |
| B135 | 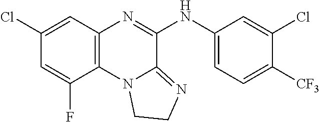 [SMILES: Fc1cc(Cl)cc2c1N1CCN=C1C(Nc1ccc(C(F)(F)F)c(Cl)c1)=N2] | 7-chloro-N-(3-chloro-4- (trifluoromethyl) phenyl)-9-fluoro-1,2- dihydroimidazo[1,2-a] quinoxalin-4-amine | 416.8 |
| B136 | 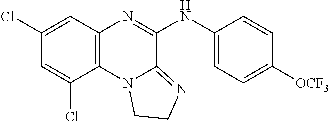 [SMILES: FC(F)(F)Oc1ccc(NC2=Nc3cc(Cl)cc(Cl)c3N3CCN=C23)cc1] | 7,9-dichloro-N-(4- (trifluoromethoxy)phenyl)- 1,2-dihydroimidazo[1,2-a] quinoxalin-4-amine | 414.8 |
| B137 | 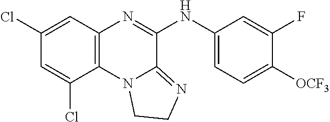 [SMILES: Fc1cc(NC2=Nc3cc(Cl)cc(Cl)c3N3CCN=C23)ccc1OC(F)(F)F] | 7,9-dichloro-N-(3-fluoro-4- (trifluoromethoxy)phenyl)- 1,2-dihydroimidazo[1,2-a] quinoxalin-4-amine | 432.8 |
| B138 | 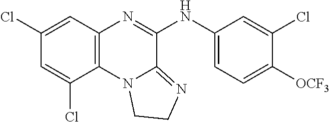 [SMILES: FC(F)(F)Oc1ccc(NC2=Nc3cc(Cl)cc(Cl)c3N3CCN=C23)cc1Cl] | 7,9-dichloro-N-(3-chloro-4- (trifluoromethoxy)phenyl)- 1,2-dihydroimidazo[1,2-a] quinoxalin-4-amine | 448.8 |
| B139 | 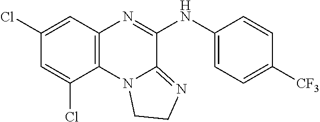 [SMILES: FC(F)(F)c1ccc(NC2=Nc3cc(Cl)cc(Cl)c3N3CCN=C23)cc1] | 7,9-dichloro-N-(4- (trifluoromethyl) phenyl)-1,2- dihydroimidazo[1,2-a] quinoxalin-4-amine | 398.8 |
| B140 | 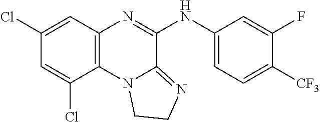 [SMILES: Fc1cc(NC2=Nc3cc(Cl)cc(Cl)c3N3CCN=C23)ccc1C(F)(F)F] | 7,9-dichloro-N-(3-fluoro-4- (trifluoromethyl)phenyl)- 1,2-dihydroimidazo[1,2-a] quinoxalin-4-amine | 416.8 |
| B141 | 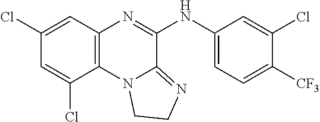 [SMILES: FC(F)(F)c1ccc(NC2=Nc3cc(Cl)cc(Cl)c3N3CCN=C23)cc1Cl] | 7,9-dichloro-N-(3-chloro-4- (trifluoromethyl)phenyl)- 1,2-dihydroimidazo[1,2-a] quinoxalin-4-amine | 432.8 |
| B142 | 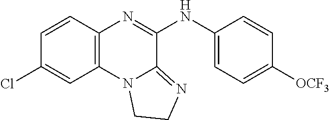 [SMILES: FC(F)(F)Oc1ccc(NC2=Nc3ccc(Cl)cc3N3CCN=C23)cc1] | 8-chloro-N-(4- (trifluoromethoxy) phenyl)-1,2- dihydroimidazo[1,2-a] quinoxalin-4-amine | 380.9 |
| B143 | 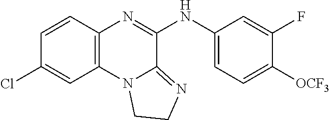 [SMILES: Fc1cc(NC2=Nc3ccc(Cl)cc3N3CCN=C23)ccc1OC(F)(F)F] | 8-chloro-N-(3-fluoro-4- (trifluoromethoxy)phenyl)- 1,2-dihydroimidazo[1,2-a] quinoxalin-4-amine | 398.9 |
| B144 | 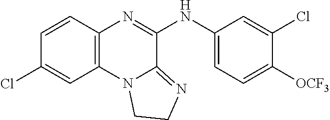 [SMILES: FC(F)(F)Oc1ccc(NC2=Nc3ccc(Cl)cc3N3CCN=C23)cc1Cl] | 8-chloro-N-(3-chloro-4- (trifluoromethoxy)phenyl)- 1,2-dihydroimidazo[1,2-a] quinoxalin-4-amine | 414.8 |
| B145 | 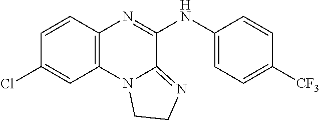 [SMILES: FC(F)(F)c1ccc(NC2=Nc3ccc(Cl)cc3N3CCN=C23)cc1] | 8-chloro-N-(4- (trifluoromethyl) phenyl)-1,2- dihydroimidazo[1,2-a] quinoxalin-4-amine | 364.9 |
| B146 | 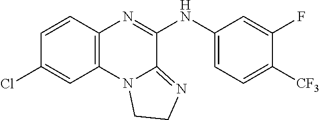 [SMILES: Fc1cc(NC2=Nc3ccc(Cl)cc3N3CCN=C23)ccc1C(F)(F)F] | 8-chloro-N-(3-Fluoro-4- (trifluoromethyl)phenyl)- 1,2-dihydroimidazo[1,2-a] quinoxalin-4-amine | 382.9 |
| B147 | 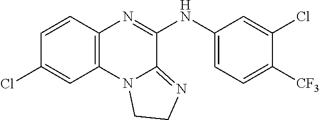 [SMILES: FC(F)(F)c1ccc(NC2=Nc3ccc(Cl)cc3N3CCN=C23)cc1Cl] | 8-chloro-N-(3-chloro-4- (trifluoromethyl)phenyl)- 1,2-dihydroimidazo[1,2-a] quinoxalin-4-amine | 398.8 |
| B148 | 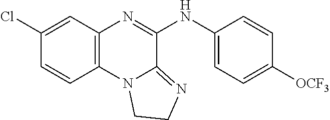 [SMILES: FC(F)(F)Oc1ccc(NC2=Nc3cc(Cl)ccc3N3CCN=C23)cc1] | 7-chloro-N-(4- (trifluoromethoxy) phenyl)-1,2- dihydroimidazo[1,2-a] quinoxalin-4-amine | 380.9 |
| B149 | 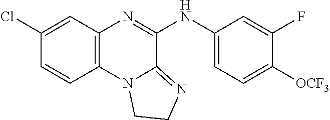 [SMILES: Fc1cc(NC2=Nc3cc(Cl)ccc3N3CCN=C23)ccc1OC(F)(F)F] | 7-chloro-N-(3-fluoro-4- (trifluoromethoxy)phenyl)- 1,2-dihydroimidazo[1,2-a] quinoxalin-4-amine | 398.9 |
| B150 | 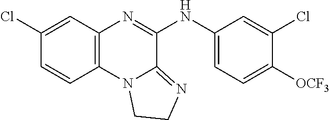 [SMILES: FC(F)(F)Oc1ccc(NC2=Nc3cc(Cl)ccc3N3CCN=C23)cc1Cl] | 7-chloro-N-(3-chloro-4- (trifluoromethoxy)phenyl)- 1,2-dihydroimidazo[1,2-a] quinoxalin-4-amine | 414.8 |
| B151 | 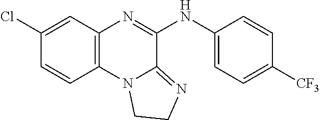 [SMILES: FC(F)(F)c1ccc(NC2=Nc3cc(Cl)ccc3N3CCN=C23)cc1] | 7-chloro-N-(4- (trifluoromethyl) phenyl)-1,2- dihydroimidazo[1,2-a] quinoxalin-4-amine | 364.9 |
| B152 | 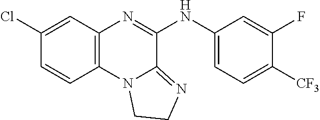 [SMILES: Fc1cc(NC2=Nc3cc(Cl)ccc3N3CCN=C23)ccc1C(F)(F)F] | 7-chloro-N-(3-fluoro-4- (trifluoromethyl)phenyl)- 1,2-dihydroimidazo[1,2-a] quinoxalin-4-amine | 382.9 |
| B153 | 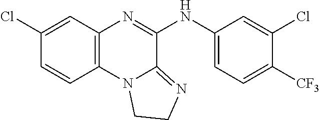 [SMILES: FC(F)(F)c1ccc(NC2=Nc3cc(Cl)ccc3N3CCN=C23)cc1Cl] | 7-chloro-N-(3-chloro-4- (trifluoromethyl)phenyl)- 1,2-dihydroimidazo[1,2-a] quinoxalin-4-amine | 398.8 |

### Example 250: Induction of miR-124 Expression in PBMC Cells

This study evaluated the ability of the compound of the present invention to influence miR-124 transcriptional activity in vitro. The experiment analyzed miR-124 expression in human PBMCs (Sailybio) using quantitative PCR (qPCR).

Thawed human PBMCs (1 mL) were added to 19 mL of culture medium (RPMI1640+10% hFBS+1‰ 2-mercaptoethanol) and the PBMCs were incubated in a cell culture incubator for 30 minutes. The PBMCs were collected and the cells (3.0*10⁶ cells/well/3 mL) were resuspended in medium containing PMA (10 ng/mL; Invivogen) and ionomycin (200 ng/mL; Invivogen). The cells were incubated in a 6-well plate for 48 hours. Diluted compound was added to the cells to achieve a final concentration of 0.1 μM. After 72 hours, the samples were centrifuged and the cells were collected.

Total miRNA was extracted from the cells using the MiPure Cell/Tissue miRNA Kit (Vazyme) according to the manufacturer's instructions, followed by purification. The purified samples were treated with genomic DNA elimination buffer, and reverse transcription was performed using a GeneAmp PCR System. The resulting cDNA was subjected to quantitative real-time PCR (qPCR) analysis on a QuantStudio™ 7 Flex Real-Time PCR System. Data analysis was performed using the QuantStudio 7 software with default settings to automatically determine Ct values, and the results were exported. In this assay, miR-191 was used as the reference gene, and the DMSO-treated group served as the control. The relative expression of miR-124 was calculated using the following formula:

$$Δ Ct = Ct ( target gene ) - Ct ( reference gene ) \\ ΔΔ Ct = Δ Ct ( treated group ) - Δ Ct ( control group ) \\ Relative mRNA expression = 2 \hat{} ( - ΔΔ Ct )$$

TABLE 1
Effects of Test Compounds (0.1 μM)
on miR-124 Expression in PBMCs

| | Fold | | Fold | | Fold |
|---|---|---|---|---|---|
| Com- | change vs | Com- | change vs | Com- | change vs |
| pound | DMSO | pound | DMSO | pound | DMSO |
| 1 | 16.04 | 41 | 24.38 | 67 | 28.83 |
| 2 | 16.48 | 42 | 14.07 | 68 | 29.53 |
| 6 | 11.93 | 43 | 18.94 | 69 | 22.87 |
| 9 | 26.88 | 44 | 17.49 | 70 | 16.44 |
| 10 | 15.39 | 45 | 17.85 | 71 | 25.33 |
| 11 | 18.42 | 46 | 9.99 | 72 | 18.96 |
| 12 | 21.98 | 47 | 9.86 | 73 | 8.59 |
| 13 | 20.90 | 48 | 20.43 | 74 | 31.61 |
| 14 | 24.72 | 49 | 19.53 | 79 | 17.82 |
| 15 | 12.66 | 50 | 18.49 | 80 | 19.78 |
| 16 | 13.37 | 51 | 23.12 | 81 | 15.27 |
| 17 | 18.99 | 52 | 22.29 | 83 | 18.33 |
| 18 | 17.60 | 54 | 14.87 | 84 | 15.54 |
| 19 | 21.21 | 55 | 22.29 | 85 | 10.43 |
| 21 | 15.54 | 56 | 17.16 | 87 | 15.81 |
| 22 | 15.27 | 58 | 27.29 | 89 | 24.32 |
| 28 | 10.40 | 59 | 20.51 | 91 | 27.56 |
| 29 | 9.32 | 61 | 12.13 | 92 | 18.78 |
| 34 | 14.73 | 62 | 15.52 | 95 | 26.99 |
| 39 | 16.75 | 63 | 18.44 | 96 | 17.70 |
| 40 | 17.47 | 66 | 22.69 | | |
| B1 | 43.82 | B67 | 23.41 | B110 | 25.53 |
| B2 | 37.35 | B75 | 29.53 | B111 | 22.49 |
| B3 | 23.46 | B76 | 18.49 | B118 | 28.81 |
| B4 | 33.15 | B77 | 23.12 | B119 | 25.37 |
| B5 | 31.28 | B78 | 14.87 | B120 | 22.64 |
| B6 | 21.97 | B79 | 22.29 | B121 | 29.53 |
| B7 | 32.03 | B80 | 17.16 | B122 | 27.33 |
| B8 | 33.48 | B87 | 15.84 | B123 | 24.26 |
| B9 | 22.62 | B88 | 20.06 | B130 | 25.57 |
| B16 | 33.39 | B89 | 22.31 | B131 | 27.01 |
| B17 | 31.93 | B90 | 24.90 | B132 | 27.59 |
| B18 | 22.65 | B91 | 25.71 | B133 | 26.32 |
| B21 | 35.25 | B92 | 27.08 | B134 | 27.54 |
| B22 | 32.54 | B93 | 19.08 | B135 | 24.93 |
| B23 | 25.21 | B94 | 26.35 | B136 | 34.11 |
| B24 | 26.68 | B95 | 24.77 | B137 | 31.13 |
| B25 | 23.43 | B96 | 23.21 | B138 | 29.94 |
| B30 | 22.49 | B97 | 26.20 | B139 | 40.58 |
| B31 | 24.42 | B98 | 22.91 | B140 | 38.37 |
| B32 | 19.73 | B99 | 17.63 | B141 | 36.20 |
| B49 | 32.05 | B100 | 19.57 | B142 | 25.59 |
| B50 | 21.34 | B101 | 24.74 | B143 | 28.36 |
| B52 | 30.23 | B102 | 27.33 | B144 | 27.75 |
| B53 | 36.78 | B103 | 25.52 | B145 | 30.02 |
| B54 | 34.07 | B104 | 28.13 | B146 | 28.73 |
| B55 | 36.33 | B105 | 27.78 | B147 | 27.91 |
| B56 | 40.72 | B106 | 16.33 | B148 | 29.35 |
| B59 | 25.39 | B107 | 24.41 | B149 | 34.69 |
| B60 | 24.78 | B108 | 22.47 | B150 | 26.37 |
| B61 | 19.86 | B109 | 26.38 | ABX464 | 13.51/16.17 |

Note:
The test results for compound ABX464 represent data obtained from two independent experiments.

As shown in the table above, compared with the positive control ABX464, the compounds of the present invention demonstrate significantly stronger activity in inducing miR-124 expression in PBMCs.

### Example 251: In Vivo Pharmacokinetic Evaluation in Mice

The compound was administered orally (p.o.) at a dose of 10 mg/kg. The test formulation was prepared at a final concentration of 0.4 mg/mL in 5% DMSO+45% PEG400+50% aqueous solution or suspension. Male ICR mice (9 per group) were sampled at three discontinuous time points (n=3 mice/time point). Approximately 80 μL of blood was collected via orbital or cardiac puncture at 5 min, 15 min, 30 min, 1 h, 2 h, 4 h, 8 h, and 24 h post-dose. All whole blood samples were collected in EDTA K₂-containing tubes. After centrifugation at 4° C. (4000 rpm, 10 min), plasma was collected and stored below −40° C. for subsequent analysis. Compound concentrations in plasma were determined by liquid chromatography-tandem mass spectrometry (LC-MS/MS), and pharmacokinetic parameters were derived from the plasma concentration-time curves.

TABLE 2
Pharmacokinetic parameters of compounds 2, 18, 19, 43, 67, 68, 70,
71, 89, B1, B4, B7, B54, B118, B121, B139 and ABX464 in mice

| | Administra- | Dose | t_{1/2} | Tₘₐₓ | Cₘₐₓ | AUC₀₋ₜ |
|---|---|---|---|---|---|---|
| Compound | tion route | (mg/kg) | (h) | (h) | (ng/mL) | (ng · h/L) |
| 2 | po | 10 | 3.45 | 4.0 | 207 | 933 |
| 18 | po | 10 | 3.78 | 2.0 | 365 | 1841 |
| 19 | po | 10 | 3.36 | 4.0 | 358 | 1638 |
| 43 | po | 10 | 3.87 | 2.0 | 348 | 1682 |
| 67 | po | 10 | 3.29 | 4.0 | 403 | 2033 |
| 68 | po | 10 | 2.17 | 0.5 | 243 | 879 |
| 70 | po | 10 | 2.32 | 0.5 | 357 | 1427 |
| 71 | po | 10 | 3.41 | 4.0 | 274 | 1369 |
| 89 | po | 10 | 3.46 | 2.0 | 454 | 1864 |
| B1 | po | 10 | 2.58 | 1.0 | 450 | 3586 |
| B4 | po | 10 | 3.26 | 2.0 | 271 | 2849 |
| B7 | po | 10 | 3.05 | 2.0 | 378 | 3235 |
| B54 | po | 10 | 1.96 | 0.5 | 346 | 1332 |
| B118 | po | 10 | 2.47 | 2.0 | 269 | 1875 |
| B121 | po | 10 | 2.12 | 1.0 | 378 | 1948 |
| B139 | po | 10 | 3.36 | 2.0 | 375 | 3264 |
| ABX464 | po | 10 | 1.08 | 0.5 | 168 | 420 |

As shown in the above table, the compounds of the present invention exhibit favorable oral absorption characteristics. Key pharmacokinetic parameters including maximum plasma concentration (Cₘₐₓ), area under the curve (AUC₀₋ₜ), and mean residence time (MRT) were superior to those of the reference compound ABX464. These optimal absorption properties are particularly significant for enhancing drug efficacy, reducing dosage requirements, and lowering treatment costs.

Further experimental studies confirmed that other inventive compounds also demonstrate excellent oral bioavailability, with their Cₘₐₓ, AUC₀₋ₜ, and MRT values consistently outperforming ABX464.

### Example 252: Preventive and Therapeutic Effects of Compounds on Inflammatory Bowel Disease (IBD) in Mice

This study evaluated the preventive and therapeutic effects of the compounds of the present invention on dextran sulfate sodium (DSS)-induced IBD in mice. The experiment utilized female C57BL/6 mice (purchased from Beijing Vital River Laboratory Animal Technology Co., Ltd.) to establish the DSS-induced IBD model.

### 1. Methods and Materials

### 1.1 Experimental Animals

Female C57BL/6 mice (6-8 weeks old) were purchased from Beijing Vital River Laboratory Animal Technology Co., Ltd. Upon arrival, the mice were housed in individually ventilated cages (IVC) under specific pathogen-free (SPF) conditions, with 2-6 mice per cage. They were provided with ad libitum access to food and water and maintained at a temperature of 20-26° C. (68-79° F.), relative humidity of 40-70%, and a 12-hour light/dark cycle (lights on from 08:00 to 20:00). After a 7-day acclimatization period, experiments were initiated.

### 1.2 Reagents and Equipment

Dextran sulfate sodium (DSS, MW=36,000-50,000): MP Biomedicals, Cat. No. 160110, Lot No. S3045;
Dimethyl sulfoxide (DMSO): Sinopharm Chemical Reagent Co., Ltd., Cat. No. 30072418, Lot No. 20230309;
Polyethylene glycol-15 hydroxystearate (Solutol HS-15): Sigma, Cat. No. 42966, Lot No. BCCJ5912;
Zoletil (Tiletamine-Zolazepam): Virbac, Lot No. 8V4RA;
Xylazine: Tokyo Chemical Industry (TCI), Lot No. R834H-MB;
Balance: METTLER TOLEDO, Model AL104;
Centrifuge: Thermo Fisher Scientific, Model 21R;

### 1.3 Experimental Design and Methods

### 1.3.1 Drug Preparation

Test compounds and ABX464 vehicle: 5% DMSO+10% Solutol HS-15+85% saline, stored at 4° C.

DSS solution: 2% (w/v) DSS in autoclaved drinking water, prepared fresh before use.

### 1.3.2 Experimental Groups and Procedures

The mice were randomly allocated into eight groups according to body weight: normal control group (negative control), model group (DSS group), ABX464 treatment group, test compound groups (administered with Compounds 19, 43, 59, 84, and 89 of the present invention at 40 mg/kg, p.o., q.d.)

| Group | Experiment design | N | Dose |
|---|---|---|---|
| Normal control | 10 days normal water | 5 | vehicle |
| Model group | 7 days 2% DSS + 3 days normal water | 5 | vehicle |
| ABX464 | 7 days 2% DSS + 3 days normal water | 5 | 40 mg/kg |
| Compound 19 | 7 days 2% DSS + 3 days normal water | 5 | 40 mg/kg |
| Compound 43 | 7 days 2% DSS + 3 days normal water | 5 | 40 mg/kg |
| Compound 59 | 7 days 2% DSS + 3 days normal water | 5 | 40 mg/kg |
| Compound 84 | 7 days 2% DSS + 3 days normal water | 5 | 40 mg/kg |
| Compound 89 | 7 days 2% DSS + 3 days normal water | 5 | 40 mg/kg |

Following a 7-day acclimation period, the model group and compound treatment groups were administered 2% DSS aqueous solution ad libitum for 7 days starting from Day 0, followed by 3 days of normal water consumption. All groups received daily oral gavage of their respective vehicles or test compounds for 10 consecutive days beginning on the trial commencement date. During the experimental period, body weight changes in mice were monitored and recorded daily, the Disease Activity Index (DAI) was scored each day with the total score calculated on the final day, and on the last experimental day all animals were humanely euthanized followed by colon collection and length measurement.

### 1.3.3 Statistical Analysis

The experimental data were expressed as mean±standard error of the mean (mean±S.E.M.). Statistical analysis was performed using GraphPad Prism or SPSS software with appropriate statistical methods. A value of P<0.05 was considered statistically significant.

### 2. Results

### 2.1 Effects of the Compounds of the Present Invention on Body Weight in DSS-Induced IBD Mice

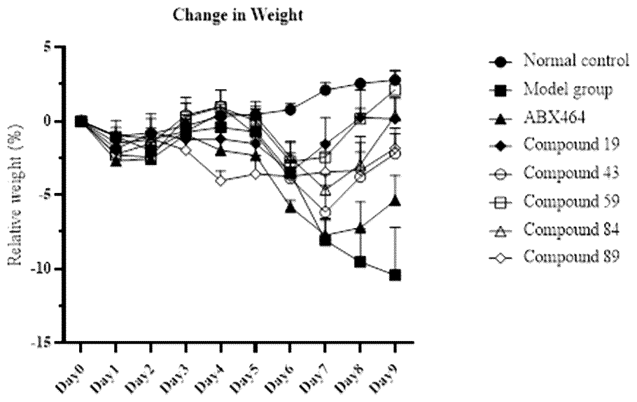

The body weight results are shown in FIG. 1. Compared with the normal control group, mice in the model group exhibited significant weight loss starting from day 5, with over 10% reduction by day 10. In contrast to the model group, all compound-treated groups showed weight recovery beginning on day 7. By day 10: Compounds 19, 59 and 84 of the present invention demonstrated weight gain percentages of 0.16%, 2.12% and 0.38%, respectively; Compounds 43 and 89 showed weight loss percentages of 2.22% and 1.85%, respectively. Notably, compared with ABX464 (5.37% weight loss), all tested compounds of the present invention displayed superior ameliorative effects on body weight reduction.

### 2.2 Effects of the Compounds of the Present Invention on DAI in DSS-Induced IBD Mice

The results of DAI scores are presented in Table 3. Compared with the normal control group, the model group showed significantly elevated DAI scores, reaching a total of 14.5 points. In contrast, the compound-treated groups (19, 43, 59, 84, and 89) of the present invention demonstrated total DAI scores of 8.33, 10.03, 8.57, 8.47, and 10.33 points, respectively, indicating marked reductions compared to the model group. Notably, compounds 19, 59, and 84 exhibited statistically significant reductions in total DAI scores by 42.55%, 40.90%, and 41.59%, respectively. Furthermore, when compared with ABX464 (which showed only a 5.72% reduction), the compounds of the present invention achieved substantially greater decreases in total DAI scores, demonstrating superior efficacy in improving DAI scores relative to the reference drug ABX464.

TABLE 3
Total Disease Activity Index (DAI) Scores

| | Normal | Model | | Compound | Compound | Compound | Compound | Compound |
|---|---|---|---|---|---|---|---|---|
| Group | control | group | ABX464 | 19 | 43 | 59 | 84 | 89 |
| DAI | 0.67 ± | 14.50 ± | 13.67 ± | 8.33 ± | 10.03 ± | 8.57 ± | 8.47 ± | 10.33 ± |
| | 0.37 | 0.87 | 1.31 | 1.80 | 2.11 | 1.10 | 0.55 | 0.94 |

### 2.3 Effects of the Compounds of the Present Invention on Colon Length in DSS-Induced IBD Mice

The colon length measurement results are shown in Table 4. Compared with the normal control group, the model group exhibited significant colon shortening, reaching only 77.32% of the normal length, indicating successful establishment of the IBD model. In contrast, administration of the compounds of the present invention (19, 43, 59, 84, and 89) significantly increased colon length to 96.27%, 99.69%, 100%, 96.89%, and 99.07% of normal control values, respectively (all P<0.05 versus model group). These results demonstrate that the compounds effectively ameliorated DSS-induced colonic damage, with near-complete restoration to normal physiological length. Furthermore, compared with ABX464 treatment, the compounds of the present invention showed comparable or slightly superior efficacy in improving colon length (particularly Compound 59, which completely restored normal colon length).

TABLE 4
Colon length of mice (cm)

| | Normal | Model | | Compound | Compound | Compound | Compound | Compound |
|---|---|---|---|---|---|---|---|---|
| Group | control | group | ABX464 | 19 | 43 | 59 | 84 | 89 |
| Colon | 6.44 ± | 4.98 ± | 6.22 ± | 6.20 ± | 6.42 ± | 6.48 ± | 6.24 ± | 6.38 ± |
| length | 0.13 | 0.21 | 0.12 | 0.20 | 0.11 | 0.10 | 0.17 | 0.12 |
| (cm) | | | | | | | | |

### 3. Conclusions

This study evaluated the preventive and therapeutic effects of the inventive compounds on DSS-induced IBD in mice. The experimental model was successfully established, with significant changes observed in key evaluation indicators, including body weight, Daily Disease Activity Index (DAI), and colon length. The final results demonstrated that the inventive compounds exhibited favorable pharmacological effects on body weight, DAI, and colon length in mice. Notably, their efficacy in improving body weight and DAI was significantly superior to that of the reference drug ABX464. Therefore, the inventive compounds show promising preventive and therapeutic effects against DSS-induced IBD in mice.

Further experiments confirmed that other compounds of the present invention (e.g., B7, B83, B97, and B108) also displayed preventive and therapeutic effects on DSS-induced IBD in mice. These compounds demonstrated favorable pharmacological effects on body weight, DAI, and colon length, all of which outperformed the reference drug ABX464.

All references mentioned in the present disclosure are cited as references in this application, just as each reference is cited separately. In addition, it should be understood that after reading the above lecture content of the present disclosure, those skilled in the art can make various changes or modifications to the present disclosure, and these equivalent forms also fall within the scope of the claims attached to the present disclosure.

1. A compound having the structure shown in Formula (1), or an isomer, crystal form, pharmaceutically acceptable salt, hydrate, or solvate thereof; 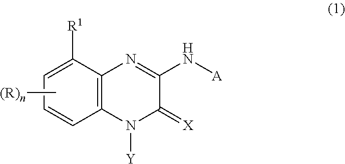
wherein:
n is 0, 1, 2 or 3;
each R is independently selected from the group consisting of: halogen, CN, nitro, C₁₋₃ alkyl, C₁₋₃ haloalkyl, C₁₋₃ alkoxy or C₁₋₃ haloalkoxy;
R¹ is selected from the group consisting of: H, CN, C₁₋₃ alkyl, C₁₋₃ alkoxy or halogen;
A is selected from the group consisting of: 6-10 membered aryl, 5-7 membered heteroaryl, benzo 5-7-membered cycloalkyl or benzo 5-7-membered heterocyclyl, wherein the aryl, heteroaryl, benzo cycloalkyl and benzo heterocyclyl can be further substituted by 0-4 substituents selected from the group consisting of: halogen, CN, C₁₋₃ alkyl, C₃₋₆ cycloalkyl, C₁₋₃ alkoxy, C₁₋₃ alkylthio, C₁₋₃ haloalkyl, C₁₋₃haloalkoxy, C₁₋₃ haloalkylthio or SF₅;
X is O, and Y is C₁₋₃ alkyl, C₁₋₃ haloalkyl or C₃₋₆ cycloalkyl; or
X and Y are taken together with the atoms to which they are bound to form a 5-membered heteroaryl or a 5-membered heterocyclyl, wherein the heteroaryl and heterocyclyl can be further substituted by 0-2 substituents of the following groups: C₁₋₃ alkyl or C₁₋₃ haloalkyl.

2. The compound according to claim 1, or an isomer, crystal form, pharmaceutically acceptable salt, hydrate, or solvate thereof, wherein at least one of R and R′ is halogen.

3. The compound according to claim 1, or an isomer, crystal form, pharmaceutically acceptable salt, hydrate, or solvate thereof, wherein each R is independently selected from the group consisting of: F, Cl, Br, Me, Et, OMe, OEt, CF₃, CN, OCF₃ or NO₂.

4. The compound according to claim 1, or an isomer, crystalline form, pharmaceutically acceptable salt, hydrate, or solvate thereof, wherein R¹ is selected from the group consisting of: H, CN, Me, Et, OMe, OEt, F, Cl, or Br.

5. The compound according to claim 1, or an isomer, crystalline form, pharmaceutically acceptable salt, hydrate, or solvate thereof, wherein A is selected from the group consisting of:

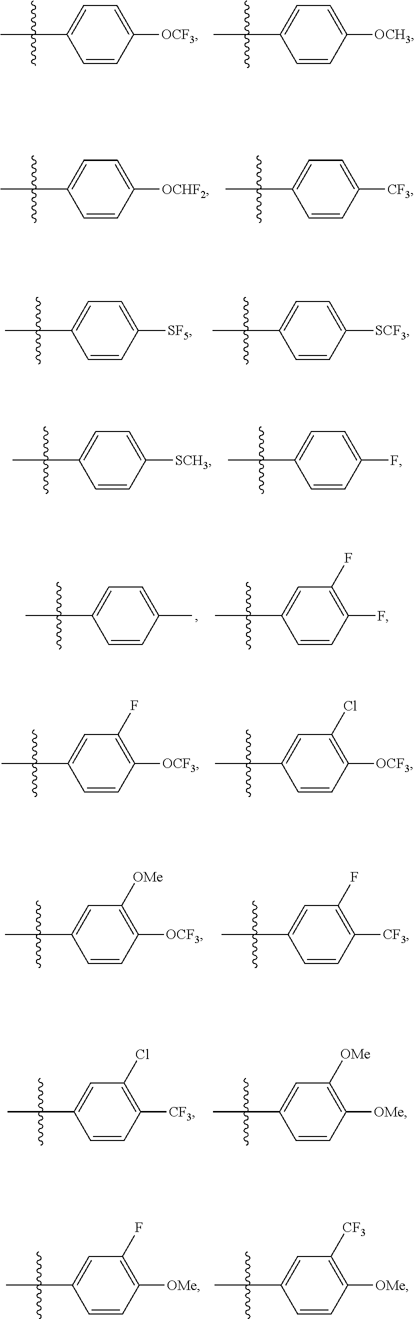

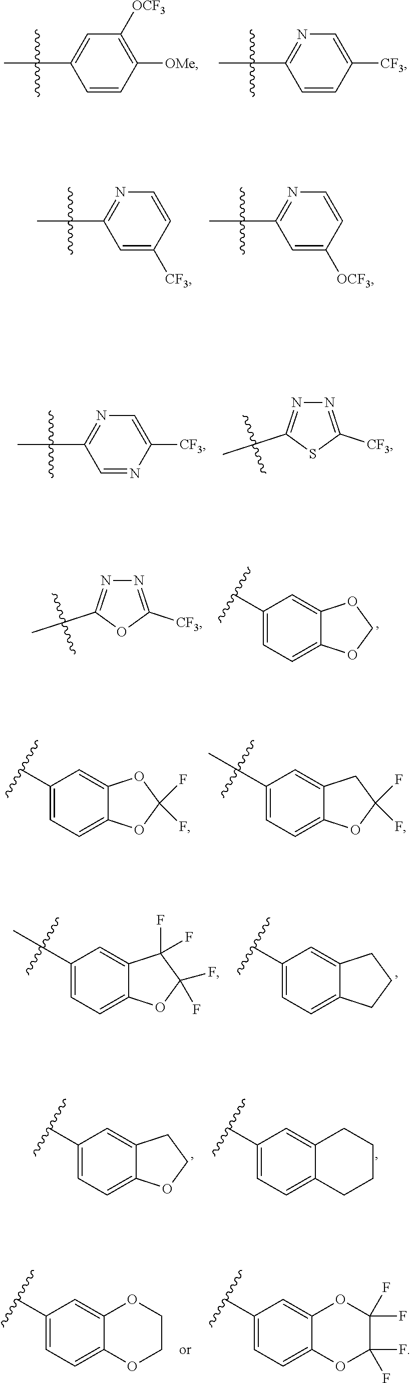

6. The compound according to claim 1, or an isomer, crystal form, pharmaceutically acceptable salt, hydrate, or solvate thereof, characterized in that the compound has the structure as shown in Formula (1A): 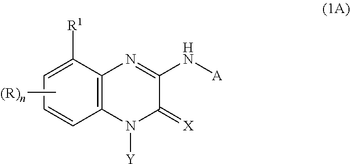
wherein:
n is 0, 1 or 2;
R is selected from the group consisting of: halogen, CN, nitro, C₁₋₃ alkyl, C₁₋₃ haloalkyl or C₁₋₃ alkoxy;
R¹ is a halogen;
A is selected from the group consisting of: 6-10 membered aryl, 5-7 membered heteroaryl or benzo 5-7-membered heterocyclyl, wherein the aryl, heteroaryl and benzo heterocyclyl can be further substituted by 0-4 substituents selected from the group consisting of: halogen, CN, C₁₋₃ alkyl, C₃₋₆ cycloalkyl, C₁₋₃ alkoxy, C₁₋₃ alkylthio, C₁₋₃ haloalkyl, C₁₋₃ haloalkoxy, C₁₋₃ haloalkylthio or SF₅;
X is O, and Y is C₁₋₃ alkyl, C₁₋₃ haloalkyl or C₃₋₆ cycloalkyl; or
X and Y are taken together with the atoms to which they are bound to form a 5-membered heteroaryl or a 5-membered heterocyclyl, wherein the heteroaryl and heterocyclyl can be further substituted by 0-2 substituents of the following groups: C₁₋₃ alkyl or C₁₋₃ haloalkyl.

7. The compound according to claim 1, or an isomer, crystal form, pharmaceutically acceptable salt, hydrate, or solvate thereof, wherein 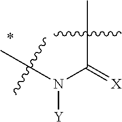
has a structure selected from the group consisting of: 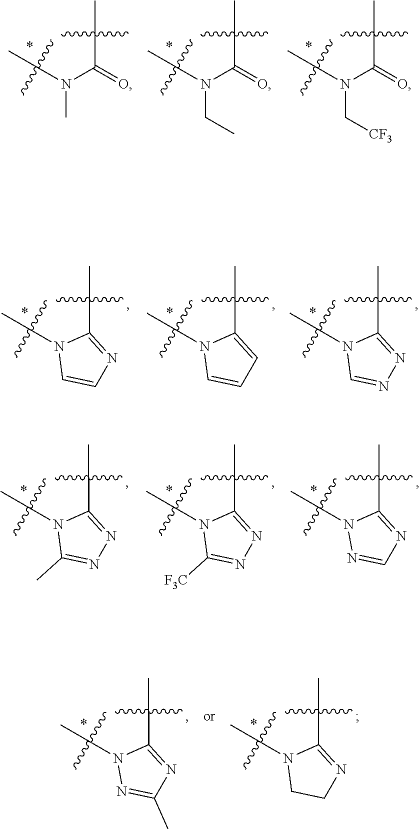
wherein “*” represents the position connected to the benzene ring.

8. The compound according to claim 1, or an isomer, crystal form, pharmaceutically acceptable salt, hydrate, or solvate thereof, characterized in that the compound has a structure selected from the group consisting of: 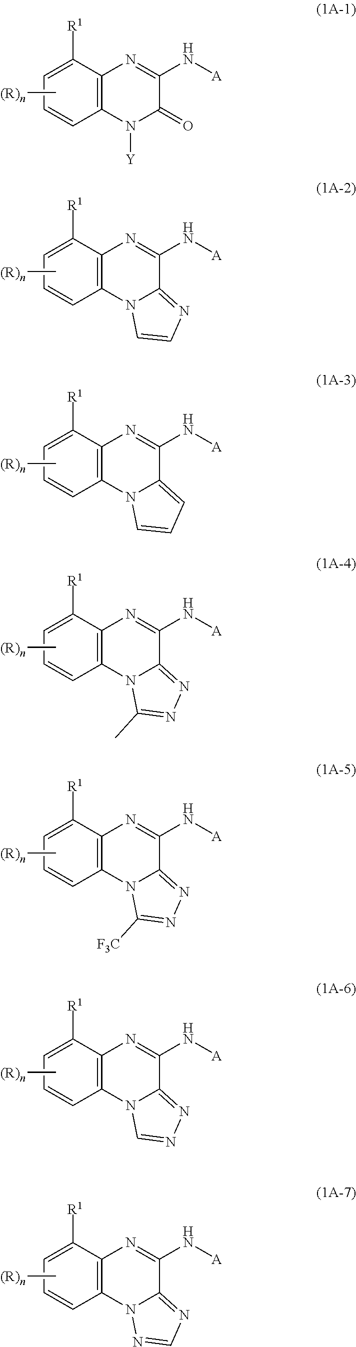 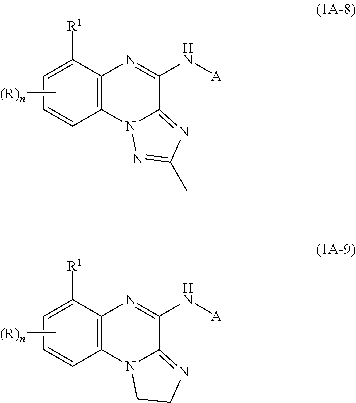
wherein n, Y, R, R and A are as defined in claim 1.

9. The compound according to claim 1, or an isomer, crystal form, pharmaceutically acceptable salt, hydrate, or solvate thereof, characterized in that the compound has the structure as shown in Formula (1B): 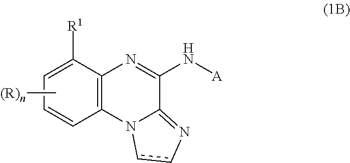
wherein:
“ [symbol] ” indicates a single bond or a double bond;
n is 1, 2 or 3;
each R is independently selected from the group consisting of: halogen, CN, C₁₋₃ alkyl, C₁₋₃ haloalkyl, C₁₋₃ alkoxy or C₁₋₃ haloalkoxy;
R¹ is selected from the group consisting of: H, CN, C₁₋₃ alkyl or C₁₋₃ alkoxy;
A is selected from the group consisting of: 6-10 membered aryl, 5-7 membered heteroaryl, benzo 5-7-membered cycloalkyl or benzo 5-7-membered heterocyclyl, wherein the aryl, heteroaryl, benzo-cycloalkyl and benzo heterocyclyl can be further substituted by 0-4 substituents selected from the group consisting of: halogen, CN, C₁₋₃ alkyl, C₃₋₆ cycloalkyl, C₁₋₃ alkoxy, C₁₋₃ alkylthio, C₁₋₃ haloalkyl, C₁₋₃ haloalkoxy, C₁₋₃ haloalkylthio or SF₅.

10. The compound according to claim 1, or an isomer, crystal form, pharmaceutically acceptable salt, hydrate, or solvate thereof, characterized in that the compound has the structure as shown in Formula (2): 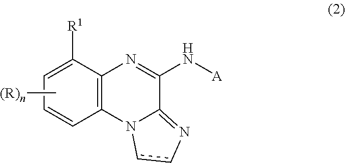
wherein:
“ [symbol] ” indicates a single bond or a double bond;
n is 0, 1 or 2;
R¹ is selected from the group consisting of: H, F, Cl or Br;
each R is independently selected from the group consisting of F, Cl or Br.

11. The compound according to claim 10, or an isomer, crystal form, pharmaceutically acceptable salt, hydrate, or solvate thereof, wherein A is selected from the group consisting of:

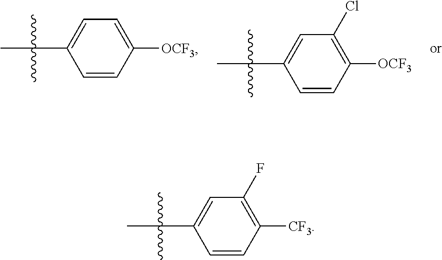 [SMILES: *c1ccc(OC(F)(F)F)cc1 ; *c1ccc(OC(F)(F)F)c(Cl)c1 ; *c1ccc(C(F)(F)F)c(F)c1]

12. The compound according to claim 10, or an isomer, crystal form, pharmaceutically acceptable salt, hydrate, or solvate, wherein n is 1 or 2 when R¹ is H.

13. The compound according to claim 1, an isomer thereof or an pharmaceutically acceptable salt thereof, wherein the compound has a structure selected from the group consisting of:

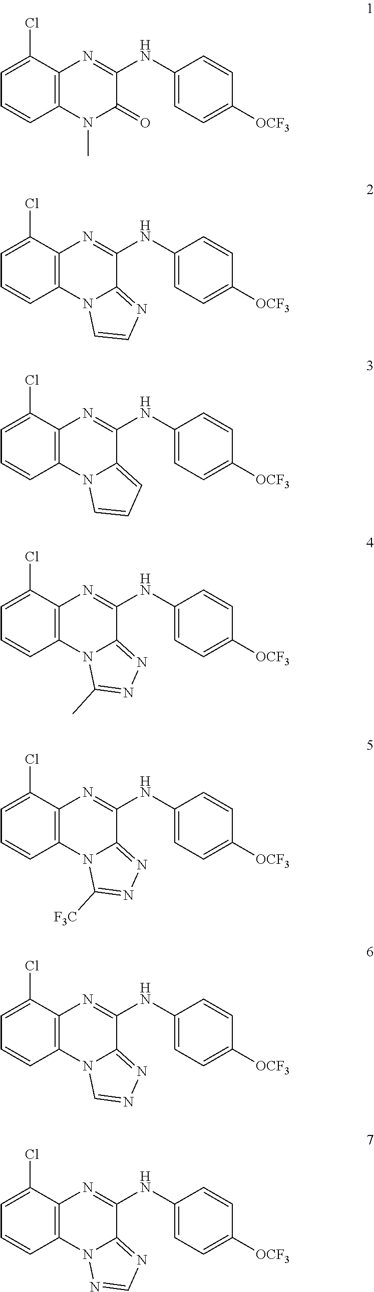

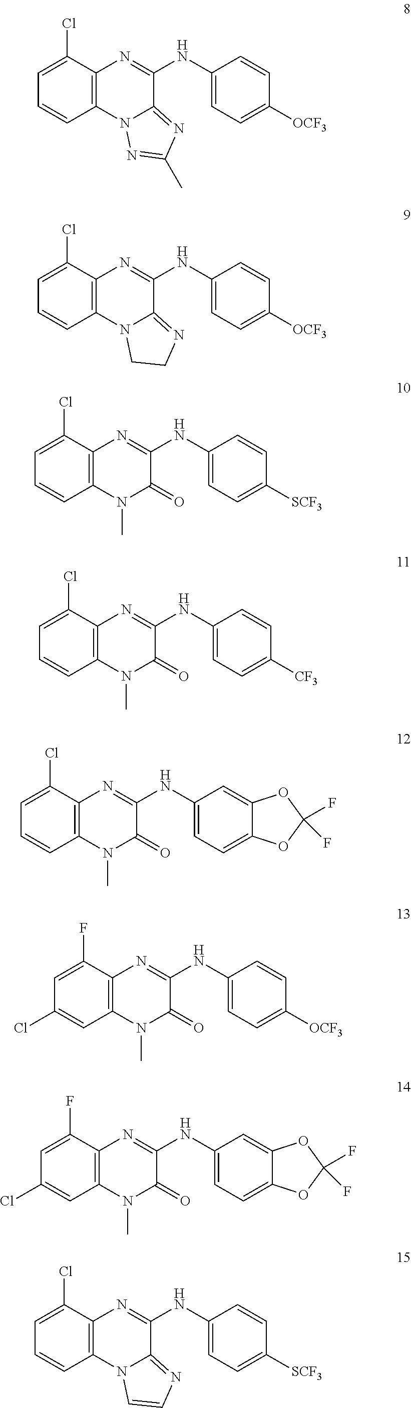

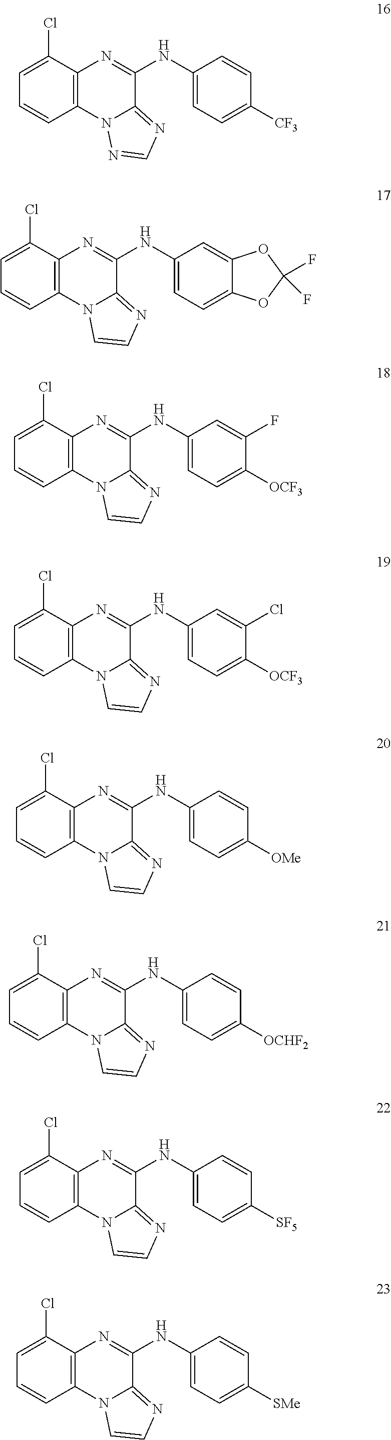

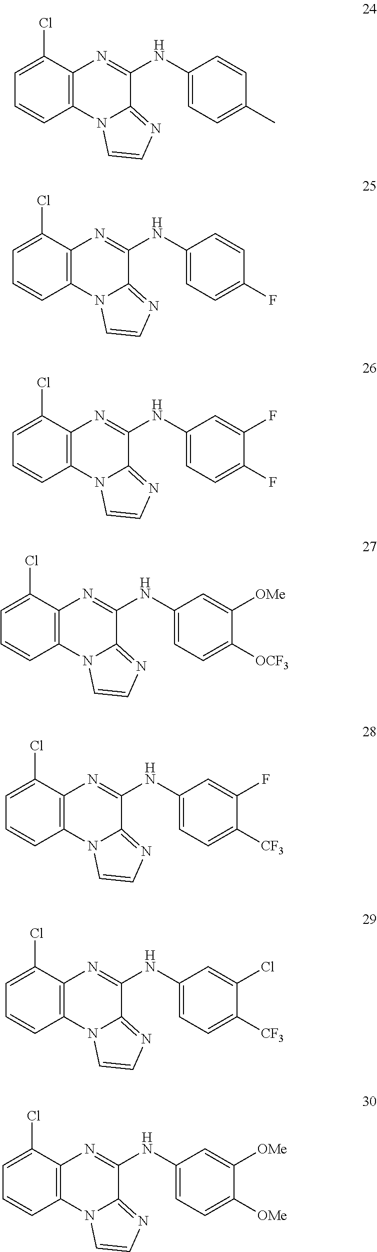

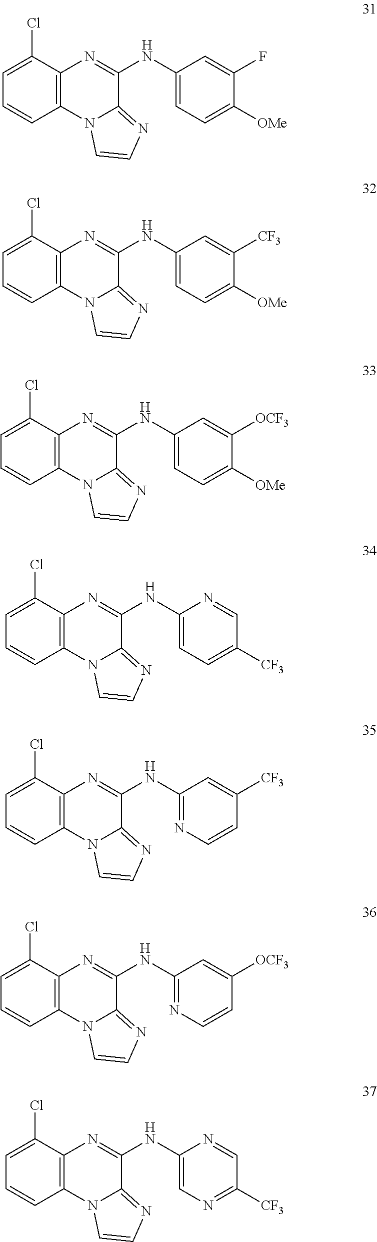

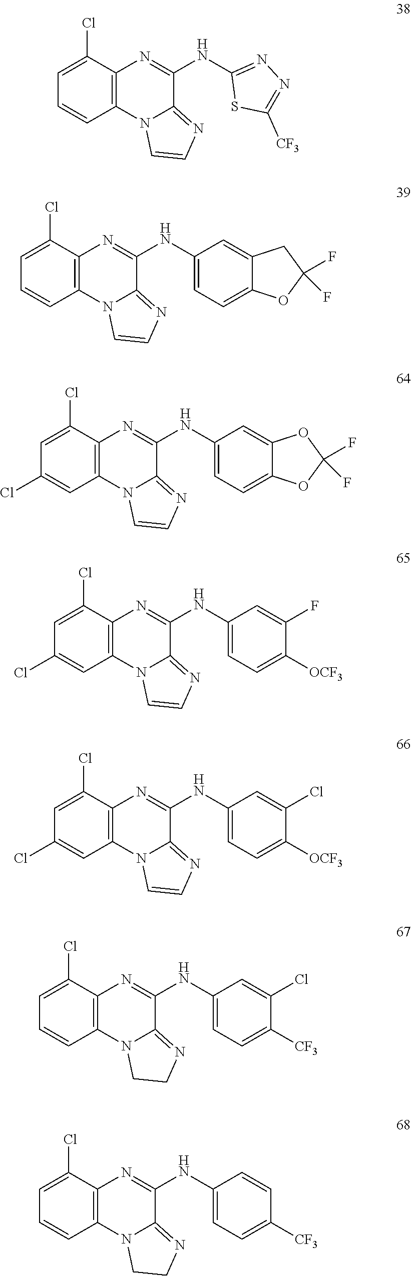

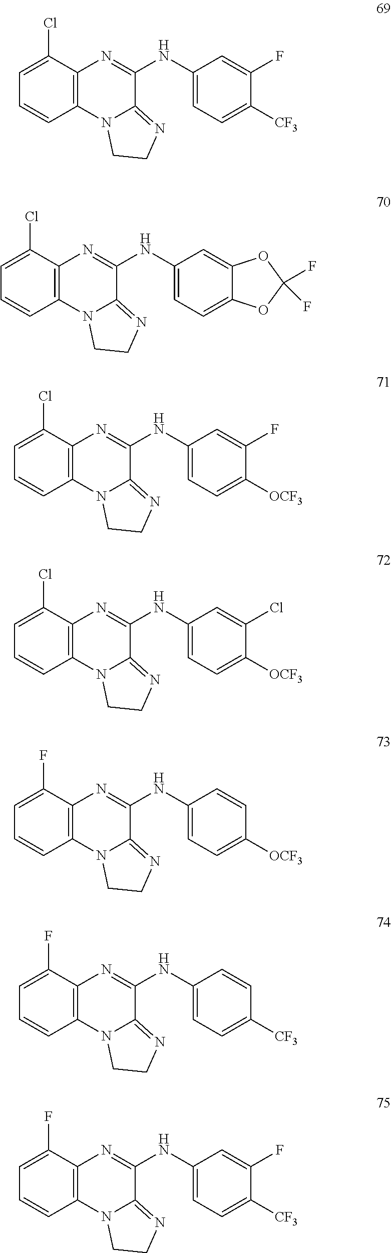

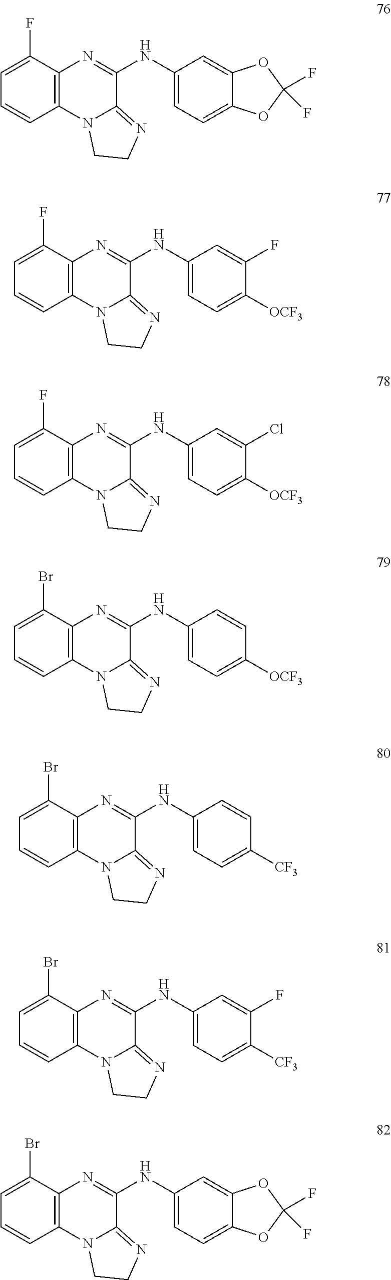

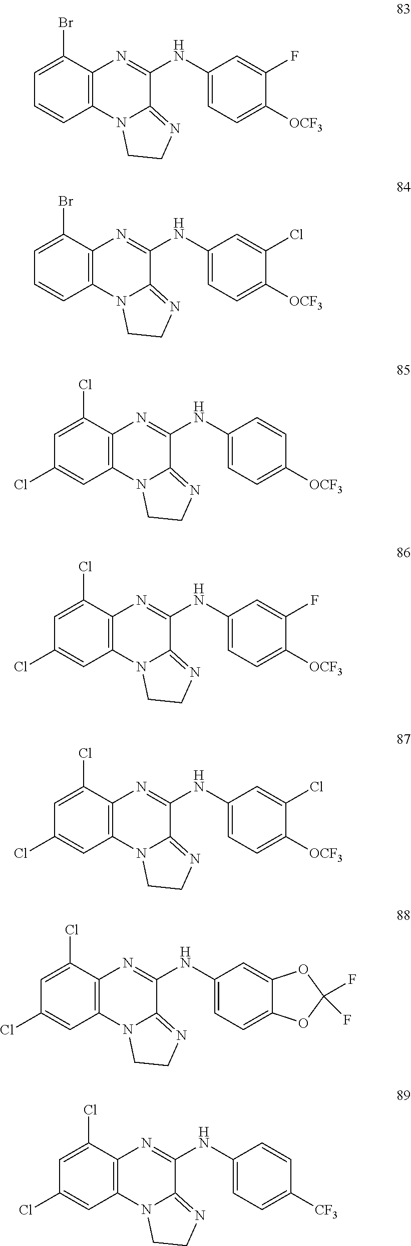

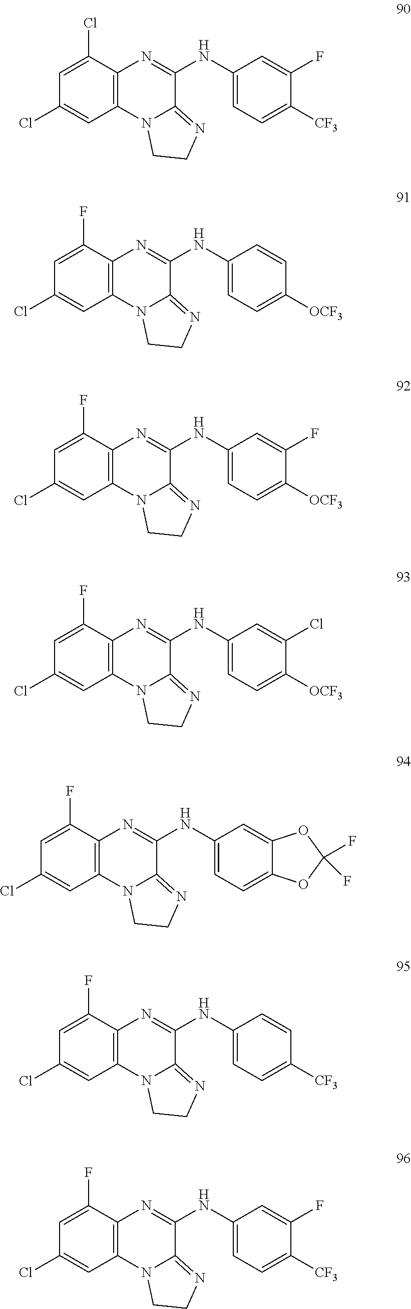

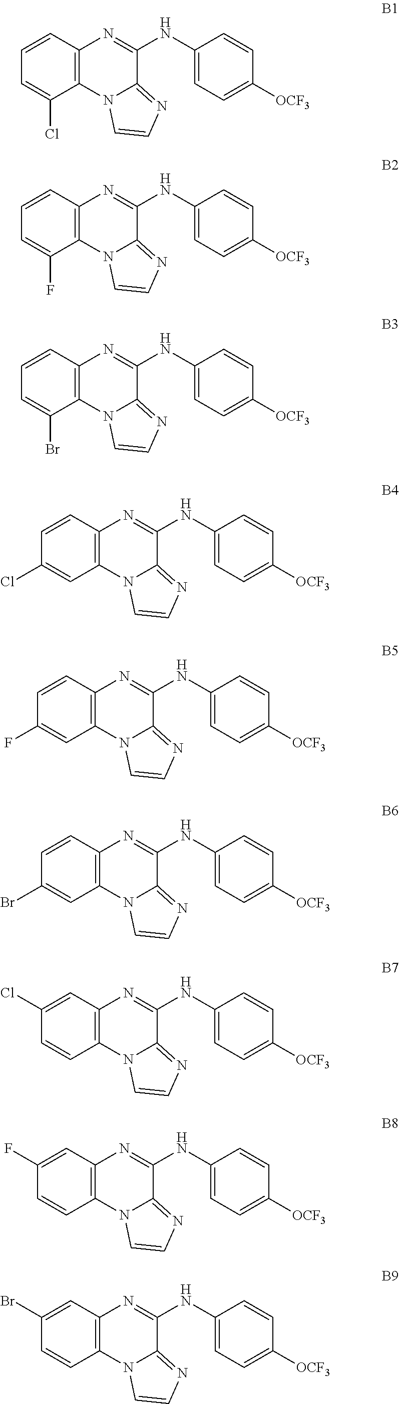

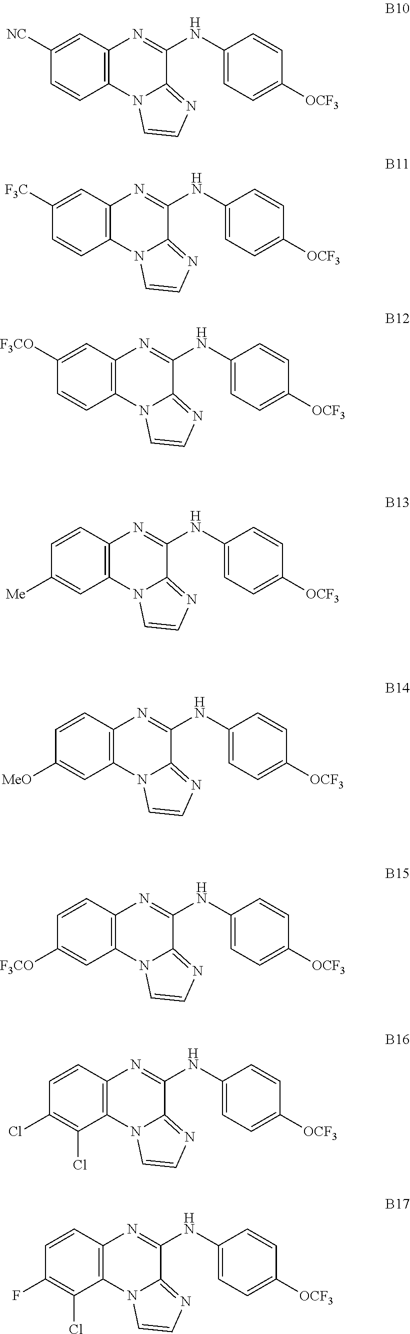

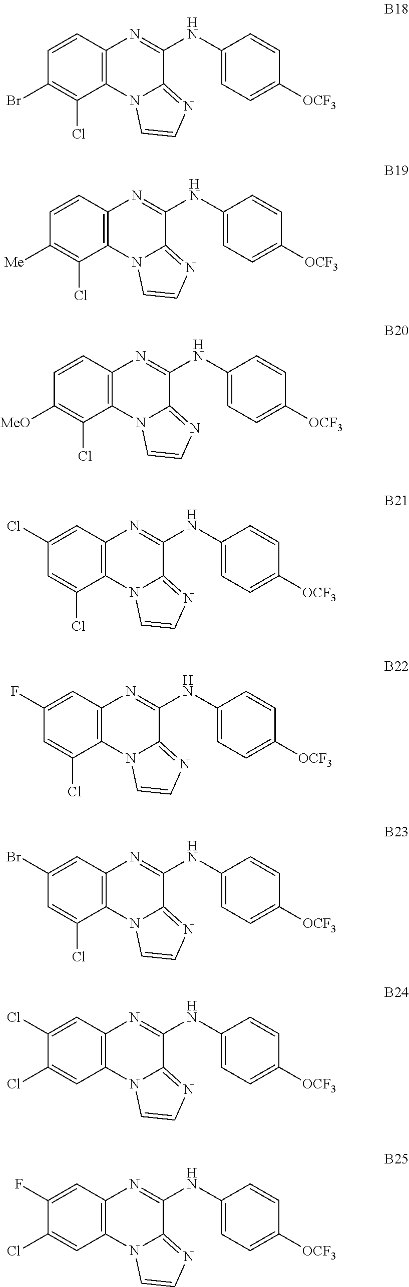

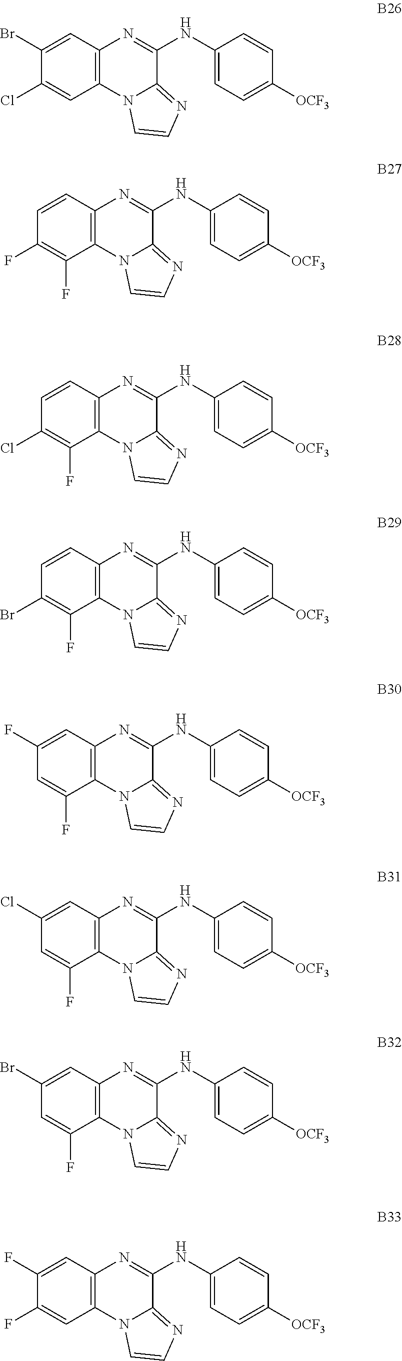

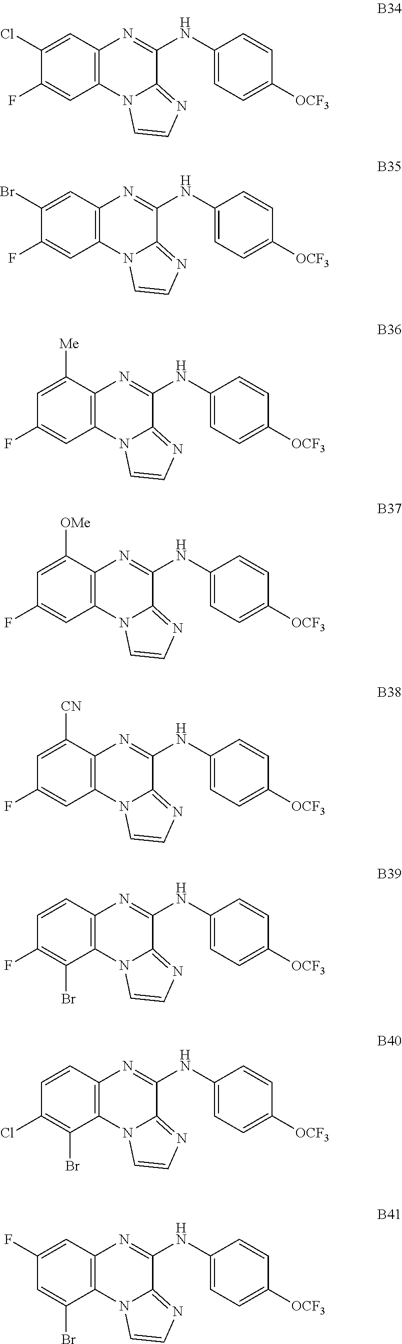

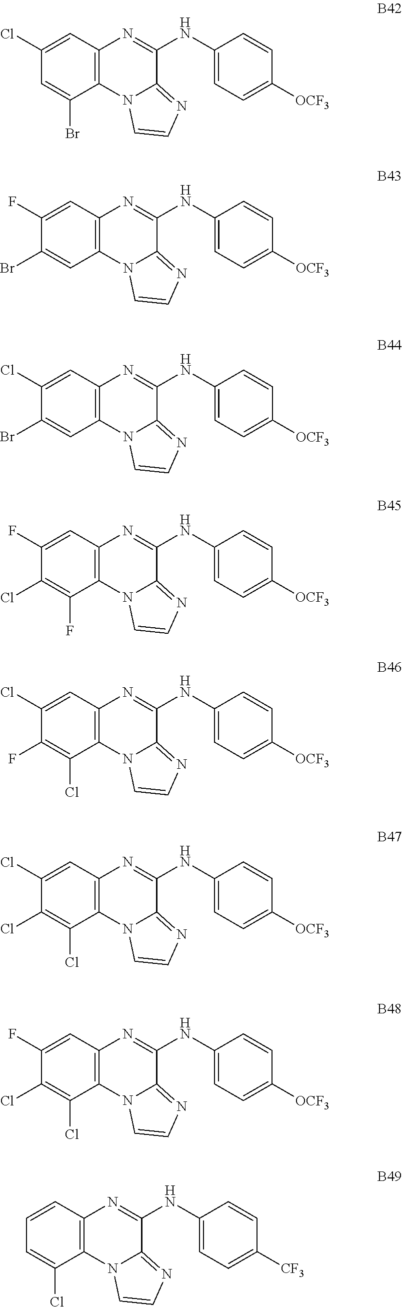

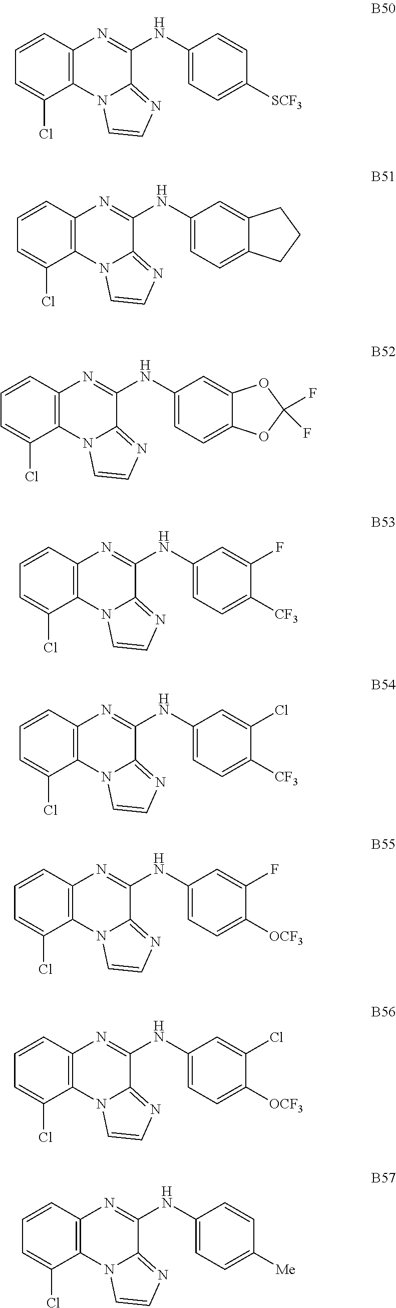

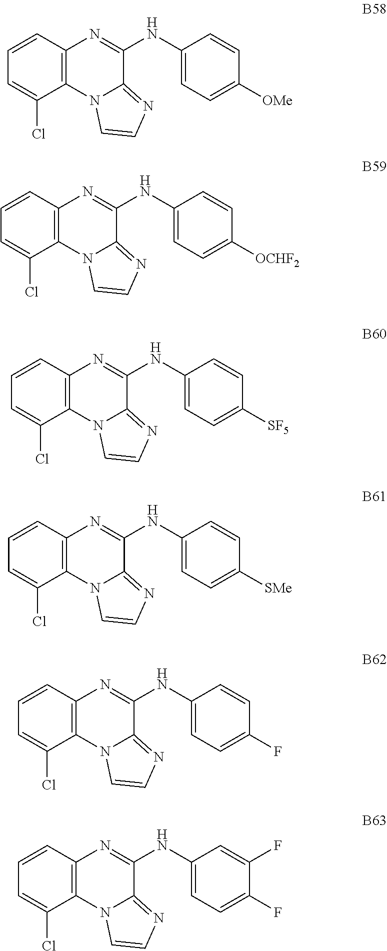

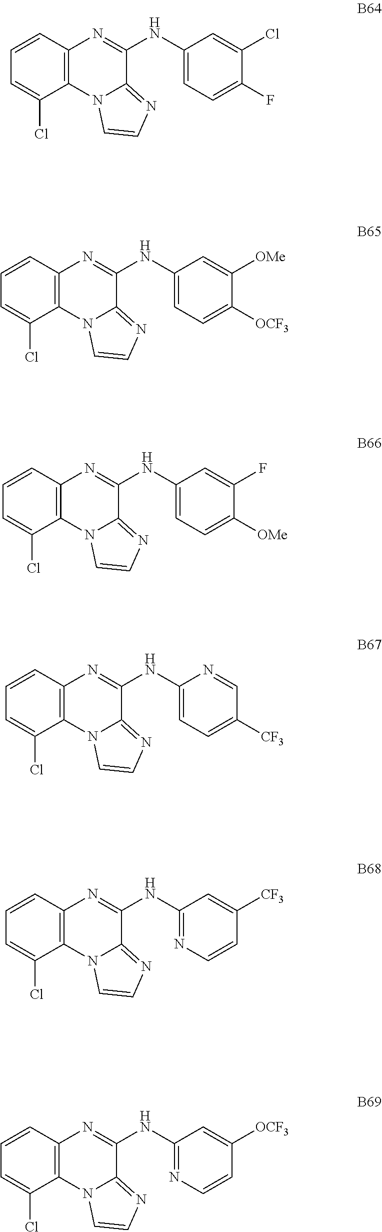 [SMILES: Fc1ccc(Nc2nc3cccc(Cl)c3n3ccnc23)cc1Cl ; COc1cc(Nc2nc3cccc(Cl)c3n3ccnc23)ccc1OC(F)(F)F ; COc1ccc(Nc2nc3cccc(Cl)c3n3ccnc23)cc1F ; FC(F)(F)c1ccc(Nc2nc3cccc(Cl)c3n3ccnc23)nc1 ; FC(F)(F)c1ccnc(Nc2nc3cccc(Cl)c3n3ccnc23)c1 ; FC(F)(F)Oc1ccnc(Nc2nc3cccc(Cl)c3n3ccnc23)c1]

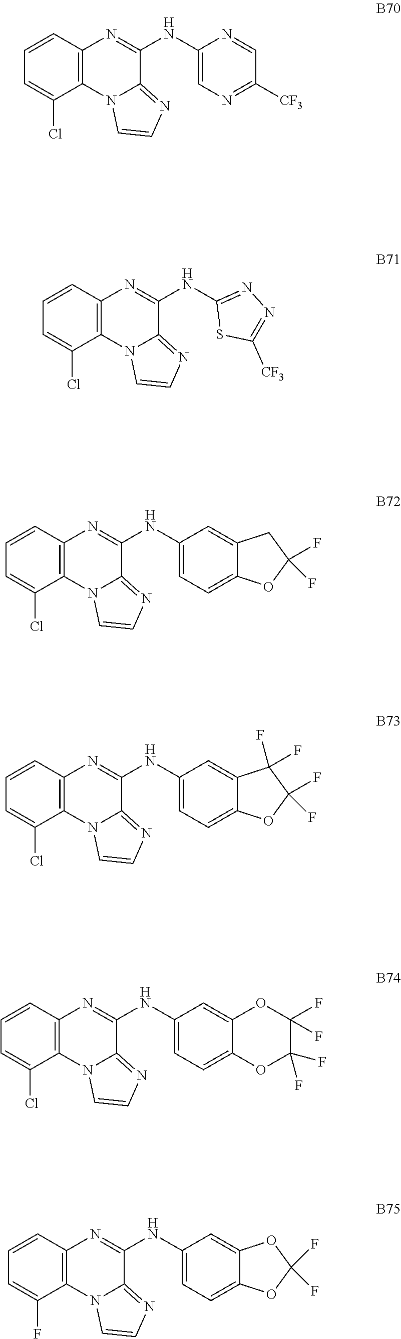 [SMILES: FC(F)(F)c1cnc(Nc2nc3cccc(Cl)c3n3ccnc23)cn1 ; FC(F)(F)c1nnc(Nc2nc3cccc(Cl)c3n3ccnc23)s1 ; FC1(F)Cc2cc(Nc3nc4cccc(Cl)c4n4ccnc34)ccc2O1 ; FC1(F)Oc2ccc(Nc3nc4cccc(Cl)c4n4ccnc34)cc2C1(F)F ; FC1(F)Oc2ccc(Nc3nc4cccc(Cl)c4n4ccnc34)cc2OC1(F)F ; Fc1cccc2nc(Nc3ccc4c(c3)OC(F)(F)O4)c3nccn3c12]

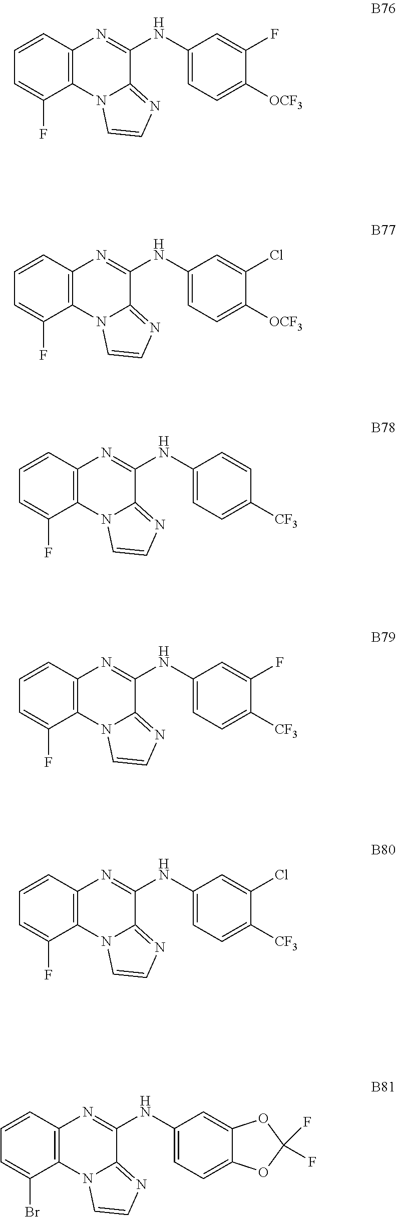 [SMILES: Fc1cc(Nc2nc3cccc(F)c3n3ccnc23)ccc1OC(F)(F)F ; Fc1cccc2nc(Nc3ccc(OC(F)(F)F)c(Cl)c3)c3nccn3c12 ; Fc1cccc2nc(Nc3ccc(C(F)(F)F)cc3)c3nccn3c12 ; Fc1cc(Nc2nc3cccc(F)c3n3ccnc23)ccc1C(F)(F)F ; Fc1cccc2nc(Nc3ccc(C(F)(F)F)c(Cl)c3)c3nccn3c12 ; FC1(F)Oc2ccc(Nc3nc4cccc(Br)c4n4ccnc34)cc2O1]

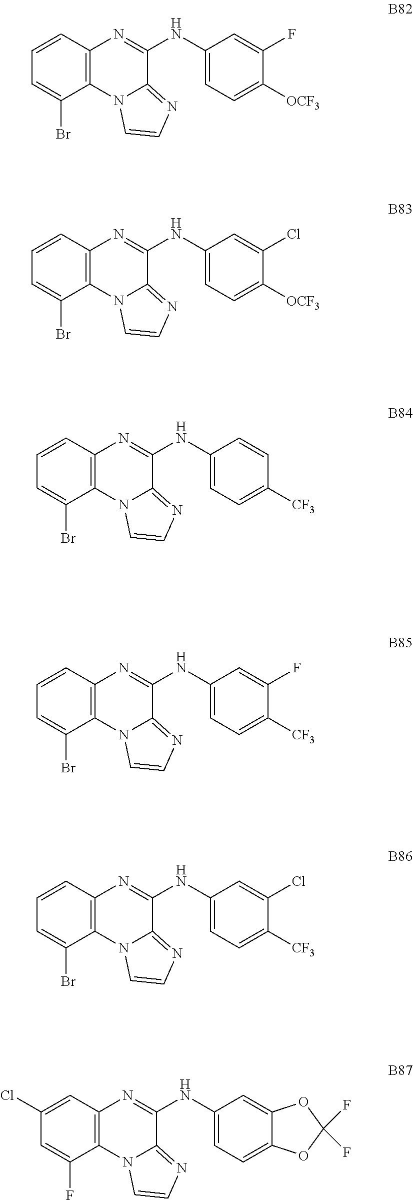 [SMILES: Fc1cc(Nc2nc3cccc(Br)c3n3ccnc23)ccc1OC(F)(F)F ; FC(F)(F)Oc1ccc(Nc2nc3cccc(Br)c3n3ccnc23)cc1Cl ; FC(F)(F)c1ccc(Nc2nc3cccc(Br)c3n3ccnc23)cc1 ; Fc1cc(Nc2nc3cccc(Br)c3n3ccnc23)ccc1C(F)(F)F ; FC(F)(F)c1ccc(Nc2nc3cccc(Br)c3n3ccnc23)cc1Cl ; Fc1cc(Cl)cc2nc(Nc3ccc4c(c3)OC(F)(F)O4)c3nccn3c12]

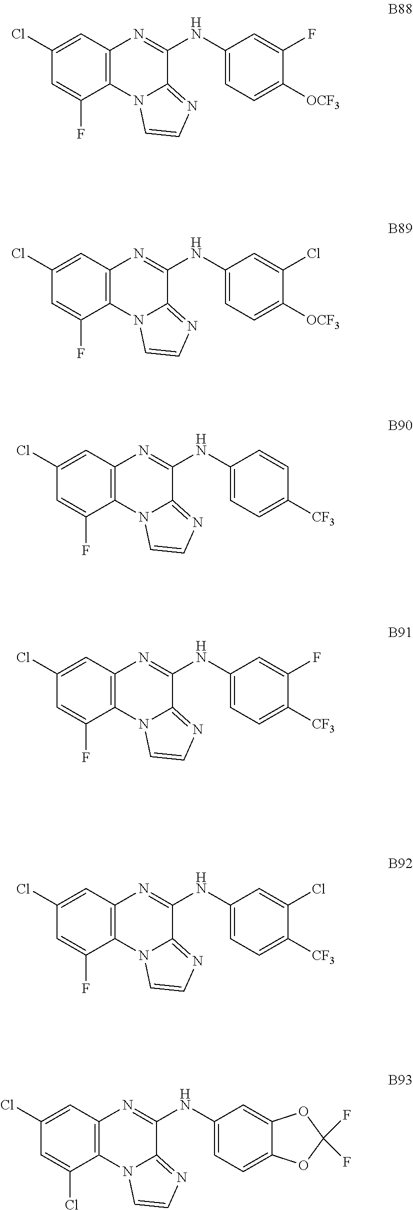 [SMILES: Fc1cc(Nc2nc3cc(Cl)cc(F)c3n3ccnc23)ccc1OC(F)(F)F ; Fc1cc(Cl)cc2nc(Nc3ccc(OC(F)(F)F)c(Cl)c3)c3nccn3c12 ; Fc1cc(Cl)cc2nc(Nc3ccc(C(F)(F)F)cc3)c3nccn3c12 ; Fc1cc(Nc2nc3cc(Cl)cc(F)c3n3ccnc23)ccc1C(F)(F)F ; Fc1cc(Cl)cc2nc(Nc3ccc(C(F)(F)F)c(Cl)c3)c3nccn3c12 ; FC1(F)Oc2ccc(Nc3nc4cc(Cl)cc(Cl)c4n4ccnc34)cc2O1]

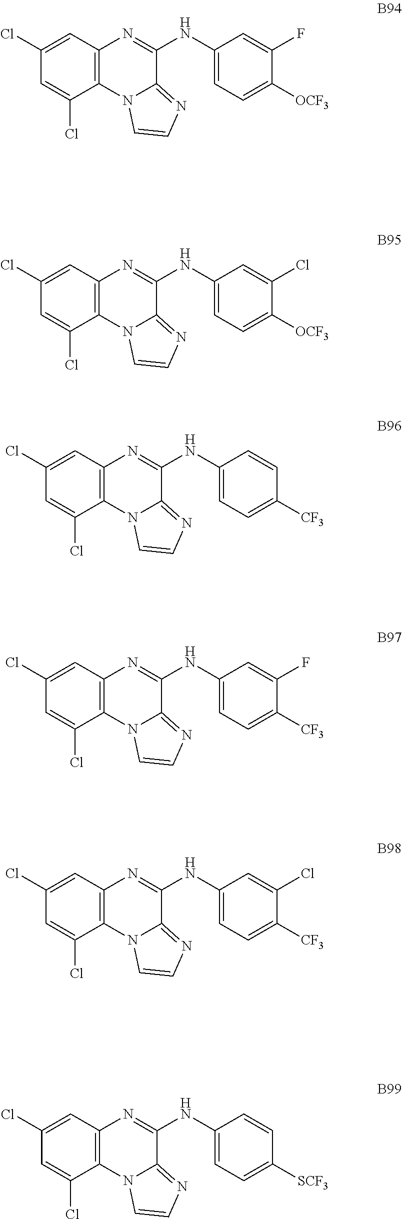

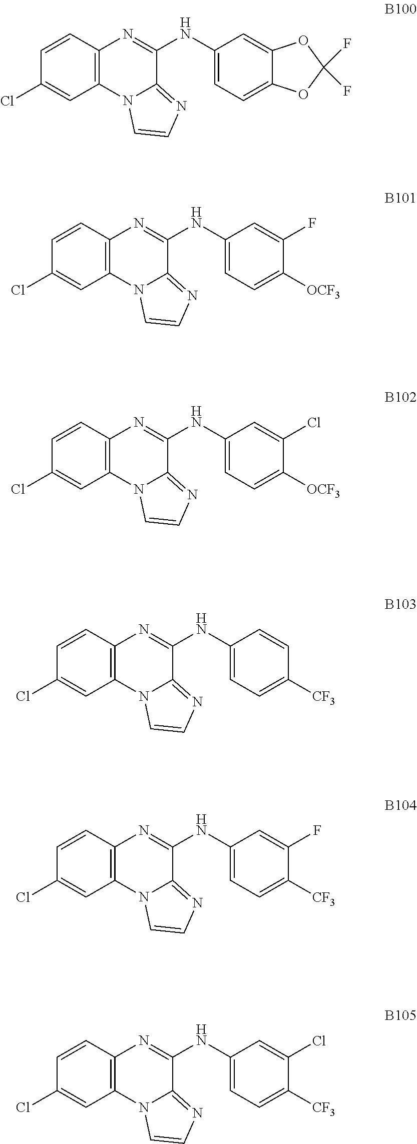 [SMILES: FC1(F)Oc2ccc(Nc3nc4ccc(Cl)cc4n4ccnc34)cc2O1 ; Fc1cc(Nc2nc3ccc(Cl)cc3n3ccnc23)ccc1OC(F)(F)F ; FC(F)(F)Oc1ccc(Nc2nc3ccc(Cl)cc3n3ccnc23)cc1Cl ; FC(F)(F)c1ccc(Nc2nc3ccc(Cl)cc3n3ccnc23)cc1 ; Fc1cc(Nc2nc3ccc(Cl)cc3n3ccnc23)ccc1C(F)(F)F ; FC(F)(F)c1ccc(Nc2nc3ccc(Cl)cc3n3ccnc23)cc1Cl]

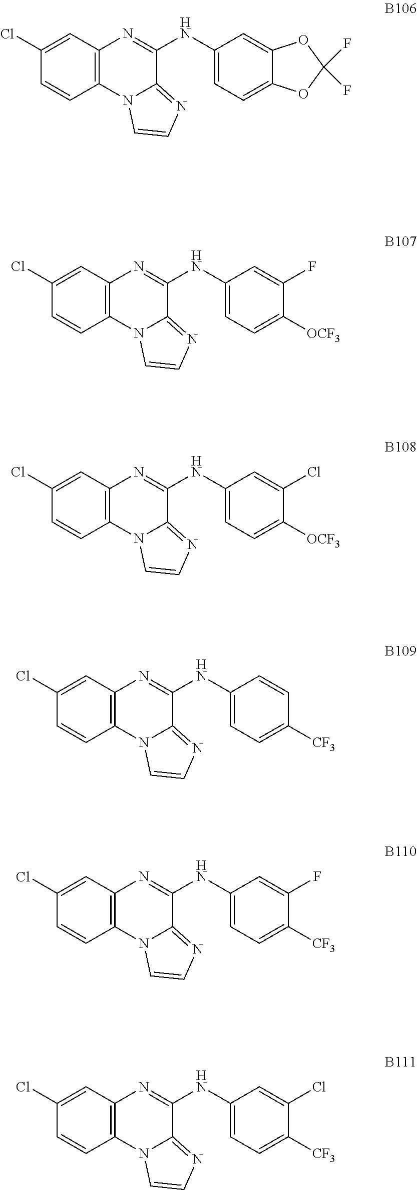 [SMILES: FC1(F)Oc2ccc(Nc3nc4cc(Cl)ccc4n4ccnc34)cc2O1 ; Fc1cc(Nc2nc3cc(Cl)ccc3n3ccnc23)ccc1OC(F)(F)F ; FC(F)(F)Oc1ccc(Nc2nc3cc(Cl)ccc3n3ccnc23)cc1Cl ; FC(F)(F)c1ccc(Nc2nc3cc(Cl)ccc3n3ccnc23)cc1 ; Fc1cc(Nc2nc3cc(Cl)ccc3n3ccnc23)ccc1C(F)(F)F ; FC(F)(F)c1ccc(Nc2nc3cc(Cl)ccc3n3ccnc23)cc1Cl]

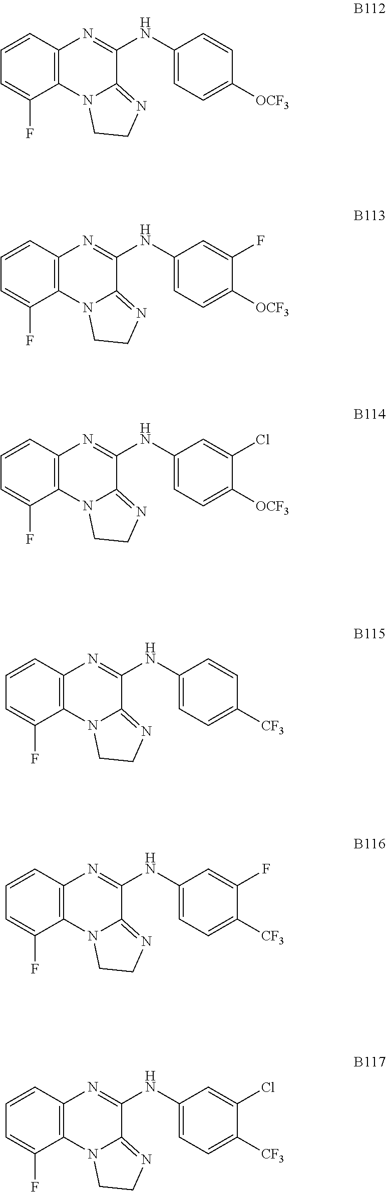 [SMILES: Fc1cccc2c1N1CCN=C1C(Nc1ccc(OC(F)(F)F)cc1)=N2 ; Fc1cc(NC2=Nc3cccc(F)c3N3CCN=C23)ccc1OC(F)(F)F ; Fc1cccc2c1N1CCN=C1C(Nc1ccc(OC(F)(F)F)c(Cl)c1)=N2 ; Fc1cccc2c1N1CCN=C1C(Nc1ccc(C(F)(F)F)cc1)=N2 ; Fc1cc(NC2=Nc3cccc(F)c3N3CCN=C23)ccc1C(F)(F)F ; Fc1cccc2c1N1CCN=C1C(Nc1ccc(C(F)(F)F)c(Cl)c1)=N2]

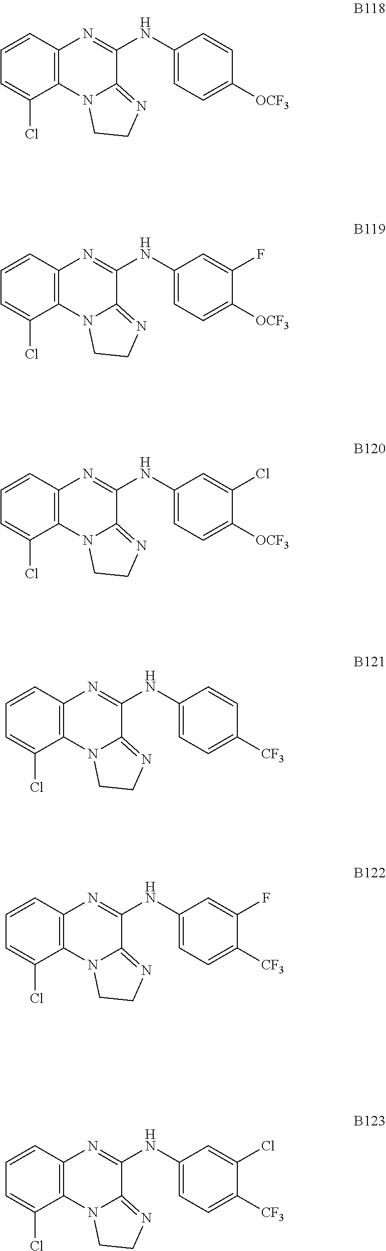 [SMILES: FC(F)(F)Oc1ccc(NC2=Nc3cccc(Cl)c3N3CCN=C23)cc1 ; Fc1cc(NC2=Nc3cccc(Cl)c3N3CCN=C23)ccc1OC(F)(F)F ; FC(F)(F)Oc1ccc(NC2=Nc3cccc(Cl)c3N3CCN=C23)cc1Cl ; FC(F)(F)c1ccc(NC2=Nc3cccc(Cl)c3N3CCN=C23)cc1 ; Fc1cc(NC2=Nc3cccc(Cl)c3N3CCN=C23)ccc1C(F)(F)F ; FC(F)(F)c1ccc(NC2=Nc3cccc(Cl)c3N3CCN=C23)cc1Cl]

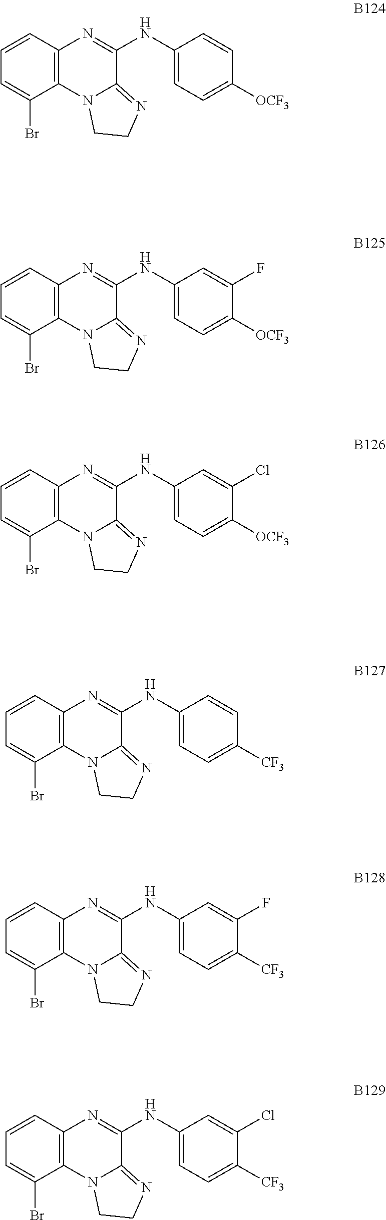 [SMILES: FC(F)(F)Oc1ccc(NC2=Nc3cccc(Br)c3N3CCN=C23)cc1 ; Fc1cc(NC2=Nc3cccc(Br)c3N3CCN=C23)ccc1OC(F)(F)F ; FC(F)(F)Oc1ccc(NC2=Nc3cccc(Br)c3N3CCN=C23)cc1Cl ; FC(F)(F)c1ccc(NC2=Nc3cccc(Br)c3N3CCN=C23)cc1 ; Fc1cc(NC2=Nc3cccc(Br)c3N3CCN=C23)ccc1C(F)(F)F ; FC(F)(F)c1ccc(NC2=Nc3cccc(Br)c3N3CCN=C23)cc1Cl]

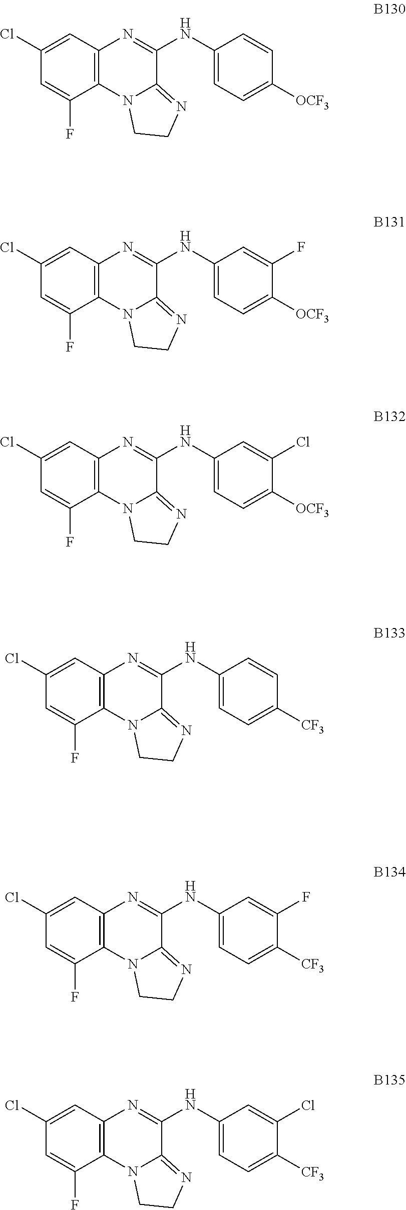 [SMILES: Fc1cc(Cl)cc2c1N1CCN=C1C(Nc1ccc(OC(F)(F)F)cc1)=N2 ; Fc1cc(NC2=Nc3cc(Cl)cc(F)c3N3CCN=C23)ccc1OC(F)(F)F ; Fc1cc(Cl)cc2c1N1CCN=C1C(Nc1ccc(OC(F)(F)F)c(Cl)c1)=N2 ; Fc1cc(Cl)cc2c1N1CCN=C1C(Nc1ccc(C(F)(F)F)cc1)=N2 ; Fc1cc(NC2=Nc3cc(Cl)cc(F)c3N3CCN=C23)ccc1C(F)(F)F ; Fc1cc(Cl)cc2c1N1CCN=C1C(Nc1ccc(C(F)(F)F)c(Cl)c1)=N2]

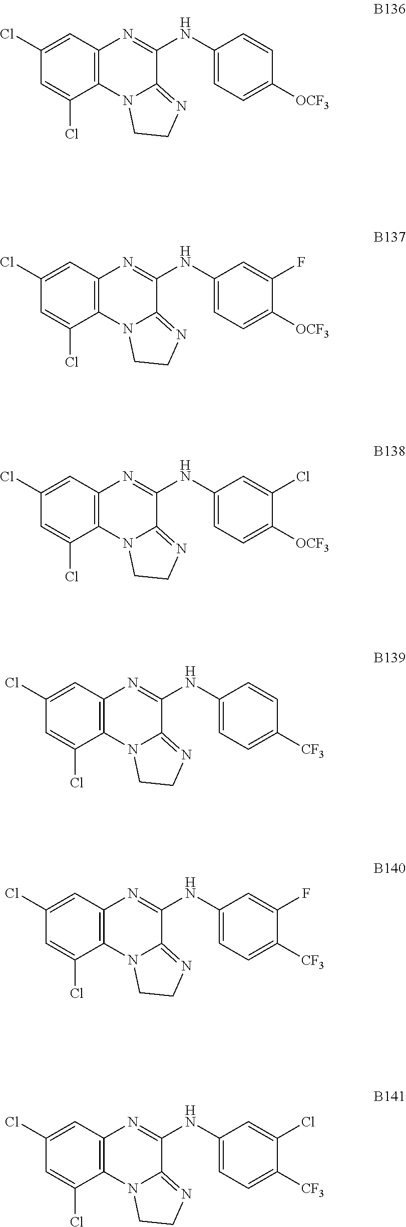 [SMILES: FC(F)(F)Oc1ccc(NC2=Nc3cc(Cl)cc(Cl)c3N3CCN=C23)cc1 ; Fc1cc(NC2=Nc3cc(Cl)cc(Cl)c3N3CCN=C23)ccc1OC(F)(F)F ; FC(F)(F)Oc1ccc(NC2=Nc3cc(Cl)cc(Cl)c3N3CCN=C23)cc1Cl ; FC(F)(F)c1ccc(NC2=Nc3cc(Cl)cc(Cl)c3N3CCN=C23)cc1 ; Fc1cc(NC2=Nc3cc(Cl)cc(Cl)c3N3CCN=C23)ccc1C(F)(F)F ; FC(F)(F)c1ccc(NC2=Nc3cc(Cl)cc(Cl)c3N3CCN=C23)cc1Cl]

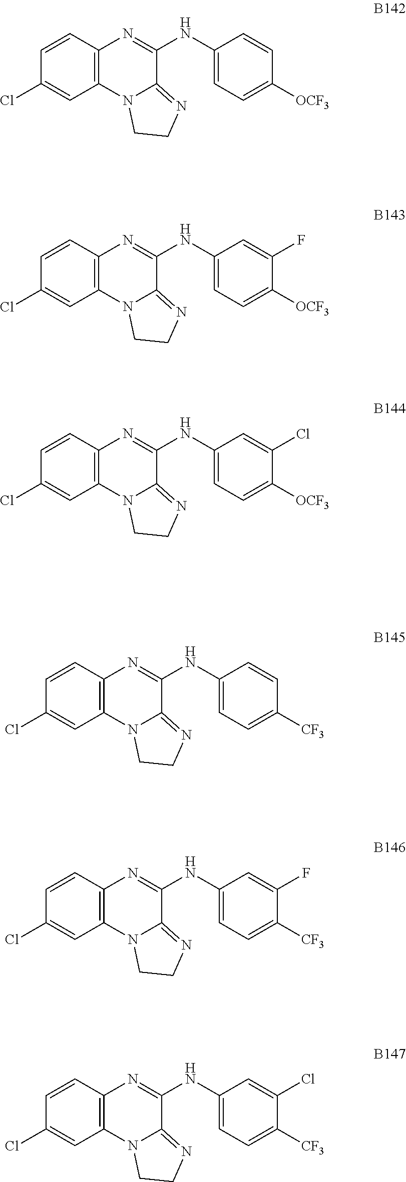 [SMILES: FC(F)(F)Oc1ccc(NC2=Nc3ccc(Cl)cc3N3CCN=C23)cc1 ; Fc1cc(NC2=Nc3ccc(Cl)cc3N3CCN=C23)ccc1OC(F)(F)F ; FC(F)(F)Oc1ccc(NC2=Nc3ccc(Cl)cc3N3CCN=C23)cc1Cl ; FC(F)(F)c1ccc(NC2=Nc3ccc(Cl)cc3N3CCN=C23)cc1 ; Fc1cc(NC2=Nc3ccc(Cl)cc3N3CCN=C23)ccc1C(F)(F)F ; FC(F)(F)c1ccc(NC2=Nc3ccc(Cl)cc3N3CCN=C23)cc1Cl]

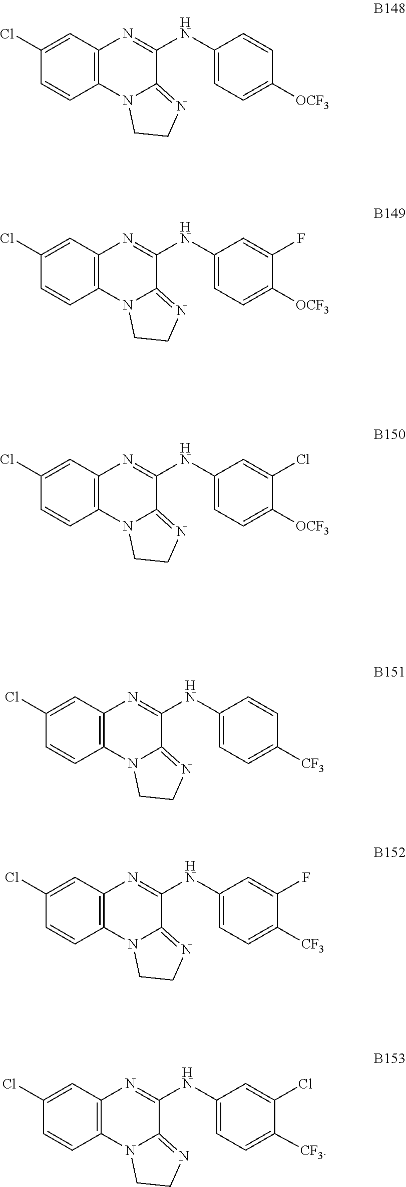 [SMILES: FC(F)(F)Oc1ccc(NC2=Nc3cc(Cl)ccc3N3CCN=C23)cc1 ; Fc1cc(NC2=Nc3cc(Cl)ccc3N3CCN=C23)ccc1OC(F)(F)F ; FC(F)(F)Oc1ccc(NC2=Nc3cc(Cl)ccc3N3CCN=C23)cc1Cl ; FC(F)(F)c1ccc(NC2=Nc3cc(Cl)ccc3N3CCN=C23)cc1 ; Fc1cc(NC2=Nc3cc(Cl)ccc3N3CCN=C23)ccc1C(F)(F)F ; FC(F)(F)c1ccc(NC2=Nc3cc(Cl)ccc3N3CCN=C23)cc1Cl]

14. A pharmaceutical composition for treating, regulating, and/or preventing a disease associated with miR-124, characterized in that the pharmaceutical composition comprises:
(1) the compound as an active ingredient according to claim 1, or an isomer, crystal form, pharmaceutically acceptable salt, hydrate, or solvate thereof; and
optionally (2) a pharmaceutically acceptable excipient or carrier.

15-16. (canceled)

17. A method for preparing a compound of Formula (1), characterized in that the method includes the steps of:
(1) in an inert solvent, the compound of formula (A) and its tautomer react to obtain the compound of formula (B): 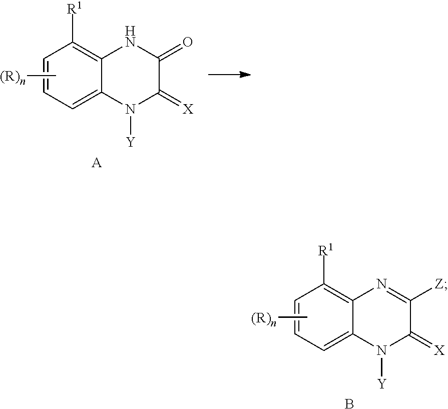
(2) in an inert solvent, the compound of formula (B) reacts with the compound of formula (d) to obtain the compound of formula (1): 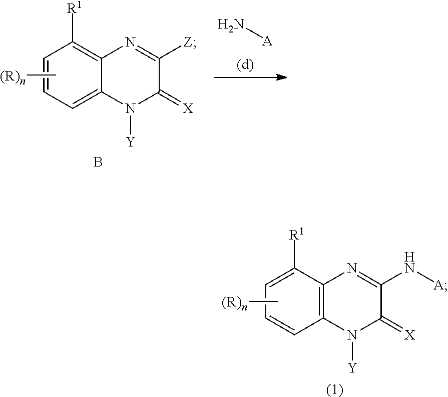
wherein Z is a halogen or OTf, X, Y, R, R¹, A and n are as defined in claim 1.

18. The method according to claim 17, characterized in that the compound of formula (1) is a compound of formula (1a),
and the method includes the following steps:
(s1) in an inert solvent, the compound of formula (A1) and its tautomer react to obtain the compound of formula (B1): 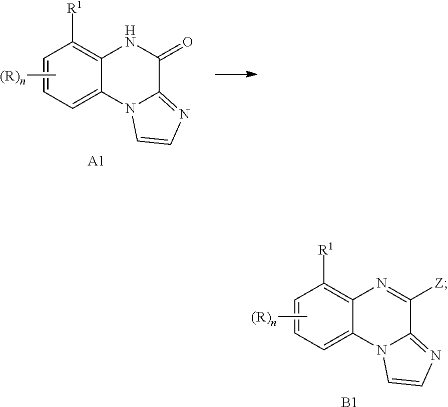
(s2) in an inert solvent, the compound of formula (B1) reacts with the compound of formula (d) to obtain the compound of formula (1a):

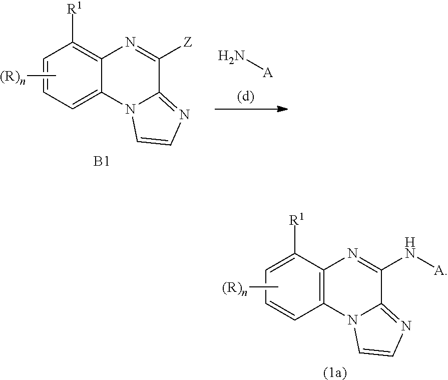

19. The method according to claim 17, characterized in that the compound of formula (1) is a compound of formula (1b),
and the method includes the following steps:
(t1) in an inert solvent, the compound of formula (C) undergoes a cyclization reaction to yield the compound of formula (D): 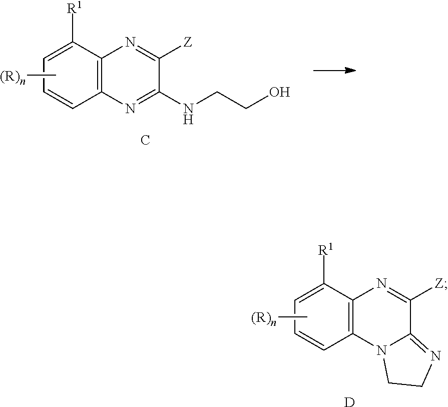
(t2) in an inert solvent, the compound of formula (D) reacts with the compound of formula (d) to obtain the compound of formula (1b):

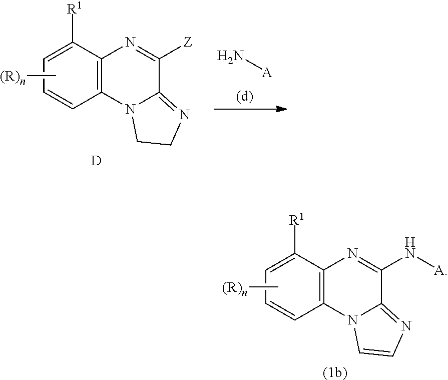

20. A method for treating, regulating, and/or preventing a disease mediated by miR-124, characterized by comprising administering to an individual in need thereof an amount of the compound according to claim 1 or an isomer, crystalline form, pharmaceutically acceptable salt, hydrate, or solvate thereof.

21. The method according to claim 20, wherein the disease mediated by miR-124 is selected from the group consisting of: Alzheimer's disease, Parkinson's disease, brain tumor, gastric cancer, liver cancer, lung cancer, colorectal cancer, pancreatic cancer, breast cancer, cervical cancer, endometrial cancer, prostate cancer, Crohn's disease, ulcerative colitis, rheumatoid arthritis, allergic rhinitis, osteoarthritis, fibrosis disease, AIDS and/or COVID-19.
